SEQUENCE LISTING (4 sequences)

SEQ ID NO: 1 - DNA, 2052 bp, synthetic construct
  source 1..2052: mol_type = other DNA
agcgtgctgacacagcctagctccgtgtctgccgcccctggccagaaagtgaccatcagctgtagcggcagcaccagcaacatcggcaacaactacgtgtcctggtatcagcagcaccccggcaaggcccccaagctgatgatctacgacgtgtccaagcggcccagcggcgtgcccgatagattttccggcagcaagagcggcaacagcgccagcctggatatcagcggcctgcagtctgaggacgaggccgactactattgcgccgcctgggacgatagcctgagcgagttcctgtttggcaccggcaccaagctgacagtgctgggcggaggcggaggatctggcggcggaggaagtggcggagggggatctcaggtgcagctggtggaaagcggcggcaacctggtgcagcctggcggatctctgagactgagctgtgccgccagcggcttcaccttcggcagcttcagcatgagctgggtgcgccaggctcctgggggaggactggaatgggtggcaggactgagcgccagaagcagcctgacccactacgccgatagcgtgaagggccggttcaccatcagccgggacaacgccaagaacagcgtgtacctgcagatgaacagcctgcgggtggaagataccgccgtgtactactgcgccagacggtcctacgacagcagcggctactggggccacttctacagctacatggacgtgtggggccagggcaccctcgtgacagtgtctgagagcaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggc

SEQ ID NO: 2 - protein, 684 aa, synthetic construct
SVLTQPSSVSAAPGQKVTISCSGSTSNIGNNYVSWYQQHPGKAPKLMIYDVSKRPSGVPDRFSGSKSGNSASLDISGLQSEDEADYYCAAWDDSLSEFLFGTGTKLTVLGGGGGSGGGGSGGGGSQVQLVESGGNLVQPGGSLRLSCAASGFTFGSFSMSWVRQAPGGGLEWVAGLSARSSLTHYADSVKGRFTISRDNAKNSVYLQMNSLRVEDTAVYYCARRSYDSSGYWGHFYSYMDVWGQGTLVTVSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPG

SEQ ID NO: 3 - protein, 687 aa, synthetic construct
DVVMTQTPLSLPVSLGDQASISCRSSQSLVHSNGNTYLRWYLQKPGQSPKVLIYKVSNRVSGVPDRFSGSGSGTDFTLKINRVEAEDLGVYFCSQSTHVPWTFGGGTKLEIKSSADDAKKDAAKKDDAKKDDAKKDGGVKLDETGGGLVQPGGAMKLSCVTSGFTFGHYWMNWVRQSPEKGLEWVAQFRNKPYNYETYYSDSVKGRFTISRDDSKSSVYLQMNNLRVEDTGIYYCTGASYGMEYLGQGTSVTVSESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPG

SEQ ID NO: 4 - DNA, 2061 bp, synthetic construct
  source 1..2061: mol_type = other DNA
gacgttgtaatgacccagacccctctgtctctccccgtaagcttgggcgaccaggcgagcatctcttgtcggtcttcccagtccctggtccattcaaacggcaatacttacttgcggtggtacttgcagaagcccggtcaatccccaaaagtgctgatatacaaggttagcaatcgggtcagtggagtgcccgaccgcttcagcggaagcggatccgggactgacttcactctgaagatcaaccgggtagaagctgaagacctgggggtgtacttctgctctcagtcaacacacgtgccatggacctttggaggtggcaccaagctggaaatcaaatcatcagcggacgatgccaaaaaagacgcggccaagaaggacgatgccaagaaggatgatgctaaaaaggatggcggagtcaaattggacgagacaggcgggggactggtgcagcccggcggtgccatgaaactgtcttgtgtgaccagcggctttaccttcgggcattattggatgaactgggtgcgacagtctccagagaaagggctcgagtgggtggcccagtttcgaaataaaccgtacaattatgagacctactattcagattctgtgaaagggcgcttcactatttcacgcgacgacagcaaaagttccgtctaccttcagatgaacaaccttagagtggaggataccggaatatactactgcacgggtgccagttatggcatggagtacttggggcaggggacatctgtgaccgtttctgagagcaagtacggaccgccctgccccccttgccctgcccccgagttcgacggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaaggctcgagggcggcggagagggcagaggaagtcttctaacatgcggtgacgtggaggagaatcccggc

(19) United States
(12) Patent Application Publication
(10) Pub. No.: US 2025/0368718 A1
(43) Pub. Date: Dec. 4, 2025

(54) METHODS OF USE FOR CAR T CELLS

(71) Applicants: Endocyte, Inc., West Lafayette, IN (US); Purdue Research Foundation, West Lafayette, IN (US); Seattle Children's Hospital (DBA Seattle Children's Research Institute), Seattle, WA (US)

(72) Inventors: Philip Stewart Low, West Lafayette, IN (US); Haiyan Chu, West Lafayette, IN (US); Yingjuan June Lu, West Lafayette, IN (US); Christopher Paul Leamon, West Lafayette, IN (US); Leroy W. Wheeler, II, West Lafayette, IN (US); Michael C. Jensen, Bainbridge Island, WA (US); James Matthaei, Seattle, WA (US)

(21) Appl. No.: 19/067,642

(22) Filed: Feb. 28, 2025

Related U.S. Application Data
(62) Division of application No. 18/459,302, filed on Aug. 31, 2023, now Pat. No. 12,269,862.
Application No. 18/459,302 is a division of application No. 16/253,562, filed on Jan. 22, 2019, now Pat. No. 11,779,602.
(60) Provisional application No. 62/620,414, filed on Jan. 22, 2018.
(60) Provisional application No. 62/620,706, filed on Jan. 23, 2018.
(60) Provisional application No. 62/656,233, filed on Apr. 11, 2018.
(60) Provisional application No. 62/724,171, filed on Aug. 29, 2018.
(60) Provisional application No. 62/735,627, filed on Sep. 24, 2018.
(60) Provisional application No. 62/736,727, filed on Sep. 26, 2018.

(51) Int. Cl. C07K 14/725; A61K 9/00; A61K 31/365; A61K 31/519; A61K 38/17; A61K 40/11; A61P 35/00; C07K 16/44; C07K 16/46; C12N 5/0783

(52) U.S. Cl. CPC C07K 14/7051; A61K 9/0019; A61K 9/0053; A61K 31/365; A61K 31/519; A61K 38/1774; A61K 40/11; A61P 35/00; C07K 16/44; C07K 16/46; C12N 5/0636; C12N 5/0638; A61K 2239/38; C07K 2317/622

(57) ABSTRACT

The present disclosure relates to methods of treating a patient with a cancer by administering to the patient a composition comprising CAR T cells wherein the CAR T cells comprise a CAR and the CAR comprises an E2 anti-fluorescein antibody fragment, and administering to the patient a small molecule linked to a targeting moiety by a linker. The disclosure also relates to compositions for use in such methods.

### CROSS-REFERENCE TO RELATED APPLICATIONS

This application is a divisional application of U.S. application Ser. No. 18/459,302, filed Aug. 31, 2023, which is a divisional application of U.S. application Ser. No. 16/253,562, filed Jan. 22, 2019, which claims priority under 35 U.S.C. 119(e) to U.S. Provisional Application No. 62/620,414, filed Jan. 22, 2018, U.S. Provisional Application No. 62/620,706, filed Jan. 23, 2018, U.S. Provisional Application No. 62/656,233, filed Apr. 11, 2018, U.S. Provisional Application No. 62/724,171, filed Aug. 29, 2018, U.S. Provisional Application No. 62/735,627, filed Sep. 24, 2018, and U.S. Provisional Application No. 62/736,727, filed Sep. 26, 2018, all of which are incorporated herein by reference in their entirety.

### SEQUENCE LISTING

This application contains a Sequence Listing which has been submitted electronically in ST26 format and hereby incorporated by reference in its entirety. Said ST26 file, created on Aug. 21, 2025, is named 1165166US3.xml and is 10,043 bytes in size.

### TECHNICAL FIELD

The present disclosure relates to methods of treating a patient with a cancer by administering to the patient a composition comprising CAR T cells wherein the CAR T cells comprise a CAR and the CAR comprises an E2 anti-fluorescein antibody fragment, and administering to the patient a small molecule linked to a targeting moiety by a linker. The disclosure also relates to compositions for use in such methods.

### BACKGROUND

Immunotherapy based on adoptive transfer of lymphocytes (e.g., T cells) into a patient is a valuable therapy in the treatment of cancer and other disease. Many important advancements have been made in the development of immunotherapies based on adoptive transfer of lymphocytes. Among the many different types of immunotherapeutic agents, one of the most promising of the immunotherapeutic agents being developed is T cells expressing chimeric antigen receptors (CAR T cells). The chimeric antigen receptor (CAR) is a genetically engineered receptor that is designed to target a specific antigen, for example, a tumor antigen. This targeting can result in cytotoxicity against the tumor, for example, such that CAR T cells expressing CARs can target and kill tumors via the specific tumor antigens.

First generation CARs are composed of a recognition region, e.g., a single chain fragment variable (scFv) region derived from an antibody for recognition and binding to the antigen expressed by the tumor, and an activation signaling domain, e.g., the CD3ζ chain of T cells can serve as a T cell activation signal in CARs. Although CAR T cells have shown positive results in vitro, they have had limited success in eliminating disease (e.g., cancer) in clinical trials. One problem has been the inability to prolong activation and expand the CAR T cell population in vivo.

To address this problem, a co-stimulation domain (e.g., CD137, CD28 or CD134) has been included in second generation CARs to achieve prolonged activation of T cells in vivo. Addition of a co-stimulation domain enhances the in vivo proliferation and survival of T cells containing CARs, and initial clinical data have shown that such constructs are promising therapeutic agents in the treatment of diseases, such as cancer.

Although improvements have been made in CAR T cell therapies, several problems remain. First, ‘off-target’ toxicity may occur due to normal cells that express the antigen targeted by the CAR T cells (e.g., a tumor-associated antigen). Second, unregulated CAR T cell activation may be found where the rapid and uncontrolled elimination of diseased cells (e.g., cancer cells) by CAR T cells induces a constellation of metabolic disturbances, called tumor lysis syndrome, or cytokine release syndrome (CRS), which can be fatal to patients. Tumor lysis syndrome and CRS can result due to administered CAR T cells that cannot be easily regulated, and are activated uncontrollably. Accordingly, although CAR T cells show great promise as a tool in the treatment of diseases, such as cancer, additional CAR T cell therapies are needed that provide reduced off-target toxicity, and more precise control of CAR T cell activation.

### SUMMARY OF THE INVENTION

The present inventors have discovered methods of reducing off-target toxicity, and more precisely controlling CAR T cell activation, providing important advancements in CAR T cell therapy. In the various embodiments described herein, a small molecule ligand linked to a targeting moiety by a linker is used as a bridge between the cancer and CAR T cells expressing a CAR wherein the CAR comprises an E2 anti-fluorescein antibody fragment. The bridge directs the CAR T cells, expressing a CAR comprising an E2 anti-fluorescein antibody fragment, to the cancer for amelioration of the cancer. In one embodiment, the “small molecule ligand” can be, for example, a folate, DUPA, an NK-1R ligand, a CAIX ligand, a ligand of gamma glutamyl transpeptidase, an NKG2D ligand, or a CCK2R ligand, each of which is a small molecule ligand that binds specifically to cancer cells (i.e., the receptor for these ligands is overexpressed on cancers compared to normal tissues).

In one embodiment, the “small molecule ligand” is linked to a “targeting moiety” that binds to the CAR expressed by CAR T cells. In various embodiments, the “targeting moiety” can be selected, for example, from fluorescein, fluorescein isothiocyanate (FITC), NHS- and/or fluorescein.

The “targeting moiety” binds to the recognition region of the genetically engineered CAR, expressing an E2 anti-fluorescein antibody fragment. Accordingly, the recognition region of the CAR (e.g., a single chain variable region (scFv) of an E2 anti-fluorescein antibody fragment, an Fab, Fv, Fc, (Fab′)2 fragment, and the like) is directed to the “targeting moiety.” Thus, the small molecule ligand linked to a targeting moiety by a linker acts as a bridge between the cancer and the CAR T cells, expressing an E2 anti-fluorescein antibody fragment, directing the CAR T cells to the cancer for amelioration of the cancer.

In one embodiment, a method of treatment of a cancer is provided. The method comprises i) administering to a patient a compound, or a pharmaceutically acceptable salt thereof, wherein the compound comprises a small molecule ligand linked to a targeting moiety by a linker, ii) administering to the patient a first dose of a CAR T cell composition comprising CAR T cells wherein the CAR T cells comprise a CAR directed to the targeting moiety and wherein the CAR comprises an E2 anti-fluorescein antibody fragment, and iii) administering to the patient a second dose of a CAR T cell composition comprising CAR T cells wherein the CAR T cells comprise the CAR directed to the targeting moiety and wherein the CAR comprises an E2 anti-fluorescein antibody fragment.

In another embodiment, a method of treatment of a cancer is provided. The method comprises i) administering to a patient a compound, or a pharmaceutically acceptable salt thereof, wherein the compound comprises a small molecule ligand linked to a targeting moiety by a linker, and ii) administering to the patient a CAR T cell composition wherein the CAR T cell composition comprises CAR T cells, wherein the CAR T cells comprise a CAR directed to the targeting moiety wherein the CAR comprises an E2 anti-fluorescein antibody fragment, and wherein the CAR T cell composition comprises a mixture of the CAR T cells and non-transformed T cells.

In yet another embodiment, a method of treatment of a cancer is provided. The method comprises i) administering to a patient a compound, or a pharmaceutically acceptable salt thereof, wherein the compound comprises a small molecule ligand linked to a targeting moiety by a linker, ii) administering to the patient a CAR T cell composition wherein the CAR T cell composition comprises CAR T cells wherein the CAR T cells comprise a CAR directed to the targeting moiety and wherein the CAR comprises an E2 anti-fluorescein antibody fragment; and iii) administering to the patient a folate, a conjugate comprising a folate wherein the conjugate comprising a folate does not comprise a targeting moiety, or an agent that inhibits activation of the CAR T cells.

In still another embodiment, a method of treatment of a cancer is provided. The method comprises i) administering to a patient a compound, or a pharmaceutically acceptable salt thereof, wherein the compound comprises a small molecule ligand linked to a targeting moiety by a linker, wherein at least a first dose and a second dose of the compound, or the pharmaceutically acceptable salt thereof, are administered to the patient, wherein the first dose and the second dose are different, wherein the second dose of the compound, or the pharmaceutically acceptable salt thereof, is about 2-fold to about 15000-fold greater in amount than the first dose of the compound, or the pharmaceutically acceptable salt thereof, and ii) administering to the patient a CAR T cell composition comprising CAR T cells wherein the CAR T cells comprise a CAR directed to the targeting moiety and wherein the CAR comprises an E2 anti-fluorescein antibody fragment.

In another illustrative embodiment, a method of treatment of a cancer is provided. The method comprises i) administering to a patient a first dose of a compound, or a pharmaceutically acceptable salt thereof, wherein the compound comprises a small molecule ligand linked to a targeting moiety by a linker, ii) administering to the patient at least a second dose of the compound, or a pharmaceutically acceptable salt thereof, wherein the second dose of the compound, or the pharmaceutically acceptable salt thereof, is at least about 50 percent lower in amount than the first dose of the compound, or the pharmaceutically acceptable salt thereof; and iii) administering to the patient a dose of a CAR T cell composition comprising CAR T cells wherein the CAR T cells comprise a CAR directed to the targeting moiety and wherein the CAR comprises an E2 anti-fluorescein antibody fragment.

In another embodiment, a method of treatment of a cancer is provided. The method comprises i) administering to a patient a compound, or a pharmaceutically acceptable salt thereof, wherein the compound comprises a small molecule ligand linked to a targeting moiety by a linker, and wherein the compound, or the pharmaceutically acceptable salt thereof, is administered at a dose of about 10 nmoles/kg of body weight of the patient to about 2500 nmoles/kg of body weight of the patient, and ii) administering to the patient a CAR T cell composition comprising CAR T cells, wherein the CAR T cells comprise a CAR, wherein the CAR comprises an E2 anti-fluorescein antibody fragment, and wherein the CAR T cells are at a dose of about 1 million of the CAR T cells to about 15 million of the CAR T cells.

In still another embodiment, a method of treatment of a cancer is provided. The method comprises i) administering continuously to a patient a compound, or a pharmaceutically acceptable salt thereof, wherein the compound comprises a small molecule ligand linked to a targeting moiety by a linker, ii) administering to the patient a CAR T cell composition comprising CAR T cells wherein the CAR T cells comprise a CAR, wherein the CAR comprises an E2 anti-fluorescein antibody fragment, and iii) ending the continuous administration of the compound, or the pharmaceutically acceptable salt thereof, to inhibit or prevent cytokine release syndrome in the patient.

In another embodiment, a method of treatment of a cancer is provided. The method comprises i) administering to a patient a compound, or a pharmaceutically acceptable salt thereof, wherein the compound comprises a small molecule ligand linked to a targeting moiety by a linker and wherein the compound, or the pharmaceutically acceptable salt thereof, is administered once weekly to the patient, and ii) administering to the patient a CAR T cell composition comprising CAR T cells wherein the CAR T cells comprise a CAR, wherein the CAR comprises an E2 anti-fluorescein antibody fragment.

In still another embodiment, a method of treatment of a cancer is provided. The method comprises i) administering to a patient a first dose of a compound, or a pharmaceutically acceptable salt thereof, wherein the compound comprises a small molecule ligand linked to a targeting moiety by a linker and wherein the compound, or the pharmaceutically acceptable salt thereof, is administered to the patient before the administration of a CAR T cell composition comprising CAR T cells wherein the CAR T cells comprise a CAR directed to the targeting moiety, ii) then administering to the patient a dose of the CAR T cell composition, and iii) then administering to the patient a second dose of the compound, or the pharmaceutically acceptable salt thereof, wherein the CAR comprises an E2 anti-fluorescein antibody fragment.

Additional embodiments are also described by the following enumerated clauses. Any of the following embodiments in combination with any applicable embodiments described in the Summary section, the Detailed Description of the Illustrative Embodiments section, the Examples section, or the claims of this patent application, are also contemplated.
1. A method of treatment of a cancer, the method comprising
   i) administering to a patient a compound, or a pharmaceutically acceptable salt thereof, wherein the compound comprises a small molecule ligand linked to a targeting moiety by a linker;
   ii) administering to the patient a first dose of a CAR T cell composition comprising CAR T cells wherein the CAR T cells comprise a CAR directed to the targeting moiety and wherein the CAR comprises an E2 anti-fluorescein antibody fragment; and
   iii) administering to the patient a second dose of a CAR T cell composition comprising CAR T cells wherein the CAR T cells comprise the CAR directed to the targeting moiety and wherein the CAR comprises an E2 anti-fluorescein antibody fragment.
2. A method of treatment of a cancer, the method comprising
   i) administering to a patient a compound, or a pharmaceutically acceptable salt thereof, wherein the compound comprises a small molecule ligand linked to a targeting moiety by a linker; and
   ii) administering to the patient a CAR T cell composition wherein the CAR T cell composition comprises CAR T cells, wherein the CAR T cells comprise a CAR directed to the targeting moiety wherein the CAR comprises an E2 anti-fluorescein antibody fragment, and wherein the CAR T cell composition comprises a mixture of the CAR T cells and non-transformed T cells.
3. A method of treatment of a cancer, the method comprising
   i) administering to a patient a compound, or a pharmaceutically acceptable salt thereof, wherein the compound comprises a small molecule ligand linked to a targeting moiety by a linker;
   ii) administering to the patient a CART cell composition wherein the CAR T cell composition comprises CAR T cells wherein the CAR T cells comprise a CAR directed to the targeting moiety and wherein the CAR comprises an E2 anti-fluorescein antibody fragment; and
   iii) administering to the patient a folate, a conjugate comprising a folate wherein the conjugate comprising a folate does not comprise a targeting moiety, or an agent that inhibits activation of the CAR T cells.
4. The method of clause 3 wherein step iii comprises administering a folate.
5. The method of any one of clauses 3 or 4 wherein step iii comprises administering folic acid or leucovorin.
6. The method of clause 3 wherein step iii comprises administering the conjugate comprising a folate.
7. The method of clause 6 wherein the conjugate comprising a folate comprises a folate linked to one or more amino acids.
8. The method of clause 7 wherein the conjugate comprising a folate has the formula

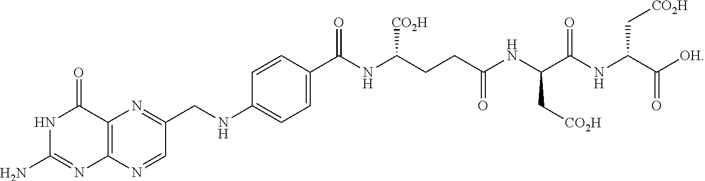 [SMILES: Nc1nc2ncc(CNc3ccc(C(=O)N[C@@H](CCC(=O)N[C@H](CC(=O)O)C(=O)N[C@H](CC(=O)O)C(=O)O)C(=O)O)cc3)nc2c(=O)[nH]1]

9. The method of any one of clauses 3 to 8 wherein the folate has the formula

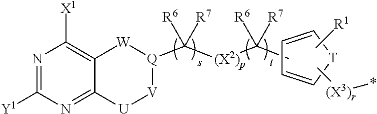

wherein X¹ and Y¹ are each-independently selected from the group consisting of halo, R², OR², SR³, and NR⁴R⁵;
U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;
X² and X³ are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyeneoxy, where Z is oxygen or sulfur,
R¹ is selected-from the group consisting of hydrogen, halo, C₁-C₁₂ alkyl, and C₁-C₁₂ alkoxy;
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group;
R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
p, r, s, and t are each independently either 0 or 1; and
* represents an optional covalent bond to the rest of the conjugate.
10. The method of clause 3 wherein the agent that inhibits activation of the CAR T cells is administered and is selected from the group consisting of a lymphocyte-specific protein tyrosine kinase inhibitor, a PI3 kinase inhibitor, an inhibitor of an IL-2 inducible T cell kinase, a JAK inhibitor, a BTK inhibitor, EC2319, and an agent that blocks CAR T cell binding to the compound, or the pharmaceutically acceptable salt thereof, but does not bind to the cancer.
11. The method of clause 10 wherein the agent that inhibits activation of the CAR T cells is administered and the agent is a lymphocyte-specific protein tyrosine kinase inhibitor.
12. The method of clause 11 wherein the lymphocyte-specific protein tyrosine kinase inhibitor is Dasatinib.
13. The method of clause 10 wherein the agent that inhibits activation of the CAR T cells is administered and the agent is a PI3 kinase inhibitor.
14. The method of clause 13 wherein the PI3 kinase inhibitor is GDC0980.
15. The method of clause 10 wherein the agent that inhibits activation of the CAR T cells is administered and the agent is an IL-2 inducible T cell kinase inhibitor.
16. The method of clause 15 wherein the IL-2 inducible T cell kinase inhibitor is BMS-509744.
17. The method of clause 10 wherein the agent that inhibits activation of the CAR T cells is administered and is an agent that blocks CAR T cell binding to the compound, or the pharmaceutically acceptable salt thereof, but does not bind to the cancer.
18. The method of clause 17 wherein the agent is fluoresceinamine, FITC, or sodium fluorescein.
19. The method of clause 18 wherein the agent is FITC.
20. The method of clause 18 wherein the agent is sodium fluorescein.
21. The method of any one of clauses 17 to 20 wherein the agent that inhibits activation of the CAR T cells is administered at a dose of about 0.01 to about 300 umoles/kg of body weight of the patient.
22. The method of any one of clauses 17 to 20 wherein the agent that inhibits activation of the CAR T cells is administered at a dose of about 0.06 to about 100 umoles/kg of body weight of the patient.
23. The method of any one of clauses 17 to 20 wherein the agent that inhibits activation of the CAR T cells is administered at a dose of about 0.06 to about 90 umoles/kg of body weight of the patient.
24. The method of any one of clauses 17 to 20 wherein the agent that inhibits activation of the CAR T cells is administered at a dose of about 0.06 to about 80 umoles/kg of body weight of the patient.
25. The method of any one of clauses 17 to 20 wherein the agent that inhibits activation of the CAR T cells is administered at a dose of about 0.06 to about 70 umoles/kg of body weight of the patient.
26. The method of any one of clauses 17 to 20 wherein the agent that inhibits activation of the CAR T cells is administered at a dose of about 0.06 to about 60 umoles/kg of body weight of the patient.
27. The method of any one of clauses 17 to 20 wherein the agent that inhibits activation of the CAR T cells is administered at a dose of about 0.06 to about 50 umoles/kg of body weight of the patient.
28. The method of any one of clauses 17 to 20 wherein the agent that inhibits activation of the CAR T cells is administered at a dose of about 0.06 to about 40 umoles/kg of body weight of the patient.
29. The method of any one of clauses 17 to 20 wherein the agent that inhibits activation of the CART cells is administered at a dose of about 0.06 to about 30 umoles/kg of body weight of the patient.
30. The method of any one of clauses 17 to 20 wherein the agent that inhibits activation of the CAR T cells is administered at a dose of about 0.06 to about 20 umoles/kg of body weight of the patient.
31. The method of any one of clauses 17 to 20 wherein the agent that inhibits activation of the CAR T cells is administered at a dose of about 0.06 to about 10 umoles/kg of body weight of the patient.
32. The method of any one of clauses 17 to 20 wherein the agent that inhibits activation of the CAR T cells is administered at a dose of about 0.06 to about 8 umoles/kg of body weight of the patient.
33. The method of any one of clauses 17 to 20 wherein the agent that inhibits activation of the CAR T cells is administered at a dose of about 0.06 to about 6 umoles/kg of body weight of the patient.
34. The method of any one of clauses 3 to 33 wherein more than one dose is administered to the patient of the folate, the conjugate comprising a folate wherein the conjugate comprising a folate does not comprise a targeting moiety, or the agent that inhibits activation of the CART cells.
35. The method of any one of clauses 3 to 34 wherein the folate, the conjugate comprising a folate wherein the conjugate comprising a folate does not comprise a targeting moiety, or the agent that inhibits activation of the CAR T cells is administered to the patient before and/or after the compound, or the pharmaceutically acceptable salt thereof.
36. The method of any one of clauses 3 to 35 wherein administration of the folate, the conjugate comprising a folate wherein the conjugate comprising a folate does not comprise a targeting moiety, or the agent that inhibits activation of the CAR T cells causes reduction in cytokine levels in the patient.
37. The method of clause 36 wherein the reduction in cytokine levels occurs by about 3 hours after administration to the patient of the folate, the conjugate comprising a folate wherein the conjugate comprising a folate does not comprise a targeting moiety, or the agent that inhibits activation of the CAR T cells.
38. The method of clause 36 wherein the reduction in cytokine levels occurs by about 6 hours after administration to the patient of the folate, the conjugate comprising a folate wherein the conjugate comprising a folate does not comprise a targeting moiety, or the agent that inhibits activation of the CAR T cells.
39. The method of any one of clauses 36 to 38 wherein the reduction in cytokine levels is a reduction to about the cytokine levels in an untreated patient.
40. The method of any one of clauses 3 to 39 wherein the compound, or the pharmaceutically acceptable salt thereof, is administered before and subsequent to administration of the folate, the conjugate comprising a folate wherein the conjugate comprising a folate does not comprise a targeting moiety, or the agent that inhibits activation of the CAR T cells.
41. The method of any one of clauses 3 to 40 wherein CAR T cell number increases in the blood of the patient after administration of the folate, the conjugate comprising a folate wherein the conjugate comprising a folate does not comprise a targeting moiety, or the agent that inhibits activation of the CAR T cells, even though cytokine levels in the patient are reduced.
42. The method of any one of clauses 3 to 41 wherein CAR T cell activation is enhanced or maintained, relative to a patient not treated with a rescue agent, after administration of the folate, the conjugate comprising a folate wherein the conjugate comprising a folate does not comprise a targeting moiety, or the agent that inhibits activation of the CAR T cells, even though cytokine levels in the treated patient are reduced.
43. The method of any one of clauses 3 to 42 wherein the cancer comprises a tumor and tumor size in the patient is not increased when the folate, the conjugate comprising a folate wherein the conjugate comprising a folate does not comprise a targeting moiety, or the agent that inhibits activation of the CAR T cells is administered to the patient.
44. The method of clause 43 wherein a complete response for the tumor is obtained.
45. The method of any one of clauses 3 to 44 wherein the folate, the conjugate comprising a folate wherein the conjugate comprising a folate does not comprise a targeting moiety, or the agent that inhibits activation of the CAR T cells the agent that inhibits activation of the CAR T cells is administered to the patient when the CRS grade reaches 1, 2, 3, or 4.
46. The method of clause 45 wherein the agent that inhibits activation of the CAR T cells is administered to the patient when the CRS grade reaches 3 or 4.
47. The method of any one of clauses 3 to 46 wherein lung edema is reduced.
48. The method of any one of clauses 1 to 47 wherein the compound, or the pharmaceutically acceptable salt thereof, is administered at a dose of about 10 nmoles/kg of body weight of the patient to about 2500 nmoles/kg of body weight of the patient; and the CAR T cells are at a dose of about 1 million of the CAR T cells to about 15 million of the CAR T cells.
49. The method of clause 48 wherein the compound, or the pharmaceutically acceptable salt thereof, is administered at a dose of about 10 nmoles/kg to about 100 nmoles/kg of body weight of the patient.
50. The method of any one of clauses 48 to 49 wherein the compound, or the pharmaceutically acceptable salt thereof, is administered at a dose of about 10 nmoles/kg to about 50 nmoles/kg of body weight of the patient.
51. The method of any one of clauses 48 to 50 wherein the compound, or the pharmaceutically acceptable salt thereof, is administered at a dose of about 10 nmoles/kg to about 20 nmoles/kg of body weight of the patient.
52. The method of clause 48 wherein the compound, or the pharmaceutically acceptable salt thereof, is administered at a dose of about 10 nmoles/kg to about 600 nmoles/kg of body weight of the patient.
53. The method of clause 48 wherein the compound, or the pharmaceutically acceptable salt thereof, is administered at a dose of about 200 nmoles/kg to about 600 nmoles/kg of body weight of the patient.
54. The method of clause 48 wherein the compound, or the pharmaceutically acceptable salt thereof, is administered at a dose of about 400 nmoles/kg to about 600 nmoles/kg of body weight of the patient.
55. The method of any one of clauses 48 to 54 wherein the CAR T cells are at a dose of about 1 million of the CAR T cells to about 12.5 million of the CAR T cells.
56. The method of any one of clauses 48 to 55 wherein the CAR T cells are at a dose of about 1 million of the CAR T cells to about 7 million of the CAR T cells.
57. The method of any one of clauses 48 to 56 wherein the CAR T cells are at a dose of about 1 million of the CAR T cells to about 5 million of the CAR T cells.
58. The method of any one of clauses 48 to 57 wherein the CAR T cells are at a dose of about 2 million of the CAR T cells to about 5 million of the CAR T cells.
59. The method of any one of clauses 1 to 58 further comprising the step of ending continuous administration of the compound, or the pharmaceutically acceptable salt thereof, to inhibit or prevent cytokine release syndrome in the patient.
60. The method of any one of clauses 1 to 59 wherein the compound, or the pharmaceutically acceptable salt thereof, is administered continuously for at least one hour to the patient.
61. The method of any one of clauses 1 to 59 wherein the compound, or the pharmaceutically acceptable salt thereof, is administered continuously for at least four hours to the patient.
62. The method of any one of clauses 1 to 59 wherein the compound, or the pharmaceutically acceptable salt thereof, is administered continuously for at least six hours to the patient.
63. The method of any one of clauses 1 to 62 wherein the compound, or the pharmaceutically acceptable salt thereof, is administered to the patient every other day.
64. The method of any one of clauses 1 to 62 wherein the compound, or the pharmaceutically acceptable salt thereof, is administered to the patient three times weekly.
65. The method of any one of clauses 1 to 62 wherein the compound, or the pharmaceutically acceptable salt thereof, is administered to the patient two times weekly.
66. The method of any one of clauses 1 to 62 wherein the compound, or the pharmaceutically acceptable salt thereof, is administered to the patient once weekly.
67. The method of any one of clauses 1 to 62 wherein compound, or the pharmaceutically acceptable salt thereof, is administered to the patient until an unacceptable loss of body weight of the patient, a fever, a drop in blood pressure, or pulmonary edema occurs.
68. A method of treatment of a cancer, the method comprising
   i) administering to a patient a compound, or a pharmaceutically acceptable salt thereof, wherein the compound comprises a small molecule ligand linked to a targeting moiety by a linker, wherein at least a first dose and a second dose of the compound, or the pharmaceutically acceptable salt thereof, are administered to the patient, wherein the first dose and the second dose are different, wherein the second dose of the compound, or the pharmaceutically acceptable salt thereof, is about 2-fold to about 15000-fold greater in amount than the first dose of the compound, or the pharmaceutically acceptable salt thereof; and
   ii) administering to the patient a CAR T cell composition comprising CAR T cells wherein the CAR T cells comprise a CAR directed to the targeting moiety and wherein the CAR comprises an E2 anti-fluorescein antibody fragment.
69. The method of clause 68 wherein at least a first dose, a second dose, and a third dose of the compound, or the pharmaceutically acceptable salt thereof, are administered to the patient, wherein the first dose, the second dose, and the third dose are different, wherein the second dose of the compound, or the pharmaceutically acceptable salt thereof, is about 2-fold to about 750-fold greater in amount than the first dose of the compound, or the pharmaceutically acceptable salt thereof, and wherein the third dose of the compound, or the pharmaceutically acceptable salt thereof, is about 800-fold to about 10000-fold greater in amount than the first dose of the compound, or the pharmaceutically acceptable salt thereof.
70. The method of clause 68 wherein at least a first dose, a second dose, a third dose, and a fourth dose of the compound, or the pharmaceutically acceptable salt thereof, are administered to the patient, wherein the first dose, the second dose, the third dose, and the fourth dose are different, wherein the second dose of the compound, or the pharmaceutically acceptable salt thereof, is about 2-fold to about 750-fold greater in amount than the first dose of the compound, or the pharmaceutically acceptable salt thereof, wherein the third dose of the compound, or the pharmaceutically acceptable salt thereof, is about 800-fold to about 7500-fold greater in amount than the first dose of the compound, or the pharmaceutically acceptable salt thereof, and wherein the fourth dose of the compound, or the pharmaceutically acceptable salt thereof, is about 8000 to about 15000-fold greater in amount than the first dose of the compound, or the pharmaceutically acceptable salt thereof.
71. The method of clause 70 wherein the second dose of the compound, or the pharmaceutically acceptable salt thereof, is about 100-fold greater in amount than the first dose of the compound, or the pharmaceutically acceptable salt thereof, wherein the third dose of the compound, or the pharmaceutically acceptable salt thereof, is about 1000-fold greater in amount than the first dose of the compound, or the pharmaceutically acceptable salt thereof, and wherein the fourth dose of the compound, or the pharmaceutically acceptable salt thereof, is about 10000-fold greater in amount than the first dose of the compound, or the pharmaceutically acceptable salt thereof.
72. The method of clause 68 wherein at least a first dose and a second dose of the compound, or the pharmaceutically acceptable salt thereof, are administered to the patient, wherein the first dose and the second dose are different, wherein the second dose of the compound, or the pharmaceutically acceptable salt thereof, is about 2-fold to about 15000-fold greater in amount than the first dose of the compound, or the pharmaceutically acceptable salt thereof.
73. A method of treatment of a cancer, the method comprising
   i) administering to a patient a first dose of a compound, or a pharmaceutically acceptable salt thereof, wherein the compound comprises a small molecule ligand linked to a targeting moiety by a linker;
   ii) administering to the patient at least a second dose of the compound, or a pharmaceutically acceptable salt thereof, wherein the second dose of the compound, or the pharmaceutically acceptable salt thereof, is at least about 50 percent lower in amount than the first dose of the compound, or the pharmaceutically acceptable salt thereof; and iii) administering to the patient a dose of a CAR T cell composition comprising CAR T cells wherein the CAR T cells comprise a CAR directed to the targeting moiety and wherein the CAR comprises an E2 anti-fluorescein antibody fragment.
74. The method of clause 73 wherein the second dose of the compound, or the pharmaceutically acceptable salt thereof, is at least about 60 percent lower in amount than the first dose of the compound, or the pharmaceutically acceptable salt thereof.
75. The method of clause 73 wherein the second dose of the compound, or the pharmaceutically acceptable salt thereof, is at least about 70 percent lower in amount than the first dose of the compound, or the pharmaceutically acceptable salt thereof.
76. The method of clause 73 wherein the second dose of the compound, or the pharmaceutically acceptable salt thereof, is at least about 80 percent lower in amount than the first dose of the compound, or the pharmaceutically acceptable salt thereof.
77. The method of clause 73 wherein the second dose of the compound, or the pharmaceutically acceptable salt thereof, is at least about 90 percent lower in amount than the first dose of the compound, or the pharmaceutically acceptable salt thereof.
78. The method of clause 73 wherein the second dose of the compound, or the pharmaceutically acceptable salt thereof, is at least about 95 percent lower in amount than the first dose of the compound, or the pharmaceutically acceptable salt thereof.
79. The method of clause 73 wherein the second dose of the compound, or the pharmaceutically acceptable salt thereof, is at least about 96 percent lower in amount than the first dose of the compound, or the pharmaceutically acceptable salt thereof.
80. The method of clause 73 wherein the second dose of the compound, or the pharmaceutically acceptable salt thereof, is at least about 97 percent lower in amount than the first dose of the compound, or the pharmaceutically acceptable salt thereof.
81. The method of clause 73 wherein the second dose of the compound, or the pharmaceutically acceptable salt thereof, is at least about 98 percent lower in amount than the first dose of the compound, or the pharmaceutically acceptable salt thereof.
82. The method of clause 73 wherein the second dose of the compound, or the pharmaceutically acceptable salt thereof, is at least about 99 percent lower in amount than the first dose of the compound, or the pharmaceutically acceptable salt thereof.
83. The method of clause 73 wherein the second dose of the compound, or the pharmaceutically acceptable salt thereof, is at least about 99.5 percent lower in amount than the first dose of the compound, or the pharmaceutically acceptable salt thereof.
84. The method of any one of clauses 73 to 83 wherein the first dose of the compound, or the pharmaceutically acceptable salt thereof, is about 100 nmoles/kg to about 1000 nmoles/kg of body weight of the patient.
85. The method of any one of clauses 73 to 83 wherein the first dose of the compound, or the pharmaceutically acceptable salt thereof, is about 100 nmoles/kg to about 900 nmoles/kg of body weight of the patient.
86. The method of any one of clauses 73 to 83 wherein the first dose of the compound, or the pharmaceutically acceptable salt thereof, is about 100 nmoles/kg to about 800 nmoles/kg of body weight of the patient.
87. The method of any one of clauses 73 to 83 wherein the first dose of the compound, or the pharmaceutically acceptable salt thereof, is about 100 nmoles/kg to about 700 nmoles/kg of body weight of the patient.
88. The method of any one of clauses 73 to 83 wherein the first dose of the compound, or the pharmaceutically acceptable salt thereof, is about 100 nmoles/kg to about 600 nmoles/kg of body weight of the patient.
89. The method of any one of clauses 73 to 83 wherein the first dose of the compound, or the pharmaceutically acceptable salt thereof, is about 200 nmoles/kg to about 600 nmoles/kg of body weight of the patient.
90. The method of any one of clauses 73 to 83 wherein the first dose of the compound, or the pharmaceutically acceptable salt thereof, is about 400 nmoles/kg to about 600 nmoles/kg of body weight of the patient.
91. The method of any one of clauses 73 to 83 wherein the first dose of the compound, or the pharmaceutically acceptable salt thereof, is about 500 nmoles/kg of body weight of the patient.
92. The method of clause 84 wherein the second dose of the compound, or the pharmaceutically acceptable salt thereof, is about 0.5 nmoles/kg to about 500 nmoles/kg of body weight of the patient.
93. The method of clause 85 wherein the second dose of the compound, or the pharmaceutically acceptable salt thereof, is about 0.5 nmoles/kg to about 450 nmoles/kg of body weight of the patient.
94. The method of clause 86 wherein the second dose of the compound, or the pharmaceutically acceptable salt thereof, is about 0.5 nmoles/kg to about 400 nmoles/kg of body weight of the patient.
95. The method of clause 87 wherein the second dose of the compound, or the pharmaceutically acceptable salt thereof, is about 0.5 nmoles/kg to about 350 nmoles/kg of body weight of the patient.
96. The method of clause 88 wherein the second dose of the compound, or the pharmaceutically acceptable salt thereof, is about 0.5 nmoles/kg to about 300 nmoles/kg of body weight of the patient.
97. The method of clause 89 wherein the second dose of the compound, or the pharmaceutically acceptable salt thereof, is about 1 nmole/kg to about 300 nmoles/kg of body weight of the patient.
98. The method of clause 90 wherein the second dose of the compound, or the pharmaceutically acceptable salt thereof, is about 2 nmoles/kg to about 300 nmoles/kg of body weight of the patient.
99. The method of clause 91 wherein the second dose of the compound, or the pharmaceutically acceptable salt thereof, is about 2 nmoles/kg to about 250 nmoles/kg of body weight of the patient
100. The method of any one of clauses 92 to 99 wherein the second dose of the compound, or the pharmaceutically acceptable salt thereof, is about 5 nmoles/kg to about 40 nmoles/kg of body weight of the patient.
101. The method of any one of clauses 92 to 99 wherein the second dose of the compound, or the pharmaceutically acceptable salt thereof, is about 40 nmoles/kg to about 150 nmoles/kg of body weight of the patient.
102. The method of any one of clauses 73 to 101 further comprising administering a third dose of the compound, or the pharmaceutically acceptable salt thereof, wherein the third dose of the compound, or the pharmaceutically acceptable salt thereof, is the same as the second dose of the compound, or the pharmaceutically acceptable salt thereof.
103. The method of clause 102 further comprising administering a fourth dose of the compound, or the pharmaceutically acceptable salt thereof, wherein the fourth dose of the compound, or the pharmaceutically acceptable salt thereof, is the same as the second dose, or the pharmaceutically acceptable salt thereof, and the third dose of the compound, or the pharmaceutically acceptable salt thereof.
104. The method of any one of clauses 73 to 103 wherein the dose(s) of the compound, or the pharmaceutically acceptable salt thereof, administered after the first dose of the compound, or the pharmaceutically acceptable salt thereof, maintain inhibition of growth of the cancer relative to the first dose of the compound, or the pharmaceutically acceptable salt thereof.
105. The method of any one of clauses 73 to 104 wherein the CAR T cells are administered at a dose of about 1 million of the CAR T cells to about 40 million of the CAR T cells.
106. The method of any one of clauses 73 to 105 wherein the dose(s) of the compound, or the pharmaceutically acceptable salt thereof, administered after the first dose of the compound, or the pharmaceutically acceptable salt thereof, are administered once weekly.
107. The method of any one of clauses 73 to 105 wherein the dose(s) of the compound, or the pharmaceutically acceptable salt thereof, are administered twice weekly.
108. The method of any one of clauses 1 to 107 wherein the CAR further comprises an IgG4 hinge domain, a CD3ζ activation domain, and a 4-1BB co-stimulation domain.
109. The method of any one of clauses 1 to 108 wherein the E2 anti-fluorescein antibody fragment is an scFv fragment.
110. The method of any one of clauses 1 to 109 wherein the CAR protein sequence has at least about 90% identity to SEQ ID NO:2.
111. The method of any one of clauses 1 to 109 wherein the CAR protein sequence has at least about 95% identity to SEQ ID NO:2.
112. The method of any one of clauses 1 to 109 wherein the CAR protein sequence has at least about 98% identity to SEQ ID NO:2.
113. The method of any one of clauses 1 to 112 wherein the CAR binds fluorescein.
114. The method of any one of clauses 1 to 109 wherein the CAR protein sequence has up to about 50 conservative amino acid substitutions and wherein the CAR binds fluorescein.
115. The method of any one of clauses 1 to 109 wherein the CAR is encoded by a polynucleotide having at least about 90% identity to SEQ ID NO:1.
116. The method of any one of clauses 1 to 109 wherein the CAR is encoded by a polynucleotide having at least about 95% identity to SEQ ID NO:1.
117. The method of any one of clauses 1 to 109 wherein the CAR is encoded by a polynucleotide having at least about 98% identity to SEQ ID NO:1.
118. The method of any one of clauses 115 to 117 wherein the CAR binds fluorescein.
119. The method of any one of clauses 1 to 118 wherein the CAR is encoded by a polynucleotide that hybridizes under high stringency conditions to a polynucleotide having SEQ ID NO:1 and wherein the CAR binds fluorescein.
120. The method of any one of clauses 1 to 119 wherein the CAR is encoded by a polynucleotide having SEQ ID NO:1, or by a degenerate variant of SEQ ID NO:1.
121. The method of any one of clauses 1 to 120 wherein the ligand is selected from the group consisting of a folate, DUPA, an NK-1R ligand, a CAIX ligand, a ligand of gamma glutamyl transpeptidase, an NKG2D ligand, and a CCK2R ligand.
122. The method of any one of clauses 1 to 121 wherein the targeting moiety is fluorescein, or a pharmaceutically acceptable salt thereof.
123. The method of any one of clauses 1 to 122 wherein the linker comprises polyethylene glycol (PEG), polyproline, a hydrophilic amino acid, a sugar, an unnatural peptidoglycan, a polyvinylpyrrolidone, pluronic F-127, or a combination thereof.
124. The method of any one of clauses 1 to 123 wherein the linker comprises PEG.
125. The method of any one of clauses 1 to 124 wherein the compound, or the pharmaceutically acceptable salt thereof, has the formula

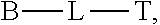

wherein B represents the small molecule ligand, L represents the linker, and T represents the targeting moiety, and wherein L comprises a structure having the formula

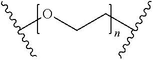

wherein n is an integer from 0 to 200.
126. The method of any one of clauses 1 to 125 wherein the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, osteosarcoma, cancer of the urethra, prostate cancer, chronic leukemia, acute leukemia, acute myelocytic leukemia, lymphocytic lymphoma, myeloid leukemia, myelomonocytic leukemia, hairy cell leukemia, myelomonocytic leukemia, hairy cell leukemia, pleural mesothelioma, cancer of the bladder. Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, and adenocarcinoma of the gastroesophageal junction.
127. The method of any one of clauses 1 to 126 wherein the cancer is a folate receptor expressing cancer.
128. The method of any one of clauses 1 to 127 wherein the cancer is an endometrial cancer.
129. The method of any one of clauses 1 to 127 wherein the cancer is a non-small cell lung cancer.
130. The method of any one of clauses 1 to 127 wherein the cancer is an ovarian cancer.
131. The method of any one of clauses 1 to 127 wherein the cancer is a triple negative breast cancer.
132. The method of any one of clauses 1 to 127 wherein the cancer is acute myelocytic leukemia.
133. The method of clause 132 wherein the cancer expresses the folate receptor-β.
134. The method of any one of clauses 1 to 133 wherein multiple doses of the CAR T cell composition are administered.
135. The method of any one of clauses 1 to 134 wherein at least two doses of the CAR T cell composition are administered.
136. The method of any one of clauses 1 to 135 wherein the patient is imaged prior to administration of the compound, or the pharmaceutically acceptable salt thereof, or prior to administration of the CAR T cell composition.
137. The method of any one of clauses 1 to 136 wherein the compound, or the pharmaceutically acceptable salt thereof, is not an antibody, and does not comprise a fragment of an antibody.
138. The method of any one of clauses 1 to 137 wherein the targeting moiety does not comprise a peptide epitope.
139. The method of any one of clauses 1 to 138 wherein cytokine release resulting in off-target toxicity in the patient does not occur and wherein CAR T cell toxicity to the cancer occurs.
140. The method of any one of clauses 1 to 138 wherein off-target tissue toxicity does not occur in the patient and wherein CAR T cell toxicity to the cancer occurs.
141. The method of any one of clauses 1 to 138 wherein the cancer comprises a tumor, wherein tumor size is reduced in the patient, and wherein off-target toxicity does not occur.
142. The method of any one of clauses 1 to 141 wherein CRS is reduced or prevented and the method results in a decrease in tumor volume in the patient.
143. The method of any one of clauses 1 to 142 wherein body weight loss due to CRS is reduced or prevented.
144. The method of any one of clauses 59 to 67 further comprising re-administering the compound, or the pharmaceutically acceptable salt thereof, to the patient.
145. The method of clause 144 wherein the subsequent administration of the compound, or the pharmaceutically acceptable salt thereof, causes CAR T cell activation and an increase in cytokine levels in the patient.
146. The method of any one of clauses 1 to 145 wherein the cancer comprises a tumor and wherein a complete response for the tumor is obtained.
147. The method of any one of clauses 1 to 146 wherein the CAR T cells have a central memory/effector memory phenotype.
148. The method of any one of clauses 1 to 147 wherein the CD8:CD4 ratio of the CAR T cells is about 1:1.

### BRIEF DESCRIPTION OF THE DRAWINGS

FIG. 1 shows the E2 construct vs. the 4M5.3 construct diagrammatically and shows a map of the E2 construct.

FIGS. 2A and 2B show an EC17 fixed dose and dose de-escalation schema.

FIGS. 3A and 3B show E2-CAR-T anti-tumor activity (30 million cells) with EC17 dose de-escalation and body weight changes. As shown, antitumor activity is maintained after NaFL rescue.

FIGS. 4A and 4B show 4M5.3-CAR-T anti-tumor activity (30 million cells) with EC17 dose de-escalation and body weight changes. As shown, 4M5.3-CAR-T was less active than E2-CAR-T. In addition, EC17 dose dependent anti-tumor activity and body weight loss were observed.

FIGS. 5A and 5B show E2-CAR-T anti-tumor activity (10 and 20 million cells) and body weight changes at a fixed EC17 dosing regimen (500 nmol/kg, SIW). As shown, antitumor activity was maintained after NaFL rescue.

FIGS. 6A and 6B show 4M5.3-CAR-T anti-tumor activity (10 and 20 million cells) and body weight changes at a fixed EC17 dosing regimen (500 nmol/kg, SIW). As shown, 4M5.3-CAR-T was less active than E2-CAR-T.

FIGS. 7A-D show the phenotypic characterization of E2 CAR T cells prior to infusion into NSG mice.

FIGS. 8A and 8B show the comparison of CAR-T differentiation phenotypes in different preparations including E2-CAR-T cells and 4M5.3-CAR-T cells, and GFP+4M5.3 CAR-T cells.

FIGS. 9A-E show binding of DIG-labeled E2-IgG to FITC.

FIGS. 10A and 10B show IHC staining of DIG-labeled E2 antibody on FITC-labeled KB cells.

FIGS. 11A and 11B show IHC staining of the bound DIG-E2 antibody in normal human organ tissues: Adrenal gland. FIG. 11A: testing tissue section pre-incubated with DIG-E2 antibody; FIG. 11B: control tissue section without pre-incubation with DIG-E2 antibody.

FIGS. 12A and 12B show IHC staining of the bound DIG-E2 antibody in normal human organ tissues: Bone marrow. FIG. 12A: testing tissue section pre-incubated with DIG-E2 antibody; FIG. 12B: control tissue section without pre-incubation with DIG-E2 antibody.

FIGS. 13A and 13B show IHC staining of the bound DIG-E2 antibody in normal human organ tissues: Breast. FIG. 13A: testing tissue section pre-incubated with DIG-E2 antibody; FIG. 13B: control tissue section without pre-incubation with DIG-E2 antibody.

FIGS. 14A and 14B show IHC staining of the bound DIG-E2 antibody in normal human organ tissues: Cerebellum tissue. FIG. 14A: testing tissue section pre-incubated with DIG-E2 antibody: FIG. 14B: control tissue section without pre-incubation with DIG-E2 antibody.

FIGS. 15A and 15B show IHC staining of the bound DIG-E2 antibody in normal human organ tissues: Cervix. FIG. 15A: testing tissue section pre-incubated with DIG-E2 antibody; FIG. 15B: control tissue section without pre-incubation with DIG-E2 antibody.

FIGS. 16A and 16B show IHC staining of the bound DIG-E2 antibody in normal human organ tissues: Colon. FIG. 16A: testing tissue section pre-incubated with DIG-E2 antibody; FIG. 16B: control tissue section without pre-incubation with DIG-E2 antibody.

FIGS. 17A and 17B show IHC staining of the bound DIG-E2 antibody in normal human organ tissues: Esophagus. FIG. 17A: testing tissue section pre-incubated with DIG-E2 antibody: FIG. 17B: control tissue section without pre-incubation with DIG-E2 antibody.

FIGS. 18A and 18B show IHC staining of the bound DIG-E2 antibody in normal human organ tissues: Eye. FIG. 18A: testing tissue section pre-incubated with DIG-E2 antibody; FIG. 18B: control tissue section without pre-incubation with DIG-E2 antibody.

FIGS. 19A and 19B show IHC staining of the bound DIG-E2 antibody in normal human organ tissues: Heart. FIG. 19A: testing tissue section pre-incubated with DIG-E2 antibody; FIG. 19B: control tissue section without pre-incubation with DIG-E2 antibody.

FIGS. 20A and 20B show IHC staining of the bound DIG-E2 antibody in normal human organ tissues: Hypophysis. FIG. 20A: testing tissue section pre-incubated with DIG-E2 antibody; FIG. 20B: control tissue section without pre-incubation with DIG-E2 antibody.

FIGS. 21A and 21B show IHC staining of the bound DIG-E2 antibody in normal human organ tissues: Kidney. FIG. 21A: testing tissue section pre-incubated with DIG-E2 antibody; FIG. 21B: control tissue section without pre-incubation with DIG-E2 antibody.

FIGS. 22A and 22B show IHC staining of the bound DIG-E2 antibody in normal human organ tissues: Larynx. FIG. 22A: testing tissue section pre-incubated with DIG-E2 antibody; FIG. 22B: control tissue section without pre-incubation with DIG-E2 antibody.

FIGS. 23A and 23B show IHC staining of the bound DIG-E2 antibody in normal human organ tissues: Spleen. FIG. 23A: testing tissue section pre-incubated with DIG-E2 antibody; FIG. 23B: control tissue section without pre-incubation with DIG-E2 antibody.

FIGS. 24A and 24B show IHC staining of the bound DIG-E2 antibody in normal human organ tissues: Liver. FIG. 24A: testing tissue section pre-incubated with DIG-E2 antibody; FIG. 24B: control tissue section without pre-incubation with DIG-E2 antibody.

FIGS. 25A and 25B show IHC staining of the bound DIG-E2 antibody in normal human organ tissues: Lung. FIG. 25A: testing tissue section pre-incubated with DIG-E2 antibody; FIG. 25B: control tissue section without pre-incubation with DIG-E2 antibody.

FIGS. 26A and 26B show IHC staining of the bound DIG-E2 antibody in normal human organ tissues: Lymph node. FIG. 26A: testing tissue section pre-incubated with DIG-E2 antibody; FIG. 26B: control tissue section without pre-incubation with DIG-E2 antibody.

FIGS. 27A and 27B show IHC staining of the bound DIG-E2 antibody in normal human organ tissues: Nerve. FIG. 27A: testing tissue section pre-incubated with DIG-E2 antibody; FIG. 27B: control tissue section without pre-incubation with DIG-E2 antibody.

FIGS. 28A and 28B show IHC staining of the bound DIG-E2 antibody in normal human organ tissues: Ovary. FIG. 28A: testing tissue section pre-incubated with DIG-E2 antibody: FIG. 28B: control tissue section without pre-incubation with DIG-E2 antibody.

FIGS. 29A and 29B show IHC staining of the bound DIG-E2 antibody in normal human organ tissues: Pancreas. FIG. 29A: testing tissue section pre-incubated with DIG-E2 antibody; FIG. 29B: control tissue section without pre-incubation with DIG-E2 antibody.

FIGS. 30A and 30B show HC staining of the bound DIG-E2 antibody in normal human organ tissues: Prostate. FIG. 30A: testing tissue section pre-incubated with DIG-E2 antibody; FIG. 30B: control tissue section without pre-incubation with DIG-E2 antibody.

FIGS. 31A and 31B show IHC staining of the bound DIG-E2 antibody in normal human organ tissues: Skin. FIG. 31A: testing tissue section pre-incubated with DIG-E2 antibody; FIG. 31B: control tissue section without pre-incubation with DIG-E2 antibody.

FIGS. 32A and 30B show IHC staining of the bound DIG-E2 antibody in normal human organ tissues: Small intestine. FIG. 32A: testing tissue section pre-incubated with DIG-E2 antibody; FIG. 32A: control tissue section without pre-incubation with DIG-E2 antibody.

FIGS. 33A and 33B show IHC staining of the bound DIG-E2 antibody in normal human organ tissues: Stomach. FIG. 33A: testing tissue section pre-incubated with DIG-E2 antibody; FIG. 33B: control tissue section without pre-incubation with DIG-E2 antibody.

FIGS. 34A and 34B show IHC staining of the bound DIG-E2 antibody in normal human organ tissues: Striated muscle. FIG. 34A: testing tissue section pre-incubated with DIG-E2 antibody, FIG. 34B: control tissue section without pre-incubation with DIG-E2 antibody.

FIGS. 35A and 35B show IHC staining of the bound DIG-E2 antibody in normal human organ tissues: Testis. FIG. 35A: testing tissue section pre-incubated with DIG-E2 antibody; FIG. 35B: control tissue section without pre-incubation with DIG-E2 antibody.

FIGS. 36A and 36B show IHC staining of the bound DIG-E2 antibody in normal human organ tissues: Thymus gland. FIG. 36A: testing tissue section pre-incubated with DIG-E2 antibody; FIG. 36B: control tissue section without pre-incubation with DIG-E2 antibody.

FIGS. 37A and 37B show IHC staining of the bound DIG-E2 antibody in normal human organ tissues: Tongue. FIG. 37A: testing tissue section pre-incubated with DIG-E2 antibody; FIG. 37B: control tissue section without pre-incubation with DIG-E2 antibody.

FIGS. 38A and 38B show IHC staining of the bound DIG-E2 antibody in normal human organ tissues: Uterus. FIG. 38A: testing tissue section pre-incubated with DIG-E2 antibody; FIG. 38B: control tissue section without pre-incubation with DIG-E2 antibody.

FIG. 39 is a chart showing T-cell activation in one day cocultures with various target cells. The percentage of E2 CAR T cells which are activated after the one-day coculture (y-axis) is graphed against FR expression level of the target cells at the time of the assay (x-axis). (▾) OV90 cells; (♦) SKOV3 cells; (▴) IGROV1 cells; (▪) HOS-Fr α cells; (●) MDA-MB-231 cells.

FIG. 40 is a graph showing target cell apoptosis in 2 day coculture with the 5 different cell types (OV90 cells; SKOV3 cells, IGROV1 cells; HOS-Fr α cells; MDA-MB-231 cells) under three different conditions. For each cell type the conditions are as follows: Left bar target cells alone +EC17; Middle bar CAR T plus target cell cocultures without EC17 pre-treatment; Right bar CAR T plus treated target cells with EC17 pre-treatment.

FIG. 41 is a chart showing functional folate receptor expression levels in 5 different cell types (OV90 cells; SKOV3 cells; IGROV1 cells; HOS-Fr α cells; MDA-MB-231 cells).

FIG. 42 is a cartoon showing the experimental timeline for tumor bearing and naïve mice.

FIG. 43A is a chart showing levels of cytokine IFN-γ was EC-17 dependent in both naïve and tumor bearing mice, and that the increase in IFN-γ was much lower in naïve mice compared to mice bearing MDA-MB-231 tumors (23-fold lower). FIG. 43B is a chart showing FACS analysis CAR-T expansion in mice bearing MDA-MB-231 tumors and no detectable CAR-T cell expansion in naïve mice.

FIG. 44A is a chart showing cytokine (IL-2) production from Mock T-Cells and anti-FLCAR T-cells at quantitatively similar levels in co-culture with the positive control cell line K562-OKT3 (left pair of bars, where Mock is the left bar and anti-FLCAR T-cells is the right bar); no cytokine was produced by either Mock or anti-FLCAR T-cells upon co-culture with K562 (middle); and anti-FLCAR T-cells were the only cells able to elicit secretion of the cytokine IL-2, but only with pretreatment of EC-17 (right, where only anti-FLCAR T-cells show a result). FIG. 44B is a chart showing cytokine (IFN-γ) production from Mock T-Cells and anti-FLCAR T-cells at quantitatively similar levels in co-culture with the positive control cell line K562-OKT3 (left pair of bars, where Mock is the left bar and anti-FLCAR T-cells is the right bar); no cytokine was produced by either Mock or anti-FLCAR T-cells upon co-culture with K562 (middle); and anti-FLCAR T-cells were the only cells able to elicit secretion of the cytokine IFN-γ, but only with pretreatment of EC-17 (right, where only anti-FLCAR T-cells show a result). FIG. 44C is a chart showing cytokine (TNF-α) production from Mock T-Cells and anti-FLCAR T-cells at quantitatively similar levels in co-culture with the positive control cell line K562-OKT3 (left pair of bars, where Mock is the left bar and anti-FLCAR T-cells is the right bar); no cytokine was produced by either Mock or anti-FLCAR T-cells upon co-culture with K562 (middle); and anti-FLCAR T-cells were the only cells able to elicit secretion of the cytokine TNF-α, but only with pretreatment of EC-17 (right, where only anti-FLCAR T-cells show a result).

FIG. 45A is a chart showing percentage lysis when CD8+ Mock T cells and anti-FLCAR T-cells are co-cultured with negative control K562 cells at a ratio of 30:1, 10:1, 3:1, or 1:1. FIG. 45B is a chart showing percentage lysis when CD8+ Mock T cells and anti-FLCAR T-cells are co-cultured with K562+OKT3 target cells at a ratio of 30:1, 10:1, 3:1, or 1:1. FIG. 45C is a chart showing percentage lysis when CD8+ Mock T cells and anti-FLCAR T-cells are co-cultured with unlabeled MDA-MB-231 cells at a ratio of 30:1, 10:1, 3:1, or 1:1. FIG. 45D is a chart showing percentage lysis when CD8+ Mock T cells and anti-FLCAR T-cells are co-cultured with EC17 labeled MDA-MB-231 cells at a ratio of 30:1, 10:1, 3:1, or 1:1. In all charts, (x) CD8+ Mock T cells; (∘)anti-FLCAR T-cells.

FIG. 46A shows the chemical structure of FITC-folate. FIG. 46B is a chart showing the dose response curve for FITC-folate.

FIG. 47A shows the chemical structure of FITC-DUPA. FIG. 47B is a chart showing the dose response curve for FITC-DUPA.

FIG. 48A shows the chemical structure of FITC-CA9. FIG. 48B is a chart showing the dose response curve for FITC-CA9.

FIG. 49A shows the chemical structure of FITC-NK1R. FIG. 49B is a chart showing the dose response curve for FITC- NK1R.

FIGS. 50A-C show binding (by FACS analysis) of bridges to tumor cells used in an in vivo model and expressing the receptor corresponding to the small molecule ligand of the bridge.

FIGS. 51A-K show EC17 induction of a potent FR-dependent tumor cell killing by CART cells.

FIGS. 52A-C show the correlation of cytolytic activity of CAR T cells with functional FR levels on tumor cells.

FIGS. 53A-D show EC17/FR-dependent CAR T cell activation and exhaustion. Expression of early (CD69), intermediate (CD137) and late (PD1) T cell activation markers detected on CAR-T cells co-cultured (E/T=1:1) with FR+ tumor cell targets pre-loaded without or with EC17 (100 nM, 30-min pulse at 37° C.) are shown. The first two open bars represent CAR-T and Mock transduced T cells only without target cells. Legend top to bottom for FIGS. 53B-D=bars left to right.

FIGS. 54A-C show EC17/FR-dependent CAR T cell exhaustion profiles. Top row: Colored pie charts representing change in the differentiation status of CAR-T cells in culture without target cells on days 0-3. Bottom row: Four sets of colored pie charts representing the differentiation status of CAR-T cells after 3 days of co-culture (E/T=1:2) with FR+(MDA-MB-231, THP-1FRβ, HOS-FRα, KB) and FR-negative (THP1-FG12, HOS-143b) tumor cells preloaded without or with EC17 (0.1 or 10 nM, 30-min pulse at 37° C.).

FIG. 55A shows a fully human CAR construct comprised of anti-FITC scFv (clone E2), a full-length IgG4 spacer (Fc derived hinge-CH2(L235D, N297Q)-CH3), CD28tm transmembrane domain, 4-1BB/CD3ζ cytoplasmic activation domains, and a non-functional truncated cell surface polypeptide of epidermal growth factor receptor (EGFRt). FIG. 55B shows examples of CD4/CD8 T cell phenotyping performed by flow cytometry on an EGFRt-sorted (left pie charts) CAR-T cell preparation and an unsorted “clinical facsimile” (right pie charts). The color keys are as shown.

FIG. 56A: Kd values of 3H-EC17 uptake by 1-R+ target cells after a 2 hour incubation at 37° C. (calculated from the numbers of molecules bound per cell). FIG. 56B: Kd value of ³H-EC17 uptake by E2-CAR-T cells (−24% EGFRt+, ˜95:5 CD8/CD4 ratio) after a 2 hour incubation at room temperate (calculated from total cell-associated radioactivity, DPM).

FIGS. 57A-F show CRS grading scale and experimental conditions applied to sodium fluorescein rescue in HOS-FRα tumor-bearing mice. Shown are changes in T cell-derived cytokines with and without rescue. p-values were calculated by Student's t-test.

FIG. 58 shows functional FR levels on tumor cell measured by a ³H-FA (folic acid)-based binding assay (100 nM, 1 hour at 37° C.).

FIGS. 59A-J show full-range EC17 dose response in specific lysis (%) of 5 FR (folate receptor)+ tumor cell lines co-cultured with EGFRt-sorted CAR-T cells for 24 hours at 1:1 E/T (Effector/Target) ratio; FIG. 59K shows maximum lysis (%) and EC50 values were obtained from the dose-response curves fitted up to 100 nM.

FIGS. 60A-L show the correlation of CAR-T cell activity with FR levels and tumor cells' natural sensitivity. FIGS. 60A-E: Kinetics of specific lysis (%) at varying E/T ratios in FR+(MDA-MB-231, KB, THP1-FRβ, OV90, HOS-FRα) and FR-negative (HOS-143b) cell lines after 16 and 48 hours of co-culture in the presence of 10 nM EC17. FIGS. 60F and 60G: Specific lysis (%) of target cells plotted against a linear scale of E/T ratios. High FR+KB cells demonstrated an early resistance at 16 hours while FR-negative HOS-143b failed to respond. FIG. 60H: Excluding KB cells, a semi-log correlation was established between specific lysis (%) at 16 hours of co-culture and functional FR levels on tumor cells. FIGS. 60I-K: 3D diagrams depicting the relationships between the levels of CAR-T cell derived Th1 cytokines (IFNγ, IL-2 and TNFα) after 44 hours of co-culture at 10 nM EC17 with varying E/T ratios. FIG. 60L: CAR-T cell derived Th1 cytokine levels plotted against FR levels of FR+ target cell in a Log 10 scale.

FIG. 61A shows bar graph to show EC17 loading status of target cells confirmed by flow cytometry and expressed as MFI (EC17 was undetectable on FR-negative cell lines) and (FIG. 61B) target cell apoptosis (%) detected by Annexin V staining after 2 days of co-culture as described in FIGS. 54A-C.

FIGS. 62A-K show pharmacokinetics and tumor uptake of CAR-T cells in vivo. FIG. 62A: Schematic diagram of the experimental layout to show animal collection schedule in relation to CAR-T cell injection and weekly EC17 doses (500 nmol/kg) in MDA-MB-231 tumor-bearing mice. A total of 15 mice received ˜4 8 million of EGFRt-sorted CAR-T cells on day 0 (3 mice had large tumors to begin with). FIGS. 62B-E: Left panel showing measurements of tumor volume and change in body weight; Middle plot showing CAR-T cell expansion in the blood 7 days after a single dose of EC17; Right bar graph showing differentiation profiles of circulating CD4/CD8 CAR-T cell subsets in mice with small and larger tumors. FIGS. 62F and 62G: Measurements of change in tumor volume and body weight. FIGS. 62H and 62I: Top plot showing CAR-T cell kinetics in the blood (solid line) versus that of tumor (dotted line). Bottom bar graph showing changes in CAR-T cell phenotypes in the blood. FIGS. 62J and 62K: Kinetic changes in the surface expression of activation markers (4-1BB, PD1) on tumor-infiltrating CAR-T cells.

FIGS. 63A-K show dietary folate effect on antitumor activity and CRS toxicity. FIGS. 63A-D: Measurements of change in tumor volume and body weight of NSG mice xenografted with large MDA-MB-231 tumors and treated with CAR-T cells (−10 million “clinical facsimile”) plus EC17 SIW at 500 nmol/kg while being maintained on FA-replete or FA-deficient diet throughout the study (n=5). FIG. 63E: Bar graph to show circulating CAR-T cells (human CD3ε+ EGFRt+) measured at the end of study on day 52 (FA-deficient) and day 59 (FA-replete), respectively. FIGS. 63F-H: Representative flow cytometric dot plots to show an absence of circulating CAR-T cells in FA-deficient mice treated with CAR-T cells only (no EC17) and elevated numbers of circulating CAR-T cells in mice treated with CAR-T cells plus EC17 on FA-deficient diet compared to mice treated identically but on FA-replete diet. FIGS. 63I-K: Loss of FR expression was detected in 2 out of 3 MDA-MB-231 tumors collected on day 59 from FA-replete animals and analyzed by flow cytometry (later confirmed by a quantitative radioligand binding assay, data not shown).

### DEFINITIONS

As used herein, “a” or “an” may mean one or more. As used herein, “about” in reference to a numeric value, including, for example, whole numbers, fractions, and percentages, generally refers to a range of numerical values (e.g., +/−5% to 10% of the recited value) that one of ordinary skill in the art would consider equivalent to the recited value (e.g., having the same function or result).

As used herein, the terms “treat,” “treating,” “treated,” or “treatment” refer to both therapeutic treatment and prophylactic or preventative treatment.

As used herein, the terms “ameliorate,” “ameliorating,” “amelioration,” or “ameliorated” in reference to cancer can mean reducing the symptoms of the cancer, reducing the size of a tumor, completely or partially removing the tumor (e.g., a complete or partial response), causing stable disease, preventing progression of the cancer (e.g., progression free survival), or any other effect on the cancer that would be considered by a physician to be a therapeutic, prophylactic, or preventative treatment of the cancer.

As used herein, the terms “administer,” administering,” or “administered” mean all means of introducing the compound, or pharmaceutically acceptable salt thereof, or CAR T cell composition, wherein the CAR T cell composition comprises CAR T cells and wherein the CAR comprises an E2 anti-fluorescein antibody fragment, to the patient, including, but not limited to, oral, intravenous, intramuscular, subcutaneous, and transdermal.

As used herein, the term “off-target toxicity” means organ damage or a reduction in the patient's weight that is unacceptable to the physician treating the patient, or any other effect on the patient that is unacceptable to the physician treating the patient, for example, B cell aplasia, fever, a drop in blood pressure, or pulmonary edema.

As used herein, the terms “transduction” and “transfection” are used equivalently and the terms mean introducing a nucleic acid into a cell by any artificial method, including viral and non-viral methods.

### DETAILED DESCRIPTION OF THE ILLUSTRATIVE EMBODIMENTS

In the various embodiments described herein, a small molecule ligand linked to a targeting moiety by a linker is used as a bridge between a cancer and CAR T cells (i.e, T cells expressing a chimeric antigen receptor), wherein the CAR T cells comprise a genetically engineered CAR directed to the targeting moiety, wherein the CAR comprises an E2 anti-fluorescein antibody fragment. The bridge directs the CAR T cells to the cancer for amelioration of the cancer. In one embodiment, the “small molecule ligand” can be a folate, a CAIX ligand, DUPA, an NK-1R ligand, a ligand of gamma glutamyl transpeptidase, an NKG2D ligand, or a CCK2R ligand, each of which is a small molecule ligand that binds specifically to a cancer cell type (i.e., the receptor for each of these ligands is overexpressed on cancers compared to normal tissues).

The “targeting moiety” linked to the small molecule ligand binds to the recognition region of the genetically engineered CAR expressed by CAR T cells, wherein the CAR comprises an E2 anti-fluorescein antibody fragment. Accordingly, the recognition region of the CAR (e.g., a single chain fragment variable region (scFv) of an E2 anti-fluorescein antibody) is directed to the “targeting moiety.” Thus, the small molecule ligand linked to a targeting moiety by a linker acts as a bridge between the cancer and the CAR T cells, wherein the CAR T cells comprise the genetically engineered CAR, directing the CAR T cells to the cancer for amelioration of the cancer.

The bridge is a small organic molecule so clearance from the bloodstream can be rapidly achieved (e.g., about 20 minutes or less). In one aspect, the CAR T cell response, wherein the CAR T cells comprise a genetically engineered CAR comprising an E2 anti-fluorescein antibody fragment, can be targeted to only those cancer cells expressing a receptor for the small molecule ligand portion of the ‘bridge,’ thereby reducing off-target toxicity to normal tissues. Additionally, this system can be ‘universal’ because one type of CAR T cell construct, wherein the CAR T cell comprises an E2 anti-fluorescein antibody fragment, can be used to target a wide variety of cancers using different ‘bridges’. Illustratively, the targeting moiety recognized by the CAR T cell, wherein the CAR T cell comprises a genetically engineered CAR comprising an E2 anti-fluorescein antibody fragment, may remain constant so that one type of CAR T cell, can be used, while the small molecule ligand that binds to the cancer can be altered to allow targeting of a wide variety of cancers.

In various embodiments described herein, the small molecule ligand linked to a targeting moiety by a linker is referred to as a “compound.”

In various embodiments, the clause “E2 anti-fluorescein antibody fragment” means a CAR comprising a fragment (e.g., an scFv fragment) of the E2 anti-fluorescein antibody. The E2 anti-fluorescein antibody is described, for example, in Vaughan, et al., Nature Biotechnol. Vol. 14(3), pp. 309-314, 1996, incorporated herein by reference. In various embodiments, the CAR can further comprise an IgG4 hinge domain, a CD31 activation domain, and/or a 4-1BB co-stimulation domain, or any other suitable domain such as the EGI-Rt domain. In still other embodiments, the CAR can be encoded by a polynucleotide having at least about 90%, 91%, 92%, 93%, 94%, 95%, 96%, 97%, 98%, 99%, or 99.5% identity to SEQ ID NO:1. In another illustrative embodiment, the CAR can be encoded by a polynucleotide that hybridizes under high stringency conditions to a polynucleotide having SEQ ID NO:1. In still another aspect, the CAR can be encoded by a polynucleotide having SEQ ID NO:1, or by a degenerate variant of SEQ ID NO:1. In other embodiments, the CAR protein sequence can have at least about 90%, 91%, 92%, 93%, 94%, 95%, 96%, 97%, 98%, or 99% identity to SEQ ID NO:2. In yet another embodiment, the CAR protein sequence can have up to about 50 conservative amino acid substitutions. In any of the embodiments described herein, the CAR binds fluorescein.

In one embodiment, a method of treatment of a cancer is provided. The method comprises i) administering to a patient a compound, or a pharmaceutically acceptable salt thereof, wherein the compound comprises a small molecule ligand linked to a targeting moiety by a linker, ii) administering to the patient a first dose of a CAR T cell composition, wherein the CAR T cell composition comprises CAR T cells, wherein the CAR T cells comprise a CAR, and wherein the CAR comprises an E2 anti-fluorescein antibody fragment, and iii) administering to the patient a second dose of the CAR T cell composition wherein the CAR T cell composition comprises CAR T cells, wherein the CAR T cells comprise a CAR, and wherein the CAR comprises an E2 anti-fluorescein antibody fragment.

In another embodiment, a method of treatment of a cancer is provided. The method comprises i) administering to a patient a compound, or a pharmaceutically acceptable salt thereof, wherein the compound comprises a small molecule ligand linked to a targeting moiety by a linker, and ii) administering to the patient a CAR T cell composition comprising CAR T cells, wherein the CAR T cells comprise a CAR, wherein the CAR comprises an E2 anti-fluorescein antibody fragment, and wherein the CAR T cell composition comprises a mixture of the CAR T cells and non-transformed T cells.

In yet another embodiment, a method of treatment of a cancer is provided. The method comprises i) administering to a patient a compound, or a pharmaceutically acceptable salt thereof, wherein the compound comprises a small molecule ligand linked to a targeting moiety by a linker, ii) administering to the patient a CAR T cell composition wherein the CAR T cell composition comprises CAR T cells wherein the CAR T cells comprise a CAR, wherein the CAR comprises an E2 anti-fluorescein antibody fragment, and iii) administering to the patient a folate, a conjugate comprising a folate wherein the conjugate comprising a folate does not comprise a targeting moiety, or a drug that inhibits activation of the CAR T cells.

In another embodiment, a method of treatment of a cancer is provided. The method comprises i) administering to a patient a compound, or a pharmaceutically acceptable salt thereof, wherein the compound comprises a small molecule ligand linked to a targeting moiety by a linker, and wherein the compound, or the pharmaceutically acceptable salt thereof, is administered at a dose of about 10 nmoles/kg of body weight of the patient to about 2500 nmoles/kg of body weight of the patient, and ii) administering to the patient a CAR T cell composition comprising CAR T cells, wherein the CAR T cells comprise a CAR, wherein the CAR comprises an E2 anti-fluorescein antibody fragment, and wherein the CAR T cells are at a dose of about 1 million of the CAR T cells to about 15 million of the CAR T cells.

In still another embodiment, a method of treatment of a cancer is provided. The method comprises i) administering continuously to a patient a compound, or a pharmaceutically acceptable salt thereof, wherein the compound comprises a small molecule ligand linked to a targeting moiety by a linker, ii) administering to the patient a CAR T cell composition comprising CAR T cells wherein the CAR T cells comprise a CAR, wherein the CAR comprises an E2 anti-fluorescein antibody fragment, and iii) ending the continuous administration of the compound, or the pharmaceutically acceptable salt thereof, to inhibit or prevent cytokine release syndrome in the patient.

In another illustrative aspect, a method of treatment of a cancer is provided. The method comprises i) administering to a patient a compound, or a pharmaceutically acceptable salt thereof, wherein the compound comprises a small molecule ligand linked to a targeting moiety by a linker, wherein at least a first dose and a second dose of the compound, or the pharmaceutically acceptable salt thereof, are administered to the patient, wherein the first dose and the second dose are different, wherein the second dose of the compound, or the pharmaceutically acceptable salt thereof, is about 2-fold to about 15000-fold greater in amount than the first dose of the compound, or the pharmaceutically acceptable salt thereof, and ii) administering to the patient a CAR T cell composition comprising CAR T cells wherein the CAR T cells comprise a CAR, wherein the CAR comprises an E2 anti-fluorescein antibody fragment.

In another embodiment, a method of treatment of a cancer is provided. The method comprises i) administering to a patient a compound, or a pharmaceutically acceptable salt thereof, wherein the compound comprises a small molecule ligand linked to a targeting moiety by a linker and wherein the compound, or the pharmaceutically acceptable salt thereof, is administered once weekly to the patient, and ii) administering to the patient a CAR T cell composition comprising CAR T cells wherein the CAR T cells comprise a CAR, wherein the CAR comprises an E2 anti-fluorescein antibody fragment.

In yet another embodiment, a method of treatment of a cancer is provided. The method comprises i) administering to a patient a first dose of a compound, or a pharmaceutically acceptable salt thereof, wherein the compound comprises a small molecule ligand linked to a targeting moiety by a linker, ii) administering to the patient at least a second dose of the compound, or a pharmaceutically acceptable salt thereof, wherein the second dose of the compound, or the pharmaceutically acceptable salt thereof, is at least about 50 percent lower in amount than the first dose of the compound, or the pharmaceutically acceptable salt thereof, and iii) administering to the patient a dose of a CAR T cell composition comprising CAR T cells wherein the CAR T cells comprise a CAR and wherein the CAR comprises an E2 anti-fluorescein antibody fragment.

Several embodiments are described by the following enumerated clauses. Any of the following embodiments in combination with any applicable embodiments described in the Summary section of this patent application, in the Detailed Description of the Illustrative Embodiments section, the Examples section, or the claims of this patent application, are also contemplated.
1. A method of treatment of a cancer, the method comprising
   i) administering to a patient a compound, or a pharmaceutically acceptable salt thereof, wherein the compound comprises a small molecule ligand linked to a targeting moiety by a linker;
   ii) administering to the patient a first dose of a CAR T cell composition comprising CAR T cells wherein the CAR T cells comprise a CAR directed to the targeting moiety and wherein the CAR comprises an E2 anti-fluorescein antibody fragment; and
   iii) administering to the patient a second dose of a CAR T cell composition comprising CAR T cells wherein the CAR T cells comprise the CAR directed to the targeting moiety and wherein the CAR comprises an E2 anti-fluorescein antibody fragment.
2. A method of treatment of a cancer, the method comprising
   i) administering to a patient a compound, or a pharmaceutically acceptable salt thereof, wherein the compound comprises a small molecule ligand linked to a targeting moiety by a linker; and
   ii) administering to the patient a CAR T cell composition wherein the CAR T cell composition comprises CAR T cells, wherein the CAR T cells comprise a CAR directed to the targeting moiety wherein the CAR comprises an E2 anti-fluorescein antibody fragment, and wherein the CAR T cell composition comprises a mixture of the CAR T cells and non-transformed T cells.
3. A method of treatment of a cancer, the method comprising
   i) administering to a patient a compound, or a pharmaceutically acceptable salt thereof, wherein the compound comprises a small molecule ligand linked to a targeting moiety by a linker;
   ii) administering to the patient a CAR T cell composition wherein the CAR T cell composition comprises CAR T cells wherein the CAR T cells comprise a CAR directed to the targeting moiety and wherein the CAR comprises an E2 anti-fluorescein antibody fragment; and
   iii) administering to the patient a folate, a conjugate comprising a folate wherein the conjugate comprising a folate does not comprise a targeting moiety, or an agent that inhibits activation of the CAR T cells.
4. The method of clause 3 wherein step iii comprises administering a folate.
5. The method of any one of clauses 3 or 4 wherein step iii comprises administering folic acid or leucovorin.
6. The method of clause 3 wherein step iii comprises administering the conjugate comprising a folate.
7. The method of clause 6 wherein the conjugate comprising a folate comprises a folate linked to one or more amino acids.
8. The method of clause 7 wherein the conjugate comprising a folate has the formula

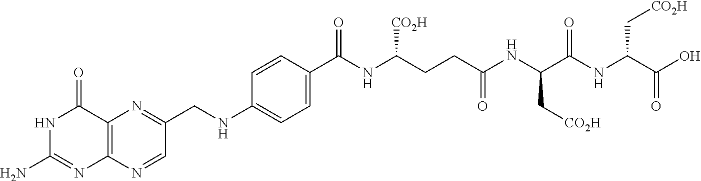 [SMILES: Nc1nc2ncc(CNc3ccc(C(=O)N[C@@H](CCC(=O)N[C@H](CC(=O)O)C(=O)N[C@H](CC(=O)O)C(=O)O)C(=O)O)cc3)nc2c(=O)[nH]1]

9. The method of any one of clauses 3 to 8 wherein the folate has the formula

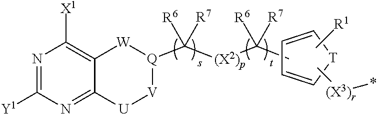

wherein X¹ and Y¹ are each-independently selected from the group consisting of halo, R², OR², SR³, and NR⁴R⁵;
U, V, and W represent divalent moieties each independently selected from the group consisting of (R^{6a})C═, —N═, —(R^{6a})C(R^{7a})—, and —N(R^{4a})—; Q is selected from the group
consisting of C and CH; T is selected from the group consisting of S, O, N, and —C═C—;
X² and X³ are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂-, —N(R^{4a})S(O)₂—, —C(R^{6b})(R^{7b})—, —N(C≡CH)—, —N(CH₂C≡CH)—, C₁-C₁₂ alkylene, and C₁-C₁₂ alkyleneoxy, where Z is oxygen or sulfur;
R¹ is selected-from the group consisting of hydrogen, halo, C₁-C₁₂ alkyl, and C₁-C₁₂ alkoxy;
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group;
R^{6a} and R^{7a} are each independently selected from the group consisting of hydrogen, halo, C¹-C¹² alkyl, and C¹-C¹² alkoxy; or R⁶ and R⁷ are taken together to form a carbonyl group;
p, r, s, and t are each independently either 0 or 1; and
* represents an optional covalent bond to the rest of the conjugate.
10. The method of clause 3 wherein the agent that inhibits activation of the CAR T cells is administered and is selected from the group consisting of a lymphocyte-specific protein tyrosine kinase inhibitor, a PI3 kinase inhibitor, an inhibitor of an IL-2 inducible T cell kinase, a JAK inhibitor, a BTK inhibitor, EC2319, and an agent that blocks CAR T cell binding to the compound, or the pharmaceutically acceptable salt thereof, but does not bind to the cancer.
11. The method of clause 10 wherein the agent that inhibits activation of the CAR T cells is administered and the agent is a lymphocyte-specific protein tyrosine kinase inhibitor.
12. The method of clause 11 wherein the lymphocyte-specific protein tyrosine kinase inhibitor is Dasatinib.
13. The method of clause 10 wherein the agent that inhibits activation of the CAR T cells is administered and the agent is a PI3 kinase inhibitor.
14. The method of clause 13 wherein the PI3 kinase inhibitor is GDC0980.
15. The method of clause 10 wherein the agent that inhibits activation of the CAR T cells is administered and the agent is an IL-2 inducible T cell kinase inhibitor.
16. The method of clause 15 wherein the IL-2 inducible T cell kinase inhibitor is BMS-509744.
17. The method of clause 10 wherein the agent that inhibits activation of the CAR T cells is administered and is an agent that blocks CAR T cell binding to the compound, or the pharmaceutically acceptable salt thereof, but does not bind to the cancer.
18. The method of clause 17 wherein the agent is fluoresceinamine, FITC, or sodium fluorescein.
19. The method of clause 18 wherein the agent is FITC.
20. The method of clause 18 wherein the agent is sodium fluorescein.
21. The method of any one of clauses 17 to 20 wherein the agent that inhibits activation of the CAR T cells is administered at a dose of about 0.01 to about 300 umoles/kg of body weight of the patient.
22. The method of any one of clauses 17 to 20 wherein the agent that inhibits activation of the CAR T cells is administered at a dose of about 0.06 to about 100 umoles/kg of body weight of the patient.
23. The method of any one of clauses 17 to 20 wherein the agent that inhibits activation of the CAR T cells is administered at a dose of about 0.06 to about 90 umoles/kg of body weight of the patient.
24. The method of any one of clauses 17 to 20 wherein the agent that inhibits activation of the CAR T cells is administered at a dose of about 0.06 to about 80 umoles/kg of body weight of the patient.
25. The method of any one of clauses 17 to 20 wherein the agent that inhibits activation of the CAR T cells is administered at a dose of about 0.06 to about 70 umoles/kg of body weight of the patient.
26. The method of any one of clauses 17 to 20 wherein the agent that inhibits activation of the CAR T cells is administered at a dose of about 0.06 to about 60 umoles/kg of body weight of the patient.
27. The method of any one of clauses 17 to 20 wherein the agent that inhibits activation of the CAR T cells is administered at a dose of about 0.06 to about 50 umoles/kg of body weight of the patient.
28. The method of any one of clauses 17 to 20 wherein the agent that inhibits activation of the CAR T cells is administered at a dose of about 0.06 to about 40 umoles/kg of body weight of the patient.
29. The method of any one of clauses 17 to 20 wherein the agent that inhibits activation of the CAR T cells is administered at a dose of about 0.06 to about 30 umoles/kg of body weight of the patient.
30. The method of any one of clauses 17 to 20 wherein the agent that inhibits activation of the CART cells is administered at a dose of about 0.06 to about 20 umoles/kg of body weight of the patient.
31. The method of any one of clauses 17 to 20 wherein the agent that inhibits activation of the CAR T cells is administered at a dose of about 0.06 to about 10 umoles/kg of body weight of the patient.
32. The method of any one of clauses 17 to 20 wherein the agent that inhibits activation of the CAR T cells is administered at a dose of about 0.06 to about 8 umoles/kg of body weight of the patient.
33. The method of any one of clauses 17 to 20 wherein the agent that inhibits activation of the CAR T cells is administered at a dose of about 0.06 to about 6 umoles/kg of body weight of the patient.
34. The method of any one of clauses 3 to 33 wherein more than one dose is administered to the patient of the folate, the conjugate comprising a folate wherein the conjugate comprising a folate does not comprise a targeting moiety, or the agent that inhibits activation of the CAR T cells.
35. The method of any one of clauses 3 to 34 wherein the folate, the conjugate comprising a folate wherein the conjugate comprising a folate does not comprise a targeting moiety, or the agent that inhibits activation of the CAR T cells is administered to the patient before and/or after the compound, or the pharmaceutically acceptable salt thereof.
36. The method of any one of clauses 3 to 35 wherein administration of the folate, the conjugate comprising a folate wherein the conjugate comprising a folate does not comprise a targeting moiety, or the agent that inhibits activation of the CAR T cells causes reduction in cytokine levels in the patient.
37. The method of clause 36 wherein the reduction in cytokine levels occurs by about 3 hours after administration to the patient of the folate, the conjugate comprising a folate wherein the conjugate comprising a folate does not comprise a targeting moiety, or the agent that inhibits activation of the CAR T cells.
38. The method of clause 36 wherein the reduction in cytokine levels occurs by about 6 hours after administration to the patient of the folate, the conjugate comprising a folate wherein the conjugate comprising a folate does not comprise a targeting moiety, or the agent that inhibits activation of the CAR T cells.
39. The method of any one of clauses 36 to 38 wherein the reduction in cytokine levels is a reduction to about the cytokine levels in an untreated patient.
40. The method of any one of clauses 3 to 39 wherein the compound, or the pharmaceutically acceptable salt thereof, is administered before and subsequent to administration of the folate, the conjugate comprising a folate wherein the conjugate comprising a folate does not comprise a targeting moiety, or the agent that inhibits activation of the CAR T cells.
41. The method of any one of clauses 3 to 40 wherein CAR T cell number increases in the blood of the patient after administration of the folate, the conjugate comprising a folate wherein the conjugate comprising a folate does not comprise a targeting moiety, or the agent that inhibits activation of the CAR T cells, even though cytokine levels in the patient are reduced.
42. The method of any one of clauses 3 to 41 wherein CAR T cell activation is enhanced or maintained, relative to a patient not treated with a rescue agent, after administration of the folate, the conjugate comprising a folate wherein the conjugate comprising a folate does not comprise a targeting moiety, or the agent that inhibits activation of the CAR T cells, even though cytokine levels in the treated patient are reduced.
43. The method of any one of clauses 3 to 42 wherein the cancer comprises a tumor and tumor size in the patient is not increased when the folate, the conjugate comprising a folate wherein the conjugate comprising a folate does not comprise a targeting moiety, or the agent that inhibits activation of the CAR T cells is administered to the patient.
44. The method of clause 43 wherein a complete response for the tumor is obtained.
45. The method of any one of clauses 3 to 44 wherein the folate, the conjugate comprising a folate wherein the conjugate comprising a folate does not comprise a targeting moiety, or the agent that inhibits activation of the CAR T cells the agent that inhibits activation of the CAR T cells is administered to the patient when the CRS grade reaches 1, 2, 3, or 4.
46. The method of clause 45 wherein the agent that inhibits activation of the CAR T cells is administered to the patient when the CRS grade reaches 3 or 4.
47. The method of any one of clauses 3 to 46 wherein lung edema is reduced.
48. The method of any one of clauses 1 to 47 wherein the compound, or the pharmaceutically acceptable salt thereof, is administered at a dose of about 10 nmoles/kg of body weight of the patient to about 2500 nmoles/kg of body weight of the patient; and the CAR T cells are at a dose of about 1 million of the CAR T cells to about 15 million of the CAR T cells.
49. The method of clause 48 wherein the compound, or the pharmaceutically acceptable salt thereof, is administered at a dose of about 10 nmoles/kg to about 100 nmoles/kg of body weight of the patient.
50. The method of any one of clauses 48 to 49 wherein the compound, or the pharmaceutically acceptable salt thereof, is administered at a dose of about 10 nmoles/kg to about 50 nmoles/kg of body weight of the patient.
51. The method of any one of clauses 48 to 50 wherein the compound, or the pharmaceutically acceptable salt thereof, is administered at a dose of about 10 nmoles/kg to about 20 nmoles/kg of body weight of the patient.
52. The method of clause 48 wherein the compound, or the pharmaceutically acceptable salt thereof, is administered at a dose of about 10 nmoles/kg to about 600 nmoles/kg of body weight of the patient.
53. The method of clause 48 wherein the compound, or the pharmaceutically acceptable salt thereof, is administered at a dose of about 200 nmoles/kg to about 600 nmoles/kg of body weight of the patient.
54. The method of clause 48 wherein the compound, or the pharmaceutically acceptable salt thereof, is administered at a dose of about 400 nmoles/kg to about 600 nmoles/kg of body weight of the patient.
55. The method of any one of clauses 48 to 54 wherein the CAR T cells are at a dose of about 1 million of the CAR T cells to about 12.5 million of the CAR T cells.
56. The method of any one of clauses 48 to 55 wherein the CAR T cells are at a dose of about 1 million of the CAR T cells to about 7 million of the CAR T cells.
57. The method of any one of clauses 48 to 56 wherein the CAR T cells are at a dose of about 1 million of the CAR T cells to about 5 million of the CAR T cells.
58. The method of any one of clauses 48 to 57 wherein the CAR T cells are at a dose of about 2 million of the CAR T cells to about 5 million of the CAR T cells.
59. The method of any one of clauses 1 to 58 further comprising the step of ending continuous administration of the compound, or the pharmaceutically acceptable salt thereof, to inhibit or prevent cytokine release syndrome in the patient.
60. The method of any one of clauses 1 to 59 wherein the compound, or the pharmaceutically acceptable salt thereof, is administered continuously for at least one hour to the patient.
61. The method of any one of clauses 1 to 59 wherein the compound, or the pharmaceutically acceptable salt thereof, is administered continuously for at least four hours to the patient.
62. The method of any one of clauses 1 to 59 wherein the compound, or the pharmaceutically acceptable salt thereof, is administered continuously for at least six hours to the patient.
63. The method of any one of clauses 1 to 62 wherein the compound, or the pharmaceutically acceptable salt thereof, is administered to the patient every other day.
64. The method of any one of clauses 1 to 62 wherein the compound, or the pharmaceutically acceptable salt thereof, is administered to the patient three times weekly.
65. The method of any one of clauses 1 to 62 wherein the compound, or the pharmaceutically acceptable salt thereof, is administered to the patient two times weekly.
66. The method of any one of clauses 1 to 62 wherein the compound, or the pharmaceutically acceptable salt thereof, is administered to the patient once weekly.
67. The method of any one of clauses 1 to 62 wherein compound, or the pharmaceutically acceptable salt thereof, is administered to the patient until an unacceptable loss of body weight of the patient, a fever, a drop in blood pressure, or pulmonary edema occurs.
68. A method of treatment of a cancer, the method comprising
   i) administering to a patient a compound, or a pharmaceutically acceptable salt thereof, wherein the compound comprises a small molecule ligand linked to a targeting moiety by a linker, wherein at least a first dose and a second dose of the compound, or the pharmaceutically acceptable salt thereof, are administered to the patient, wherein the first dose and the second dose are different, wherein the second dose of the compound, or the pharmaceutically acceptable salt thereof, is about 2-fold to about 15000-fold greater in amount than the first dose of the compound, or the pharmaceutically acceptable salt thereof; and
   ii) administering to the patient a CAR T cell composition comprising CAR T cells wherein the CAR T cells comprise a CAR directed to the targeting moiety and wherein the CAR comprises an E2 anti-fluorescein antibody fragment.
69. The method of clause 68 wherein at least a first dose, a second dose, and a third dose of the compound, or the pharmaceutically acceptable salt thereof, are administered to the patient, wherein the first dose, the second dose, and the third dose are different, wherein the second dose of the compound, or the pharmaceutically acceptable salt thereof, is about 2-fold to about 750-fold greater in amount than the first dose of the compound, or the pharmaceutically acceptable salt thereof, and wherein the third dose of the compound, or the pharmaceutically acceptable salt thereof, is about 800-fold to about 10000-fold greater in amount than the first dose of the compound, or the pharmaceutically acceptable salt thereof.
70. The method of clause 68 wherein at least a first dose, a second dose, a third dose, and a fourth dose of the compound, or the pharmaceutically acceptable salt thereof, are administered to the patient, wherein the first dose, the second dose, the third dose, and the fourth dose are different, wherein the second dose of the compound, or the pharmaceutically acceptable salt thereof, is about 2-fold to about 750-fold greater in amount than the first dose of the compound, or the pharmaceutically acceptable salt thereof, wherein the third dose of the compound, or the pharmaceutically acceptable salt thereof, is about 800-fold to about 7500-fold greater in amount than the first dose of the compound, or the pharmaceutically acceptable salt thereof, and wherein the fourth dose of the compound, or the pharmaceutically acceptable salt thereof, is about 8000 to about 15000-fold greater in amount than the first dose of the compound, or the pharmaceutically acceptable salt thereof.
71. The method of clause 70 wherein the second dose of the compound, or the pharmaceutically acceptable salt thereof, is about 100-fold greater in amount than the first dose of the compound, or the pharmaceutically acceptable salt thereof, wherein the third dose of the compound, or the pharmaceutically acceptable salt thereof, is about 1000-fold greater in amount than the first dose of the compound, or the pharmaceutically acceptable salt thereof, and wherein the fourth dose of the compound, or the pharmaceutically acceptable salt thereof, is about 10000-fold greater in amount than the first dose of the compound, or the pharmaceutically acceptable salt thereof.
72. The method of clause 68 wherein at least a first dose and a second dose of the compound, or the pharmaceutically acceptable salt thereof, are administered to the patient, wherein the first dose and the second dose are different, wherein the second dose of the compound, or the pharmaceutically acceptable salt thereof, is about 2-fold to about 15000-fold greater in amount than the first dose of the compound, or the pharmaceutically acceptable salt thereof.
73. A method of treatment of a cancer, the method comprising
   i) administering to a patient a first dose of a compound, or a pharmaceutically acceptable salt thereof, wherein the compound comprises a small molecule ligand linked to a targeting moiety by a linker;
   ii) administering to the patient at least a second dose of the compound, or a pharmaceutically acceptable salt thereof, wherein the second dose of the compound, or the pharmaceutically acceptable salt thereof, is at least about 50 percent lower in amount than the first dose of the compound, or the pharmaceutically acceptable salt thereof; and
   iii) administering to the patient a dose of a CAR T cell composition comprising CAR T cells wherein the CAR T cells comprise a CAR directed to the targeting moiety and wherein the CAR comprises an E2 anti-fluorescein antibody fragment.
74. The method of clause 73 wherein the second dose of the compound, or the pharmaceutically acceptable salt thereof, is at least about 60 percent lower in amount than the first dose of the compound, or the pharmaceutically acceptable salt thereof.
75. The method of clause 73 wherein the second dose of the compound, or the pharmaceutically acceptable salt thereof, is at least about 70 percent lower in amount than the first dose of the compound, or the pharmaceutically acceptable salt thereof.
76. The method of clause 73 wherein the second dose of the compound, or the pharmaceutically acceptable salt thereof, is at least about 80 percent lower in amount than the first dose of the compound, or the pharmaceutically acceptable salt thereof.
77. The method of clause 73 wherein the second dose of the compound, or the pharmaceutically acceptable salt thereof, is at least about 90 percent lower in amount than the first dose of the compound, or the pharmaceutically acceptable salt thereof.
78. The method of clause 73 wherein the second dose of the compound, or the pharmaceutically acceptable salt thereof, is at least about 95 percent lower in amount than the first dose of the compound, or the pharmaceutically acceptable salt thereof.
79. The method of clause 73 wherein the second dose of the compound, or the pharmaceutically acceptable salt thereof, is at least about 96 percent lower in amount than the first dose of the compound, or the pharmaceutically acceptable salt thereof.
80. The method of clause 73 wherein the second dose of the compound, or the pharmaceutically acceptable salt thereof, is at least about 97 percent lower in amount than the first dose of the compound, or the pharmaceutically acceptable salt thereof.
81. The method of clause 73 wherein the second dose of the compound, or the pharmaceutically acceptable salt thereof, is at least about 98 percent lower in amount than the first dose of the compound, or the pharmaceutically acceptable salt thereof.
82. The method of clause 73 wherein the second dose of the compound, or the pharmaceutically acceptable salt thereof, is at least about 99 percent lower in amount than the first dose of the compound, or the pharmaceutically acceptable salt thereof.
83. The method of clause 73 wherein the second dose of the compound, or the pharmaceutically acceptable salt thereof, is at least about 99.5 percent lower in amount than the first dose of the compound, or the pharmaceutically acceptable salt thereof.
84. The method of any one of clauses 73 to 83 wherein the first dose of the compound, or the pharmaceutically acceptable salt thereof, is about 100 nmoles/kg to about 1000 nmoles/kg of body weight of the patient.
85. The method of any one of clauses 73 to 83 wherein the first dose of the compound, or the pharmaceutically acceptable salt thereof, is about 100 nmoles/kg to about 900 nmoles/kg of body weight of the patient.
86. The method of any one of clauses 73 to 83 wherein the first dose of the compound, or the pharmaceutically acceptable salt thereof, is about 100 nmoles/kg to about 800 nmoles/kg of body weight of the patient.
87. The method of any one of clauses 73 to 83 wherein the first dose of the compound, or the pharmaceutically acceptable salt thereof, is about 100 nmoles/kg to about 700 nmoles/kg of body weight of the patient.
88. The method of any one of clauses 73 to 83 wherein the first dose of the compound, or the pharmaceutically acceptable salt thereof, is about 100 nmoles/kg to about 600 nmoles/kg of body weight of the patient.
89. The method of any one of clauses 73 to 83 wherein the first dose of the compound, or the pharmaceutically acceptable salt thereof, is about 200 nmoles/kg to about 600 nmoles/kg of body weight of the patient.
90. The method of any one of clauses 73 to 83 wherein the first dose of the compound, or the pharmaceutically acceptable salt thereof, is about 400 nmoles/kg to about 600 nmoles/kg of body weight of the patient.
91. The method of any one of clauses 73 to 83 wherein the first dose of the compound, or the pharmaceutically acceptable salt thereof, is about 500 nmoles/kg of body weight of the patient.
92. The method of clause 84 wherein the second dose of the compound, or the pharmaceutically acceptable salt thereof, is about 0.5 nmoles/kg to about 500 nmoles/kg of body weight of the patient.
93. The method of clause 85 wherein the second dose of the compound, or the pharmaceutically acceptable salt thereof, is about 0.5 nmoles/kg to about 450 nmoles/kg of body weight of the patient.
94. The method of clause 86 wherein the second dose of the compound, or the pharmaceutically acceptable salt thereof, is about 0.5 nmoles/kg to about 400 nmoles/kg of body weight of the patient.
95. The method of clause 87 wherein the second dose of the compound, or the pharmaceutically acceptable salt thereof, is about 0.5 nmoles/kg to about 350 nmoles/kg of body weight of the patient 96. The method of clause 88 wherein the second dose of the compound, or the pharmaceutically acceptable salt thereof, is about 0.5 nmoles/kg to about 300 nmoles/kg of body weight of the patient.
97. The method of clause 89 wherein the second dose of the compound, or the pharmaceutically acceptable salt thereof, is about 1 nmole/kg to about 300 nmoles/kg of body weight of the patient.
98. The method of clause 90 wherein the second dose of the compound, or the pharmaceutically acceptable salt thereof, is about 2 nmoles/kg to about 300 nmoles/kg of body weight of the patient.
99. The method of clause 91 wherein the second dose of the compound, or the pharmaceutically acceptable salt thereof, is about 2 nmoles/kg to about 250 nmoles/kg of body weight of the patient.
100. The method of any one of clauses 92 to 99 wherein the second dose of the compound, or the pharmaceutically acceptable salt thereof, is about 5 nmoles/kg to about 40 nmoles/kg of body weight of the patient.
101. The method of any one of clauses 92 to 99 wherein the second dose of the compound, or the pharmaceutically acceptable salt thereof, is about 40 nmoles/kg to about 150 nmoles/kg of body weight of the patient.
102. The method of any one of clauses 73 to 101 further comprising administering a third dose of the compound, or the pharmaceutically acceptable salt thereof, wherein the third dose of the compound, or the pharmaceutically acceptable salt thereof, is the same as the second dose of the compound, or the pharmaceutically acceptable salt thereof.
103. The method of clause 102 further comprising administering a fourth dose of the compound, or the pharmaceutically acceptable salt thereof, wherein the fourth dose of the compound, or the pharmaceutically acceptable salt thereof, is the same as the second dose, or the pharmaceutically acceptable salt thereof, and the third dose of the compound, or the pharmaceutically acceptable salt thereof.
104. The method of any one of clauses 73 to 103 wherein the dose(s) of the compound, or the pharmaceutically acceptable salt thereof, administered after the first dose of the compound, or the pharmaceutically acceptable salt thereof, maintain inhibition of growth of the cancer relative to the first dose of the compound, or the pharmaceutically acceptable salt thereof.
105. The method of any one of clauses 73 to 104 wherein the CAR T cells are administered at a dose of about 1 million of the CAR T cells to about 40 million of the CAR T cells.
106. The method of any one of clauses 73 to 105 wherein the dose(s) of the compound, or the pharmaceutically acceptable salt thereof, administered after the first dose of the compound, or the pharmaceutically acceptable salt thereof, are administered once weekly.
107. The method of any one of clauses 73 to 105 wherein the dose(s) of the compound, or the pharmaceutically acceptable salt thereof, are administered twice weekly.
108. The method of any one of clauses 1 to 107 wherein the CAR further comprises an IgG4 hinge domain, a CD3ζ activation domain, and a 4-1BB co-stimulation domain.
109. The method of any one of clauses 1 to 108 wherein the E2 anti-fluorescein antibody fragment is an scFv fragment.
110. The method of any one of clauses 1 to 109 wherein the CAR protein sequence has at least about 90% identity to SEQ ID NO:2.
111. The method of any one of clauses 1 to 109 wherein the CAR protein sequence has at least about 95% identity to SEQ ID NO:2.
112. The method of any one of clauses 1 to 109 wherein the CAR protein sequence has at least about 98% identity to SEQ ID NO:2.
113. The method of any one of clauses 1 to 112 wherein the CAR binds fluorescein.
114. The method of any one of clauses 1 to 109 wherein the CAR protein sequence has up to about 50 conservative amino acid substitutions and wherein the CAR binds fluorescein.
115. The method of any one of clauses 1 to 109 wherein the CAR is encoded by a polynucleotide having at least about 90% identity to SEQ ID NO:1.
116. The method of any one of clauses 1 to 109 wherein the CAR is encoded by a polynucleotide having at least about 95% identity to SEQ ID NO:1.
117. The method of any one of clauses 1 to 109 wherein the CAR is encoded by a polynucleotide having at least about 98% identity to SEQ ID NO:1.
118. The method of any one of clauses 115 to 117 wherein the CAR binds fluorescein.
119. The method of any one of clauses 1 to 118 wherein the CAR is encoded by a polynucleotide that hybridizes under high stringency conditions to a polynucleotide having SEQ ID NO:1 and wherein the CAR binds fluorescein.
120. The method of any one of clauses 1 to 119 wherein the CAR is encoded by a polynucleotide having SEQ ID NO: 1, or by a degenerate variant of SEQ ID NO:1.
121. The method of any one of clauses 1 to 120 wherein the ligand is selected from the group consisting of a folate, DUPA, an NK-1R ligand, a CAIX ligand, a ligand of gamma glutamyl transpeptidase, an NKG2D ligand, and a CCK2R ligand.
122. The method of any one of clauses 1 to 121 wherein the targeting moiety is fluorescein, or a pharmaceutically acceptable salt thereof.
123. The method of any one of clauses 1 to 122 wherein the linker comprises polyethylene glycol (PEG), polyproline, a hydrophilic amino acid, a sugar, an unnatural peptidoglycan, a polyvinylpyrrolidone, pluronic F-127, or a combination thereof.
124. The method of any one of clauses 1 to 123 wherein the linker comprises PEG.
125. The method of any one of clauses 1 to 124 wherein the compound, or the pharmaceutically acceptable salt thereof, has the formula

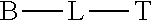

wherein B represents the small molecule ligand, L represents the linker, and T represents the targeting moiety, and wherein L comprises a structure having the formula

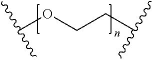

wherein n is an integer from 0 to 200.
126. The method of any one of clauses 1 to 125 wherein the cancer is selected from the group consisting of lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, osteosarcoma, cancer of the urethra, prostate cancer, chronic leukemia, acute leukemia, acute myelocytic leukemia, lymphocytic lymphoma, myeloid leukemia, myclomonocytic leukemia, hairy cell leukemia, myelomonocytic leukemia, hairy cell leukemia, pleural mesothelioma, cancer of the bladder, Burkitt's lymphoma, cancer of the ureter, cancer of the kidney, renal cell carcinoma, carcinoma of the renal pelvis, neoplasms of the central nervous system (CNS), primary CNS lymphoma, spinal axis tumors, brain stem glioma, pituitary adenoma, and adenocarcinoma of the gastroesophageal junction.
127. The method of any one of clauses 1 to 126 wherein the cancer is a folate receptor expressing cancer.
128. The method of any one of clauses 1 to 127 wherein the cancer is an endometrial cancer.
129. The method of any one of clauses 1 to 127 wherein the cancer is a non-small cell lung cancer.
130. The method of any one of clauses 1 to 127 wherein the cancer is an ovarian cancer.
131. The method of any one of clauses 1 to 127 wherein the cancer is a triple negative breast cancer.
132. The method of any one of clauses 1 to 127 wherein the cancer is acute myelocytic leukemia.
133. The method of clause 132 wherein the cancer expresses the folate receptor-r3.
134. The method of any one of clauses 1 to 133 wherein multiple doses of the CAR T cell composition are administered.
135. The method of any one of clauses 1 to 134 wherein at least two doses of the CAR T cell composition are administered.
136. The method of any one of clauses 1 to 135 wherein the patient is imaged prior to administration of the compound, or the pharmaceutically acceptable salt thereof, or prior to administration of the CAR T cell composition.
137. The method of any one of clauses 1 to 136 wherein the compound, or the pharmaceutically acceptable salt thereof, is not an antibody, and does not comprise a fragment of an antibody.
138. The method of any one of clauses 1 to 137 wherein the targeting moiety does not comprise a peptide epitope.
139. The method of any one of clauses 1 to 138 wherein cytokine release resulting in off-target toxicity in the patient does not occur and wherein CAR T cell toxicity to the cancer occurs.
140. The method of any one of clauses 1 to 138 wherein off-target tissue toxicity does not occur in the patient and wherein CAR T cell toxicity to the cancer occurs.
141. The method of any one of clauses 1 to 138 wherein the cancer comprises a tumor, wherein tumor size is reduced in the patient, and wherein off-target toxicity does not occur.
142. The method of any one of clauses 1 to 141 wherein CRS is reduced or prevented and the method results in a decrease in tumor volume in the patient.
143. The method of any one of clauses 1 to 142 wherein body weight loss due to CRS is reduced or prevented.
144. The method of any one of clauses 59 to 67 further comprising re-administering the compound, or the pharmaceutically acceptable salt thereof, to the patient.
145. The method of clause 144 wherein the subsequent administration of the compound, or the pharmaceutically acceptable salt thereof, causes CAR T cell activation and an increase in cytokine levels in the patient.
146. The method of any one of clauses 1 to 145 wherein the cancer comprises a tumor and wherein a complete response for the tumor is obtained.
147. The method of any one of clauses 1 to 146 wherein the CAR T cells have a central memory/effector memory phenotype.
148. The method of any one of clauses 1 to 147 wherein the CD8:CD4 ratio of the CAR T cells is about 1:1.

Thus, in one embodiment, a method of treatment of a cancer is provided. The method comprises i) administering to a patient a compound, or a pharmaceutically acceptable salt thereof, wherein the compound comprises a small molecule ligand linked to a targeting moiety by a linker, ii) administering to the patient a first dose of a CAR T cell composition, wherein the CAR T cell composition comprises CAR T cells, wherein the CAR T cells comprise a CAR, and wherein the CAR comprises an E2 anti-fluorescein antibody fragment, and iii) administering to the patient a second dose of the CAR T cell composition wherein the CAR T cell composition comprises CAR T cells, wherein the CAR T cells comprise a CAR, and wherein the CAR comprises an E2 anti-fluorescein antibody fragment.

In another embodiment, a method of treatment of a cancer is provided. The method comprises i) administering to a patient a compound, or a pharmaceutically acceptable salt thereof, wherein the compound comprises a small molecule ligand linked to a targeting moiety by a linker, and ii) administering to the patient a CAR T cell composition comprising CAR T cells, wherein the CAR T cells comprise a CAR, wherein the CAR comprises an E2 anti-fluorescein antibody fragment, and wherein the CAR T cell composition comprises a mixture of the CAR T cells and non-transformed T cells.

In yet another embodiment, a method of treatment of a cancer is provided. The method comprises i) administering to a patient a compound, or a pharmaceutically acceptable salt thereof, wherein the compound comprises a small molecule ligand linked to a targeting moiety by a linker, ii) administering to the patient a CAR T cell composition wherein the CAR T cell composition comprises CAR T cells wherein the CAR T cells comprise a CAR, wherein the CAR comprises an E2 anti-fluorescein antibody fragment, and iii) administering to the patient a folate, a conjugate comprising a folate wherein the conjugate comprising a folate does not comprise a targeting moiety, or a drug that inhibits activation of the CAR T cells.

In another embodiment, a method of treatment of a cancer is provided. The method comprises i) administering to a patient a compound, or a pharmaceutically acceptable salt thereof, wherein the compound comprises a small molecule ligand linked to a targeting moiety by a linker, and wherein the compound, or the pharmaceutically acceptable salt thereof, is administered at a dose of about 10 nmoles/kg of body weight of the patient to about 2500 nmoles/kg of body weight of the patient, and ii) administering to the patient a CAR T cell composition comprising CAR T cells, wherein the CAR T cells comprise a CAR, wherein the CAR comprises an E2 anti-fluorescein antibody fragment, and wherein the CAR T cells are at a dose of about 1 million of the CAR T cells to about 15 million of the CAR T cells.

In still another embodiment, a method of treatment of a cancer is provided. The method comprises i) administering continuously to a patient a compound, or a pharmaceutically acceptable salt thereof, wherein the compound comprises a small molecule ligand linked to a targeting moiety by a linker, ii) administering to the patient a CAR T cell composition comprising CAR T cells wherein the CAR T cells comprise a CAR, wherein the CAR comprises an E2 anti-fluorescein antibody fragment, and iii) ending the continuous administration of the compound, or the pharmaceutically acceptable salt thereof, to inhibit or prevent cytokine release syndrome in the patient.

In another illustrative aspect, a method of treatment of a cancer is provided. The method comprises i) administering to a patient a compound, or a pharmaceutically acceptable salt thereof, wherein the compound comprises a small molecule ligand linked to a targeting moiety by a linker, wherein at least a first dose and a second dose of the compound, or the pharmaceutically acceptable salt thereof, are administered to the patient, wherein the first dose and the second dose are different, wherein the second dose of the compound, or the pharmaceutically acceptable salt thereof, is about 2-fold to about 15000-fold greater in amount than the first dose of the compound, or the pharmaceutically acceptable salt thereof, and ii) administering to the patient a CAR T cell composition comprising CAR T cells wherein the CAR T cells comprise a CAR, wherein the CAR comprises an E2 anti-fluorescein antibody fragment.

In another embodiment, a method of treatment of a cancer is provided. The method comprises i) administering to a patient a compound, or a pharmaceutically acceptable salt thereof, wherein the compound comprises a small molecule ligand linked to a targeting moiety by a linker and wherein the compound, or the pharmaceutically acceptable salt thereof, is administered once weekly to the patient, and ii) administering to the patient a CAR T cell composition comprising CAR T cells wherein the CAR T cells comprise a CAR, wherein the CAR comprises an E2 anti-fluorescein antibody fragment.

In yet another embodiment, a method of treatment of a cancer is provided. The method comprises i) administering to a patient a first dose of a compound, or a pharmaceutically acceptable salt thereof, wherein the compound comprises a small molecule ligand linked to a targeting moiety by a linker, ii) administering to the patient at least a second dose of the compound, or a pharmaceutically acceptable salt thereof, wherein the second dose of the compound, or the pharmaceutically acceptable salt thereof, is at least about 50 percent lower in amount than the first dose of the compound, or the pharmaceutically acceptable salt thereof, and iii) administering to the patient a dose of a CAR T cell composition comprising CAR T cells wherein the CAR T cells comprise a CAR and wherein the CAR comprises an E2 anti-fluorescein antibody fragment.

Accordingly, various embodiments are provided in the preceding paragraphs and in the clause list above, and all applicable embodiments described in this “Detailed Description of Illustrative Embodiments,” the Summary section, the Examples, and the claims apply to the these embodiments.

As described herein, a “patient” can be a human or, in the case of veterinary applications, the patient can be a laboratory, an agricultural, a domestic, or a wild animal. In various aspects, the patient can be a laboratory animal such as a rodent (e.g., mouse, rat, hamster, etc.), a rabbit, a monkey, a chimpanzee, a domestic animal such as a dog, a cat, or a rabbit, an agricultural animal such as a cow, a horse, a pig, a sheep, a goat, or a wild animal in captivity such as a bear, a panda, a lion, a tiger, a leopard, an elephant, a zebra, a giraffe, a gorilla, a dolphin, or a whale.

In various embodiments, the cancer to be treated can be selected from a carcinoma, a sarcoma, an osteosarcoma, a lymphoma, a melanoma, a mesothelioma, a nasopharyngeal carcinoma, a leukemia, an adenocarcinoma, or a myeloma. In other embodiments, the cancer may be lung cancer, bone cancer, pancreatic cancer, skin cancer, cancer of the head, cancer of the neck, cutaneous melanoma, intraocular melanoma uterine cancer, ovarian cancer, endometrial cancer, rectal cancer, stomach cancer, colon cancer, breast cancer, triple negative breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, non-small cell lung cancer, cancer of the adrenal gland, sarcoma of soft tissue, osteosarcoma, cancer of the urethra, prostate cancer, chronic leukemia, acute leukemia, including acute myelocytic leukemia, a lymphocytic lymphoma, myeloid leukemia, myelomonocytic leukemia, hairy cell leukemia, pleural mesothelioma, cancer of the bladder, Burkitt's lymphoma, cancer of the ureter, cancer of the kidney, renal cell carcinoma, carcinoma of the renal pelvis, a neoplasm of the central nervous system (CNS), primary CNS lymphoma, a spinal axis tumor, a brain stem glioma, a pituitary adenoma, and an adenocarcinoma of the gastroesophageal junction.

In some aspects of these embodiments, the cancer is a folate receptor expressing cancer. In another embodiment, the cancer is a folate receptor α-expressing cancer. In yet another embodiment, the cancer is a folate receptor β-expressing cancer. In some aspects of these embodiments, the cancer is an endometrial cancer, a non-small cell lung cancer, an ovarian cancer, or a triple-negative breast cancer. In another embodiment, the cancer being treated is a tumor. In another embodiment, the cancer is malignant. In another embodiment, the cancer is acute myelocytic leukemia. In yet another embodiment, the cancer is acute myelocytic leukemia and the cancer expresses the folate receptor-β. In still another embodiment, the cancer is acute myelocytic leukemia and the CAR-T cells have a central memory/effector memory phenotype. In yet another embodiment, the CD8:CD4 ratio of the CAR T cells is about 1:1, about a 1.2 to 1 ratio, about a 1 to 1.2 ratio, about a 1.3 to 1 ratio, about a 1 to 1.3 ratio, about a 1.4 to 1 ratio, about a 1 to 1.4 ratio, about a 1.5 to 1 ratio, or about a 1 to 1.5 ratio. In another embodiment where the cancer is acute myelocytic leukemia or another cancer, the CAR T cells associated with the tumor can have increased CD25 expression relative to the CAR T cells not associated with the tumor.

In one embodiment, the “small molecule ligand” can be a folate, DUPA (a ligand bound by PSMA-positive human prostate cancer cells and other cancer cell types), an NK-1R ligand (receptors for NK-1R the ligand found, for example, on cancers of the colon and pancreas), a CAIX ligand (receptors for the CAIX ligand found, for example, on renal, ovarian, vulvar, and breast cancers), a ligand of gamma glutamyl transpeptidase (the transpeptidase overexpressed, for example, in ovarian cancer, colon cancer, liver cancer, astrocytic gliomas, melanomas, and leukemias), an NKG2D ligand (receptors for the NKG2D ligand found, for example, on cancers of the lung, colon, kidney, prostate, and on T and B cell lymphomas), or a CCK2R ligand (receptors for the CCK2R ligand found on cancers of the thyroid, lung, pancreas, ovary, brain, stomach, gastrointestinal stroma, and colon, among others), each of which is a small molecule ligand that binds specifically to a cancer cell type (i.e., the receptor for each of these ligands can be overexpressed on cancers compared to normal tissues).

In one embodiment, the small molecule ligand may have a mass of less than about 10,000 Daltons, less than about 9000 Daltons, less than about 8,000 Daltons, less than about 7000 Daltons, less than about 6000 Daltons, less than about 5000 Daltons, less than about 4500 Daltons, less than about 4000 Daltons, less than about 3500 Daltons, less than about 3000 Daltons, less than about 2500 Daltons, less than about 2000 Daltons, less than about 1500 Daltons, less than about 1000 Daltons, or less than about 500 Daltons. In another embodiment, the small molecule ligand may have a mass of about 1 to about 10,000 Daltons, about 1 to about 9000 Daltons, about 1 to about 8,000 Daltons, about 1 to about 7000 Daltons, about 1 to about 6000 Daltons, about 1 to about 5000 Daltons, about 1 to about 4500 Daltons, about 1 to about 4000 Daltons, about 1 to about 3500 Daltons, about 1 to about 3000 Daltons, about 1 to about 2500 Daltons, about 1 to about 2000 Daltons, about 1 to about 1500 Daltons, about 1 to about 1000 Daltons, or about 1 to about 500 Daltons.

In one embodiment, a DUPA derivative can be the ligand of the small molecule ligand linked to a targeting moiety, and DUPA derivatives are described in WO 2015/057852, incorporated herein by reference.

In one embodiment, the small molecule ligand in the context of the “small molecule ligand linked to a linker” is a folate. In various embodiments, the folate can be folic acid, a folic acid analog, or another folate receptor-binding molecule. In various embodiments, analogs of folate that can be used include folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refers to the art recognized analogs having a carbon atom substituted for one or two nitrogen atoms in the naturally occurring folic acid structure. For example, the deaza analogs include the 1-deaza, 3-deaza, 5-deaza, 8-deaza, and 10-deaza analogs. The dideaza analogs include, for example, 1,5 dideaza, 5,10-dideaza, 8,10-dideaza, and 5,8-dideaza analogs. The foregoing folic acid analogs are conventionally termed “folates,” reflecting their capacity to bind to folate receptors. Other folate receptor-binding analogs include aminopterin, amethopterin (methotrexate), N10-methylfolate, 2-deamino-hydroxyfolate, deaza analogs such as 1-deazamethopterin or 3-deazamethopterin, and 3′,5′-dichloro-4-amino-4-deoxy-N10-methylpteroylglutamic acid (dichloromethotrexate).

In another embodiment, the small molecule ligand in the context of the “small molecule ligand linked to a linker” can have the formula

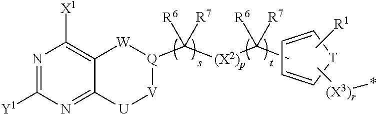

wherein X¹ and Y¹ are each-independently selected from the group consisting of halo, R², OR², SR³, and NR⁴R⁵;
U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;
X² and X³ are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —O(Z)N(R^{4b})—, —N(R^{4b})C(Z)O—, —N(R^{4b})C(Z)N(R^{5b})—, —S(O)—, —S(O)₂—, —N(R^{4a})S(O)₂—, —C(R^{6b})(R^{7b})—, —N(C≡CH)—, —N(CH²C═CH)—, C₁-C₁₂ alkylene, and C₁-C₁₂ alkyeneoxy, where Z is oxygen or sulfur,
R¹ is selected-from the group consisting of hydrogen, halo, C₁-C₁₂ alkyl, and C₁-C₁₂ alkoxy;
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂ alkanoyl, C₁-C₁₂ alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group;
R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
p, r, s, and t are each independently either 0 or 1; and
* represents an optional covalent bond to the rest of the conjugate.

In one aspect, the “targeting moiety” that binds to the CAR comprising an E2 anti-fluorescein antibody fragment and expressed by CAR T cells can be selected, for example, from fluorescein, fluorescein isothiocyanate (FITC), and NHS-fluorescein. The identity of the targeting moiety is limited only in that it should be recognized and bound by the CAR comprising an E2 anti-fluorescein antibody fragment, preferably with specificity, and that it have a relatively low molecular weight. In various aspects, exemplary targeting moieties are haptens, including small molecular weight organic molecules.

In one illustrative embodiment, the targeting moiety can have the following illustrative structure:

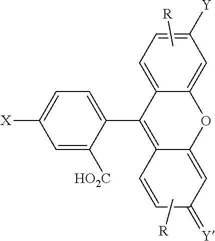

where X is oxygen, nitrogen, or sulfur, and where X is attached to linker L; Y is OR^{a}, NR^{a}₂, or NR^{a}₃⁺; and Y′ is O, NR^{a}, or NR^{a}₂⁺; where each R is independently selected in each instance from H, fluoro, sulfonic acid, sulfonate, and salts thereof, and the like; and R^{a} is hydrogen or alkyl.

In one illustrative aspect, the linker can comprise polyethylene glycol (PEG), polyproline, a hydrophilic amino acid, a sugar, an unnatural peptidoglycan, a polyvinylpyrrolidone, pluronic F-127, or a combination thereof.

In another illustrative aspect, the linker in the compound, or pharmaceutically acceptable salt thereof, described herein can comprise a direct linkage (e.g., a reaction between the isothiocyanate group of FITC and a free amine group of a small molecule ligand) or the linkage can be through an intermediary linker. In one embodiment, if present, an intermediary linker can be any biocompatible linker known in the art, such as a divalent linker. In one illustrative embodiment, the divalent linker can comprise about 1 to about 30 carbon atoms. In another illustrative embodiment, the divalent linker can comprise about 2 to about 20 carbon atoms. In other embodiments, lower molecular weight divalent linkers (i.e., those having an approximate molecular weight of about 30 to about 300 Daltons) are employed. In another embodiment, linkers lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

In various embodiments, the small molecule ligand linked to a targeting moiety can be of the formula

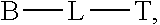

wherein B represents the small molecule ligand, L represents the linker, and T represents the targeting moiety, and wherein L comprises a structure having the formula

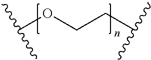

wherein n is an integer from 0 to 200. In another embodiment, n can be an integer from 0 to 150, 0 to 110, 0 to 100, 0 to 90, 0 to 80, 0 to 70, 0 to 60, 0 to 50, 0 to 40, 0 to 30, 0 to 20, 0 to 15, 0 to 14, 0 to 13, 0 to 12, 0 to 11, 0 to 10, 0 to 9, 0 to 8, 0 to 7, 0 to 6, 0 to 5, 0 to 4, 0 to 3, 0 to 2, 0 to 1, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 15 to 31, 15 to 32, 15 to 33, 15 to 34, 15 to 35, 15 to 36, 15 to 37, 15 to 38, 15 to 39, 15 to 40, 15 to 50, 15 to 60, 15 to 70, 15 to 80, 15 to 90, 15 to 100, 15 to 110, 15 to 120, 15 to 130, 15 to 140, 15 to 150, or n can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 60, 70, 80, 90, 100, 108, 110, 120, 130, 140, or 150.

In another embodiment, the linker may be a divalent linker that may include one or more spacers. Illustrative spacers are shown in the following table. The following non-limiting, illustrative spacers are described where * indicates the point of attachment to the small molecule ligand or to the targeting moiety, or to other divalent linker portions.

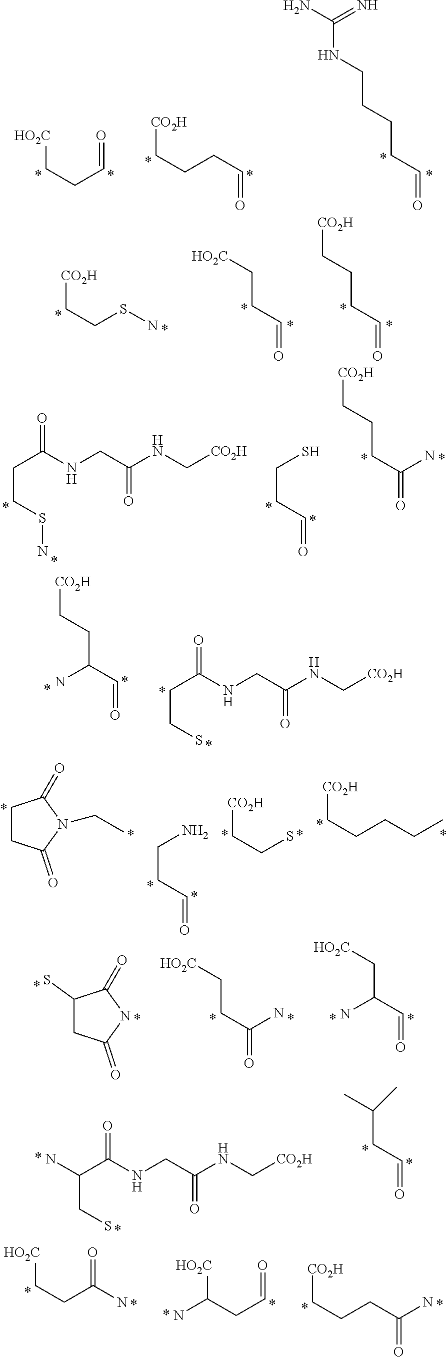

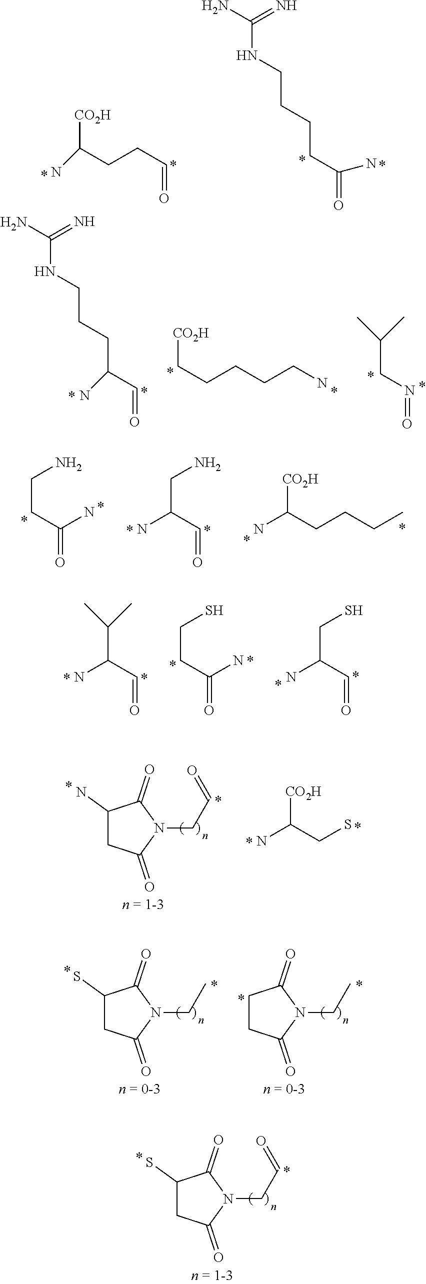

In other embodiments, the small molecule ligand linked to a targeting moiety (bridge) can have any of the following structures.

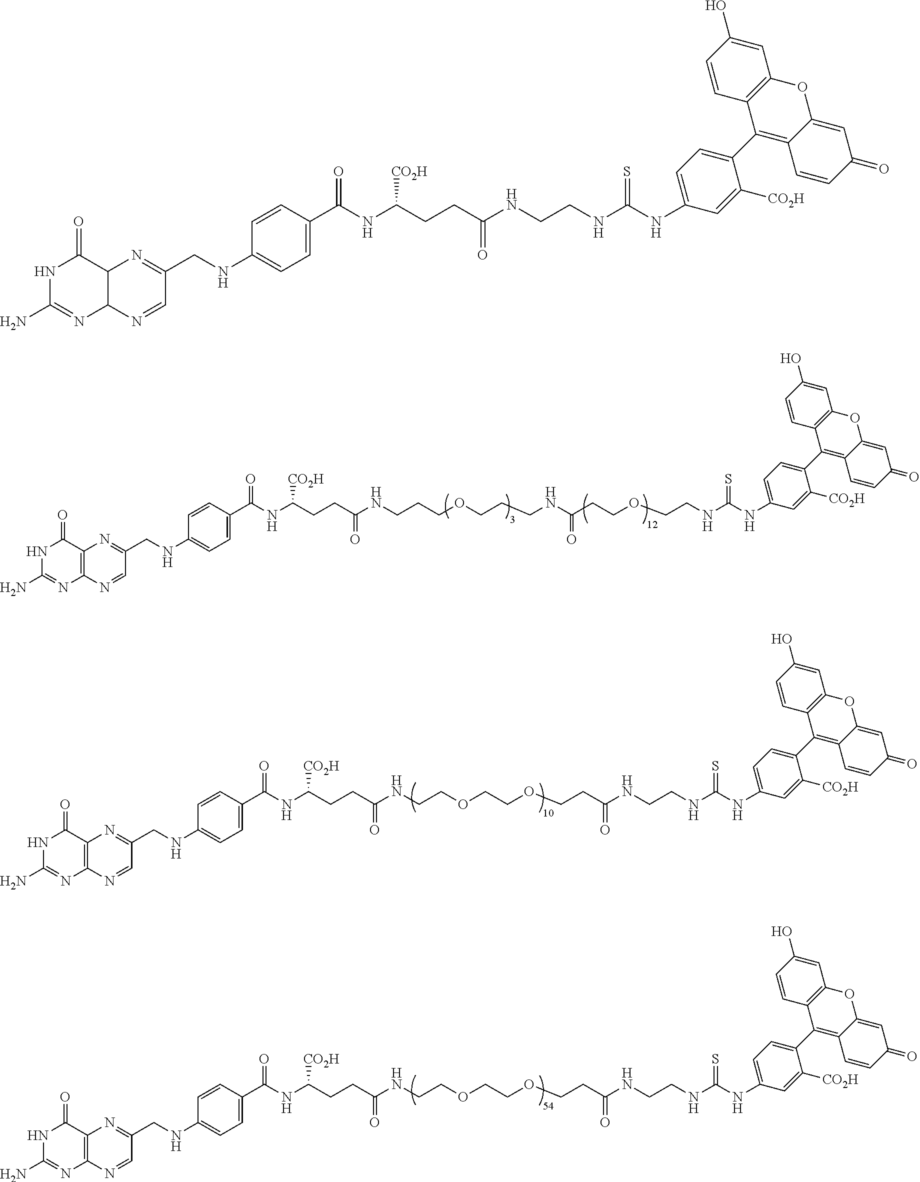

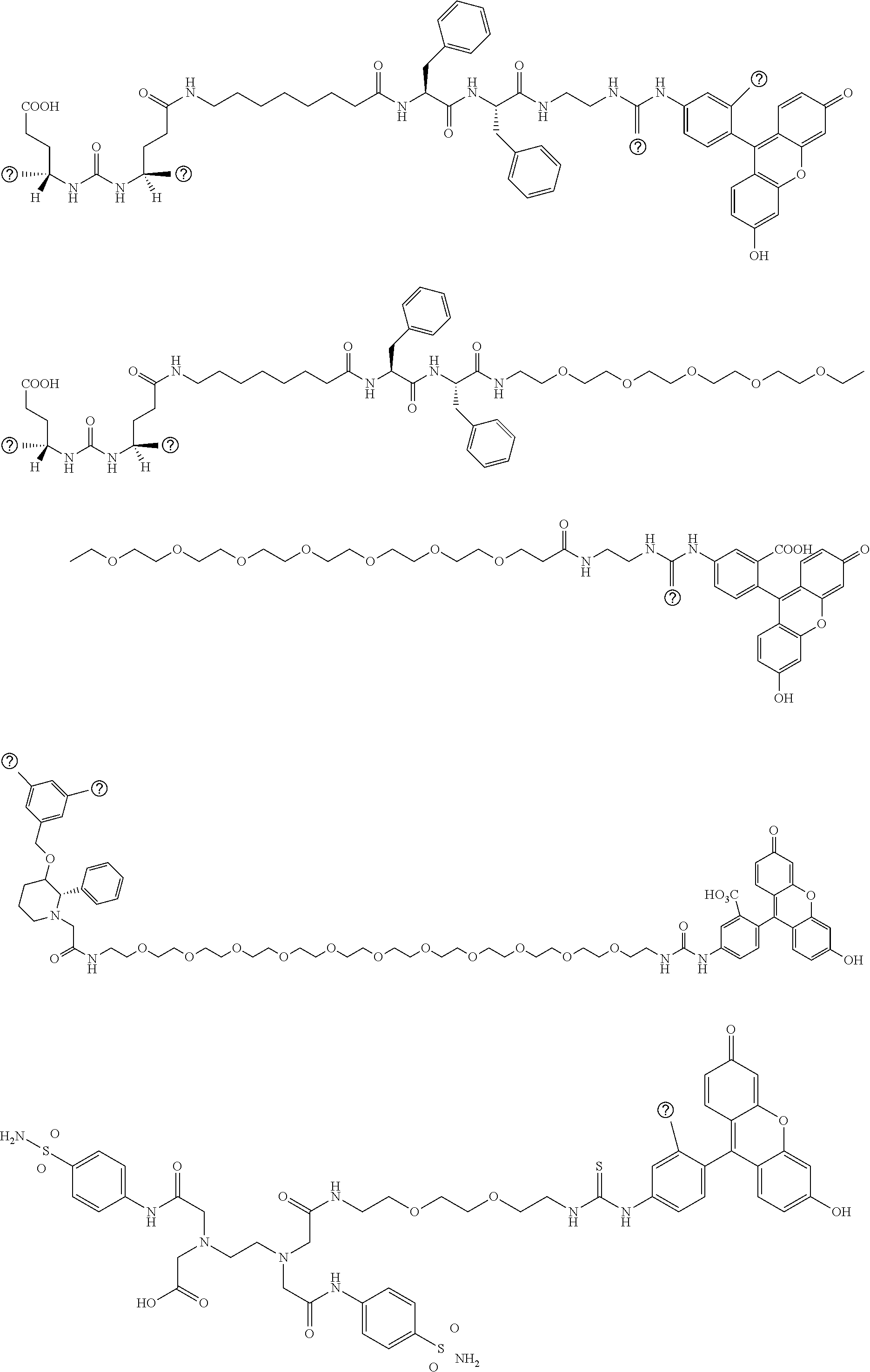

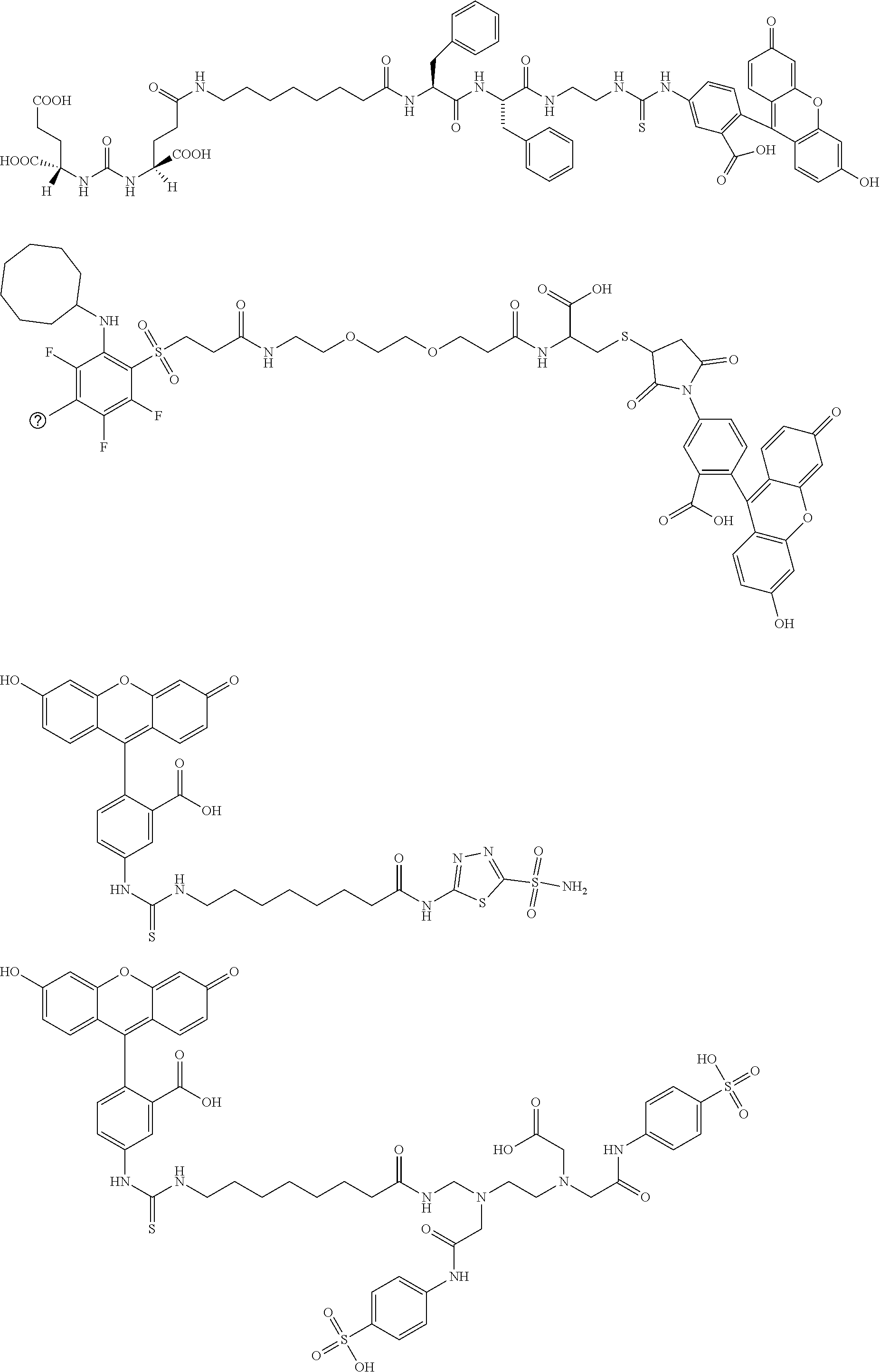

In other embodiments, the compound, or the pharmaceutically acceptable salt thereof, is not an antibody, and does not comprise a fragment of an antibody. In yet another embodiment, the targeting moiety does not comprise a peptide epitope.

In one illustrative embodiment, the small molecule ligand linked to a targeting moiety by a linker (the bridge) comprises fluorescein isothiocyanate (FITC) linked to the small molecule ligand. In one aspect, the cancer may overexpress a receptor for the small molecule ligand. In another aspect, for example, cytotoxic T cells, or another type of T cell, can be transformed to express a CAR that comprises an E2 anti-fluorescein antibody fragment. In this aspect, the CAR may target FITC, decorating the cancer with FITC molecules as a result of binding of the small molecule ligand to the cancer. Thus, toxicity to normal, non-target cells can be avoided. In this embodiment, when the E2 anti-fluorescein antibody fragment CAR expressing T cells bind FITC, the CAR T cells are activated and the cancer is ameliorated.

A “pharmaceutically acceptable salt” of a small molecule ligand linked to a targeting moiety by a linker is contemplated. As used herein, the term “pharmaceutically acceptable salt” refers to those salts whose counter ions may be used in pharmaceuticals. In various embodiments, such salts include, but are not limited to 1) acid addition salts, which can be obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid or malonic acid and the like; or 2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, trimethamine, N-methylglucamine, and the like. Pharmaceutically acceptable salts are well-known to those skilled in the art, and any such pharmaceutically acceptable salt is contemplated in connection with the embodiments described herein.

In various embodiments, suitable acid addition salts are formed from acids which form non-toxic salts. Illustrative examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

In various embodiments, suitable base salts are formed from bases which form non-toxic salts. Illustrative examples include the arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts.

In one illustrative aspect, the compound, or a pharmaceutically salt thereof, described herein may contain one or more chiral centers, or may otherwise be capable of existing as multiple stereoisomers. Accordingly, various embodiments may include pure stereoisomers as well as mixtures of stereoisomers, such as enantiomers, diastereomers, and enantiomerically or diastereomerically enriched mixtures. In one aspect, the compound, or pharmaceutically acceptable salt thereof, described herein may be capable of existing as geometric isomers. Accordingly, various embodiments may include pure geometric isomers or mixtures of geometric isomers.

In some aspects, the compound, or pharmaceutically acceptable salt thereof, described herein may exist in unsolvated forms as well as solvated forms, including hydrated forms. In general, the solvated forms are equivalent to unsolvated forms and are encompassed within the scope of the present invention.

The methods described herein also utilize T lymphocytes (e.g., cytotoxic T lymphocytes) engineered to express a chimeric antigen receptor (CAR) comprising an E2 anti-fluorescein antibody fragment, that recognizes and binds to the targeting moiety (e.g., fluorescein, fluorescein isothiocyanate (FITC), and NHS-fluorescein) of the bridge. In one embodiment, the CARs described herein comprise three domains including 1) a recognition region (e.g., a single chain fragment variable (scFv) region of an E2 anti-fluorescein antibody, an Fab fragment, and the like) which recognizes and binds to the targeting moiety with specificity, 2) a co-stimulation domain which enhances the proliferation and survival of the T lymphocytes, and 3) an activation signaling domain which generates a T lymphocyte activation signal.

In various aspects, as non-limiting examples, scFv regions of antibodies that bind a fluorescein, fluorescein isothiocyanate (FITC), and NHS-fluorescein can be used. In illustrative non-limiting embodiments, the scFv regions can be prepared from (i) the E2 antibody known in the art that binds the targeting moiety, and (iii) sequence variants derived from the scFv regions of such antibodies, e.g., scFv regions having at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity with the amino acid sequence of the scFv region from which they are derived.

In one aspect, the co-stimulation domain serves to enhance the proliferation and survival of the cytotoxic T lymphocytes upon binding of the CAR to a targeting moiety. Suitable co-stimulation domains include, but are not limited to, CD28, CD137 (4-1BB), a member of the tumor necrosis factor (TNF) receptor family, CD134 (0X40), a member of the TNFR-superfamily of receptors, CD27, CD30, CD150, DAP10, NKG2D, and CD278 (ICOS), a CD28-superfamily co-stimulatory molecule expressed on activated T cells, or combinations thereof. In one illustrative embodiment, the co-stimulation domains of the engineered CAR comprise CD 28 and CD137. A skilled artisan will understand that sequence variants of these co-stimulation domains can be used without adversely impacting the invention, where the variants have the same or similar activity as the domain upon which they are modeled. In various embodiments, such variants can have at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity to the amino acid sequence of the domain from which they are derived.

In an illustrative embodiment, the activation signaling domain serves to activate T lymphocytes (e.g., cytotoxic T lymphocytes) upon binding of the CAR to a targeting moiety. In various embodiments, suitable activation signaling domains include the T cell CD3 chain, CD3 delta receptor protein, mbl receptor protein, B29 receptor protein, and Fc receptor y. The skilled artisan will understand that sequence variants of these activation signaling domains can be used where the variants have the same or similar activity as the domain upon which they are modeled. In various embodiments, the variants have at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity with the amino acid sequence of the domain from which they are derived.

In one aspect, constructs encoding the CARs comprising the E2 anti-fluorescein antibody fragment are prepared using genetic engineering techniques. Such techniques are described in detail in Sambrook et al., “Molecular Cloning: A Laboratory Manual”, 3rd Edition, Cold Spring Harbor Laboratory Press, (2001), incorporated herein by reference, and Green and Sambrook, “Molecular Cloning: A Laboratory Manual”, 4th Edition, Cold Spring Harbor Laboratory Press, (2012), incorporated herein by reference.

As examples, a plasmid or viral expression vector (e.g., a lentiviral vector, a retrovirus vector, sleeping beauty, and piggyback (transposon/transposase systems that include a non-viral mediated CAR gene delivery system)) can be prepared that encodes a fusion protein comprising a recognition region, one or more co-stimulation domains, and an activation signaling domain, in frame and linked in a 5′ to 3′ direction. In other embodiments, other arrangements are acceptable and include a recognition region, an activation signaling domain, and one or more co-stimulation domains. In one embodiment, the placement of the recognition region in the fusion protein will generally be such that display of the region on the exterior of the cell is achieved. In one embodiment, the CARs comprising the E2 anti-fluorescein antibody fragment may include additional elements, such as a signal peptide (e.g., CD8a signal peptide) to ensure proper export of the fusion protein to the cell surface, a transmembrane domain to ensure the fusion protein is maintained as an integral membrane protein (e.g., CD8α transmembrane domain, CD28 transmembrane domain, or CD3 transmembrane domain), and a hinge domain (e.g., CD8α hinge or IgG4 hinge) that imparts flexibility to the recognition region and allows strong binding to the targeting moiety, and any other suitable domain.

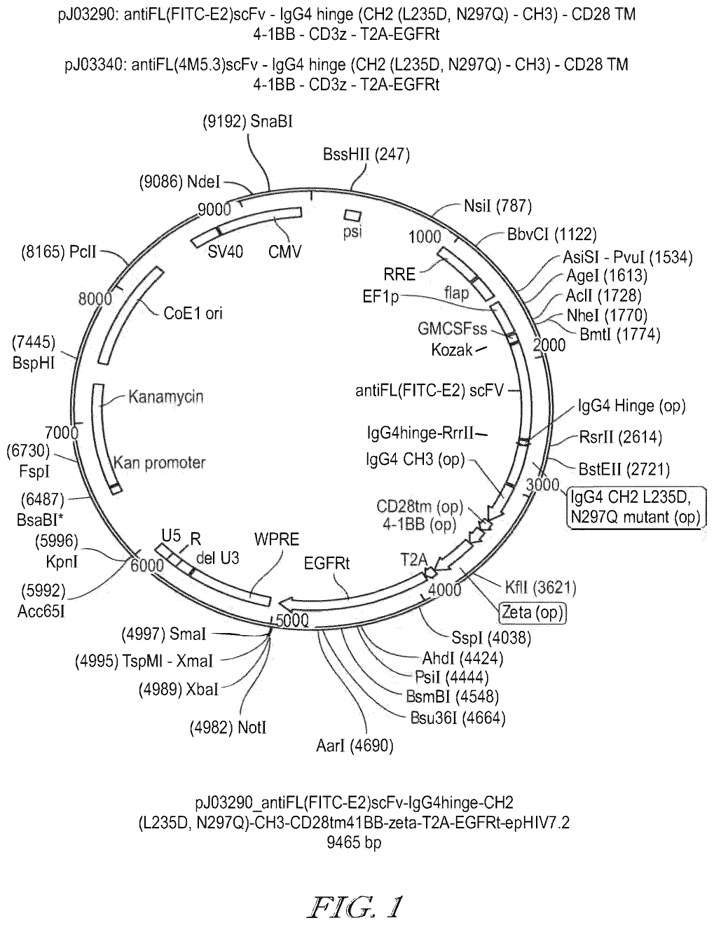

A diagram of an exemplary CAR construct wherein the expressed CAR comprises the E2 anti-fluorescein antibody fragment is shown in FIG. 1 where the fusion protein sequence is incorporated into an expression vector and where the CAR comprises an E2 anti-fluorescein antibody fragment, an IgG4 hinge domain, a CD28 transmembrane domain, and where the co-stimulation domain is CD137 (4-1BB), and the activation signaling domain is CD3. The CAR can comprise additional suitable domains. An exemplary nucleic acid sequence of a CAR insert is provided as SEQ ID NO:1 and the exemplary encoded amino acid sequence is provided as SEQ ID NO:2. As used herein, “SEQ ID NO:1” means the sequence beginning at the underlined “agc” codon and ending with the underlined “ggc” codon. This portion of the longer sequence, encodes the CAR that is inserted into the T cell membrane. The other portions of the longer sequence include coding sequence for signal peptides, the EGFRt domain, etc. which are not part of the CAR that is inserted into the membrane and which functions as the chimeric antigen receptor. As used herein, “SEQ ID NO:2” means the sequence beginning at the underlined “S” and ending with the underlined “G”. This portion of the longer sequence is the amino acid sequence for the CAR that is inserted into the T cell membrane. The other portions of the longer sequence include amino acid sequences for signal peptides, the EGFRt domain, etc. which are not part of the CAR inserted into the membrane and which functions as the chimeric antigen receptor. In yet another embodiment, SEQ ID NO:2 can comprise or consist of humanized, or human amino acid sequences. SEQ ID NOS:1 and 2 are as described above. The start and stop codons in the longer nucleic acid sequence are underlined and the longer sequence is an exemplary sequence that can be used for transduction of T cells for use in the methods as described herein.

(E2 anti-fluorescein antibody fragment CAR
nucleic acid sequence (insert))
SEQ ID NO: 1
atgcttctcctggtgacaagccttctgctctgtgagttaccacacccag
cattcctcctgatcccaagcgtgctgacacagcctagctccgtgtctgc
cgcccctggccagaaagtgaccatcagctgtagcggcagcaccagcaac
atcggcaacaactacgtgtcctggtatcagcagcaccccggcaaggccc
ccaagctgatgatctacgacgtgtccaagcggcccagcggcgtgcccga
tagattttccggcagcaagagcggcaacagcgccagcctggatatcagc
ggcctgcagtctgaggacgaggccgactactattgcgccgcctgggacg
atagcctgagcgagttcctgtttggcaccggcaccaagctgacagtgct
gggcggaggcggaggatctggcggcggaggaagtggcggagggggatct
caggtgcagctggtggaaagcggcggcaacctggtgcagcctggcggat
ctctgagactgagctgtgccgccagcggcttcaccttcggcagcttcag
catgagctgggtgcgccaggctcctgggggaggactggaatgggtggca
ggactgagcgccagaagcagcctgacccactacgccgatagcgtgaagg
gccggttcaccatcagccgggacaacgccaagaacagcgtgtacctgca
gatgaacagcctgcgggtggaagataccgccgtgtactactgcgccaga
cggtcctacgacagcagcggctactggggccacttctacagctacatgg
acgtgtggggccagggcaccctcgtgacagtgtctgagagcaagtacgg
accgccctgccccccttgccctgcccccgagttcgacggcggacccagc
gtgttcctgttcccccccaagcccaaggacaccctgatgatcagccgga
cccccgaggtgacctgcgtggtggtggacgtgagccaggaagatcccga
ggtccagttaattggtacgtggacggcgtggaagtgcacaacgccaaga
ccaagcccagagaggaacagttccagagcacctaccgggtggtgtctgt
gctgaccgtgctgcaccaggactggctgaacggcaaagaatacaagtgc
aaggtgtccaacaagggcctgcccagcagcatcgaaaagaccatcagca
aggccaagggccagcctcgcgagccccaggtgtacaccctgcctccctc
ccaggaagagatgaccaagaaccaggtgtccctgacctgcctggtgaag
ggcttctaccccagcgacatcgccgtggagtgggagagcaacggccagc
ctgagaacaactacaagaccacccctcccgtgctggacagcgacggcag
cttcttcctgtacagccggctgaccgtggacaagagccggtggcaggaa
ggcaacgtctttagctgcagcgtgatgcacgaggccctgcacaaccact
acacccagaagagcctgagcctgtccctgggcaagatgttctgggtgct
ggtggtggtggggggggctggcctgctacagcctgctggtgacagtggc
cttcatcatcttttgggtgaaacggggcagaaagaaactcctgtatata
ttcaaacaaccatttatgagaccagtacaaactactcaagaggaagatg
gctgtagctgccgatttccagaagaagaagaaggaggatgtgaactgcg
ggtgaagttcagcagaagcgccgacgcccctgcctaccagcagggccag
aatcagctgtacaacgagctgaacctgggcagaagggaagagtacgacg
tcctggataagcggagaggccgggaccctgagatgggcggcaagcctcg
gcggaagaacccccaggaaggcctgtataacgaactgcagaaagacaag
atggccgaggcctacagcgagatcggcatgaagggcgagcggagggggg
caagggccacgacggcctgtatcagggcctgtccaccgccaccaaggat
acctacgacgccctgcacatgcaggccctgcccccaaggctcgagggcg
gcggagagggcagaggaagtcttctaacatgcggtgacgtggaggagaa
tcccggcctaggatgcttctcctggtgacaagccttctgctctgtgagt
taccacacccagcattcctcctgatcccacgcaaagtgtgtaacggaat
aggtattggtgaatttaaagactcactctccataaatgctacgaatatt
aaacacttcaaaaactgcacctccatcagtggcgatctccacatcctgc
cggtggcatttaggggtgactccttcacacatactcctcctctggatcc
acaggaactggatattctgaaaaccgtaaaggaaatcacagggtttttg
ctgattcaggcttggcctgaaaacaggacggacctccatgcctttgaga
acctagaaatcatacgcggcaggaccaagcaacatggtcagttttctct
tgcagtcgtcagcctgaacataacatccttgggattacgctccctcaag
gagataagtgatggagatgtgataatttcaggaaacaaaaatttgtgct
atgcaaatacaataaactggaaaaaactgtttgggacctccggtcagaa
aaccaaaattataagcaacagaggtgaaaacagctgcaaggccacaggc
caggtctgccatgccttgtgctcccccgagggctgctggggcccggagc
ccagggactgcgtctcttgccggaatgtcagccgaggcagggaatgcgt
ggacaagtgcaaccttctggagggtgagccaagggagtttgtggagaac
tctgagtgcatacagtgccacccagagtgcctgcctcaggccatgaaca
tcacctgcacaggacggggaccagacaactgtatccagtgtgcccacta
cattgacggcccccactgcgtcaagacctgcccggcaggagtcatggga
gaaaacaacaccctggtctggaagtacgcagacgccggccatgtgtgcc
acctgtgccatccaaactgcacctacggatgcactgggccaggtcttga
aggctgtccaacgaatgggcctaagatcccgtccatcgccactgggatg
gtgggggccctcctcttgctgctggtggtggccctggggatcggcctct
tcatgtga
(E2 anti-fluorescein antibody fragment CAR amino
acid sequence (insert))
SEQ ID NO: 2
M L L L V T S L L L C E L P H P A F L L I P S V L
T Q P S S V S A A P G Q K V T I S C S G S T S N I
G N N Y V S W Y Q Q H P G K A P K L M I Y D V S K
R P S G V P D R F S G S K S G N S A S L D I S G L
Q S E D E A D Y Y C A A W D D S L S E F L F G T G
T K L T V L G G G G G S G G G G S G G G G S Q V Q
L V E S G G N L V Q P G G S L R L S C A A S G F T
F G S F S M S W V R Q A P G G G L E W V A G L S A
R S S L T H Y A D S V K G R F T I S R D N A K N S
V Y L Q M N S L R V E D T A V Y Y C A R R S Y D S
S G Y W G H F Y S Y M D V W G Q G T L V T V S E S
K Y G P P C P P C P A P E F D G G P S V F L F P P
K P K D T L M I S R T P E V T C V V V D V S Q E D
P E V Q F N W Y V D G V E V H N A K T K P R E E Q
F Q S T Y R V V S V L T V L H Q D W L N G K E Y K
C K V S N K G L P S S I E K T I S K A K G Q P R E
P Q V Y T L P P S Q E E M T K N Q V S L T C L V K
G F Y P S D I A V E W E S N G Q P E N N Y K T T P
P V L D S D G S F F L Y S R L T V D K S R W Q E G
N V F S C S V M H E A L H N H Y T Q K S L S L S L
G K M F W V L V V V G G V L A C Y S L L V T V A F
I I F W V K R G R K K L L Y I F K Q P F M R P V Q
T T Q E E D G C S C R F P E E E E G G C E L R V K
F S R S A D A P A Y Q Q G Q N Q L Y N E L N L G R
R E E Y D V L D K R R G R D P E M G G K P R R K N
P Q E G L Y N E L Q K D K M A E A Y S E I G M K G
E R R R G K G H D G L Y Q G L S T A T K D T Y D A
L H M Q A L P P R L E G G G E G R G S L L T C G D
V E E N P G P R M L L L V T S L L L C E L P H P A
F L L I P R K V C N G I G I G E F K D S L S I N A
T N I K H F K N C T S I S G D L H I L P V A F R G
D S F T H T P P L D P Q E L D I L K T V K E I T G
F L L I Q A W P E N R T D L H A F E N L E I I R G
R T K Q H G Q F S L A V V S L N I T S L G L R S L
K E I S D G D V I I S G N K N L C Y A N T I N W K
K L F G T S G Q K T K I I S N R G E N S C K A T G
Q V C H A L C S P E G C W G P E P R D C V S C R N
V S R G R E C V D K C N L L E G E P R E F V E N S
E C I Q C H P E C L P Q A M N I T C T G R G P D N
C I Q C A H Y I D G P H C V K T C P A G V M G E N
N T L V W K Y A D A G H V C H L C H P N C T Y G C
T G P G L E G C P T N G P K I P S I A T G M V G A
L L L L L V V A L G I G L F M

In one embodiment, the CAR, expressing the E2 anti-fluorescein antibody fragment, comprises a recognition region and the recognition region is a single chain fragment variable (scFv) region of the E2 anti-fluorescein antibody, a co-stimulation domain and the co-stimulation domain is CD137 (4-1BB), and an activation signaling domain and the activation signaling domain is a T cell CD3ζ chain. In another embodiment, the CAR can further comprise any additional suitable domains. It is well-known to the skilled artisan that an anti-FITC scFv and an anti-fluorescein scFv are equivalent terms.

In various embodiments, the “E2 anti-fluorescein antibody fragment” can be a CAR comprising a fragment (e.g., an scFv fragment) of the E2 anti-fluorescein antibody. The E2 anti-fluorescein antibody is described, for example, in Vaughan, et al., Nature Biotechnol. Vol. 14(3), pp. 309-314, 1996, incorporated herein by reference. In one embodiment, the CAR expressing the E2 anti-fluorescein antibody fragment can have a binding affinity for fluorescein of about 0.7 nM to about 0.8 nM, about 0.72 nM to about 0.8 nM, about 0.73 nM to about 0.8 nM, about 0.72 nM to about 7.8 nM, about 0.73 to about 0.77 nM, or about 0.75 nM.

In various embodiments, the CAR can further comprise an IgG4 hinge domain, a CD3 activation domain, and/or a 4-1BB co-stimulation domain, and other suitable domains. In still other embodiments, the CAR can be encoded by a polynucleotide having at least about 90%, 91%, 92%, 93%, 94%, 95%, 96%, 97%, 98%, 99%, or 99.5% identity to SEQ ID NO: 1. In another illustrative embodiment, the CAR can be encoded by a polynucleotide that hybridizes under high stringency conditions to a polynucleotide having SEQ ID NO:1. In still another aspect, the CAR can be encoded by a polynucleotide having SEQ ID NO:1, or by a degenerate variant of SEQ ID NO:1. In other embodiments, the CAR protein sequence can have at least about 90%, 91%, 92%, 93%, 94%, 95%, 96%, 97%, 98%, 99%, or 99.5% identity to SEQ ID NO:2. In yet another embodiment, the CAR protein sequence can have up to about 50 conservative amino acid substitutions. In any of the embodiments described herein, the CAR binds fluorescein.

In one embodiment, T lymphocytes (e.g., cytotoxic T lymphocytes) can be genetically engineered to express a CAR construct, wherein the CAR construct expresses an E2 anti-fluorescein antibody fragment, by transfecting a population of the T lymphocytes with an expression vector encoding the CAR construct expressing the E2 anti-fluorescein antibody fragment. Suitable methods for preparing a transduced population of T lymphocytes expressing the E2 anti-fluorescein antibody fragment are well-known to the skilled artisan, and are described in Sambrook et al., “Molecular Cloning: A Laboratory Manual,” 3rd Edition, Cold Spring Harbor Laboratory Press, (2001), incorporated herein by reference, and Green and Sambrook, “Molecular Cloning: A Laboratory Manual”, 4th Edition. Cold Spring Harbor Laboratory Press, (2012), incorporated herein by reference.

In one embodiment, CAR T cells comprising a nucleic acid of SEQ ID NO:1 can be used as described herein. In another embodiment. CAR T cells comprising a polypeptide of SEQ ID NO:2 can be used as described herein. In another illustrative aspect, a nucleic acid (e.g., an isolated nucleic acid) comprising SEQ ID NO:1 and encoding a chimeric antigen receptor can be used to prepare the CAR T cells for use as described herein.

In yet another embodiment, a chimeric antigen receptor polypeptide comprising SEQ ID NO:2 can be used to prepare the CAR T cells for use as described herein. In another embodiment, a vector comprising SEQ ID NO:1 can be used to prepare the CAR T cells for use as described herein. In another aspect, a lentiviral vector is provided comprising SEQ ID NO:1 can be used to prepare the CAR T cells for use as described herein. In yet another embodiment, SEQ ID NO:2 can comprise or consist of humanized, or human amino acid sequences.

In each of these embodiments, variant nucleic acid sequences or amino acid sequences having at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO:1 are contemplated. In another embodiment, the nucleic acid sequence can be a variant nucleic acid sequence having at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO:1 as long as the variant sequence encodes a polypeptide of SEQ ID NO:2. In another embodiment, the nucleic acid sequence or the amino acid sequence can be a variant nucleic acid or amino acid sequence having at least about 80%, at least about 90%, at least about 91%, at least about 92%, at least about 93%, at least about 94%, at least about 95%, at least about 96%, at least about 97%, at least about 98%, at least about 99%, or at least about 99.5% sequence identity to SEQ ID NO:1 along a stretch of 200 nucleic acids or, for SEQ ID NO:2, along a stretch of 200 amino acids. In one embodiment, determination of percent identity or similarity between sequences can be done, for example, by using the GAP program (Genetics Computer Group, software; now available via Accelrys on http://www.accelrys.com), and alignments can be done using, for example, the ClustalW algorithm (VNTI software, InforMax Inc.). A sequence database can be searched using the nucleic acid or amino acid sequence of interest. Algorithms for database searching are typically based on the BLAST software (Altschul et al., 1990). In some embodiments, the percent identity can be determined along the full-length of the nucleic acid or amino acid sequence.

Also within the scope of the invention, nucleic acids complementary to the nucleic acid represented by SEQ ID NO:1 can be used to prepare the CAR T cells for use as described herein, and those that hybridize to the nucleic acid represented by SEQ ID NO:1, or those that hybridize to its complement under highly stringent conditions can be used. In accordance with the invention “highly stringent conditions” means hybridization at 65° C. in 5×SSPE and 50% formamide, and washing at 65° C. in 0.5×SSPE. Conditions for high stringency, low stringency and moderately stringent hybridization are described in Sambrook et al., “Molecular Cloning: A Laboratory Manual”, 3rd Edition, Cold Spring Harbor Laboratory Press, (2001), incorporated herein by reference, and Green and Sambrook, “Molecular Cloning: A Laboratory Manual”, 4th Edition. Cold Spring Harbor Laboratory Press, (2012), incorporated herein by reference. In some illustrative aspects, hybridization occurs along the full-length of the nucleic acid.

In still other embodiments, the CAR for use in the methods described herein can be encoded by a polynucleotide having at least about 90%, 91%, 92%, 93%, 94%, 95%, 96%, 97%, 98%, 99%, or 99.5% identity to SEQ ID NO: 1. In another illustrative embodiment, the CAR can be encoded by a polynucleotide that hybridizes under high stringency conditions to a polynucleotide having SEQ ID NO:1. In still another aspect, the CAR can be encoded by a polynucleotide having SEQ ID NO:1, or by a degenerate variant of SEQ ID NO: 1. As used herein, a degenerate variant refers to the genetic code having more than one codon to specify any particular amino acid. The degenerate variant codons specifying each amino acid are well-known in the art. In yet another aspect, a substitution can be made to optimize the level of production of the polypeptide in a particular prokaryotic or eukaryotic host cell (i.e., a codon-usage variant).

In other embodiments, the CAR protein sequence can have at least about 90%, 91%, 92%, 93%, 94%, 95%, 96%, 97%, 98%, or 99% identity to SEQ ID NO:2. In yet another embodiment, the CAR protein sequence can have up to about 50 conservative amino acid substitutions. In one embodiment, the CAR protein sequence that expresses the E2 anti-fluorescein antibody fragment, can have up to about 5 conservative amino acid substitutions, up to about 10 conservative amino acid substitutions, up to about 15 conservative amino acid substitutions, up to about 20 conservative amino acid substitutions, up to about 25 conservative amino acid substitutions, up to about 30 conservative amino acid substitutions, up to about 35 conservative amino acid substitutions, up to about 40 conservative amino acid substitutions, up to about 45 conservative amino acid substitutions, up to about 50 conservative amino acid substitutions, up to about 55 conservative amino acid substitutions, up to about 60 conservative amino acid substitutions, up to about 65 conservative amino acid substitutions, up to about 70 conservative amino acid substitutions, or up to about 75 conservative amino acid substitutions. As is well known to those skilled in the art, altering any non-critical amino acid of a polypeptide by conservative amino acid substitution should not significantly alter the activity of that polypeptide because the side-chain of the replacement amino acid should be able to form similar bonds and contacts as the side chain of the amino acid which has been replaced. In any of the embodiments described herein, the CAR binds fluorescein.

In one illustrative aspect, non-conservative substitutions are possible provided that these do not excessively affect the fluorescein-binding activity of the E2 anti-fluorescein antibody fragment polypeptide. As is well-known in the art, a “conservative substitution” of an amino acid or a “conservative substitution variant” of a polypeptide refers to an amino acid substitution which maintains: 1) the secondary structure of the polypeptide; 2) the charge or hydrophobicity of the amino acid; and 3) the bulkiness of the side chain or any one or more of these characteristics. In one embodiment, the conservative amino acid substitution can be with an amino acid analog. Illustratively, the well-known terminologies “hydrophilic residues” relate to serine or threonine. “Hydrophobic residues” refer to leucine, isoleucine, phenylalanine, valine or alanine, or the like. “Positively charged residues” relate to lysine, arginine, omithine, or histidine. “Negatively charged residues” refer to aspartic acid or glutamic acid. Residues having “bulky side chains” refer to phenylalanine, tryptophan or tyrosine, or the like. An exemplary list of conservative amino acid substitutions is given in TABLE 1.

TABLE 1

| For Amino Acid | Replace With |
|---|---|
| Alanine | D-Ala, Gly, Aib, β-Ala, L-Cys, D-Cys |
| Arginine | D-Arg, Lys, D-Lys, Orn D-Orn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr |
| Glutamine | D-Gln, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, β-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Orn, D-Orn |
| Methionine | D-Met, S-Me-Cys, Ile, D-Ile, Leu, D-Leu, Val, |
| | D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, Val, |
| | D-Val |
| Tyrosine | D-Tyr, Phe, D-Phe, His, D-His, Trp, D-Trp |
| Valine | D-Val, Leu, D-Leu, Ile, D-Ile, Met, D-Met |

In one embodiment, the T lymphocytes (e.g., cytotoxic T lymphocytes used to prepare CAR T cells expressing the E2 anti-fluorescein antibody fragment, or non-transformed T cells), used in the methods described herein, can be autologous cells, although heterologous cells can also be used, such as when the patient being treated has received high-dose chemotherapy or radiation treatment to destroy the patient's immune system. In one embodiment, allogenic cells can be used.

In one aspect, the T lymphocytes can be obtained from a patient by means well-known in the art. For example, T cells (e.g., cytotoxic T cells or non-transformed T cells) can be obtained by collecting peripheral blood from the patient, subjecting the blood to Ficoll density gradient centrifugation, and then using a negative T cell isolation kit (such as EasySep™ T Cell Isolation Kit) to isolate a population of T cells from the peripheral blood. In one illustrative embodiment, the population of T lymphocytes (e.g., cytotoxic T cells or non-transformed T cells) need not be pure and may contain other cells such as other types of T cells (in the case of cytotoxic T cells, for example), monocytes, macrophages, natural killer cells, and B cells. In one aspect, the population being collected can comprise at least about 90% of the selected cell type, at least about 91%, 92%, 93%, 94%, 95%, 96%, 97%, 98%, 99%, or 100% of the selected cell type.

In one embodiment, after the T lymphocytes (e.g., cytotoxic T cells used to prepare CAR T cells expressing the E2 anti-fluorescein antibody fragment) are obtained, the cells are cultured under conditions that promote the activation of the cells. In this embodiment, the culture conditions may be such that the cells can be administered to a patient without concern for reactivity against components of the culture medium. For example, the culture conditions may not include bovine serum products, such as bovine serum albumin In one illustrative aspect, the activation can be achieved by introducing known activators into the culture medium, such as anti-CD3 antibodies in the case of cytotoxic T cells. Other suitable activators include anti-CD28 antibodies. In one aspect, the population of lymphocytes can be cultured under conditions promoting activation for about 1 to about 4 days. In one embodiment, the appropriate level of activation can be determined by cell size, proliferation rate, or activation markers determined by flow cytometry.

In one illustrative embodiment, after the population of T lymphocytes (e.g., cytotoxic T lymphocytes used to prepare CAR T cells expressing the E2 anti-fluorescein antibody fragment) has been cultured under conditions promoting activation, the cells can be transfected with an expression vector encoding a CAR comprising the E2 anti-fluorescein antibody fragment. Suitable vectors and transfection methods for use in various embodiments are described above. In one aspect, after transfection, the cells can be immediately administered to the patient or the cells can be cultured for at least about 1, 2, 3, 4, 5, 6, 7, 8, 9, 10, 11, 12, 13, 14, 15, 16, 17, 18 or more days, or between about 5 and about 12 days, between about 6 and about 13 days, between about 7 and about 14 days, or between about 8 and about 15 days, for example, to allow time for the cells to recover from the transfection. In one aspect, suitable culture conditions can be similar to the conditions under which the cells were cultured for activation either with or without the agent that was used to promote activation.

Thus, as described above, in one illustrative aspect, the methods of treatment described herein can further comprise 1) obtaining a population of autologous or heterologous T lymphocytes (e.g., cytotoxic T lymphocytes used to prepare CAR T cells expressing the E2 anti-fluorescein antibody fragment), 2) culturing the T lymphocytes under conditions that promote the activation of the cells, and 3) transfecting the lymphocytes with an expression vector encoding a CAR comprising an E2 anti-fluorescein antibody fragment to form CAR T cells expressing the E2 anti-fluorescein antibody fragment.

In one illustrative embodiment, when the cells have been transfected and activated, a composition comprising the CAR T cells, wherein the CAR T cells comprise a CAR comprising an E2 anti-fluorescein antibody fragment can be prepared and administered to the patient, with or without non-transformed T cells. In one embodiment, culture media that lacks any animal products, such as bovine serum, can be used to culture the CAR T cells expressing the E2 anti-fluorescein antibody fragment and/or the non-transformed T cells. In another embodiment, tissue culture conditions typically used by the skilled artisan to avoid contamination with bacteria, fungi and mycoplasma can be used. In an exemplary embodiment, prior to being administered to a patient, the cells (e.g., CAR T cells expressing the E2 anti-fluorescein antibody fragment and/or non-transformed T cells) are pelleted, washed, and are resuspended in a pharmaceutically acceptable carrier or diluent. Exemplary compositions comprising CAR-expressing T lymphocytes expressing the E2 anti-fluorescein antibody fragment (e.g., cytotoxic T lymphocytes) or non-transformed T cells include compositions comprising the cells in sterile 290 mOsm saline, in infusible cryomedia (containing Plasma-Lyte A, dextrose, sodium chloride injection, human serum albumin and DMSO), in 0.9% NaCl with 2% human serum albumin, or in any other sterile 290 mOsm infusible materials. Alternatively, in another embodiment, depending on the identity of the culture medium, the CAR T cells expressing the E2 anti-fluorescein antibody fragment, or non-transformed T cells, can be administered in the culture media as the composition, or concentrated and resuspended in the culture medium before administration. In various embodiments, the CAR T cell composition wherein the CAR comprises the E2 anti-fluorescein antibody fragment, with or without non-transformed T cells, can be administered to the patient via any suitable means, such as parenteral administration, e.g., intradermally, subcutaneously, intramuscularly, intraperitoneally, intravenously, or intrathecally.

In one aspect, the total number of CAR T cells expressing the E2 anti-fluorescein antibody fragment and the concentration of the cells in the composition administered to the patient will vary depending on a number of factors including the type of T lymphocytes (e.g., cytotoxic T lymphocytes) being used, the binding specificity of the CAR that comprises the E2 anti-fluorescein antibody fragment, the identity of the targeting moiety and the small molecule ligand, the identity of the cancer, the location of the cancer in the patient, the means used to administer the compositions to the patient, and the health, age and weight of the patient being treated. In various embodiments, suitable compositions comprising transduced CAR T cells expressing the E2 anti-fluorescein antibody fragment include those having a volume of about 0.1 ml to about 200 ml and about 0.1 ml to about 125 ml.

In various embodiments, the transduced CAR T cells, wherein the CAR T cells comprise a CAR comprising an E2 anti-fluorescein antibody fragment, administered to the patient can comprise from about 1×10⁵ to about 1×10¹⁵ or 1×10⁶ to about 1×10¹⁵ transduced CAR T cells expressing the E2 anti-fluorescein antibody fragment. In various embodiments about 1×10⁵ to about 1×10¹⁹, about 1×10⁶ to about 1×10¹⁹, about 1×10⁶ to about 1×10¹⁹, about 1×10⁶ to about 1×10⁸, about 1×10⁶ to about 2×10⁷, about 1×10⁶ to about 3×10⁷, about 1×10⁶ to about 1.5×10⁷, about 1×10⁶ to about 1×10⁷, about 1×10⁶ to about 9×10⁶, about 1×10⁶ to about 8×10⁶, about 1×10⁶ to about 7×10⁶, about 1×10⁶ to about 6×10⁶, about 1×10⁶ to about 5×10⁶, about 1×10⁶ to about 4×10⁶, about 1×10⁶ to about 3×10⁶, about 1×10⁶ to about 2×10⁶, about 2×10⁶ to about 6×10⁶, about 2×10⁶ to about 5×10⁶, about 3×10⁶ to about 6×10⁶, about 4×10⁶ to about 6×10⁶, about 4×10⁶ to about 1×10⁷, about 1×10⁶ to about 1×10⁷, about 1×10⁶ to about 1.5×10⁷, about 1×10⁶ to about 2×10⁷, about 0.2×10⁶ to about 1×10⁷, about 0.2×10⁶ to about 1.5×10⁷, about 0.2×10⁶ to about 2×10⁷, or about 5×10⁶ CAR T cells expressing the E2 anti-fluorescein antibody fragment can be administered to the patient. In one aspect, in any embodiment described herein, a single dose or multiple doses of the CAR T cells expressing the E2 anti-fluorescein antibody fragment can be administered to the patient. In any of the embodiments described in this paragraph or herein, the CAR T cell numbers can be in kg of patient body weight. In any embodiment described herein, the CAR T cells expressing the E2 anti-fluorescein antibody fragment can be administered before the compound, or the pharmaceutically acceptable salt thereof. As would be understood, the designations i), ii), and iii), etc. for steps of any method described herein do not indicate an order unless otherwise stated.

In the various embodiments described herein, non-transformed T cells can also be administered with the CAR T cells expressing the E2 anti-fluorescein antibody fragment and can be administered in amounts described herein for the CAR T cells expressing the E2 anti-fluorescein antibody fragment, and the non-transformed T cells. In one aspect, a mixture of CAR T cells, wherein the CAR T cells comprise a CAR comprising the E2 anti-fluorescein antibody fragment, and non-transformed T cells can be administered a single time or multiple times, or combinations of doses of pure CAR T cells expressing the E2 anti-fluorescein antibody fragment and mixtures of CAR T cells expressing the E2 anti-fluorescein antibody fragment, and non-transformed T cells, can be administered (e.g., a dose of CAR T cells expressing the E2 anti-fluorescein antibody fragment followed by one or more doses of a mixture of CAR T cells expressing the E2 anti-fluorescein antibody fragment and non-transformed T cells). As is clear to the skilled artisan from the disclosure herein, a “mixture” of CAR T cells expressing the E2 anti-fluorescein antibody fragment and non-transformed T cells as described herein, means that CAR T cells expressing the E2 anti-fluorescein antibody fragment are mixed with non-transformed T cells that have not been exposed to a construct used for expression of a CAR comprising an E2 anti-fluorescein antibody fragment.

In other embodiments, the dose of the CAR T cells expressing the E2 anti-fluorescein antibody fragment administered to the patient in the CAR T cell composition is selected from the group consisting of about 1 million, about 2 million, about 3 million, about 4 million, about 5 million, about 6 million, about 7 million, about 8 million, about 9 million, about 10 million, about 11 million, about 12 million, about 12 5 million, about 13 million, about 14 million, and about 15 million of the CAR T cells expressing the E2 anti-fluorescein antibody fragment.

In still other illustrative embodiments, the CAR T cell composition comprising CAR T cells expressing the E2 anti-fluorescein antibody fragment is administered by injection into the patient's bloodstream, and the CAR T cells expressing the E2 anti-fluorescein antibody fragment in the patient's bloodstream are at least 5 percent, at least 7 percent, at least 10 percent, at least 11 percent, at least 12 percent, at least 13 percent, at least 14 percent, or at least 15 percent of the patient's total T cells in the patient's bloodstream by about four weeks after injection of the CAR T cell composition, at least 20 percent, 25 percent, 30 percent, 35 percent, 40 percent, or 50 percent by about 3 weeks after injection of the CAR T cell composition, at least 60 percent, 65 percent, 70 percent, 75 percent, or 80 percent by about 2 weeks after injection of the CAR T cell composition, or at least 85 percent, 90 percent, or 95 by about 1 week after injection of the CAR T cell composition.

In embodiments described herein, the CAR T cell composition can comprise CAR T cells expressing the E2 anti-fluorescein antibody fragment, without any other cell type, or non-transformed T cells can be administered to the patient in combination with CAR T cells expressing the E2 anti-fluorescein antibody fragment. For embodiments where multiple doses of the CAR T cell composition are administered, any dose can comprise CAR T cells expressing the E2 anti-fluorescein antibody fragment or a mixture of CAR T cells expressing the E2 anti-fluorescein antibody fragment and non-transformed T cells. In various embodiments, the non-transformed T cells can be administered in amounts described herein for the CAR T cells expressing the E2 anti-fluorescein antibody fragment.

In another embodiment, any dose of the CAR T cell composition can comprise a mixture of the CAR T cells expressing the E2 anti-fluorescein antibody fragment, and non-transformed T cells in a ratio selected from about 1:5 of the CAR T cells expressing the E2 anti-fluorescein antibody fragment to the non-transformed T cells, about 1:4 of the CAR T cells expressing the E2 anti-fluorescein antibody fragment to the non-transformed T cells, about 1:3 of the CAR T cells expressing the E2 anti-fluorescein antibody fragment to the non-transformed T cells, about 1:2 of the CAR T cells expressing the E2 anti-fluorescein antibody fragment to the non-transformed T cells, and about 1:1 of the CAR T cells expressing the E2 anti-fluorescein antibody fragment to the non-transformed T cells.

In still other embodiments, any dose of the CAR T cell composition can comprise a mixture of the CAR T cells expressing the E2 anti-fluorescein antibody fragment, and non-transformed T cells in a ratio of from about 1:1 to about 1:5 of the CAR T cells expressing the E2 anti-fluorescein antibody fragment to the non-transformed T cells, or the CAR T cell composition can comprise a mixture of about 10 million of the CAR T cells expressing the E2 anti-fluorescein antibody fragment and about 40 million non-transformed T cells, about 15 million of the CAR T cells expressing the E2 anti-fluorescein antibody fragment and about 35 million of the non-transformed T cells, about 20 million of the CAR T cells expressing the E2 anti-fluorescein antibody fragment and about 30 million of the non-transformed T cells, or about 25 million of the CAR T cells expressing the E2 anti-fluorescein antibody fragment and about 25 million of the non-transformed T cells.

The compound, or pharmaceutically acceptable salt thereof, or CAR T cell composition, wherein the composition comprises CAR T cells comprising a CAR comprising an E2 anti-fluorescein antibody fragment, described herein can be administered to the patient using any suitable method known in the art. As described herein, the term “administering” or “administered” includes all means of introducing the compound, or pharmaceutically acceptable salt thereof, or CAR T cell composition comprising CAR T cells having a CAR comprising an E2 anti-fluorescein antibody fragment to the patient, including, but not limited to, oral, intravenous, intramuscular, subcutaneous, transdermal, and the like. In one aspect, the compound, or pharmaceutically acceptable salt thereof, described herein may be administered in unit dosage forms and/or formulations containing conventional nontoxic pharmaceutically-acceptable carriers, adjuvants, and vehicles.

In one aspect, the compound, or pharmaceutically acceptable salt thereof, or CAR T cell composition wherein the CAR T cell composition comprises CAR T cells expressing a CAR comprising an E2 anti-fluorescein antibody fragment, as described herein may be administered directly into the blood stream, into muscle, or into an internal organ. In various embodiments, suitable routes for such parenteral administration include intravenous, intraarterial, intraperitoneal, intrathecal, epidural, intracerebroventricular, intraurethral, intrasternal, intracranial, intratumoral, intramuscular and subcutaneous delivery. In one embodiment, means for parenteral administration include needle (including microneedle) injectors, needle-free injectors and infusion techniques.

In one illustrative aspect, parenteral formulations are typically aqueous solutions which may contain carriers or excipients such as salts, carbohydrates and buffering agents (preferably at a pH of from 3 to 9), but they may be more suitably formulated as a sterile non-aqueous solution or as a dried form to be used in conjunction with a suitable vehicle such as sterile, pyrogen-free water or sterile saline. In other embodiments, any of the liquid formulations described herein may be adapted for parenteral administration as described herein. The preparation under sterile conditions, by lyophilization to produce a sterile lyophilized powder for a parenteral formulation, may readily be accomplished using standard pharmaceutical techniques well-known to those skilled in the art. In one embodiment, the solubility of the compound, or pharmaceutically acceptable salt thereof, used in the preparation of a parenteral formulation may be increased by the use of appropriate formulation techniques, such as the incorporation of solubility-enhancing agents.

The amount of the compound, or pharmaceutically acceptable salt thereof, to be administered to the patient can vary significantly depending on the cancer being treated, the route of administration of the compound, or pharmaceutically acceptable salt thereof, and the tissue distribution. The amount to be administered to a patient can be based on body surface area, mass, and physician assessment. In various embodiments, amounts to be administered can range, for example, from about 0.05 mg to about 30 mg, 0.05 mg to about 25.0 mg, about 0.05 mg to about 20.0 mg, about 0.05 mg to about 15.0 mg, about 0.05 mg to about 10.0 mg, about 0.05 mg to about 9.0 mg, about 0.05 mg to about 8.0 mg, about 0.05 mg to about 7.0 mg, about 0.05 mg to about 6.0 mg, about 0.05 mg to about 5.0 mg, about 0.05 mg to about 4.0 mg, about 0.05 mg to about 3.0 mg, about 0.05 mg to about 2.0 mg, about 0.05 mg to about 1.0 mg, about 0.05 mg to about 0.5 mg, about 0.05 mg to about 0.4 mg, about 0.05 mg to about 0.3 mg, about 0.05 mg to about 0.2 mg, about 0.05 mg to about 0.1 mg, about 0.01 mg to about 2 mg, about 0.3 mg to about 10 mg, about 0.1 mg to about 20 mg, or about 0.8 to about 3 mg. One of skill in the art will readily appreciate that the dose may vary within the various ranges provided above based on the factors noted above, and may be at the physician's discretion.

In other embodiments, the dose of the compound, or pharmaceutically acceptable salt thereof, can range, for example, from about 50 nmoles/kg to about 3000 nmoles/kg of patient body weight, about 50 nmoles/kg to about 2000 nmoles/kg, about 50 nmoles/kg to about 1000 nmoles/kg, about 50 nmoles/kg to about 900 nmoles/kg, about 50 nmoles/kg to about 800 nmoles/kg, about 50 nmoles/kg to about 700 nmoles/kg, about 50 nmoles/kg to about 600 nmoles/kg, about 50 nmoles/kg to about 500 nmoles/kg, about 50 nmoles/kg to about 400 nmoles/kg, about 50 nmoles/kg to about 300 nmoles/kg, about 50 nmoles/kg to about 200 nmoles/kg, about 50 nmoles/kg to about 100 nmoles/kg, about 100 nmoles/kg to about 300 nmoles/kg, about 100 nmoles/kg to about 500 nmoles/kg, about 100 nmoles/kg to about 1000 nmoles/kg, about 100 nmoles/kg to about 2000 nmoles/kg of patient body weight. In other embodiments, the dose may be about 1 nmoles/kg, about 5 nmoles/kg, about 10 nmoles/kg, about 20 nmoles kg, about 25 nmoles/kg, about 30 nmoles/kg, about 40 nmoles/kg, about 50 nmoles/kg, about 60 nmoles/kg, about 70 nmoles/kg, about 80 nmoles/kg, about 90 nmoles/kg, about 100 nmoles/kg, about 150 nmoles/kg, about 200 nmoles/kg, about 250 nmoles/kg, about 300 nmoles/kg, about 350 nmoles/kg, about 400 nmoles/kg, about 450 nmoles/kg, about 500 nmoles/kg, about 600 nmoles/kg, about 700 nmoles/kg, about 800 nmoles/kg, about 900 nmoles/kg, about 1000 nmoles/kg, about 2000 nmoles/kg, about 2500 nmoles/kg or about 3000 nmoles/kg of body weight of the patient.

In various other embodiments, the dose of the compound, or the pharmaceutically acceptable salt thereof, may range from, for example, about 10 nmoles/kg to about 10000 nmoles/kg, from about 10 nmoles/kg to about 5000 nmoles/kg, from about 10 nmoles/kg to about 3000 nmoles/kg, about 10 nmoles/kg to about 2500 nmoles/kg, about 10 nmoles/kg to about 2000 nmoles/kg, about 10 nmoles/kg to about 1000 nmoles/kg, about 10 nmoles/kg to about 900 nmoles/kg, about 10 nmoles/kg to about 800 nmoles/kg, about 10 nmoles/kg to about 700 nmoles/kg, about 10 nmoles/kg to about 600 nmoles/kg, about 10 nmoles/kg to about 500 nmoles/kg, about 10 nmoles/kg to about 400 nmoles/kg, about 10 nmoles/kg to about 300 nmoles/kg, about 10 nmoles/kg to about 200 nmoles/kg, about 10 nmoles/kg to about 150 nmoles/kg, about 10 nmoles/kg to about 100 nmoles/kg, about 10 nmoles/kg to about 90 nmoles/kg, about 10 nmoles/kg to about 80 nmoles/kg, about 10 nmoles/kg to about 70 nmoles/kg, about 10 nmoles/kg to about 60 nmoles/kg, about 10 nmoles/kg to about 50 nmoles/kg, about 10 nmoles/kg to about 40 nmoles/kg, about 10 nmoles/kg to about 30 nmoles/kg, about 10 nmoles/kg to about 20 nmoles/kg, about 200 nmoles/kg to about 900 nmoles/kg, about 200 nmoles/kg to about 800 nmoles/kg, about 200 nmoles/kg to about 700 nmoles/kg, about 200 nmoles/kg to about 600 nmoles/kg, about 200 nmoles/kg to about 500 nmoles/kg, about 250 nmoles/kg to about 600 nmoles/kg, about 300 nmoles/kg to about 600 nmoles/kg, about 300 nmoles/kg to about 500 nmoles/kg, or about 400 nmoles/kg to about 600 nmoles/kg, of body weight of the patient. In various other embodiments, the dose of the compound, or the pharmaceutically acceptable salt thereof, may range from, for example, about 1 nmoles/kg to about 10000 nmoles/kg, from about 1 nmoles/kg to about 5000 nmoles/kg, from about 1 nmoles/kg to about 3000 nmoles/kg, about 1 nmoles/kg to about 2500 nmoles/kg, about 1 nmoles/kg to about 2000 nmoles/kg, about 1 nmoles/kg to about 1000 nmoles/kg, about 1 nmoles/kg to about 900 nmoles/kg, about 1 nmoles/kg to about 800 nmoles/kg, about 1 nmoles/kg to about 700 nmoles/kg, about 1 nmoles/kg to about 600 nmoles/kg, about 1 nmoles/kg to about 500 nmoles/kg, about 1 nmoles/kg to about 400 nmoles/kg, about 1 nmoles/kg to about 300 nmoles/kg, about 1 nmoles/kg to about 200 nmoles/kg, about 1 nmoles/kg to about 150 nmoles/kg, about 1 nmoles/kg to about 100 nmoles/kg, about 1 nmoles/kg to about 90 nmoles/kg, about 1 nmoles/kg to about 80 nmoles/kg, about 1 nmoles/kg to about 70 nmoles/kg, about 1 nmoles/kg to about 60 nmoles/kg, about 1 nmoles/kg to about 50 nmoles/kg, about 1 nmoles/kg to about 40 nmoles/kg, about 1 nmoles/kg to about 30 nmoles/kg, or about 1 nmoles/kg to about 20 nmoles/kg, In yet other embodiments, the dose may be about 0.1 nmoles/kg, about 0.2 nmoles/kg, about 0.3 nmoles/kg, about 0.4 nmoles kg, or about 0.5 nmoles/kg, about 0.1 nmoles/kg to about 1000 nmoles/kg, about 0.1 nmoles/kg to about 900 nmoles/kg, about 0.1 nmoles/kg to about 850 nmoles/kg, about 0.1 nmoles/kg to about 800 nmoles/kg, about 0.1 nmoles/kg to about 700 nmoles/kg, about 0.1 nmoles/kg to about 600 nmoles/kg, about 0.1 nmoles/kg to about 500 nmoles/kg, about 0.1 nmoles/kg to about 400 nmoles/kg, about 0.1 nmoles/kg to about 300 nmoles/kg, about 0.1 nmoles/kg to about 200 nmoles/kg, about 0.1 nmoles/kg to about 100 nmoles/kg, about 0.1 nmoles/kg to about 50 nmoles/kg, about 0.1 nmoles/kg to about 10 nmoles/kg, or about 0.1 nmoles/kg to about 1 nmoles/kg of body weight of the patient. In other embodiments, the dose may be about 0.3 nmoles/kg to about 1000 nmoles/kg, about 0.3 nmoles/kg to about 900 nmoles/kg, about 0.3 nmoles/kg to about 850 nmoles/kg, about 0.3 nmoles/kg to about 800 nmoles/kg, about 0.3 nmoles/kg to about 700 nmoles/kg, about 0.3 nmoles/kg to about 600 nmoles/kg, about 0.3 nmoles/kg to about 500 nmoles/kg, about 0.3 nmoles/kg to about 400 nmoles/kg, about 0.3 nmoles/kg to about 300 nmoles/kg, about 0.3 nmoles/kg to about 200 nmoles/kg, about 0.3 nmoles/kg to about 100 nmoles/kg, about 0.3 nmoles/kg to about 50 nmoles/kg, about 0.3 nmoles/kg to about 10 nmoles/kg, or about 0.3 nmoles/kg to about 1 nmoles/kg of body weight of the patient. In these embodiments, “kg” is kilograms of body weight of the patient. In one aspect, a single dose or multiple doses of the compound, or pharmaceutically acceptable salt thereof, may be administered to the patient.

In another embodiment, between about 20 μg/kg of body weight of the patient and about 3 mg/kg of body weight of the patient of the compound, or the pharmaceutically acceptable salt thereof, can be administered to the patient. In another aspect, amounts can be from about 0.2 mg/kg of body weight of the patient and about 0.4 mg/kg of body weight of the patient, or can be about 50 μg/kg of body weight of the patient. In one aspect, a single dose or multiple doses of the compound, or the pharmaceutically acceptable salt thereof, may be administered to the patient.

In one embodiment, the small molecule ligand linked to the targeting moiety (the compound) can be administered to the patient before the CAR T cell composition comprising CAR T cells wherein the CAR T cells have a CAR comprising an E2 anti-fluorescein antibody fragment. In another embodiment, the small molecule ligand linked to the targeting moiety (bridge) can be administered to the patient at the same time as the CAR T cell composition comprising CAR T cells wherein the CAR T cells have a CAR comprising an E2 anti-fluorescein antibody fragment, but in different formulations, or in the same formulation. In yet another embodiment, the small molecule ligand linked to the targeting moiety can be administered to the patient after the CAR T cell composition comprising CAR T cells wherein the CAR T cells have a CAR comprising an E2 anti-fluorescein antibody fragment.

In one illustrative aspect, the timing between the administration of the CAR T cells having a CAR comprising an E2 anti-fluorescein antibody fragment, and the small molecule linked to the targeting moiety (bridge) may vary widely depending on factors that include the type of CAR T cells expressing the E2 anti-fluorescein antibody fragment being used, the binding specificity of the CAR having the E2 anti-fluorescein antibody fragment, the identity of the targeting moiety and the small molecule ligand, the identity of the cancer, the location in the patient of the cancer, the means used to administer to the patient the CAR T cells expressing the E2 anti-fluorescein antibody fragment and the small molecule ligand linked to the targeting moiety, and the health, age, and weight of the patient. In one aspect, the small molecule ligand linked to the targeting moiety can be administered before or after the CART cells, such as within about 3, 6, 9, 12, 15, 18, 21, 24, 27, 30, 33, 36, 39, 42, 45, 48, or 51 hours, or within about 0.5, 1, 1.5, 2, 2.5, 3, 4 5, 6, 7, 8, 9, 10 or more days.

In one embodiment, any applicable dosing schedule known in the art can be used for administration of the compound, or the pharmaceutically acceptable salt thereof, or for the CAR T cell composition wherein the CAR T cell composition comprises CAR T cells having a CAR, wherein the CAR comprises an E2 anti-fluorescein antibody fragment. For example, once per day dosing (a.k.a. qd), twice per day dosing (a.k.a. bid), three times per day dosing (a.k.a. tid), twice per week dosing (a.k.a. BIW), three times per week dosing (a.k.a. TIW), once weekly dosing, and the like, can be used. In one aspect, the dosing schedule selected for the compound, or the pharmaceutically acceptable salt thereof, and the CAR T cell composition wherein the CAR T cell composition comprises CAR T cells having a CAR, wherein the CAR comprises an E2 anti-fluorescein antibody fragment, can take into consideration the concentration of the compound, or the pharmaceutically acceptable salt thereof, and the number of CAR T cells expressing the E2 anti-fluorescein antibody fragment administered, to regulate the cytotoxicity of the CAR T cell composition wherein the CAR T cell composition comprises CAR T cells having a CAR, wherein the CAR comprises an E2 anti-fluorescein antibody fragment, and to control CRS.

In one embodiment, to prevent or inhibit cytokine release syndrome (CRS) in the patient, a method of treatment of a cancer is provided. The method comprises i) administering to a patient a compound, or a pharmaceutically acceptable salt thereof, wherein the compound comprises a small molecule ligand linked to a targeting moiety by a linker, ii) administering to the patient a CAR T cell composition wherein the CAR T cell composition comprises CAR T cells wherein the CAR T cells comprise a CAR, wherein the CAR comprises an E2 anti-fluorescein antibody fragment, and iii) administering to the patient a folate, a conjugate comprising a folate wherein the conjugate comprising a folate does not comprise a targeting moiety, or a drug that inhibits activation of the CAR T cells.

In this method embodiment, the step of administering to the patient a folate, a conjugate comprising a folate wherein the conjugate comprising a folate does not comprise a targeting moiety, or a drug that inhibits activation of the CAR T cells expressing the E2 anti-fluorescein antibody fragment can be used to prevent or inhibit CRS in the patient. In this embodiment, any of a folate, a conjugate comprising a folate wherein the conjugate comprising a folate does not comprise a targeting moiety, or a drug that inhibits activation of the CAR T cells expressing the E2 anti-fluorescein antibody fragment can be referred to herein as “a rescue agent”. In one embodiment, a folate, such as folic acid, can be administered to prevent or inhibit CRS in the patient. In this embodiment, the folate inhibits interaction of the bridge (i.e., the small molecule ligand linked to the targeting moiety by a linker) with the receptors for the bridge on the tumor inhibiting tumor lysis and preventing or inhibiting CRS in the patient.

In another embodiment, the rescue agent can reduce CRS in as quickly as about 30 minutes, about 1 hour, about 2 hours, about 3 hours, about 4 hours, about 5 hours, about 6 hours, about 7 hours, about 8 hours, about 9 hours, or about 10 hours after administration of the rescue agent. For example, the grade of CRS can be reduced (e.g., grade 3 to grade 2, grade 4 to grade 3, etc.).

An exemplary grading system is shown below with columns 1-6 from left to right which are grades 0, 1, 2, 3, 4, and 5:

CRS Grading Scale

| 0 | 1 | 2 | 3 | 4 | 5 |
|---|---|---|---|---|---|
| Normal | ~pilo | pilo | pilo, greasy | moribund | Death |
| | ~greasy | greasy | bunched, thin | bunched, pale | |
| | still | less | not active, unless | lethargic | |
| | active | active | stimulated | | |

In one embodiment, the folate administered as an inhibitor of binding of the bridge to the tumor can be, for example, folic acid, a folic acid analog, or another folate receptor-binding molecule. In various embodiments, analogs of folate that can be used include folinic acid, pteropolyglutamic acid, and folate receptor-binding pteridines such as tetrahydropterins, dihydrofolates, tetrahydrofolates, and their deaza and dideaza analogs. The terms “deaza” and “dideaza” analogs refers to the art recognized analogs having a carbon atom substituted for one or two nitrogen atoms in the naturally occurring folic acid structure. For example, the deaza analogs include the 1-deaza, 3-deaza, 5-deaza, 8-deaza, and 10-deaza analogs. The dideaza analogs include, for example, 1,5 dideaza, 5,10-dideaza, 8,10-dideaza, and 5,8-dideaza analogs. The foregoing folic acid analogs are conventionally termed “folates,” reflecting their capacity to bind to folate receptors. Other folate receptor-binding analogs include aminopterin, amethopterin (methotrexate), N10-methylfolate, 2-deamino-hydroxyfolate, deaza analogs such as 1-deazamethopterin or 3-deazamethopterin, and 3′,5′-dichloro-4-amino-4-deoxy-N10-methylpteroylglutamic acid (dichloromethotrexate).

In another embodiment, the folate administered as an inhibitor of binding of the bridge to the tumor has the formula

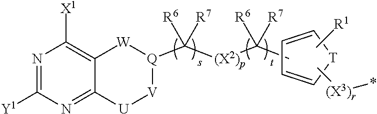

wherein X¹ and Y¹ are each-independently selected from the group consisting of halo, R², OR², SR³, and NR⁴R⁵;
U, V, and W represent divalent moieties each independently selected from the group consisting of —(R^{6a})C═, —N═, —(R^{6a})C(R^{7a})—, and —N(R^{4a})—; Q is selected from the group consisting of C and CH; T is selected from the group consisting of S, O, N, and —C═C—;
X² and X³ are each independently selected from the group consisting of oxygen, sulfur, —C(Z)—, —C(Z)O—, —OC(Z)—, —N(R^{4b})—, —C(Z)N(R^{4b})—, —N(R^{4b})C(Z)—, —OC(Z)N(R^{4b})—, —N(R^{4b})C(Z)O—, —N(R^{4b})C(Z)N(R^{5b})—, —S(O)—, —S(O)₂—, —N(R^{4a})S(O)₂—, <C(R^{6b})(R^{7b})—, —N(C≡CH)—, —N(CH₂C≡CH)—, C₁-C₁₂ alkylene, and C₁-C₁₂ alkylenoxy, where Z is oxygen or sulfur;
R¹ is selected-from the group consisting of hydrogen, halo, C¹-C¹²-alkyl, and C₁-C¹² alkoxy;
R², R³, R⁴, R^{4a}, R^{4b}, R⁵, R^{5b}, R^{6b}, and R^{7b} are each independently selected from the group consisting of hydrogen, halo, C₁-C₁₂ alkyl, C₁-C₁₂ alkoxy, C₁-C₁₂alkanoyl, C₁-C₁₂alkenyl, C₁-C₁₂ alkynyl, (C₁-C₁₂ alkoxy)carbonyl, and (C₁-C₁₂ alkylamino)carbonyl;
R⁶ and R⁷ are each independently selected from the group consisting of hydrogen, halo, C₁-C₁₂ alkyl, and C₁-C₁₂ alkoxy; or, R⁶ and R⁷ are taken together to form a carbonyl group;
R^{6a} and R^{7a} are each independently selected from the group consisting of hydrogen, halo, C₁-C₁₂ alkyl, and C₁-C₁₂ alkoxy; or R^{6a} and R^{7a} are taken together to form a carbonyl group;
p, r, s, and t are each independently either 0 or 1; and
* represents an optional covalent bond to the rest of the conjugate.

In yet another embodiment, a conjugate comprising a folate can be administered to prevent or inhibit cytokine release syndrome (CRS) in the patient. CRS is a term well-known in the art and this syndrome can cause detrimental effects to the patient, including, but not limited to weight loss, high fever, pulmonary edema, and a dangerous drop in blood pressure.

In this embodiment, the conjugate comprising a folate does not comprise a targeting moiety, and, thus, the conjugate inhibits interaction of the bridge with the tumor to prevent tumor lysis and reduce CRS in the patient. In this embodiment, the folate moiety in the conjugate comprising a folate can comprise any of the folates described in the preceding paragraphs linked to a chemical moiety that does not comprise a targeting moiety. In one aspect, the conjugate comprising a folate can comprise a folate linked to one or more amino acids that do not comprise a targeting moiety. Illustratively, the conjugate comprising a folate can have the formula

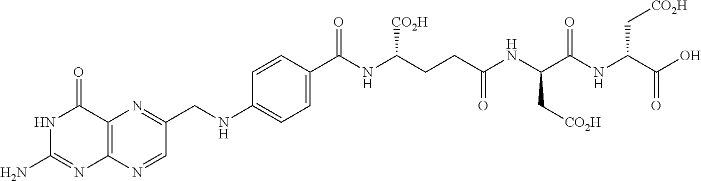 [SMILES: Nc1nc2ncc(CNc3ccc(C(=O)N[C@@H](CCC(=O)N[C@H](CC(=O)O)C(=O)N[C@H](CC(=O)O)C(=O)O)C(=O)O)cc3)nc2c(=O)[nH]1]

This compound can also be referred to as “EC923”. In these embodiments, the folate or the conjugate comprising a folate can be administered to the patient in molar excess relative to the bridge (i.e., the small molecule ligand linked to a targeting moiety by a linker), such as a 10-fold excess, a 100-fold excess, a 200-fold excess a 300-fold excess a 400-fold excess a 500-fold excess a 600-fold excess a 700-fold excess a 800-fold excess a 900-fold excess, a 1000-fold excess, or a 10,000-fold excess of the folate or the conjugate comprising a folate relative to the small molecule ligand linked to a targeting moiety by a linker. The amount of the folate or the conjugate comprising a folate relative to the amount of the small molecule ligand linked to a targeting moiety by a linker needed to inhibit interaction of the bridge with the tumor can be determined by the skilled artisan.

In another embodiment, a rescue agent that inhibits activation of the CAR T cells expressing the E2 anti-fluorescein antibody fragment, can be administered to the patient to inhibit CAR T cell activation and to inhibit or prevent CRS in the patient. In one aspect the agent can be selected from the group consisting of a lymphocyte-specific protein tyrosine kinase inhibitor (e.g., Dasatinib), a PI3 kinase inhibitor (e.g., GDC0980), an inhibitor of an IL-2 inducible T cell kinase (e.g., BMS-509744), JAK inhibitors, BTK inhibitors, Tocilizumab, SIP agonists (e.g., Siponimod and Ozanimod) and an agent that blocks CAR T cell binding to the bridge, but does not bind to the cancer (e.g., fluoresceinamine, FITC, or sodium fluorescein). It is understood by the skilled artisan that FITC (i.e., fluorescein) can be in the form of a salt (e.g., sodium fluorescein), or in its unsalted form, under physiological conditions or, for example, in a buffer at physiological pH. Accordingly, in one embodiment, when fluorescein is administered to a patient it may be in equilibrium between its salted form (e.g., sodium fluorescein) and its unsalted form. In another embodiment, a rescue agent that inhibits activation of CAR T cells can be a compound of the formula

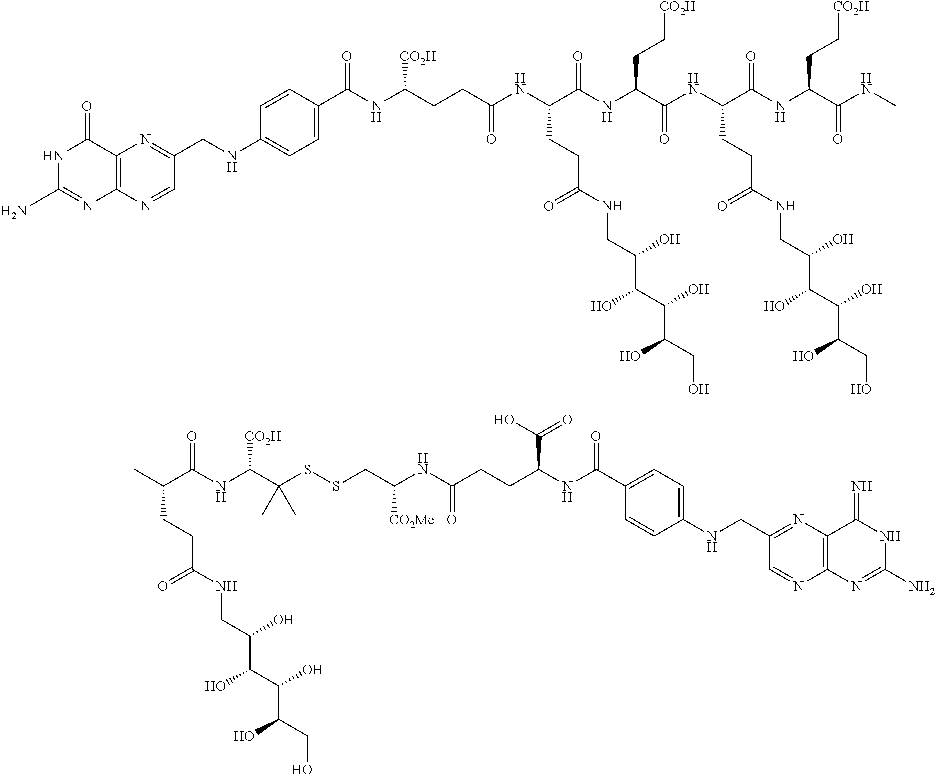

In various embodiments, the rescue agent can be administered at a concentration of from about 0.001 nM to about 100 mM, about 0.01 nM to about 100 mM, about 1 nM to about 100 mM, about 10 nM to about 100 mM, about 50 nM to about 100 mM, or from about 100 nM to about 100 mM in any appropriate volume, including, for example, 0.1 ml, 0.2 ml, 0.3 ml, 0.4 ml, 0.5 ml, 0.6 ml, 0.7 ml, 0.8 ml, 0.9 ml, 1 ml, 2 ml, 3 ml, 4 ml, 5 ml, 10 ml, 100 ml, or 1000 ml. In other embodiments, the rescue agent can be administered at a dose of about 0.01 to about 300 umoles/kg of body weight of the patient, about 0.06 to about 100 umoles/kg of body weight of the patient, about 0.06 to about 90 umoles/kg of body weight of the patient, about 0.06 to about 80 umoles/kg of body weight of the patient, about 0.06 to about 70 umoles/kg of body weight of the patient, about 0.06 to about 60 umoles/kg of body weight of the patient, about 0.06 to about 50 umoles/kg of body weight of the patient, about 0.06 to about 40 umoles/kg of body weight of the patient, about 0.06 to about 30 umoles/kg of body weight of the patient, about 0.06 to about 20 umoles/kg of body weight of the patient, about 0.06 to about 10 umoles/kg of body weight of the patient, about 0.06 to about 8 umoles/kg of body weight of the patient, or about 0.06 to about 6 umoles/kg of body weight of the patient.

In these embodiments, the rescue agent can be administered to the patient in molar excess relative to the compound, or its pharmaceutically acceptable salt (i.e., the small molecule ligand linked to a targeting moiety by a linker), such as about a 10-fold excess, about a 20-fold excess, about a 30-fold excess, about a 40-fold excess, about a 50-fold excess, about a 60-fold excess, about a 70-fold excess, about a 80-fold excess, about a 90-fold excess, about a 100-fold excess, about a 200-fold excess, about a 300-fold excess, about a 400-fold excess, about a 500-fold excess, about a 600-fold excess, about a 700-fold excess, about a 800-fold excess, about a 900-fold excess, about a 1000-fold excess, or about a 10,000-fold excess of the rescue agent relative to the small molecule ligand linked to a targeting moiety by a linker. The amount of the rescue agent relative to the amount of the small molecule ligand linked to a targeting moiety by a linker (bridge) needed to inhibit interaction of the compound, or its pharmaceutically acceptable salt, with the tumor and/or the CAR T cells expressing the E2 anti-fluorescein antibody fragment can be determined by the skilled artisan.

In another embodiment, more than one dose can be administered to the patient of the folate, the conjugate comprising a folate wherein the conjugate comprising a folate does not comprise a targeting moiety, or the agent that inhibits activation of the CAR T cells expressing the E2 anti-fluorescein antibody fragment.

In the ‘rescue agent’ embodiments described herein, the folate, the conjugate comprising a folate wherein the conjugate comprising a folate does not comprise a targeting moiety, or the agent that inhibits activation of the CAR T cells expressing the E2 anti-fluorescein antibody fragment, can be administered to the patient before and/or after the compound, or the pharmaceutically acceptable salt thereof. In another aspect, the compound, or the pharmaceutically acceptable salt thereof, can be administered before and subsequent to administration of the folate, the conjugate comprising a folate wherein the conjugate comprising a folate does not comprise a targeting moiety, or the agent that inhibits activation of the CAR T cells expressing the E2 anti-fluorescein antibody fragment. In this embodiment, the subsequent administration of the compound, or the pharmaceutically acceptable salt thereof, can cause activation of the CAR T cells expressing the E2 anti-fluorescein antibody fragment and an increase in cytokine levels in the patient.

In another embodiment, administration of the folate, the conjugate comprising a folate wherein the conjugate comprising a folate does not comprise a targeting moiety, or the agent that inhibits activation of the CAR T cells expressing the E2 anti-fluorescein antibody fragment, can cause reduction in cytokine levels in the patient. In yet another embodiment, the reduction in cytokine levels can occur by about 1 hour, by about 2 hours, by about 3 hours, by about 4 hours, by about 5 hours, by about 6 hours, by about 7 hours, or by about 8 hours after administration to the patient of the folate, the conjugate comprising a folate wherein the conjugate comprising a folate does not comprise a targeting moiety, or the agent that inhibits activation of the CAR T cells expressing the E2 anti-fluorescein antibody fragment. In another embodiment, the reduction in cytokine levels is a reduction to about the cytokine levels in an untreated patient.

In another illustrative embodiment, the number of the CAR T cells expressing the E2 anti-fluorescein antibody fragment can increase in the blood of the patient after administration of the folate, the conjugate comprising a folate wherein the conjugate comprising a folate does not comprise a targeting moiety, or the agent that inhibits activation of the CAR T cells expressing the E2 anti-fluorescein antibody fragment, even though cytokine levels in the patient are reduced. In another illustrative aspect, activation of the CAR T cells expressing the E2 anti-fluorescein antibody fragment can be enhanced or maintained, relative to a patient not treated with a rescue agent, after administration of the folate, the conjugate comprising a folate wherein the conjugate comprising a folate does not comprise a targeting moiety, or the rescue agent that inhibits activation of the CAR T cells wherein the CAR T cells comprise a CAR comprising the E2 anti-fluorescein antibody fragment, even though cytokine levels in the treated patient are reduced. In still another embodiment, the cancer comprises a tumor and tumor size in the patient is not increased when the folate, the conjugate comprising a folate wherein the conjugate comprising a folate does not comprise a targeting moiety, or the rescue agent that inhibits activation of the CAR T cells expressing the E2 anti-fluorescein antibody fragment is administered to the patient. In this embodiment, a complete response for the tumor can be obtained.

In other embodiments, the rescue agent that inhibits activation of the CAR T cells expressing the E2 anti-fluorescein antibody fragment is administered to the patient when the CRS grade reaches 1, 2, 3, or 4 or when the CRS grade reaches 3 or 4. In another aspect, lung edema is reduced in the patient when the rescue agent is administered.

In one embodiment described herein a method of treatment of a cancer is provided, and the method comprises i) administering continuously to a patient a compound, or a pharmaceutically acceptable salt thereof, wherein the compound comprises a small molecule ligand linked to a targeting moiety by a linker, ii) administering to the patient a CAR T cell composition comprising CAR T cells wherein the CAR T cells comprise a CAR comprising an E2 anti-fluorescein antibody fragment, and iii) ending the continuous administration of the compound, or the pharmaceutically acceptable salt thereof, to inhibit or prevent cytokine release syndrome in the patient.

In accordance with this embodiment, the term “continuously” can mean administering the compound, or the pharmaceutically acceptable salt thereof, to the patient, for example, at least one hour, at least four hours, at least six hours, at least eight hours, at least ten hours, at least twelve hours, or at least twenty-four hours, or can mean a regimen of daily or weekly administration, such as once a day, two times a day, three times a day, every day, every other day, one time weekly, two times weekly, three times weekly, or any other suitable regimen that would be considered continuous administration by a person skilled in the art. In another aspect, the term “continuously” can mean any combination of the embodiments described in this paragraph.

In this method embodiment, the step of “ending the continuous administration” of the compound, or the pharmaceutically acceptable salt thereof, to inhibit or prevent cytokine release syndrome in the patient, can mean, for example, discontinuing administration after administration for a continuous period of time, such as hours or days, or discontinuing a treatment regimen, such as the daily or weekly regimens described above. In another embodiment, the step of “ending the continuous administration” can mean, for example, administration until an unacceptable loss of body weight for the patient occurs, or until any other unacceptable side effect occurs such as a high fever, a drop in blood pressure, or pulmonary edema. In this embodiment, the step of “ending the continuous administration” of the compound, or the pharmaceutically acceptable salt thereof, does not mean a single treatment with the compound, or the pharmaceutically acceptable salt thereof, with no subsequent treatment with the compound, or the pharmaceutically acceptable salt thereof. In this method embodiment “to inhibit or prevent” cytokine release syndrome (CRS) means eliminating CRS or reducing or ameliorating the symptoms of CRS.

In one embodiment of the embodiment involving ending the continuous administration” of the compound, or the pharmaceutically acceptable salt thereof, the method can further comprise step iv) of re-administering the compound, or the pharmaceutically acceptable salt thereof, to the patient. In one embodiment, the compound, or the pharmaceutically acceptable salt thereof, can be administered, for example, once weekly and one dose can be omitted. In another embodiment, the compound, or the pharmaceutically acceptable salt thereof, can be administered on alternate days (i.e., every other day) and one or more (e.g., two, three, fours, etc.) doses can be omitted. In another embodiment, the compound, or the pharmaceutically acceptable salt thereof, can be administered twice weekly and one or more (e.g., two, three, fours, etc.) doses can be omitted. In another embodiment, the compound, or the pharmaceutically acceptable salt thereof, can be administered Monday, Tuesday, and the following Monday, and then dosing can be stopped for two weeks and the cycle repeated. In another embodiment, any of the regimen embodiments described above can be used and one or more (e.g., two, three, four, etc.) doses can be omitted. In these embodiments, the omitted doses can prevent or reduce CRS in the patient.

In yet another illustrative aspect, a method of treatment of a cancer is provided. The method comprises i) administering to a patient a compound, or a pharmaceutically acceptable salt thereof, wherein the compound comprises a small molecule ligand linked to a targeting moiety by a linker, wherein at least a first dose and a second dose of the compound, or the pharmaceutically acceptable salt thereof, are administered to the patient, wherein the first dose and the second dose are different, wherein the second dose of the compound, or the pharmaceutically acceptable salt thereof, is about 2-fold to about 15000-fold greater in amount than the first dose of the compound, or the pharmaceutically acceptable salt thereof, and ii) administering to the patient a CAR T cell composition comprising CAR T cells wherein the CAR T cells comprise a CAR comprising an E2 anti-fluorescein antibody fragment.

In this embodiment, the dose of the compound, or the pharmaceutically acceptable salt thereof, can be escalated gradually to inhibit or prevent cytokine release syndrome in the patient. For example, at least a first dose and a second dose of the compound, or the pharmaceutically acceptable salt thereof, can be administered to the patient, wherein the first dose and the second dose are different, wherein the second dose of the compound, or the pharmaceutically acceptable salt thereof, is about 20-fold to about 15000-fold greater, 2-fold to about 15000-fold greater in amount than the first dose of the compound, or the pharmaceutically acceptable salt thereof. In other embodiments, the second dose, or a subsequent dose, can be about 2-fold to about 5-fold, about 2-fold to about 10-fold, about 2-fold to about 20-fold, about 2-fold to about 30-fold, about 2-fold to about 40-fold, about 2-fold to about 50-fold, about 2-fold to about 60-fold, about 2-fold to about 70-fold, about 2-fold to about 80-fold, about 2-fold to about 90-fold, about 2-fold to about 100-fold, about 2-fold to about 15000-fold, about 2-fold to about 10000-fold, about 5-fold to about 9000-fold, about 5-fold to about 8000-fold, about 5-fold to about 7000-fold, about 5-fold to about 6000-fold, about 5-fold to about 5000-fold, about 5-fold to about 4000-fold, about 5-fold to about 3000-fold, about 5-fold to about 4000-fold, about 5-fold to about 3000-fold, about 5-fold to about 2000-fold, about 5-fold to about 1000-fold, about 5-fold to about 750-fold, about 2-fold to about 750-fold, about 5-fold to about 500-fold, about 5-fold to about 100-fold, about 800-fold to about 15000-fold, about 800-fold to about 10000-fold, about 800-fold to about 9000-fold, about 800-fold to about 8000-fold, about 800-fold to about 7000-fold, about 800-fold to about 6000-fold, about 800-fold to about 5000-fold, about 800-fold to about 4000-fold, about 800-fold to about 3000-fold, about 800-fold to about 2000-fold, about 800-fold to about 1000-fold, about 8000-fold to about 15000-fold, about 8000-fold to about 10000-fold, about 8000-fold to about 9000-fold, about 15000-fold, about 10000-fold, about 9000-fold, about 8000-fold, about 7000-fold, about 6000-fold, about 5000-fold, about 4000-fold, about 3000-fold, about 2000-fold, about 1000-fold, about 500-fold, about 400-fold, about 300-fold, about 200-fold, about 100-fold, about 90-fold, about 80-fold, about 70-fold, about 60-fold, about 50-fold, about 40-fold, about 30-fold, about 20-fold, about 10-fold, about 5-fold, or about 2-fold greater in amount than the first dose of the compound, or the pharmaceutically acceptable salt thereof.

In another illustrative embodiment of the dose escalation method, at least a first dose, a second dose, and a third dose of the compound, or the pharmaceutically acceptable salt thereof, can be administered to the patient, wherein the first dose, the second dose, and the third dose are different, wherein the second dose of the compound, or the pharmaceutically acceptable salt thereof, is about 2-fold to about 750-fold greater in amount than the first dose of the compound, or the pharmaceutically acceptable salt thereof, and wherein the third dose of the compound, or the pharmaceutically acceptable salt thereof, is about 800-fold to about 10000-fold greater in amount than the first dose of the compound, or the pharmaceutically acceptable salt thereof.

In another aspect of the dose escalation method, at least a first dose, a second dose, a third dose, and a fourth dose of the compound, or the pharmaceutically acceptable salt thereof, can be administered to the patient, wherein the first dose, the second dose, the third dose, and the fourth dose are different, wherein the second dose of the compound, or the pharmaceutically acceptable salt thereof, is about 2-fold to about 750-fold greater in amount than the first dose of the compound, or the pharmaceutically acceptable salt thereof, wherein the third dose of the compound, or the pharmaceutically acceptable salt thereof, is about 800-fold to about 7500-fold greater in amount than the first dose of the compound, or the pharmaceutically acceptable salt thereof, and wherein the fourth dose of the compound, or the pharmaceutically acceptable salt thereof, is about 8000 to about 15000-fold greater in amount than the first dose of the compound, or the pharmaceutically acceptable salt thereof.

In still another embodiment, the second dose of the compound, or the pharmaceutically acceptable salt thereof, can be about 100-fold greater in amount than the first dose of the compound, or the pharmaceutically acceptable salt thereof, the third dose of the compound, or the pharmaceutically acceptable salt thereof, can be about 1000-fold greater in amount than the first dose of the compound, or the pharmaceutically acceptable salt thereof, and the fourth dose of the compound, or the pharmaceutically acceptable salt thereof, can be about 10000-fold greater in amount than the first dose of the compound, or the pharmaceutically acceptable salt thereof. In an exemplary embodiment, the first dose of the compound, or the pharmaceutically acceptable salt thereof, is 0.05 nmoles/kg, the second dose is 5 nmoles/kg, the third dose is 50 nmoles/kg, and the fourth dose is 500 nmoles/kg. In the dose escalation embodiments described herein, the first, second, third, fourth, and any subsequent doses of the compound, or the pharmaceutically acceptable salt thereof, can be administered multiple times (e.g., the first dose at 0.05 nmoles/kg can be administered several times before the subsequent escalated doses are administered).

In another embodiment described herein, a method of treatment of a cancer is provided. The method comprises i) administering to a patient a first dose of a compound, or a pharmaceutically acceptable salt thereof, wherein the compound comprises a small molecule ligand linked to a targeting moiety by a linker, ii) administering to the patient at least a second dose of the compound, or a pharmaceutically acceptable salt thereof, wherein the second dose of the compound, or the pharmaceutically acceptable salt thereof, is at least about 50 percent lower in amount than the first dose of the compound, or the pharmaceutically acceptable salt thereof, and iii) administering to the patient a dose of a CAR T cell composition comprising CAR T cells wherein the CAR T cells comprise a CAR comprising an E2 anti-fluorescein antibody fragment.

In various embodiments of this dose de-escalation embodiment, the second dose of the compound, or the pharmaceutically acceptable salt thereof, can be at least about 60 percent lower in amount than the first dose of the compound, or the pharmaceutically acceptable salt thereof, at least about 70 percent lower in amount than the first dose of the compound, or the pharmaceutically acceptable salt thereof, at least about 80 percent lower in amount than the first dose of the compound, or the pharmaceutically acceptable salt thereof, at least about 90 percent lower in amount than the first dose of the compound, or the pharmaceutically acceptable salt thereof, at least about 95 percent lower in amount than the first dose of the compound, or the pharmaceutically acceptable salt thereof, at least about 96 percent lower in amount than the first dose of the compound, or the pharmaceutically acceptable salt thereof, at least about 97 percent lower in amount than the first dose of the compound, or the pharmaceutically acceptable salt thereof, at least about 98 percent lower in amount than the first dose of the compound, or the pharmaceutically acceptable salt thereof, at least about 99 percent lower in amount than the first dose of the compound, or the pharmaceutically acceptable salt thereof, or at least about 99.5 percent lower in amount than the first dose of the compound, or the pharmaceutically acceptable salt thereof.

In various embodiments of the dose de-escalation embodiment described herein, the first dose of the compound, or the pharmaceutically acceptable salt thereof, can be about 100 nmoles/kg to about 1000 nmoles/kg of body weight of the patient, about 100 nmoles/kg to about 900 nmoles/kg of body weight of the patient, about 100 nmoles/kg to about 800 nmoles/kg of body weight of the patient, about 100 nmoles/kg to about 700 nmoles/kg of body weight of the patient, about 100 nmoles/kg to about 600 nmoles/kg of body weight of the patient, about 200 nmoles/kg to about 600 nmoles/kg of body weight of the patient, about 400 nmoles/kg to about 600 nmoles/kg of body weight of the patient, or about 500 nmoles/kg of body weight of the patient.

In various embodiments of the dose de-escalation embodiment described herein, the second dose of the compound, or the pharmaceutically acceptable salt thereof, can be about 0.5 nmoles/kg to about 500 nmoles/kg of body weight of the patient, about 0.5 nmoles/kg to about 450 nmoles/kg of body weight of the patient, about 0.5 nmoles/kg to about 400 nmoles/kg of body weight of the patient, about 0.5 nmoles/kg to about 350 nmoles/kg of body weight of the patient, about 0.5 nmoles/kg to about 300 nmoles/kg of body weight of the patient, about 1 nmole/kg to about 300 nmoles/kg of body weight of the patient, about 2 nmoles/kg to about 300 nmoles/kg of body weight of the patient, about 2 nmoles/kg to about 250 nmoles/kg of body weight of the patient, about 5 nmoles/kg to about 40 nmoles/kg of body weight of the patient, or about 40 nmoles/kg to about 150 nmoles/kg of body weight of the patient.

In additional embodiments of the dose de-escalation embodiment described herein, the method can further comprise administering a third dose of the compound, or the pharmaceutically acceptable salt thereof, wherein the third dose of the compound, or the pharmaceutically acceptable salt thereof, is the same as the second dose of the compound, or the pharmaceutically acceptable salt thereof. In another embodiment, the method can further comprise administering a fourth dose of the compound, or the pharmaceutically acceptable salt thereof, wherein the fourth dose of the compound, or the pharmaceutically acceptable salt thereof, is the same as the second dose, or the pharmaceutically acceptable salt thereof, and the third dose of the compound, or the pharmaceutically acceptable salt thereof. In yet another embodiment, the dose(s) of the compound, or the pharmaceutically acceptable salt thereof, administered after the first dose of the compound, or the pharmaceutically acceptable salt thereof, can maintain inhibition of growth of the cancer relative to the first dose of the compound, or the pharmaceutically acceptable salt thereof.

In other embodiments of the dose de-escalation embodiment described herein, the CAR T cells expressing the E2 anti-fluorescein antibody fragment can be administered at a dose of about 1 million of the CAR T cells expressing the E2 anti-fluorescein antibody fragment to about 40 million of the CAR T cells expressing the E2 anti-fluorescein antibody fragment. In still other embodiments, the dose(s) of the compound or the pharmaceutically acceptable salt thereof, administered after the first dose of the compound, or the pharmaceutically acceptable salt thereof, can be administered once or twice weekly.

In still other embodiments of the dose de-escalation embodiment described herein, the method can further comprise the step of administering to the patient a folate, a conjugate comprising a folate wherein the conjugate comprising a folate does not comprise a targeting moiety, or an agent that inhibits activation of the CAR T cells expressing the E2 anti-fluorescein antibody fragment. In another embodiment, the agent that inhibits activation of the CAR T cells expressing the E2 anti-fluorescein antibody fragment is administered to the patient and the agent is an agent that blocks binding of the CAR T cells expressing the E2 anti-fluorescein antibody fragment to the compound, or the pharmaceutically acceptable salt thereof, but does not bind to the cancer, and the agent is fluoresceinamine, sodium fluorescein, or fluorescein. In yet another embodiment, the agent is sodium fluorescein.

In another embodiment, a method of treatment of a cancer is provided. The method comprises i) administering to a patient a first dose of a compound, or a pharmaceutically acceptable salt thereof, wherein the compound comprises a small molecule ligand linked to a targeting moiety by a linker and wherein the compound, or the pharmaceutically acceptable salt thereof, is administered to the patient at least about one hour prior to the administration of a CAR T cell composition comprising CAR T cells wherein the CAR T cells comprise a CAR comprising an E2 anti-fluorescein antibody fragment, ii) then administering to the patient a dose of the CAR T cell composition comprising CAR T cells wherein the CAR T cells comprise a CAR comprising an E2 anti-fluorescein antibody fragment, and iii) then administering to the patient a second dose of the compound, or the pharmaceutically acceptable salt thereof. In various embodiments of this pre-treatment embodiment, the first dose of the compound, or the pharmaceutically acceptable salt thereof, can be administered to the patient at least about two hours prior to the administration of the CAR T cell composition, at least about four hours prior to the administration of the CAR T cell composition, at least about eight hours prior to the administration of the CAR T cell composition, at least about twelve hours prior to the administration of the CAR T cell composition, at least about sixteen hours prior to the administration of the CAR T cell composition, at least about twenty hours prior to the administration of the CAR T cell composition, or at least about twenty-four hours prior to the administration of the CAR T cell composition, in each case wherein the CAR T cell composition comprises CAR T cells comprising a CAR comprising an E2 anti-fluorescein antibody fragment.

In various embodiments of this pre-treatment embodiment, the second dose of the compound, or the pharmaceutically acceptable salt thereof, can be administered to the patient by at least about twenty-four hours after the administration of the CAR T cell composition, by at least about twenty hours after the administration of the CAR T cell composition, by at least about eighteen hours after the administration of the CAR T cell composition, by at least about sixteen hours after the administration of the CAR T cell composition, by at least about fourteen hours after the administration of the CAR T cell composition, by at least about twelve hours after the administration of the CAR T cell composition, by at least about ten hours after the administration of the CAR T cell composition, by at least about eight hours after the administration of the CAR T cell composition, by at least about six hours after the administration of the CAR T cell composition, by at least about four hours after the administration of the CAR T cell composition, or by at least about two hours after the administration of the CAR T cell composition, in each case wherein the CAR T cell composition comprises CAR T cells comprising a CAR comprising an E2 anti-fluorescein antibody fragment.

In any of the embodiments described herein where a folate is the ligand linked to the targeting moiety by a linker, the patient can be put on a folate deficient diet prior to treatment with the methods described herein, or the patient can be administered a folate in the diet. In the embodiment where the patient is administered folate, the dose can range, for example, from about 50 nmol/kg to about 3000 nmol/kg of patient body weight, about 50 nmol/kg to about 2000 nmol/kg, about 50 nmol/kg to about 1000 nmol/kg, about 50 nmol/kg to about 900 nmol/kg, about 50 nmol/kg to about 800 nmol/kg, about 50 nmol/kg to about 700 nmol/kg, about 50 nmol/kg to about 600 nmol/kg, about 50 nmol/kg to about 500 nmol/kg, about 50 nmol/kg to about 400 nmol/kg, about 50 nmol/kg to about 300 nmol/kg, about 50 nmol/kg to about 200 nmol/kg, about 50 nmol/kg to about 100 nmol/kg, about 100 nmol/kg to about 300 nmol/kg, about 100 nmol/kg to about 500 nmol/kg, about 100 nmol/kg to about 1000 nmol/kg, about 100 nmol/kg to about 2000 nmol/kg of patient body weight. In other embodiments, the dose may be about 100 nmol/kg, about 150 nmol/kg, about 200 nmol/kg, about 250 nmol/kg, about 300 nmol/kg, about 350 nmol/kg, about 400 nmol/kg, about 450 nmol/kg, about 500 nmol/kg, about 600 nmol/kg, about 700 nmol/kg, about 800 nmol/kg, about 900 nmol/kg, about 1000 nmol/kg, about 2000 nmol/kg, or about 3000 nmol/kg of patient body weight. In these embodiments, “kg” is kilograms of patient body weight. In one aspect, the folate can be administered, for example, daily, weekly, biweekly, three times a week, or using any suitable regimen for administration of the folate.

In various embodiments described herein, the CAR T cells can persist in elevated numbers of circulating CAR T cells for as long as about 10 days, as long as about 15 days, as long as about 20 days, as long as about 25 days, as long as about 30 days, as long as about 35 days, as long as about 40 days, as long as about 45 days, as long as about 50 days, as long as about 55 days, as long as about 60 days, as long as about 65 days, as long as about 70 days, as long as about 75 days, or as long as about 80 days post CAR T cell administration.

In various embodiments described herein, half-maximal effective concentrations (EC50) for the compound, or the pharmaceutically acceptable salt thereof, can be about 1 pM to about 2 nM, about 1 pM to about 5 nM, about 1 pM to about 10 nM, about 1 pM to about 20 nM, about 1 pM to about 30 nM, about 1 pM to about 40 nM, about 1 pM to about 50 nM, about 1 pM to about 60 nM, about 1 pM to about 70 nM, about 1 pM to about 80 nM, about 1 pM to about 90 nM, about 1 pM to about 100 nM, about 1 pM to about 200 nM, about 1 pM to about 300 nM, about 1 pM to about 400 nM, about 1 pM to about 500 nM, about 1 pM to about 600 nM, about 1 pM to about 700 nM, about 1 pM to about 800 nM, about 1 pM to about 900 nM, about 1 pM to about 1 nM, about 1 pM to about 900 pM, about 1 pM to about 800 pM, about 1 pM to about 700 pM, about 1 pM to about 600 pM, about 1 pM to about 500 pM, about 1 pM to about 400 pM, about 1 pM to about 300 pM, about 1 pM to about 200 pM, about 1 pM to about 100 pM, about 1 pM to about 90 pM, about 1 pM to about 80 pM, about 1 pM to about 70 pM, about 1 pM to about 60 pM, about 1 pM to about 50 pM, about 1 pM to about 40 pM, about 1 pM to about 30 pM, about 1 pM to about 20 pM, about 1 pM to about 10 pM, or about 1 pM to about 5 pM.

In various embodiments described herein, the Kd for binding of the compound, or the pharmaceutically acceptable salt thereof, to the CAR T cells can be about 1 nM to about 100 nM, about 1 nM to about 200 nM, about 1 nM to about 300 nM, about 1 nM to about 400 nM, about 1 nM to about 500 nM, about 1 nM to about 600 nM, about 1 nM to about 700 nM, about 1 nM to about 800 nM, about 1 nM to about 900 nM, about 100 nM to about 500 nM, about 100 nM to about 400 nM, about 100 nM to about 300 nM, about 100 nM to about 200 nM, about 100 nM to about 150 nM, or about 130 nM.

In various illustrative embodiments described herein, the compound, or the pharmaceutically acceptable salt thereof, can be first administered to the patient about 1, about 2, about 3, about 4, about 5, about 6, about 7, about 8, about 9, or about 10 days before or after the CAR T cells, or on any appropriate day before or after the CAR T cells.

In the various embodiments described herein, EGFRt-sorted or unsorted CAR T cells can be used. In another embodiment, a “clinical facsimile” batch of CAR T cells can be used with a low differentiation profile. In another embodiment, a “research batch” of CAR T cells can be used. The “clinical facsimile” batch (˜39% EGFRt+) can comprise CD4+ subsets at about 66% T_{SCM} and about 32% T_{CM} and CD8 subsets at about 95% T_{SCM} and about 3% T_{CM}. The research batch (˜23% EGFRt+) can comprise CD4 subsets at about 32% T_{SCM}, about 53% T_{CM}, about 11% T_{EM} and about 3.7% T_{EFF} and CD8 subsets at about 44% T_{SCM}, about 0.28% T_{CM}, about 3.4% T_{EM} and about 52% T_{EFF}.

In various additional embodiments of this pre-treatment embodiment, cytokine release resulting in off-target toxicity in the patient does not occur, but toxicity of the CAR T cells, expressing the E2 anti-fluorescein antibody fragment, to the cancer occurs or off-target tissue toxicity does not occur in the patient, but toxicity of the CAR T cells expressing the E2 anti-fluorescein antibody fragment to the cancer occurs, or the cancer comprises a tumor, and tumor size is reduced in the patient, but off-target toxicity does not occur, or reduction in tumor size in the patient is greater than in a patient not pre-treated with the compound, or the pharmaceutically acceptable salt thereof, prior to administration of the CAR T cell composition comprising CAR T cells comprising a CAR comprising an E2 anti-fluorescein antibody fragment. As would be understood by a skilled artisan, the “target” can be the cancer (for example a tumor).

In another embodiment, a method of treatment of a cancer is provided. The method comprises i) administering to a patient a compound, or a pharmaceutically acceptable salt thereof, wherein the compound comprises a small molecule ligand linked to a targeting moiety by a linker, and ii) administering to the patient a CAR T cell composition wherein the CAR T cell composition comprises CAR T cells and wherein the CAR T cells comprise a CAR comprising an E2 anti-fluorescein antibody fragment, and wherein the small molecule ligand is a PSMA ligand and the targeting moiety is FITC. In this embodiment, the small molecule ligand linked to a targeting moiety by a linker can have the formula

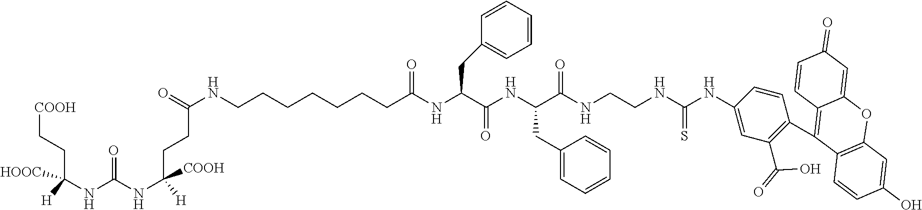 [SMILES: O=C(O)CC[C@H](NC(=O)N[C@@H](CCC(=O)NCCCCCCCC(=O)N[C@@H](Cc1ccccc1)C(=O)N[C@@H](Cc1ccccc1)C(=O)NCCNC(=S)Nc1ccc(-c2c3ccc(=O)cc-3oc3cc(O)ccc23)c(C(=O)O)c1)C(=O)O)C(=O)O]

In yet another embodiment, a method of treatment of a cancer is provided. The method comprises i) administering to a patient a compound, or a pharmaceutically acceptable salt thereof, wherein the compound comprises a small molecule ligand linked to a targeting moiety by a linker, and ii) administering to the patient a CAR T cell composition wherein the CAR T cell composition comprises CAR T cells and wherein the CAR T cells comprise a CAR comprising an E2 anti-fluorescein antibody fragment, and wherein the small molecule ligand is a CAIX ligand and the targeting moiety is FITC. In this embodiment, the small molecule ligand linked to a targeting moiety by a linker can have the formula

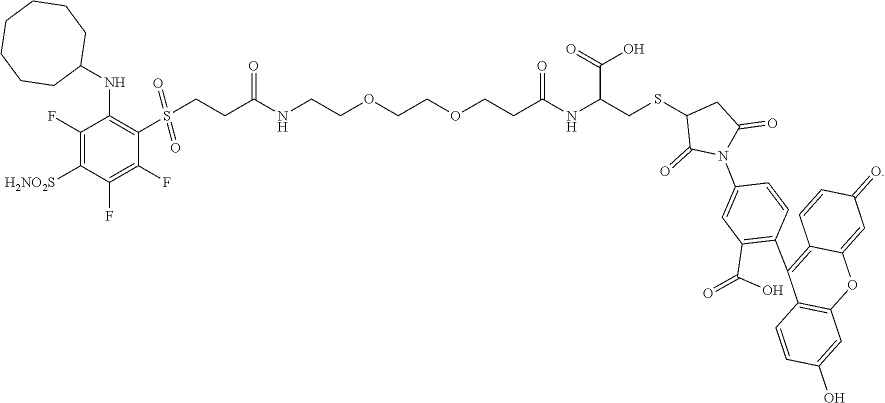 [SMILES: NS(=O)(=O)c1c(F)c(F)c(S(=O)(=O)CCC(=O)NCCOCCOCCC(=O)NC(CSC2CC(=O)N(c3ccc(-c4c5ccc(=O)cc-5oc5cc(O)ccc45)c(C(=O)O)c3)C2=O)C(=O)O)c(NC2CCCCCCC2)c1F]

In still another embodiment, a method of treatment of a cancer is provided. The method comprises i) administering to a patient a first compound, or a pharmaceutically acceptable salt thereof, wherein the first compound, or the pharmaceutically acceptable salt thereof, comprises a PSMA ligand linked to FITC by a linker, ii) administering to the patient a second compound, or a pharmaceutically acceptable salt thereof, wherein the second compound, or the pharmaceutically acceptable salt thereof, comprises a CAIX ligand linked to FITC by a linker, and iii) administering to the patient a CAR T cell composition wherein the CAR T cell composition comprises CAR T cells and wherein the CAR T cells comprise a CAR comprising an E2 anti-fluorescein antibody fragment. In this embodiment, the first compound can have the formula

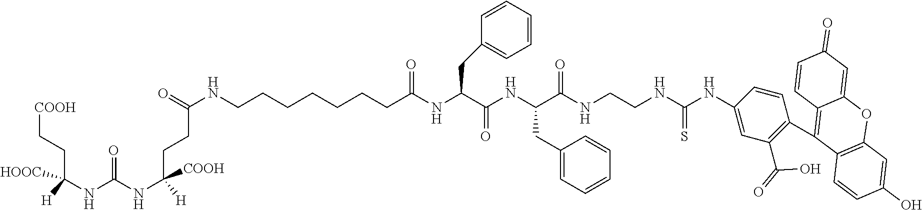 [SMILES: O=C(O)CC[C@H](NC(=O)N[C@@H](CCC(=O)NCCCCCCCC(=O)N[C@@H](Cc1ccccc1)C(=O)N[C@@H](Cc1ccccc1)C(=O)NCCNC(=S)Nc1ccc(-c2c3ccc(=O)cc-3oc3cc(O)ccc23)c(C(=O)O)c1)C(=O)O)C(=O)O]

and the second compound can have the formula

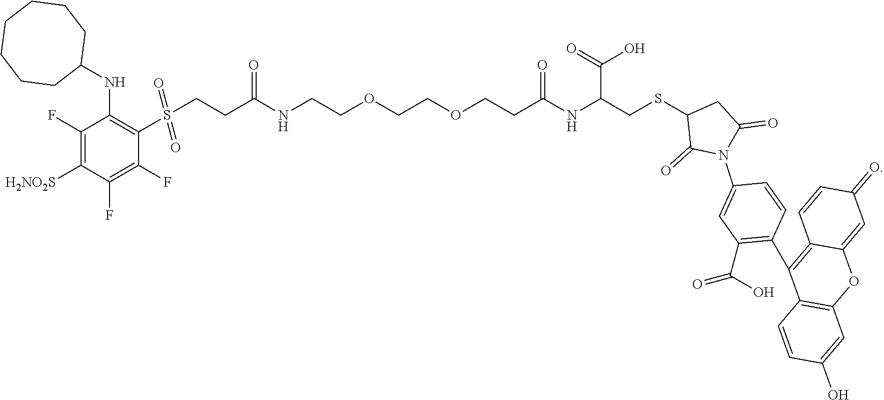 [SMILES: NS(=O)(=O)c1c(F)c(F)c(S(=O)(=O)CCC(=O)NCCOCCOCCC(=O)NC(CSC2CC(=O)N(c3ccc(-c4c5ccc(=O)cc-5oc5cc(O)ccc45)c(C(=O)O)c3)C2=O)C(=O)O)c(NC2CCCCCCC2)c1F]

In one embodiment of the methods described herein, the cancer is imaged prior to administration to the patient of the compound, or the pharmaceutically acceptable salt thereof, or prior to administration of the CAR T cell composition to the patient, wherein the CAR T cell composition comprises CAR T cells comprising a CAR comprising an E2 anti-fluorescein antibody fragment. In one illustrative embodiment, imaging occurs by PET imaging. In other illustrative embodiments imaging occurs by MRI imaging or SPECT/CT imaging. The imaging method can be any suitable imaging method known in the art. In one embodiment, the imaging method can involve the use of the small molecule ligand described herein, but linked to an imaging agent suitable for the types of imaging described herein.

In any of the embodiments described herein, cytokine release resulting in off-target toxicity in the patient may not occur even though toxicity to the cancer of the CAR T cells expressing the E2 anti-fluorescein antibody fragment, occurs. In any embodiment described herein, off-target tissue toxicity may not occur in the patient even though toxicity to the cancer of the CAR T cells expressing the E2 anti-fluorescein antibody fragment, occurs. In any embodiment described herein, the cancer may comprise a tumor, and tumor size may be reduced in the patient, even though off-target toxicity does not occur. In any of the embodiments described herein, CRS can be reduced or prevented and the method can result in a decrease in tumor volume in the patient. In any embodiment described herein, body weight loss due to CRS can be reduced or prevented. In any embodiment described herein, the cancer can comprise a tumor and a complete response for the tumor can be obtained.

In another embodiment of the methods described herein, any of the methods described herein can be used alone, or any of the methods described herein can be used in combination with any other method or methods described herein.

### EXAMPLES

### Example 1

### EC17 Dose De-Escalation can Reduce Therapy Related Toxicity

The effect of EC17 dose de-escalation was investigated on the anti-tumor activity and the toxicity (body weight changes) of CAR-T therapy. Two different anti-fluorescein scFv were used for the CAR constructions (FIG. 1). The first construct of CAR contains the following domains: antiFL(FITC-E2)scFv-IgG4 hinge (CH2 (L235D, N297Q)-CH3)-CD28 TM-4-1BB-CD3z-T2A-EGFRt. The second construct of CAR contains the following domains: antiFL(FITC-4M5.3)scFv-IgG4 hinge (CH2 (L235D, N297Q)-CH3)-CD28 TM-4-1BB-CD3z-T2A-EGFRt. The nucleic acid sequence and amino acid sequence of the CAR of each construct are shown as SEQ ID NOS:1 to 4. Human T cells were isolated, activated and transduced with a lentiviral vector carrying the appropriate CAR gene. CD4+ T cells with the CAR-modification and CD8+ T cells with the CAR-modification were isolated and cultured separately in vitro for about 7 days. CD4+ CAR-T cells and CD8+ CAR-T cells were mixed at a 1:1 ratio before i.v. administration for animal studies.

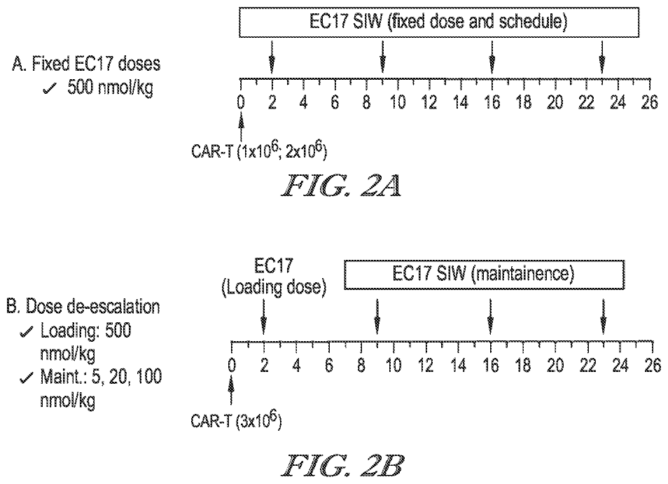

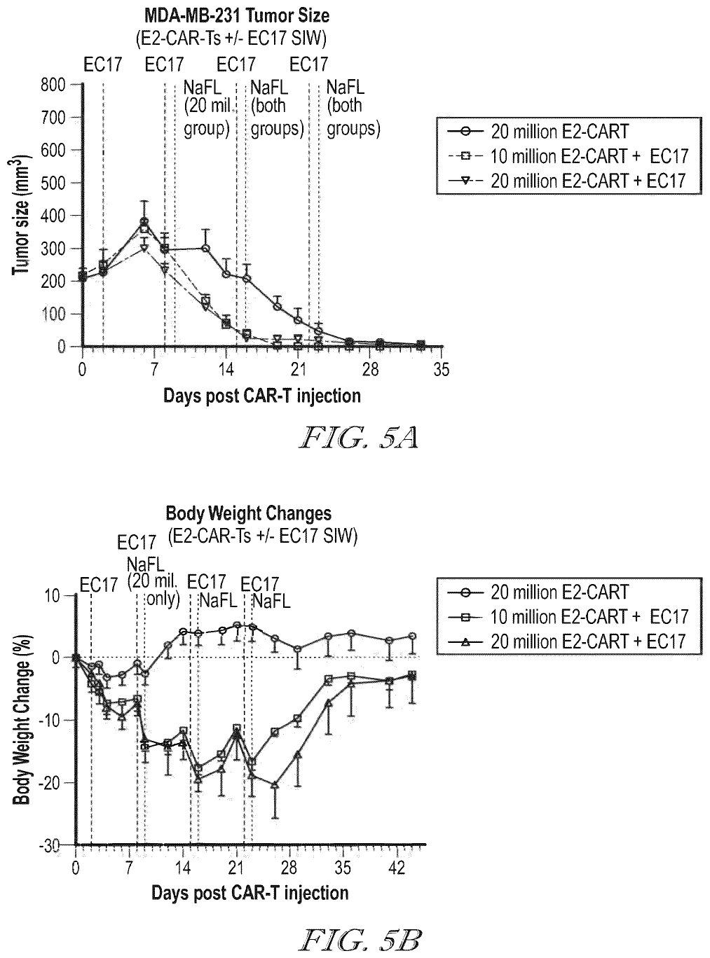

Mice bearing MDA-MB-231 tumors (150-250 mmi) were used for in vivo studies to evaluate E2-CAR-T cells. In the first part of the study, 10 or 20 million E2-CAR-T cells were administered, and 500 nmol/kg EC17 was i.v. dosed weekly at days 2, 9, 16, 23, etc. post CAR-T administration (as shown in FIG. 2A). Mice administered with 20 million E2-CAR-T cells showed severe CRS after the second EC17 dose and had to be rescued with 6 umol/kg sodium fluorescein (NaFL). Both 10 million and 20 million CAR-T groups showed severe CRS after the third and the fourth EC17 doses and had to be rescued with NaFL (as indicated in FIGS. 5A and 5B). Both of these two groups showed severe body weight loss (˜20%) during E2-CAR-T therapy, indicating that 10 or 20 million E2 CAR-T/EC17 500 nmol/kg SIW therapy had severe toxicity. (Although 20 million E2-CAR-T cells alone also showed anti-tumor activity (FIG. 5A, open circle), this was probably caused by allogeneic HLA-mismatched TCRs commonly found in “young” T cells cultured in-vitro for a shorter time.)

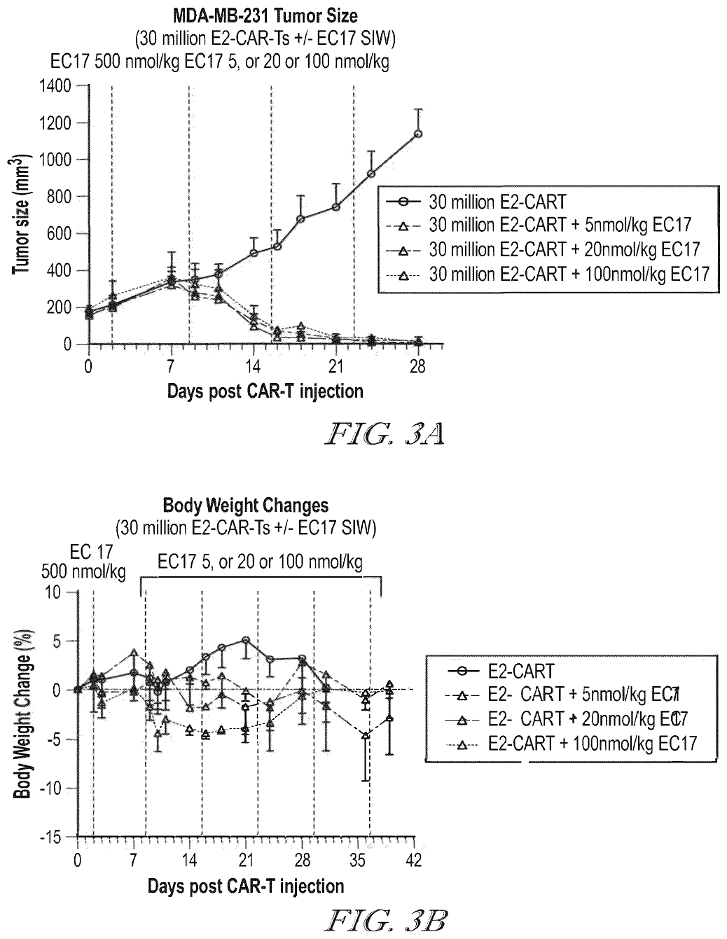

In the second part of the study, E2-CAR-T cells used were cultured in vitro for about 2 weeks, 30 million E2-CAR-T cells were administered and EC17 dosing was modified to de-escalation. As shown in FIG. 2B, 500 nmol/kg EC17 was dosed 2 days post CAR-T administration, then a lower dose of EC17 (5, or 20 or 100 nmol/kg) was administered weekly (at days 9, 16, 23, etc. post CAR-T administration). As shown in FIG. 3A, all three EC17 de-escalation groups reached a complete response, indicating that bridge de-escalation does not affect the anti-tumor activity of CAR-T. More importantly, none of these three EC17 de-escalation groups showed any severe CRS. As shown in FIG. 3B, all de-escalation groups showed very mild body weight loss although more CAR-T cells (30 million) were administered compared to the first part of study (10 or 20 million CAR-T cells). These data indicate that bridge de-escalation (one high-level loading dose followed with a reduced-level of maintenance doses) can reduce the toxicity while maintaining good anti-tumor activity of CAR-T cells.

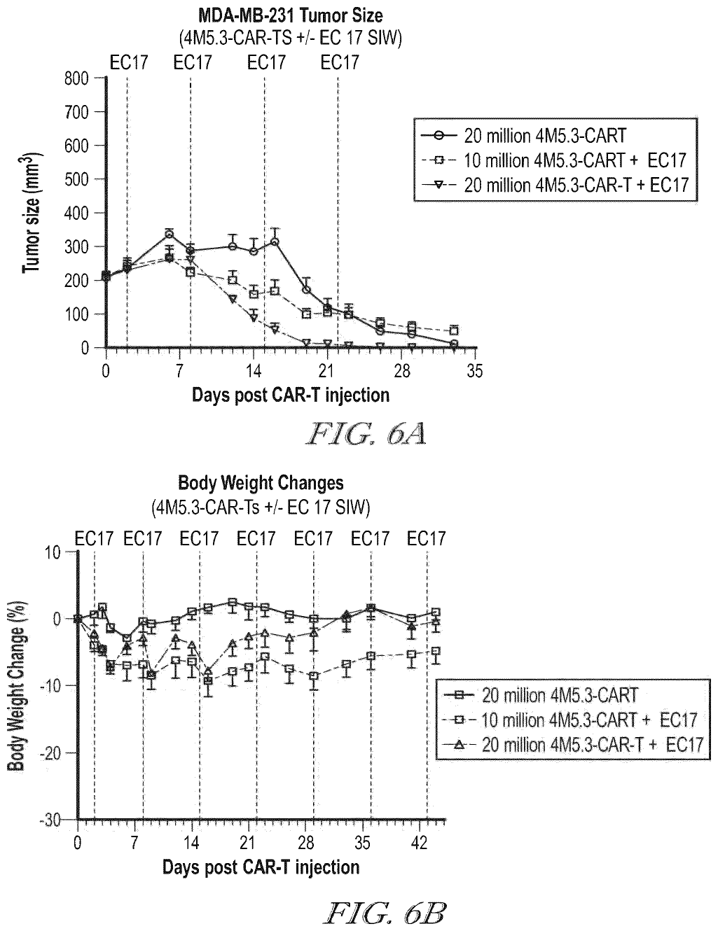

The second construct 4M5.3-CAR (antiFL(FITC-4M5.3) scFv-IgG4 hinge (CH2 (L235D, N297Q)-CH3)-CD28 TM-4-1BB-CD3z-T2A-EGFRt) was also evaluated. Mice bearing MDA-MB-231 tumors (150-250 mm³) were used for in vivo studies to evaluate 4M5.3-CAR-T cells. In the first part of the study, 10 or 20 million 4M5.3-CAR-T cells were administered, and 500 nmol/kg EC17 was i.v. dosed weekly at days 2, 9, 16, 23, etc. post CAR-T administration (as shown in FIG. 2A). Mice administered with either 10 or 20 million 4M5.3-CAR-T cells did not show severe CRS or body weight loss after administering an EC17 dose (FIG. 6B), but their tumors were all reduced and eventually disappeared (FIG. 6A). 20 million CAR-T cells (upper line) showed better anti-tumor activity than 10 million CAR-T cells (middle line).

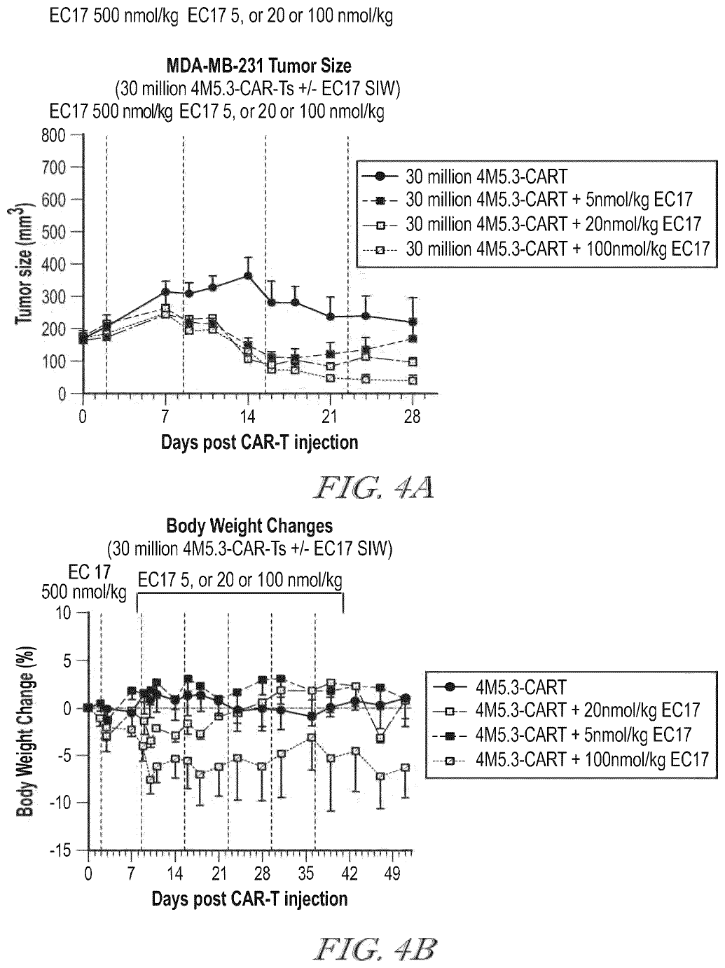

In the second part of the study, 4M5.3-CAR-T cells used were cultured in vitro for about 2 weeks. 30 million 4M5.3-CAR-T cells were administered and EC17 dosing was modified to de-escalation. As shown in FIG. 2B, 500 nmol/kg EC17 was dosed 2 days post CAR-T administration, then a lower dose of EC17 (5, or 20 or 100 nmol/kg) was administered weekly (at days 9, 16, 23, etc. post CAR-T administration). As shown in FIG. 4A, 100 nmol/kg EC17 de-escalation group showed the best anti-tumor activity and all tumors were cured (bottom line), whereas neither 5 nmole/kg EC17 group (second line from the top) nor 20 nmol/kg EC17 group (third line from the top) showed a complete response to the CAR-T therapy. The data indicate that CAR-T activity is controlled by the EC-17 dose used for de-escalation. Furthermore, as shown in FIG. 4B, body weight loss of mice in the three de-escalation groups was also dependent on EC17 dose. The 100 nmol/kg EC17 dose de-escalation group showed more body weight loss than the other two groups with either 5 or 20 nmol/lg EC17. These data indicate that bridge de-escalation (one high-level loading dose followed with reduced-level of maintenance doses) can control both anti-tumor activity and the toxicity of CAR-T therapy.

The only difference between E2-CAR and 4M5.3-CAR is the scFv sequences used. By comparing these two CARs with the same conditions, 10 million E2-CAR-T cells were found to have better anti-tumor activity than 10 million 4M5.3-CAR-T cells; 30 million E2-CAR-T cells also showed better anti-tumor activity than 30 million 4M5.3-CAR-T cells when EC17 dose de-escalation (5, or 20, or 100 nmol/kg) was tested.

As used herein, “SEQ ID NO:4” means the sequence beginning at the underlined “gac” codon and ending with the underlined “ggc” codon. This portion of the longer sequence, encodes the exemplary 4M5.3 CAR. The CAR is inserted into the T cell membrane. The other portions of the longer sequence include coding sequence for signal peptides, the EGFRt domain, etc. which are not part of the CAR that is inserted into the membrane and which functions as the chimeric antigen receptor. As used herein, “SEQ ID NO:3” means the sequence beginning at the underlined “D” and ending with the underlined “G”. This portion of the longer sequence is the amino acid sequence for the CAR that is inserted into the T cell membrane. The other portions of the longer sequence include amino acid sequences for signal peptides, the EGFRt domain, etc. which are not part of the CAR inserted into the membrane and which functions as the chimeric antigen receptor. In yet another embodiment, SEQ ID NO:3 can comprise or consist of humanized, or human amino acid sequences. SEQ ID NOS:3 and 4 are as described above. The start and stop codons in the longer nucleic acid sequence are underlined and the longer sequence is an exemplary sequence that can be used for transduction of T cells to prepare the 4M5.3 CAR.

[4M5.3-CAR amino acid sequence (insert)]
SEQ ID NO: 3
MLLLVTSLLLCELPHPAFLLIPDVVMTQTPLSLPVSLGDQASISCRSSQ
SLVHSNGNTYLRWYLQKPGQSPKVLIYKVSNRVSGVPDRFSGSGSGTDF
TLKINRVEAEDLGVYFCSQSTHVPWTFGGGTKLEIKSSADDAKKDAAKK
DDAKKDDAKKDGGVKLDETGGGLVQPGGAMKLSCVTSGFTFGHYVVMNW
VRQSPEKGLEWVAQFRNKPYNYETYYSDSVKGRFTISRDDSKSSVYLQM
NNLRVEDTGIYYCTGASYGMEYLGQGTSVTVSESKYGPPCPPCPAPEFD
GGPSVFLFPPKPKDTLMISRTPEVTCVVVDVSQEDPEVQFNWYVDGVEV
HNAKTKPREEQFQSTYRVVSVLTVLHQDWLNGKEYKCKVSNKGLPSSIE
KTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEA
LHNHYTQKSLSLSLGKMFWVLVVVGGVLACYSLLVTVAFIIFWVKRGRK
KLLYIFKQPFMRPVQTTQEEDGCSCRFPEEEEGGCELRVKFSRSADAPA
YQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALP
PRLEGGGEGRGSLLTCGDVEENPGPRMLLLVTSLLLCELPHPAFLLIPR
KVCNGIGIGEFKDSLSINATNIKHFKNCTSISGDLHILPVAFRGDSFTH
TPPLDPQELDILKTVKEITGFLLIQAWPENRTDLHAFENLEIIRGRTKQ
HGQFSLAVVSLNITSLGLRSLKEISDGDVIISGNKNLCYANTINWKKLF
GTSGQKTKIISNRGENSCKATGQVCHALCSPEGCWGPEPRDCVSCRNVS
RGRECVDKCNLLEGEPREFVENSECIQCHPECLPQAMNITCTGRGPDNC
IQCAHYIDGPHCVKTCPAGVMGENNTLVWKYADAGHVCHLCHPNCTYGC
TGPGLEGCPTNGPKIPSIATGMVGALLLLLVVALGIGLFM
[4M5.3-CAR nucleotide acid sequence (insert)]
SEQ ID NO: 4
ATG(START)cttctcctggtgacaagccttctgctctgtgagttacca
cacccagcattcctcctgatcccagacgttgtaatgacccagacccctc
tgtctctccccgtaagcttgggcgaccaggcgagcatctcttgtcggtc
ttcccagtccctggtccattcaaacggcaatacttacttgcggtggtac
ttgcagaagcccggtcaatccccaaaagtgctgatatacaaggttagca
atcgggtcagtggagtgcccgaccgcttcagcggaagcggatccgggac
tgacttcactctgaagatcaaccgggtagaagctgaagacctgggggtg
tacttctgctctcagtcaacacacgtgccatggacctttggaggtggca
ccaagctggaaatcaaatcatcagcggacgatgccaaaaaagacgcggc
caagaaggacgatgccaagaaggatgatgctaaaaaggatggcggagtc
aaattggacgagacaggcgggggactggtgcagcccggcggtgccatga
aactgtcttgtgtgaccagcggctttaccttcgggcattattggatgaa
ctgggtgcgacagtctccagagaaagggctcgagtgggtggcccagttt
cgaaataaaccgtacaattatgagacctactattcagattctgtgaaag
ggcgcttcactatttcacgcgacgacagcaaaagttccgtctaccttca
gatgaacaaccttagagtggaggataccggaatatactactgcacgggt
gccagttatggcatggagtacttggggcaggggacatctgtgaccgttt
ctgagagcaagtacggaccgccctgccccccttgccctgcccccgagtt
cgacggcggacccagcgtgttcctgttcccccccaagcccaaggacacc
ctgatgatcagccggacccccgaggtgacctgcgtggtggtggacgtga
gccaggaagatcccgaggtccagttcaattggtacgtggacggcgtgga
agtgcacaacgccaagaccaagcccagagaggaacagttccagagcacc
taccgggtggtgtctgtgctgaccgtgctgcaccaggactggctgaacg
gcaaagaatacaagtgcaaggtgtccaacaagggcctgcccagcagcat
cgaaaagaccatcagcaaggccaagggccagcctcgcgagccccaggtg
tacaccctgcctccctcccaggaagagatgaccaagaaccaggtgtccc
tgacctgcctggtgaagggcttctaccccagcgacatcgccgtggagtg
ggagagcaacggccagcctgagaacaactacaagaccacccctcccgtg
ctggacagcgacggcagcttcttcctgtacagccggctgaccgtggaca
agagccggtggcaggaaggcaacgtctttagctgcagcgtgatgcacga
ggccctgcacaaccactacacccagaagagcctgagcctgtccctgggc
aagatgttctgggtgctggtggtggtggggggggctggcctgctacagc
ctgctggtgacagtggccttcatcatcttttgggtgaaacggggcagaa
agaaactcctgtatatattcaaacaaccatttatgagaccagtacaaac
tactcaagaggaagatggctgtagctgccgatttccagaagaagaagaa
ggaggatgtgaactgcgggtgaagttcagcagaagcgccgacgcccctg
cctaccagcagggccagaatcagctgtacaacgagctgaacctgggcag
aagggaagagtacgacgtcctggataagcggagaggccgggaccctgag
atgggcggcaagcctcggcggaagaacccccaggaaggcctgtataacg
aactgcagaaagacaagatggccgaggcctacagcgagatcggcatgaa
gggcgagcggaggcggggcaagggccacgacggcctgtatcagggcctg
tccaccgccaccaaggatacctacgacgccctgcacatgcaggccctgc
ccccaaggctcgagggcggcggagagggcagaggaagtcttctaacatg
cggtgacgtggaggagaatcccggccctaggatgcttctcctggtgaca
agccttctgctctgtgagttaccacacccagcattcctcctgatcccac
gcaaagtgtgtaacggaataggtattggtgaatttaaagactcactctc
cataaatgctacgaatattaaacacttcaaaaactgcacctccatcagt
ggcgatctccacatcctgccggtggcatttaggggtgactccttcacac
atactcctcctctggatccacaggaactggatattctgaaaaccgtaaa
ggaaatcacagggtttttgctgattcaggcttggcctgaaaacaggacg
gacctccatgcctttgagaacctagaaatcatacgcggcaggaccaagc
aacatggtcagttttctcttgcagtcgtcagcctgaacataacatcctt
gggattacgctccctcaaggagataagtgatggagatgtgataatttca
ggaaacaaaaatttgtgctatgcaaatacaataaactggaaaaaactgt
ttgggacctccggtcagaaaaccaaaattataagcaacagaggtgaaaa
cagctgcaaggccacaggccaggtctgccatgccttgtgctcccccgag
ggctgctggggcccggagcccagggactgcgtctcttgccggaatgtca
gccgaggcagggaatgcgtggacaagtgcaaccttctggagggtgagcc
aagggagtttgtggagaactctgagtgcatacagtgccacccagagtgc
ctgcctcaggccatgaacatcacctgcacaggacggggaccagacaact
gtatccagtgtgcccactacattgacggcccccactgcgtcaagacctg
cccggcaggagtcatgggagaaaacaacaccctggtctggaagtacgca
gacgccggccatgtgtgccacctgtgccatccaaactgcacctacggat
gcactgggccaggtcttgaaggctgtccaacgaatgggcctaagatccc
gtccatcgccactgggatggtgggggccctcctcttgctgctggtggtg
gccctggggatcggcctcttcatgTGA(STOP CODON)

### Example 2

Characterization of Cart Cell Differentiation Phenotypes Prior to Infusion into NSG Mice

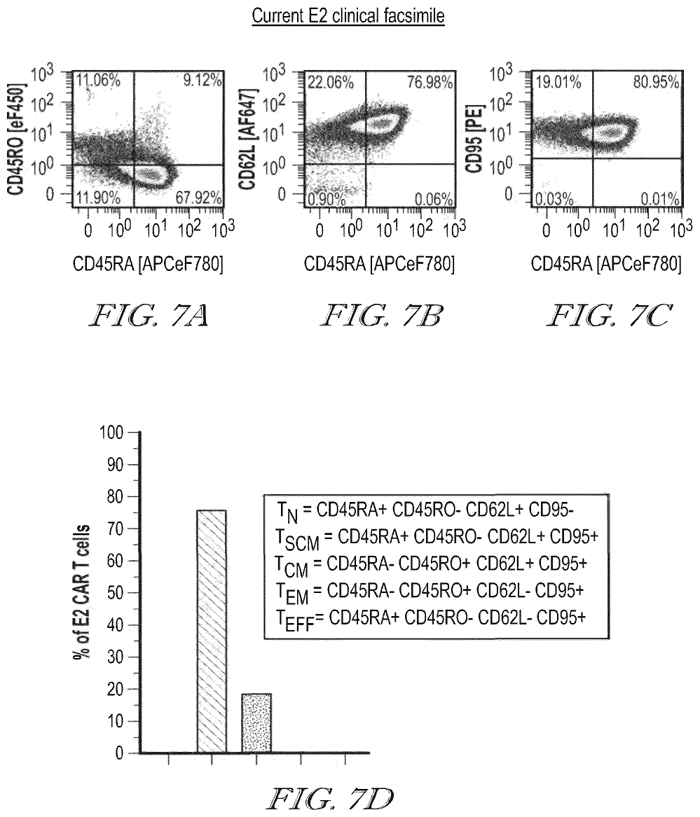

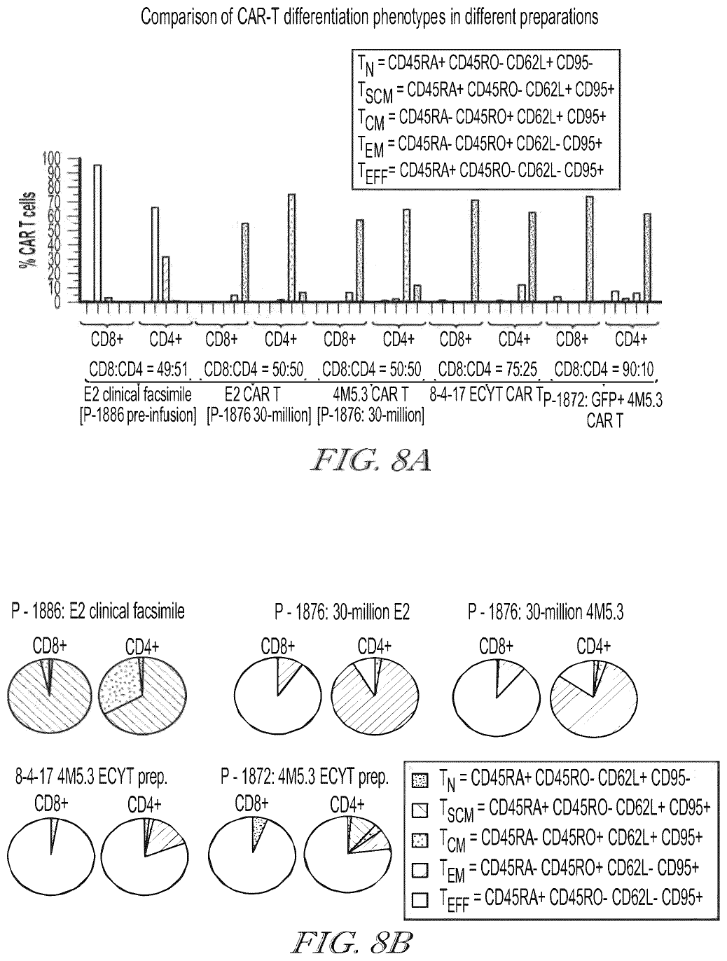

Multi-color flow cytometry was used to determine the phenotypes of CART cell products to be infused into tumor-bearing mice. The differentiation status of CD4+ and CD8+ subsets was analyzed using specific combinations of surface markers as shown in FIGS. 7A-C. The unsorted E2 clinical facsimile CAR T cell product was made predominantly of the self-renewing Tscm/Tcm phenotype (FIG. 7D). In FIG. 7D, the highest bar is “Tscm” and the lower bar is “Tcm”. In addition to differences in CD8:CD4 ratios shown in FIG. 8A, the current E2 clinical facsimile CAR T cells were different from the previously sorted E2/4M5.3 CAR-T cells and early GFP+4M5.3 CAR T in vivo study preparations (summarized as pie charts in FIG. 8B). In FIG. 8A, in the 10 groups from left to right, the bars represent the following—1 (high bar Tscm, lower bar Tcm), 2—(high bar Tscm, lower bar Tcm), 3—(high bar Teff, lower bar Tem), 4—(high bar Tem, lower bar Teff, lowest bar Tcm), 5—(high bar Teff, lower bar Tem), 6—(high bar Tem, lower bar Teff, lowest bar Tcm), 7—(high bar Teff, lower bar Tscm), 8—(high bar Teff, lower bar Tem), 9—(high bar Teff, lower bar Tscm), 10—(high bar Teff, lower bar Tsm, next lower bar Tem, lowest bar Tcm). Accordingly, CAR T cells of predominately Tscm/Tem phenotype were expected to possess a superior anti-tumor phenotype when compared to past CAR T preparations which possess a more differentiated Tem/Teff phenotype. For the same reason, sCRS arises later for the current CAR-T preparation than the previous CAR-T preparations given the same EC17 dosing regimens.

Recover of CAR T Cells after Cryopreservation

Cryopreserved CAR T cells were rapidly thawed in a 37° C. water bath then immediately placed into T cell recovery media (TexMACS™ Media containing glutamine (Miltenyi Biotec #130-097-196) supplemented with 2% human AB serum) and 50 U/mL recombinant human IL2 and cultured in vitro for approximately 3 to 5 days for T cell recovery.

### Flow Cytometry Analysis

CAR T cells were harvested from in vitro recovery media and pelleted by centrifugation at 400 g for 5 minutes. The T cells were then resuspended in flow cytometry staining solution [1% bovine serum albumin, 50 mg/mL human IgG (Equitech Bio, cat #SLH56-0001), 0.9% sodium azide in a phosphate buffered saline, pH=7.4] supplemented with both anti-mouse FcγIII/II receptor (CD16/CD32) block [clone 2.4G2; BD Bioscience, catalog #553142 at 1:100 (v/v) dilution] and anti-human Fc Block [BD Biosciences, catalog #564220 at 1:50 (v/v) dilution]. Surface marker staining was performed with the addition of the following fluorochrome conjugated monoclonal antibodies added to each sample for 20 minutes on ice in the dark: anti-human CD45RA-APCeF780 [clone HI100, ThermoFisher #47-0458-42 at 1:20 (v/v) dilution], anti-human CD45R0-eF450 [clone UCHL1, Thermofisher #48-0457-42 at 1:20 (v/v) dilution], anti-human CD8α-PECy7 [clone RPA-T8, BD Bioscience, catalog #557746 at 1:20 (v/v) dilution], anti-human CD4-Percpe7l0 [clone SK3, eBioscience catalog #464)04742 at 1:20 (v/v) dilution], biotinylated anti-human EGFR (Cetuximab; R&D Systems #FAB9577B-100 at 1:10 (v/v) followed by PE conjugated anti-biotin (BioLegend #53-9895-82 at 1:400 (v/v). After surface marker staining, T cells were washed with PBS and resuspended in cold PBS containing 3 μM propidium iodide and transferred to flow cytometry collection tubes. Flow cytometry data was collected on the Gallios flow cytometer (Beckman Coulter, Brea, CA), where a minimum of 20,000 events were collected and data was analyzed using Kaluza v1.5 software.

### Example 3

E2 Anti-FITC Antibody Doesn't Bind with any Human Normal Tissues

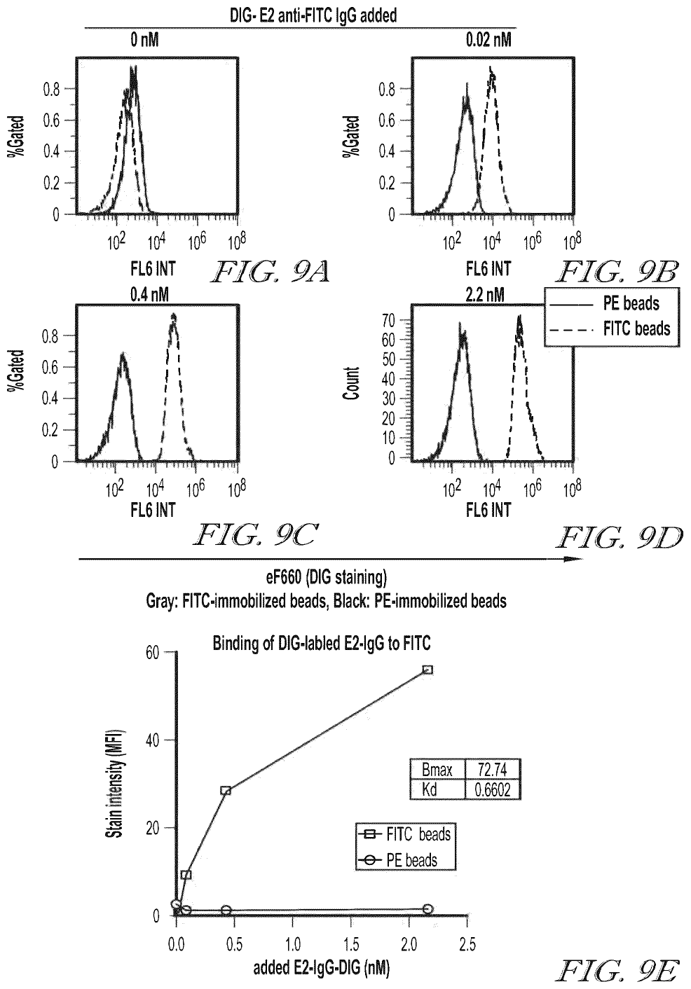

An E2 anti-FITC IgG antibody with the same variable fragment sequences as those in E2 anti-FITC CAR construct was used to test if E2 antibody has any binding with human normal tissues. A recombinant human E2 anti-FITC IgG1 Lambda 1 was expressed in HEK293 cells and purified by using a protein A affinity column. The purity of the antibody was over 98% by using SEC-HPLC analysis. The antibody was labeled with digoxigenin (DIG) using Mix-N-Stain DIG antibody labeling kit (Biotium). The binding affinity of DIG-labeled E2 antibody for FITC-immobilized beads was measured by FACS. Various concentrations of DIG-E2 IgG were incubated with FITC-immobilized beads for 30 min at room temperature. After washing, a monoclonal anti-DIG Ab was added and incubated with beads for 30 min. An eF660 conjugated anti-mouse secondary antibody was added and incubated with the washed beads. The washed beads were analyzed for the eF660 signal by FACS. The PE-immobilized beads were used as the negative control beads. The fluorescence intensity of eF660 was plotted against the concentrations of added DIG-labeled E2-IgG and shown in FIGS. 9A-D. The binding affinity of DIG-labeled E2 antibody for FITC was calculated by using PRISM software. As indicated in FIG. 9E insert, the Kd was about 0.66 nM, indicating that DIG-labeled E2 anti-FITC antibody has high binding affinity for FITC and can be used to evaluate E2 binding with human normal tissues.

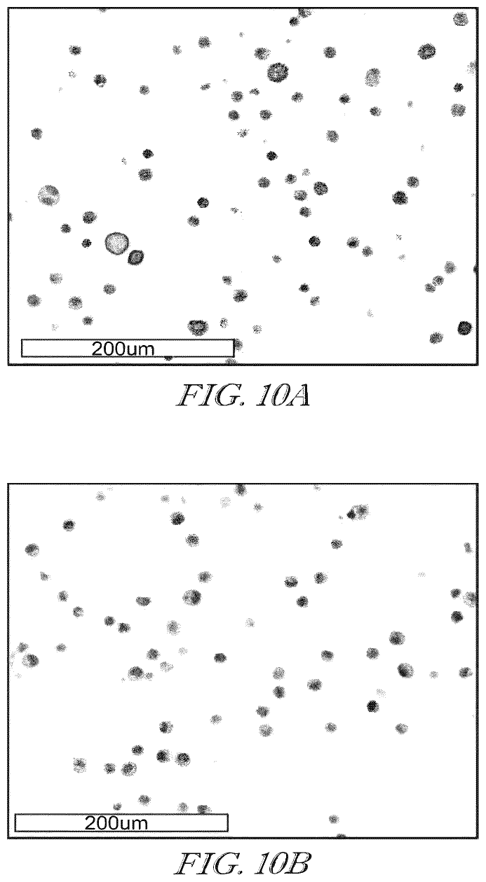

An IHC assay was developed to test the binding of E2 antibody to human tissues. Formalin-fixed, paraffin-embedded (FFEP) tissue section of the agar block with FITC-labeled KB cells was used as the positive control, while FFPE tissue section of unlabeled KB cells was used as the negative control. The tissue sections were deparaffinizaed and rehydrated, then antigen retrieval was done by incubating tissue sections with an antigen retrieval buffer (pH6.0) at 91° C. for 24 minutes, then DIG-E2 antibody was incubated with rehydrated tissue sections, and finally an anti-DIG IHC staining was performed. In brief, a monoclonal anti-DIG antibody was added and incubated, followed by a peroxidase-labeled anti-mouse secondary antibody. As shown in FIG. 10A, FITC-labeled KB cells showed positive staining, indicating that the E2 antibody binds with FITC on these labeled KB cells. The unlabeled KB cells did not show any staining (FIG. 10B).

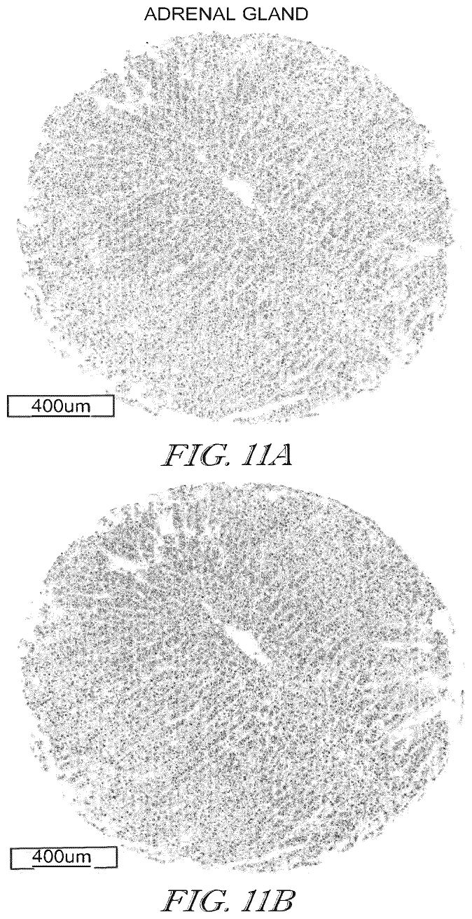

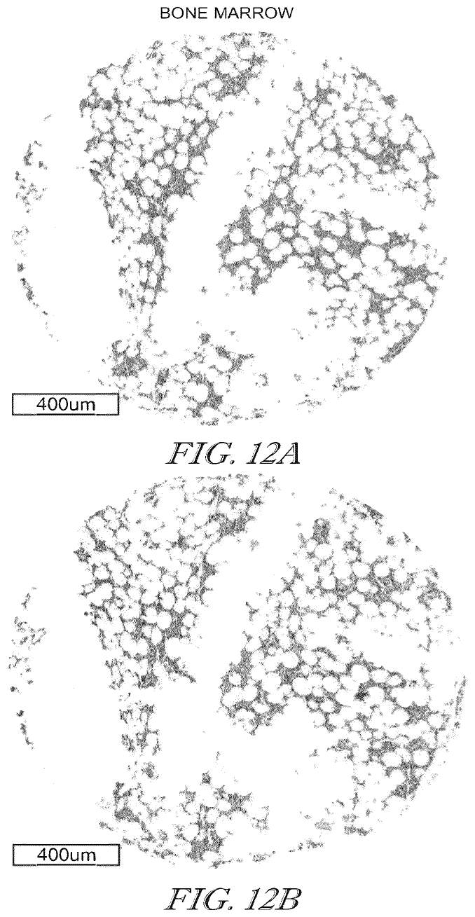

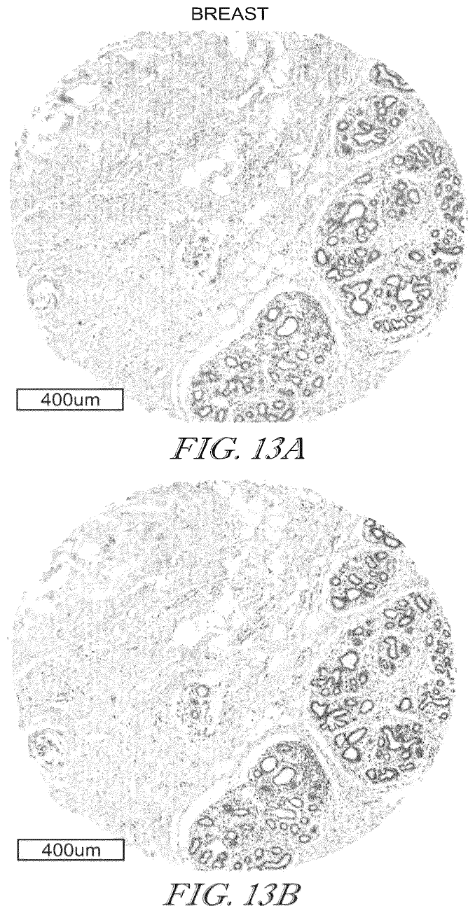

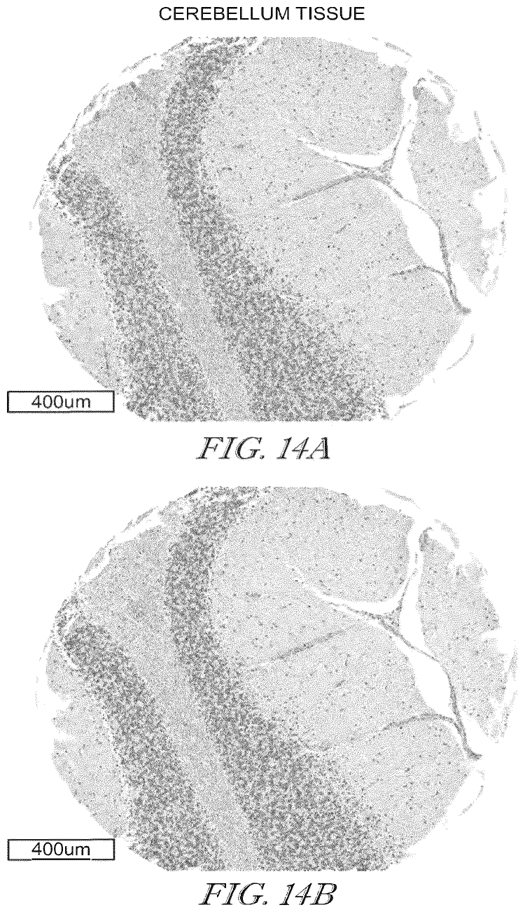

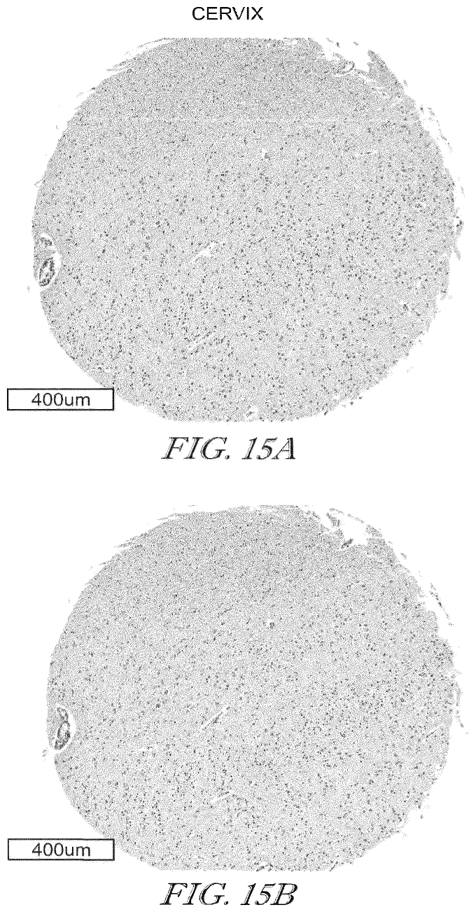

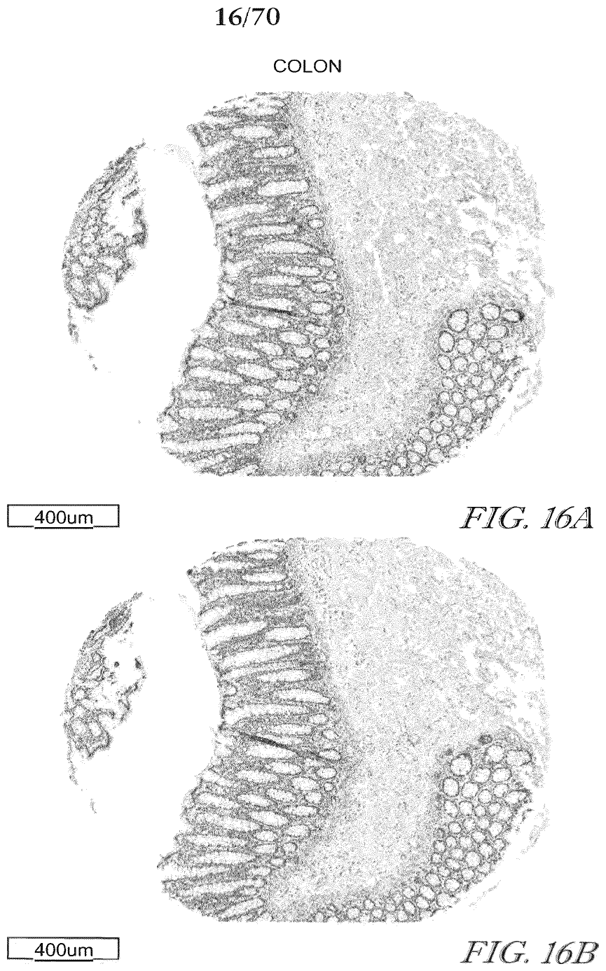

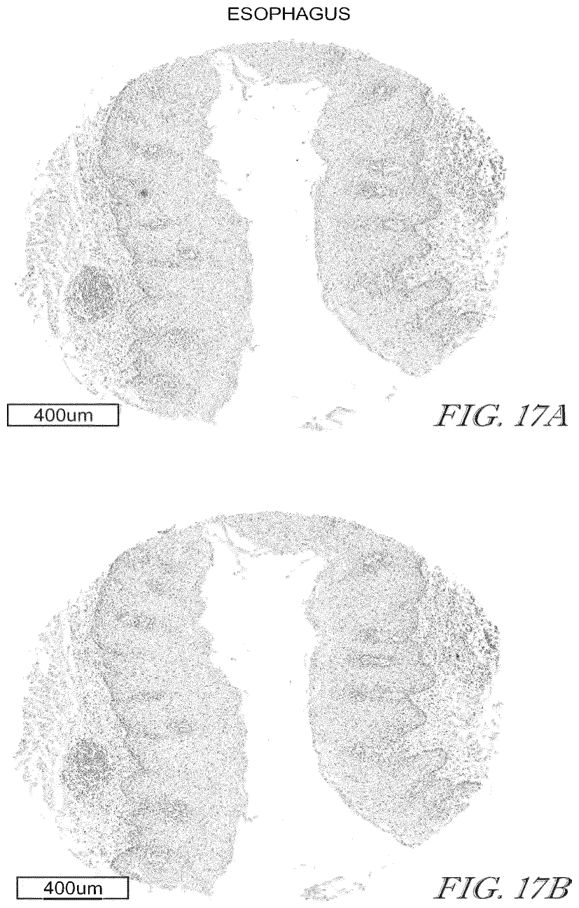

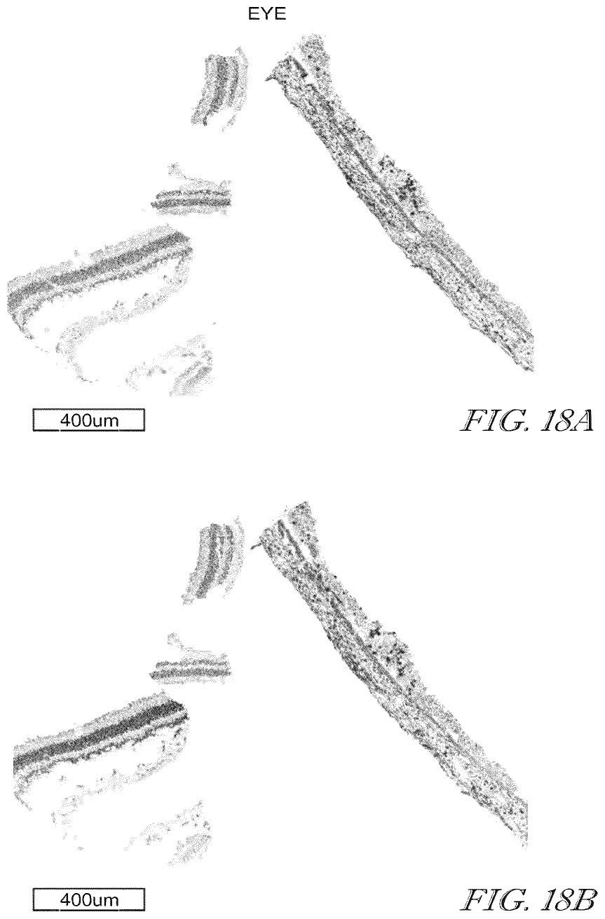

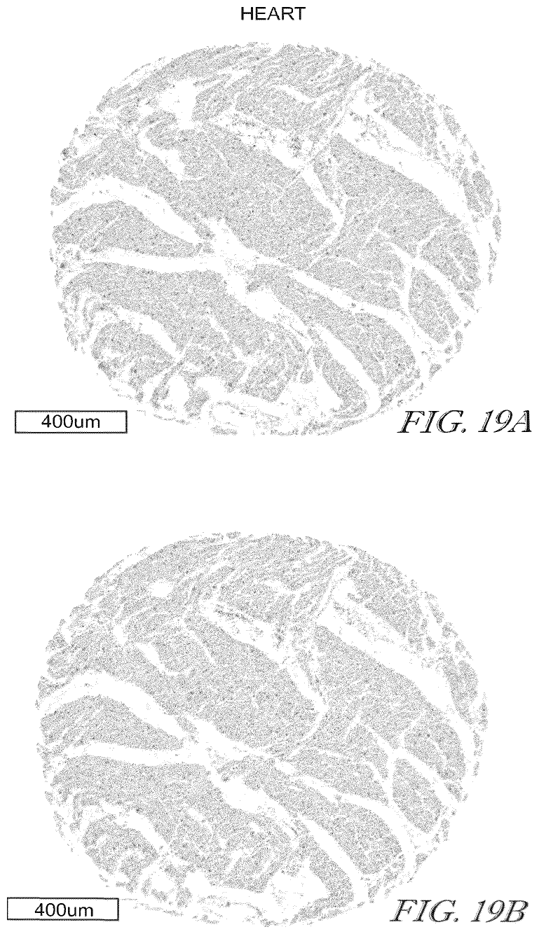

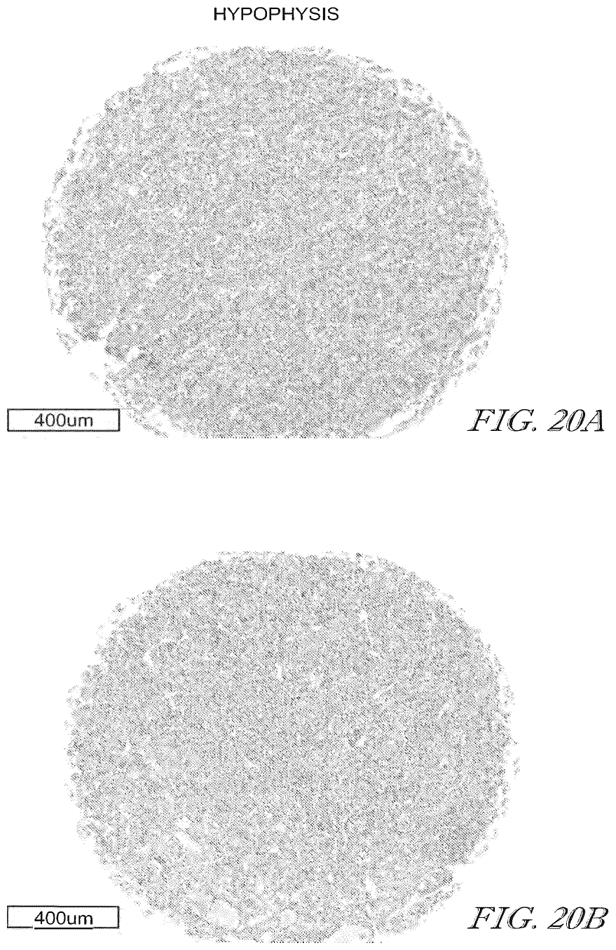

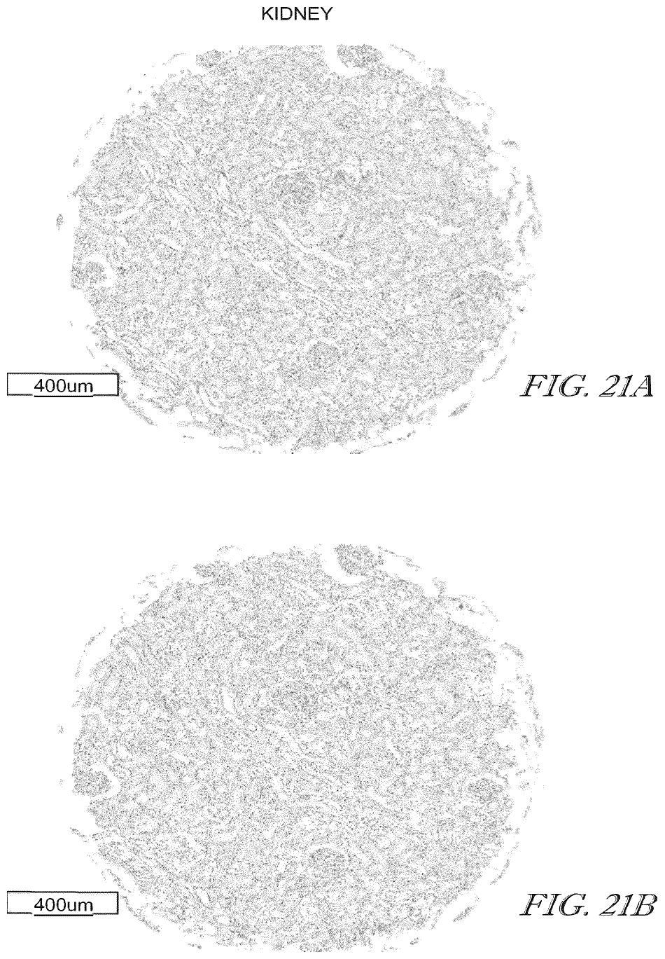

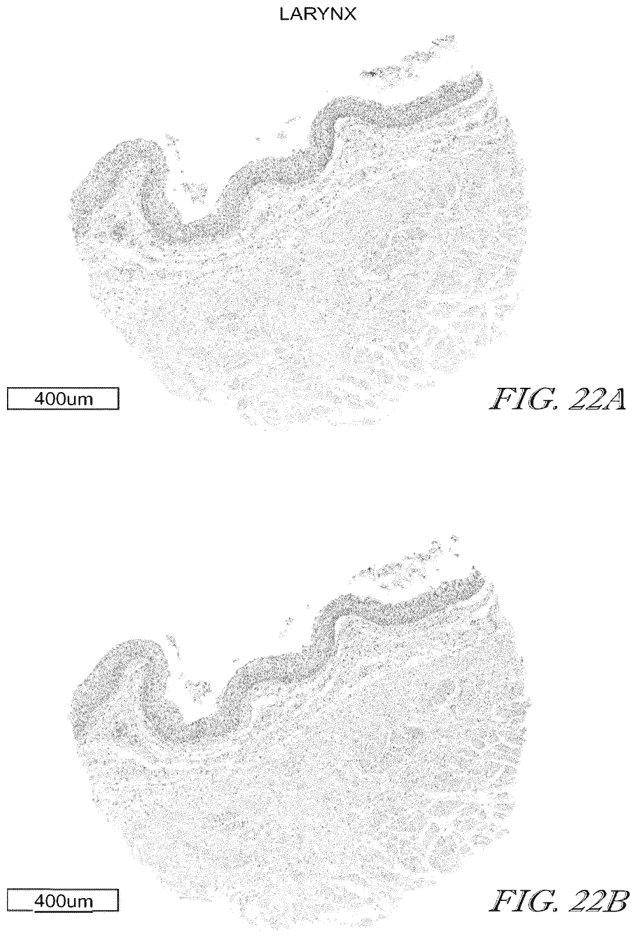

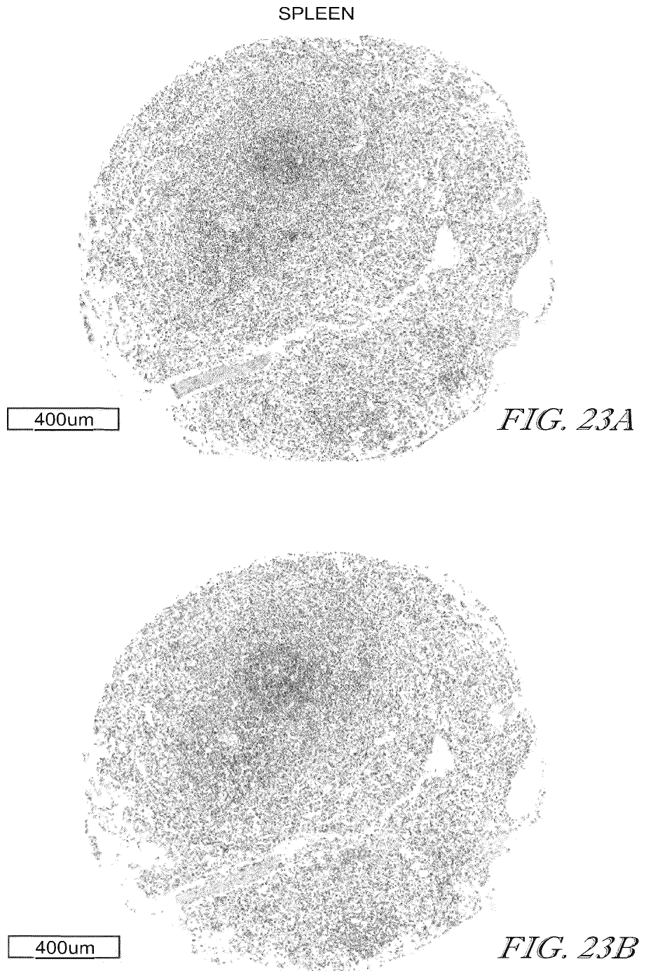

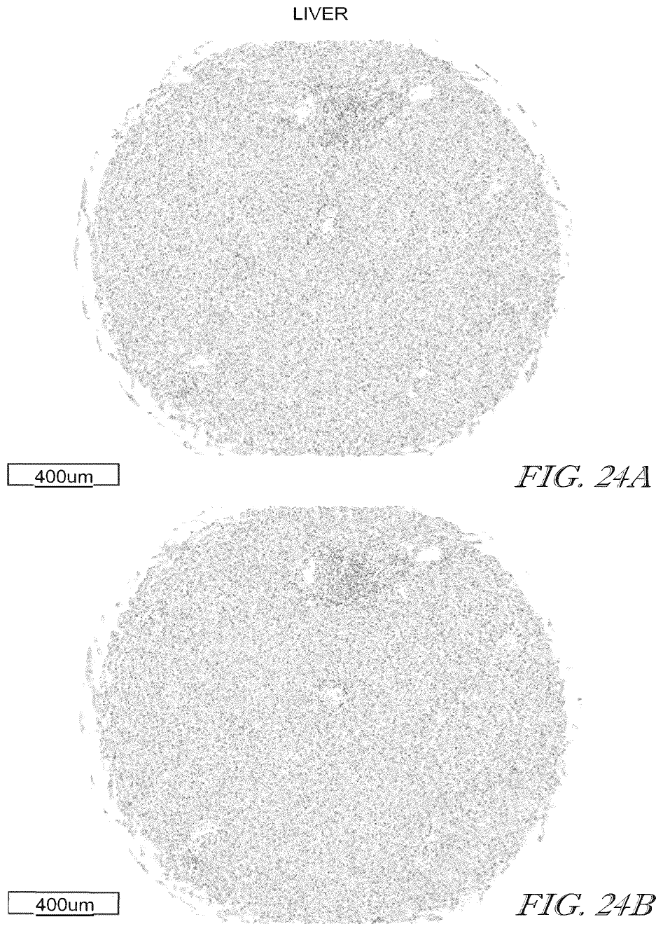

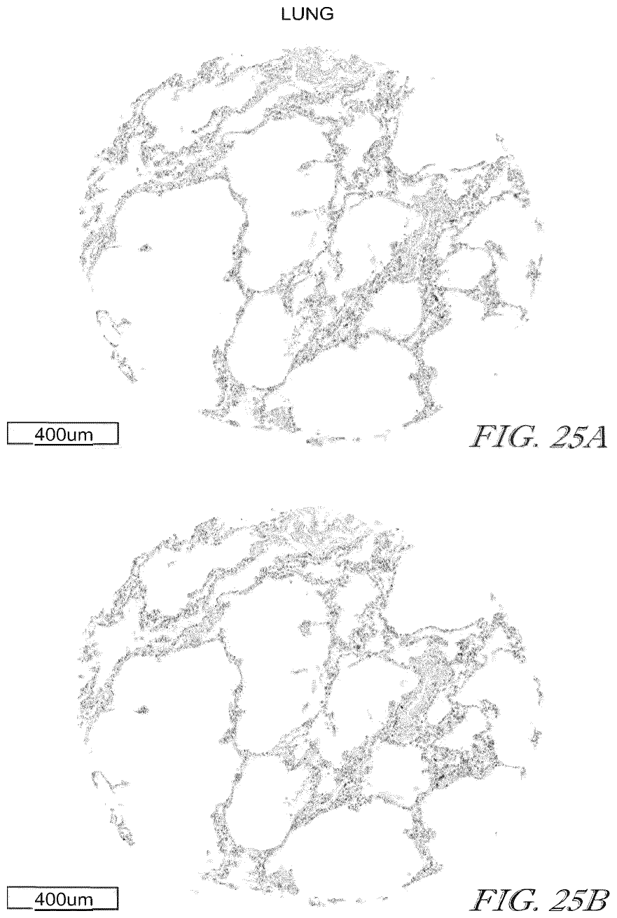

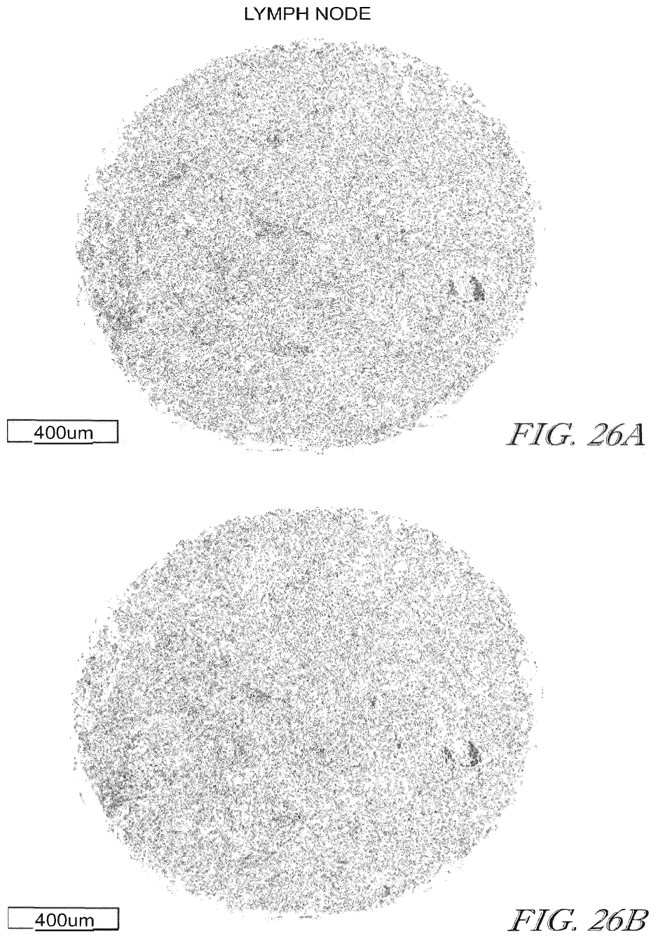

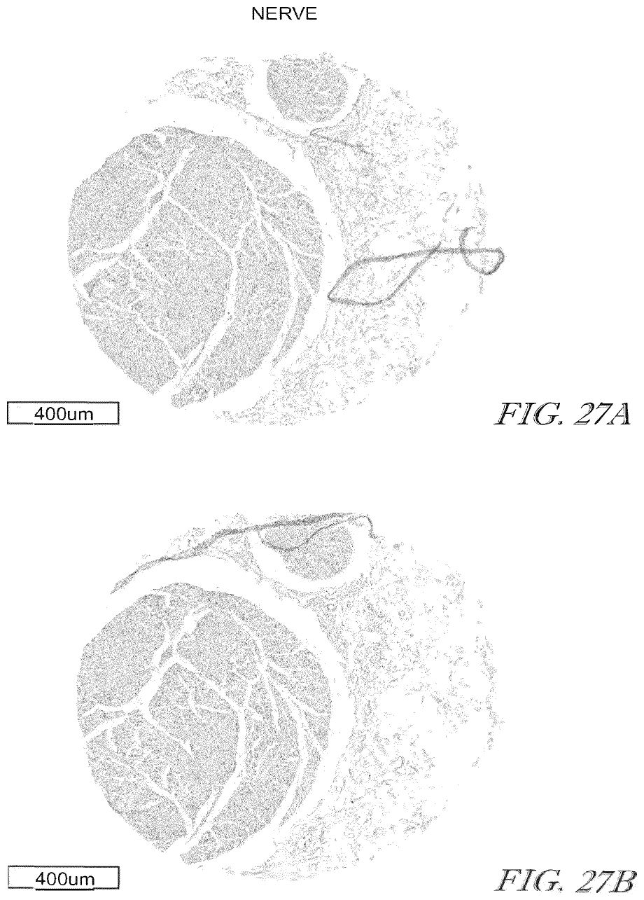

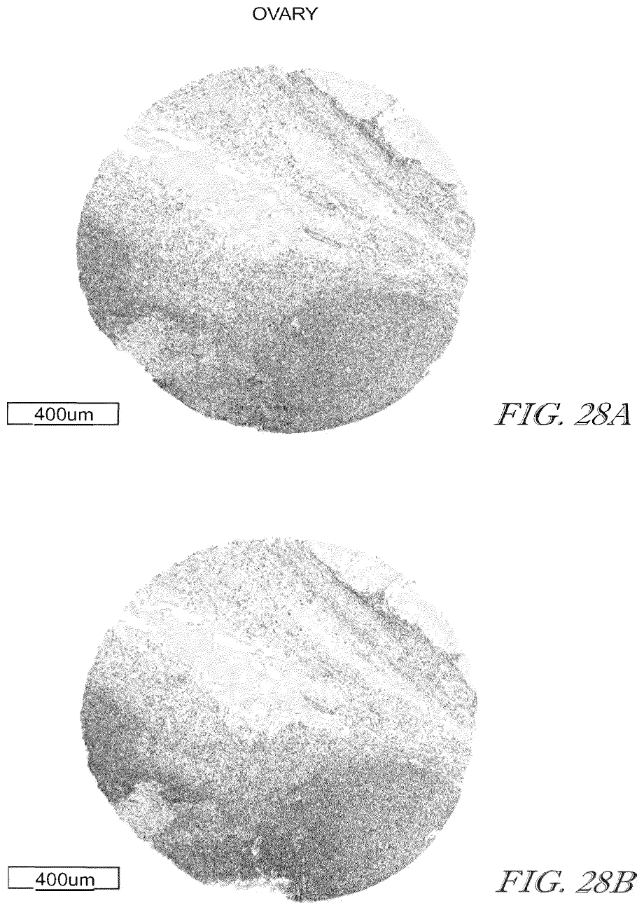

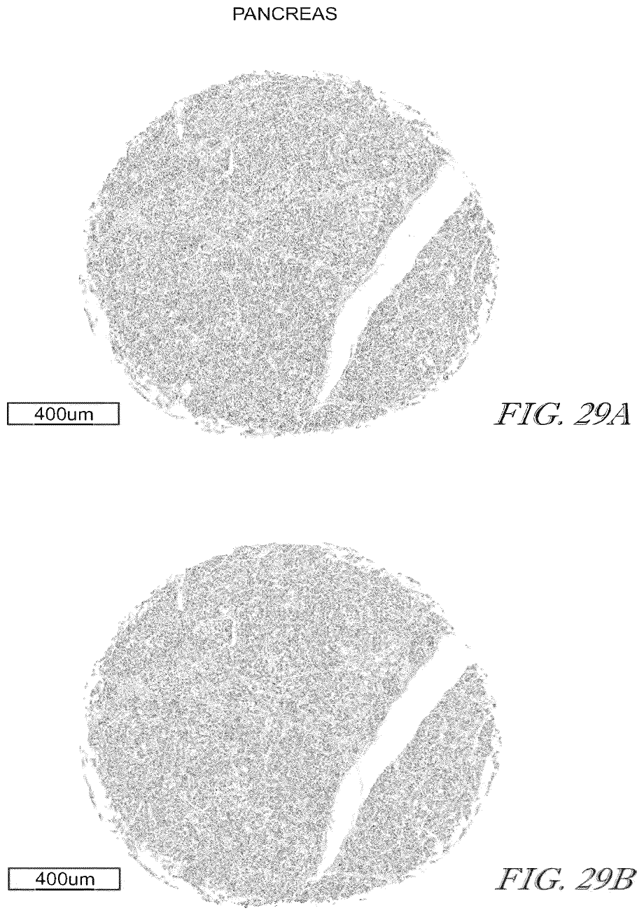

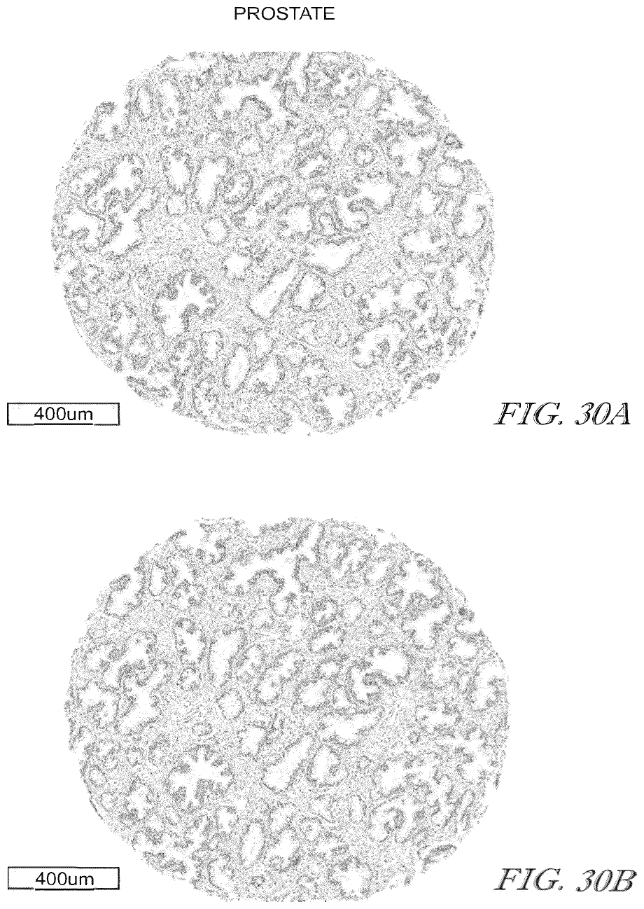

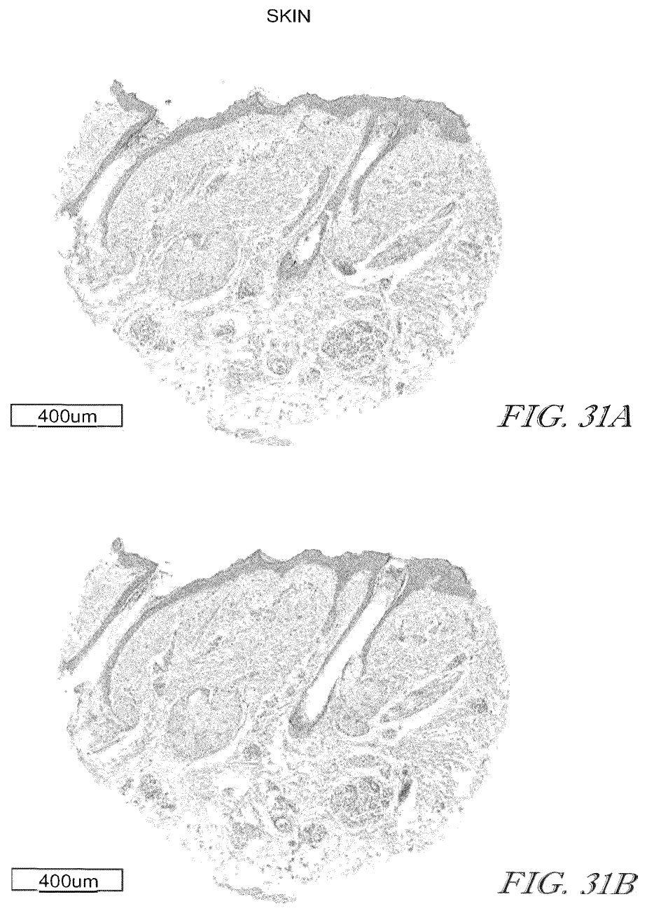

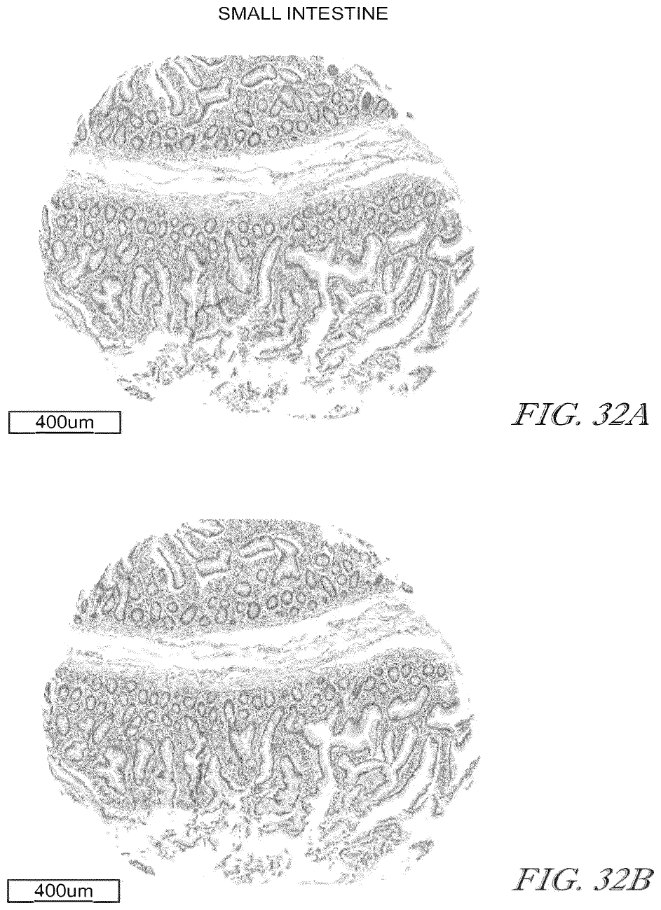

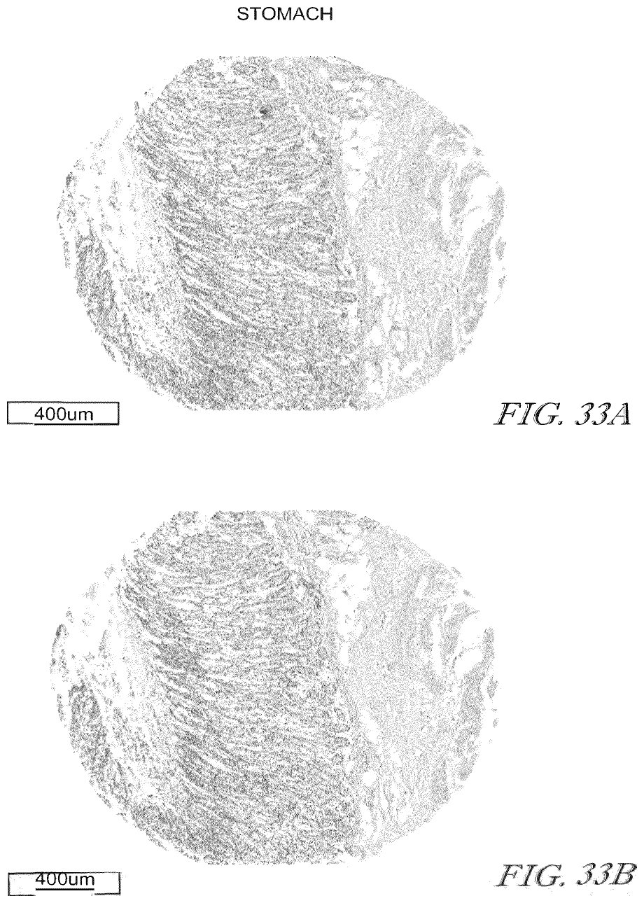

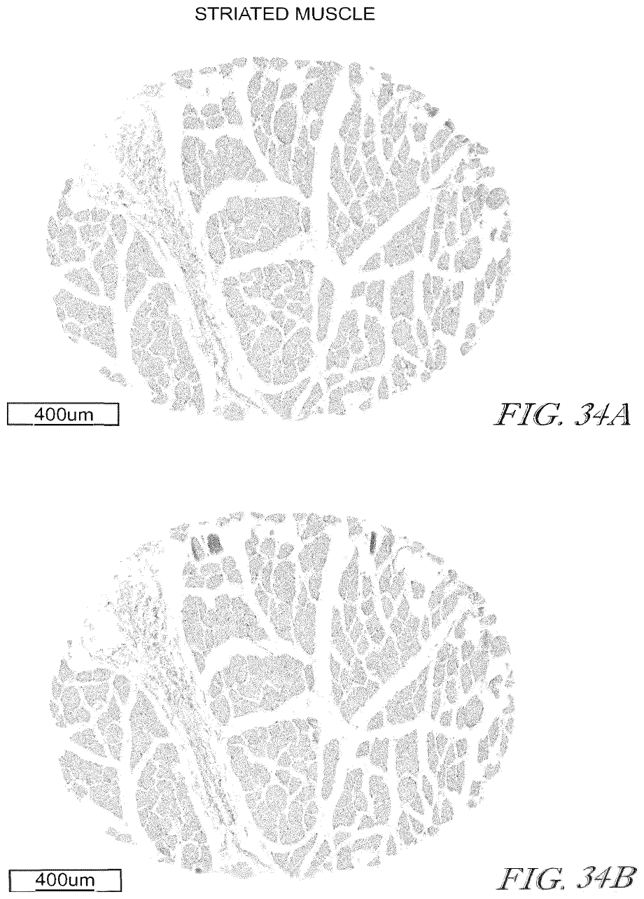

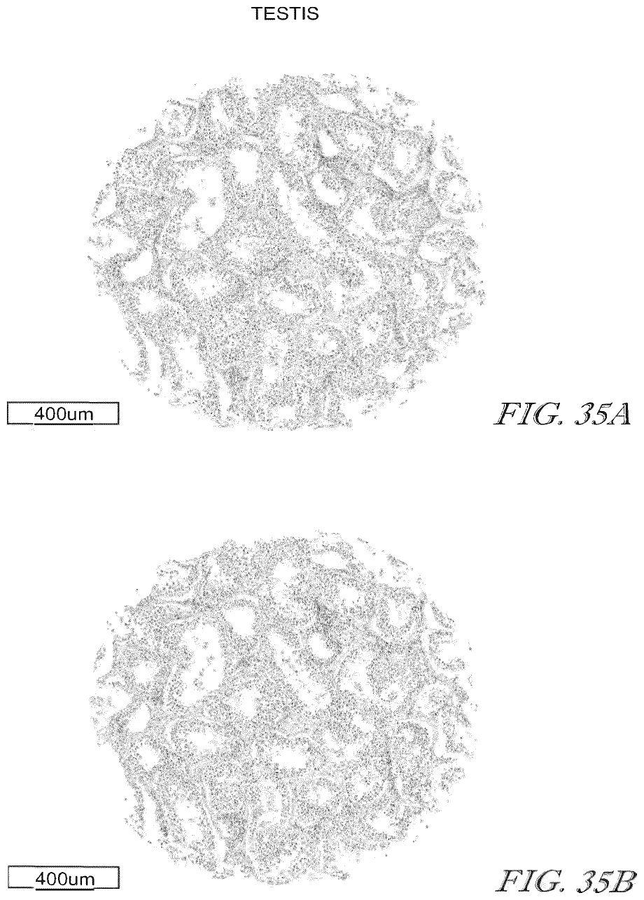

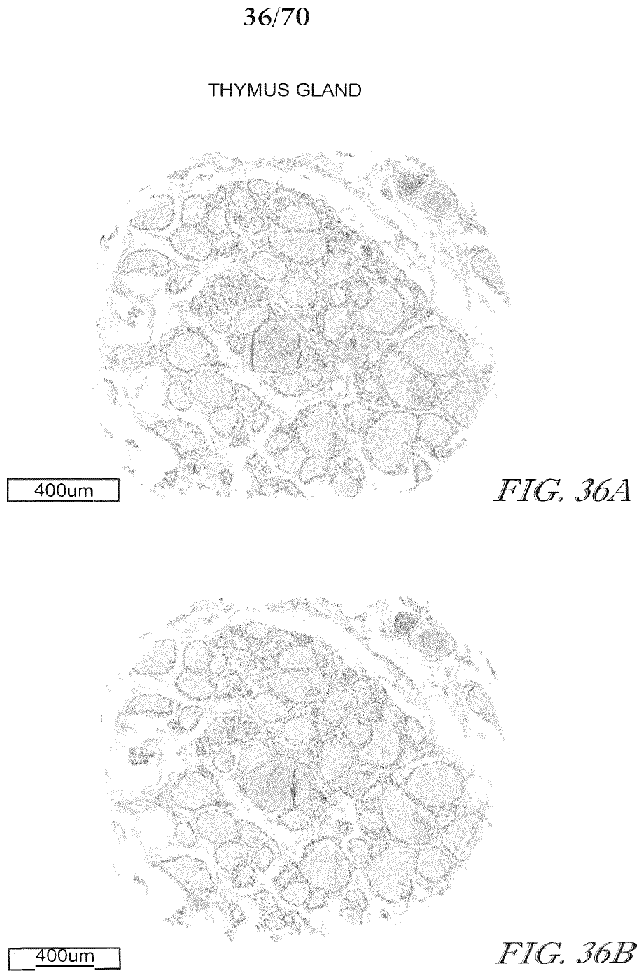

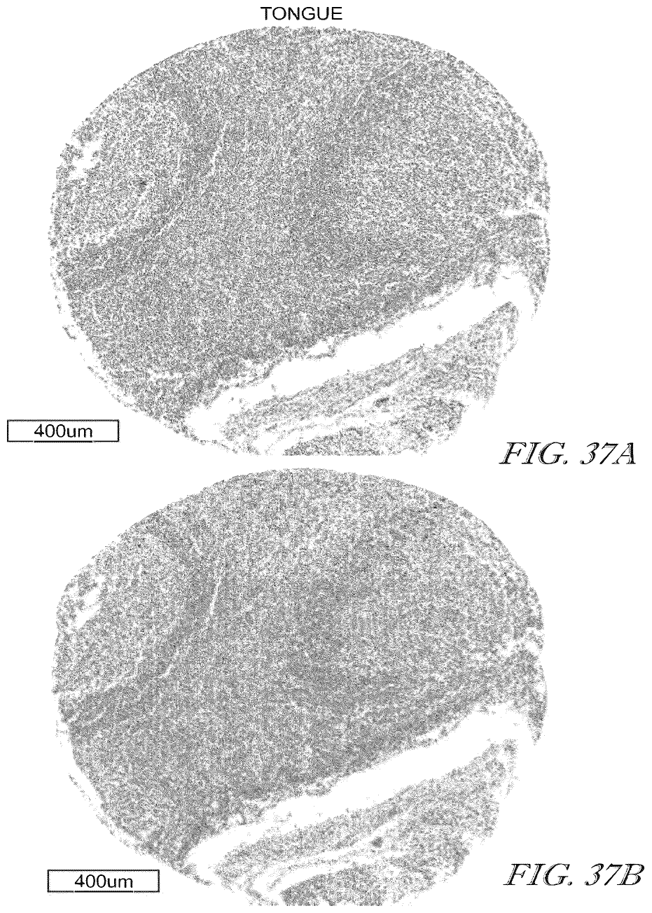

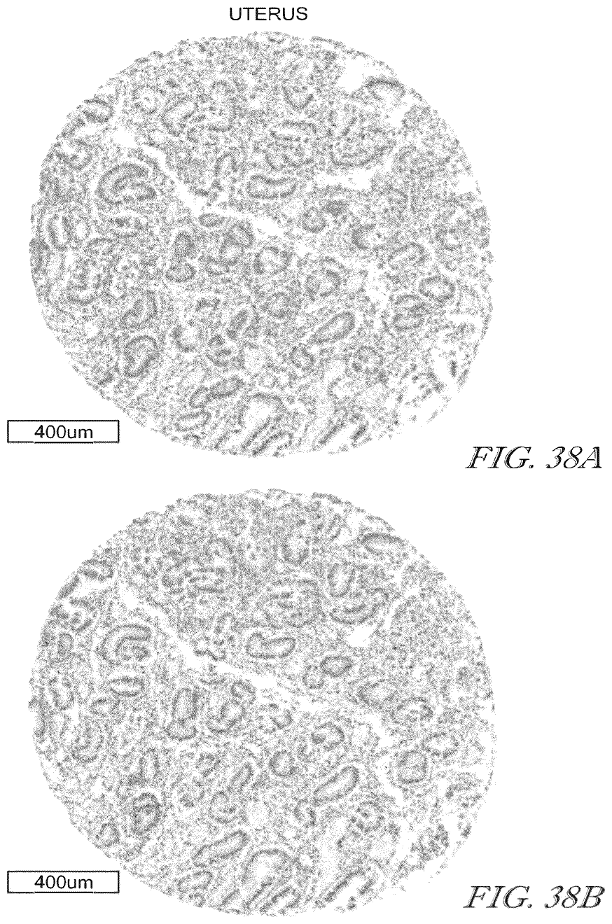

DIG-labeled E2 antibody was incubated with a human multiple organ normal tissue microarray (FDA999, US Biomax, Inc) and the binding of E2 antibody to human normal tissues was evaluated using the same IHC procedure with the same conditions described above. This multiple organ normal tissue microarray has 99 cores with 28 types of normal human organs, including adrenal gland (FIGS. 11A and 11B), bone marrow (FIGS. 12A and 12B), breast (FIGS. 13A and 13B), cerebellum tissue (FIGS. 14A and 14B), cervix (FIGS. 15A and 15B), colon (FIGS. 16A and 16B), esophagus (FIGS. 17A and 17B), eye (FIGS. 18A and 18B), heart (FIGS. 19A and 19B), hypophysis (FIGS. 20A and 20B), kidney (FIGS. 21A and 21B), larynx (FIGS. 22A and 22B), liver (FIGS. 24A and 24B), lung (FIGS. 25A and 25B), lymph node (FIGS. 26A and 26B), nerve (FIGS. 27A and 27B), ovary (FIGS. 28A and 28B), pancreas (FIGS. 29A and 29B), prostate (FIGS. 30A and 30B), skin (FIGS. 31A and 31B), small intestine (FIGS. 32A and 32B), spleen (FIGS. 23A and 23B), stomach (FIGS. 33A and 33B), striated muscle (FIGS. 34A and 34B), testis (FIGS. 35A and 35B), thymus gland (FIGS. 36A and 36B), tongue (FIGS. 37A and 37B), and uterus (FIGS. 38A and 38B). Organs taken from at least 3 normal human individuals were included in the microarray panel. As shown in FIGS. 11A-38B, no positive IHC staining was found in any of these 28 types of normal human organs tested, indicating that E2 anti-FITC antibody does not have any binding with normal human organ tissues. Some plasma cells showed certain levels of staining intensity in both the testing sections pre-incubated with DIG-E2 antibody and the negative control sections without pre-incubation with DIG-E2 antibody, indicating that this weak staining was non-specific.

In conclusion. E2 anti-FITC antibody does not bind with any tested human normal organ tissues, indicating E2 anti-FITC CAR-T cell itself should not bind to and attack any human normal tissues in vivo.

### Example 4

### Synthesis of FITC-Folate

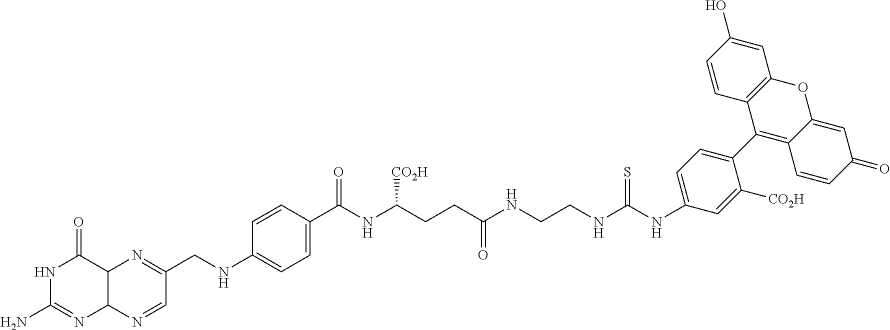 [SMILES: NC1=NC2N=CC(CNc3ccc(C(=O)N[C@@H](CCC(=O)NCCNC(=S)Nc4ccc(-c5c6ccc(=O)cc-6oc6cc(O)ccc56)c(C(=O)O)c4)C(=O)O)cc3)=NC2C(=O)N1]

Folate-γ-ethylenediamine was coupled to fluorescein isothiocyanate (FITC) isomer I (Sigma-Aldrich) in anhydrous dimethylsulfoxide (DMF) in the presence of tetramethylguanidine and diisopropylamine The crude product was loaded onto an Xterra RP18 preparative HPLC column (Waters) and eluted with gradient conditions starting with 99% 5 mM sodium phosphate (mobile phase A. pH 7.4) and 1% acetonitrile (mobile phase B) and reaching 90% A and 10% B in 10 min at a flow rate of 20 mL/min. Under these conditions, the FITC-folate main peak typically eluted at 27-50 min. The quality of the FITC-folate fraction was monitored by analytical reverse-phase HPLC with a UV detector. Fractions with greater than 98.0% purity (LCMS) were lyophilized to obtain the final FITC-folate product. As known in the art, the compound with this structure is also referred to as EC17.

### Example 5

### Synthesis of FITC-PEG12-Folate

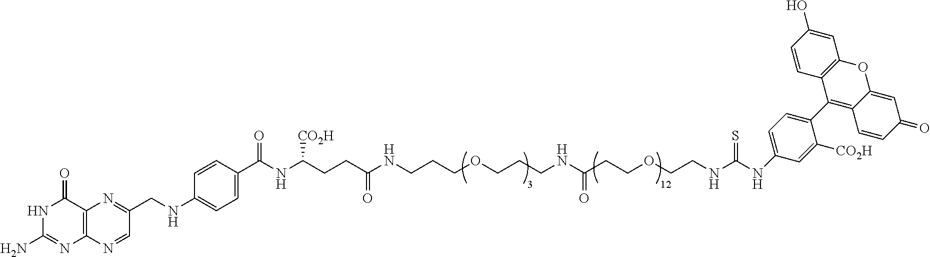

Universal polyethylene glycol (PEG) Nova Tag™ resin (0.2 g) was loaded into a peptide synthesis vessel and washed with isopropyl alcohol (i-PrOH) (3×10 mL) and dimethylformamide (DMF, 3×10 mL). 9-fluorenylmethoxycarbonyl (Fmoc) deprotection was carried out using 20% piperidine in DMF (3×10 mL). Kaiser tests were performed to assess reaction progress. To the vessel was then introduced a solution of Fmoc-L-glutamic acid 5-tert-butyl ester (Fmoc-Glu-(O-t-Bu)-OH) (23.5 mg) in DMF, N,N-diisopropylethylamine (i-Pr₂NEt) (4 equiv), and benzotriazol-1-yl-oxytripyrrolidinophosphonium hexafluorophosphate (PyBOP) (2 equiv). Fmoc deprotection was carried out using 20% piperidine in DMF (3×10 mL). To the vessel was then introduced a solution of N¹⁰-TFA-Pte-OH (22.5 mg), DMF, i-Pr₂NEt (4 equiv), and PyBOP (2 equiv). Argon was bubbled for 2 h, and the resin was washed with DMF (3×3 mL) and i-PrOH (3×3 mL). After swelling the resin in dichloromethane (DCM), a solution of 1M hydroxybenzotriazole (HOBT) in DCM/trifluoroethane (TFE) (1:1) (2×3 mL) was added. Argon was bubbled for 1 h, the solvent was removed, and the resin was washed with DMF (3×3 mL) and i-PrOH (3×3 mL). After swelling the resin in DMF, a solution of Fmoc-NH—(PEG)₁₂—COOH (46.3 mg) in DMF, i-Pr₂NEt (4 equiv), and PyBOP (2 equiv) was added. Argon was bubbled for 2 h, and the resin was washed with DMF (3×3 mL) and i-PrOH (3×3 mL). Fmoc deprotection was carried out using 20% piperidine in DMF (3×10 mL). Kaiser tests were performed to assess reaction progress. To the vessel was then introduced a solution of FITC (Life Technologies 21.4 mg) in DMF and i-Pr₂NEt (4 equiv), then Argon was bubbled for 2 h, and the resin was washed with DMF (3×3 mL) and i-PrOH (3×3 mL). Then to the vessel was added 2% NH₂NH₂ in DMF (2×2 mL). The final compound was cleaved from the resin using a TFA:H₂O: triisopropylsilane (TIS) (95:2.5:2.5) (Cleavage Solution) and concentrated under vacuum. The concentrated product was precipitated in Et₂O and dried under vacuum. The crude product was purified using preparative RP-HPLC (mobile phase: A=10 mM ammonium acetate pH=7, B=ACN; method: 0% B to 30% B in 30 min at 13 mL/min). The pure fractions were pooled and freeze-dried, providing the FITC-PEG12-Folate.

### Example 6

### Synthesis of FITC-PEG20-Folate

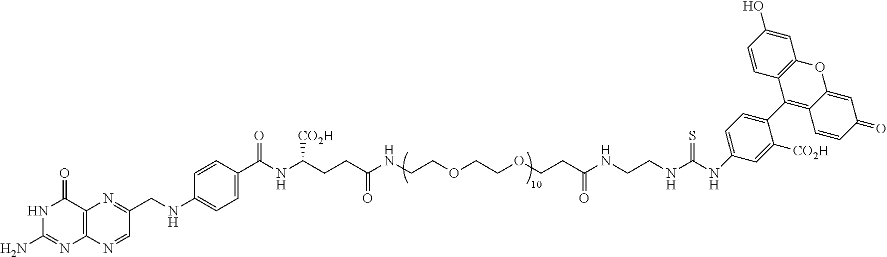

Ethylenediamine, polymer-bound (200-400 mesh)-resin (50 mg) was loaded into a peptide synthesis vessel and swollen with DCM (3 mL) followed by DMF (3 mL). To the vessel was then introduced the Fmoc-PEG₂₀-COOH solution (131 mg, 1.0 equiv) in DMF, i-Pr₂NEt (6.0 equiv), and PyBOP (4.0 equiv). Argon was bubbled for 6 h, the coupling solution was drained, and the resin was washed with DMF (3×10 mL) and i-PrOH (3×10 mL). Kaiser tests were performed to assess reaction progress. Fmoc deprotection was carried out using 20% piperidine in DMF (3×10 mL), before each amino acid coupling. The above sequence was repeated to complete the reaction with Fmoc-Glu-OtBu (72 mg, 2.0 equiv) and Tfa. Pteroic-acid (41 mg, 1.2 equiv) coupling steps. The resin was washed with 2% hydrazine in DMF 3×10 mL (5 min) to cleave the trifluoro-acetyl protecting group on pteroic acid and washed with i-PrOH (3×10 mL) followed by DMF (3×10 mL). The resin was dried under argon for 30 min The folate-peptide was cleaved from the resin using the Cleavage Solution. 10 mL of the cleavage mixture was introduced and argon was bubbled for 1.5 h. The cleavage mixture was drained into a clean flask. The resin was washed 3 times with more cleavage mixture. The combined mixture was concentrated under reduced pressure to a smaller volume (˜5 mL) and precipitated in ethyl ether.

The precipitate was collected by centrifugation, washed with ethyl ether (3 times) and dried under high vacuum. The dried Folate-PEG₂₀-EDA (1.0 equiv) was treated with FITC (50 mg, 1.5 equiv) in DMSO and DIPEA at room temperature. Progress of the reaction monitored by LCMS. After 8 h the starting material was consumed to give the product. The crude reaction mixture was purified by preparative HPLC, (mobile phase A=10 mM Ammonium Acetate. pH=7; Organic phase B=Acetonitrile; Method: 0% B to 30% B in 35 minutes at 13 mL/min) and provided FITC-PEG20-Folate in 60% yield.

### Example 7

### Synthesis of FITC-PEG108-Folate

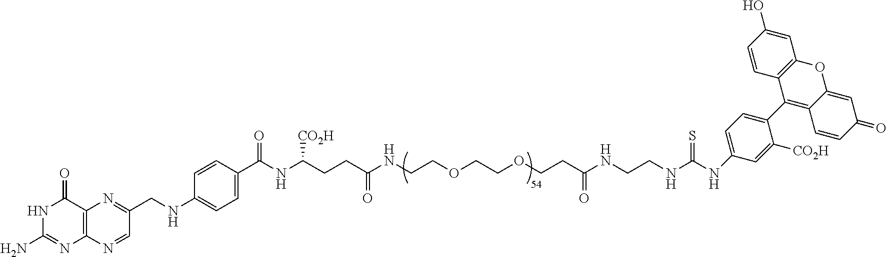

Ethylenediamine, polymer-bound (200-400 mesh)-resin (50 mg) was loaded in a peptide synthesis vessel and swollen with DCM (3 mL) followed by DMF (3 mL). To the vessel was then introduced the Fmoc-PEG₃₆-COOH solution (161 mg, 1.0 equiv) in DMF, i-Pr₂NEt (6.0 equiv), and PyBOP (4.0 equiv). Argon was bubbled for 6 h, the coupling solution was drained, and the resin was washed with DMF (3×10 mL) and i-PrOH (3×10 mL). Kaiser tests were performed to assess reaction progress. Fmoc deprotection was carried out using 20% piperidine in DMF (3×10 mL), before each amino acid coupling. The above sequence was repeated to complete reaction with 2×Fmoc-PEG₃₆-COOH (161 mg, 1.0 equiv), Fmoc-Glu-OtBu (72 mg, 2.0 equiv) and Tfa. Pteroic-acid (41.0 mg, 1.2 equiv) coupling steps. At the end the resin was washed with 2% hydrazine in DMF 3×10 mL (5 min) to cleave the trifluoro-acetyl protecting group on pteroic acid and washed with i-PrOH (3×10 mL) followed by DMF (3×10 mL). The resin was dried under argon for 30 min Folate-peptide was cleaved from the resin using the Cleavage Solution. 10 mL of the cleavage mixture was introduced and argon was bubbled for 1.5 h. The cleavage mixture was drained into a clean flask. The resin was washed 3× with more Cleavage Solution. The combined mixture was concentrated under reduced pressure to a smaller volume (˜5 mL) and precipitated in ethyl ether.

The precipitate was collected by centrifugation, washed with ethyl ether (3×) and dried under high vacuum. The dried Folate-PEG₁₀₈-EDA (1.0 equiv) was treated with FITC (50 mg, 1.5 equiv) in DMSO and DIPEA at room temperature. Reaction progress was monitored by LCMS. After 10 h starting material was consumed to give the product. The crude reaction mixture was purified by preparative HPLC, (mobile phase A=10 mM Ammonium Acetate. pH=7; Organic phase B=Acetonitrile; Method: 0% B to 30% B in 35 minutes at 13 mL/min) and provided FITC-PEG108-Folate in 64% yield.

### Example 8

### Synthesis of FITC-DUPA

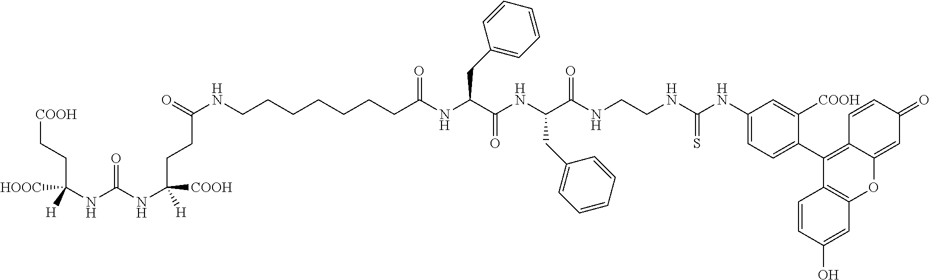 [SMILES: O=C(O)CC[C@H](NC(=O)N[C@@H](CCC(=O)NCCCCCCCC(=O)N[C@@H](Cc1ccccc1)C(=O)N[C@@H](Cc1ccccc1)C(=O)NCCNC(=S)Nc1ccc(-c2c3ccc(=O)cc-3oc3cc(O)ccc23)c(C(=O)O)c1)C(=O)O)C(=O)O]

DUPA-FITC was synthesized by solid phase methodology as follows. Universal Nova Tag™ resin (50 mg, 0.53 mM) was swollen with DCM (3 mL) followed by DMF 3 mL). A solution of 20% piperidine in DMF (3×3 mL) was added to the resin, and argon was bubbled for 5 mM. The resin was washed with DMF (3×3 mL) and isopropyl alcohol (i-PrOH, 3×3 mL). After swelling the resin in DMF, a solution of DUPA-(OtBu)-OH (1.5 equiv), HATU (2.5 equiv), and i-Pr₂NEt (4.0 equiv) in DMF was added. Argon was bubbled for 2 h, and resin was washed with DMF (3×3 mL) and i-PrOH (3×3 mL). After swelling the resin in DCM, a solution of 1 M HOBt in DCM/TFE (1:1) (2×3 mL) was added. Argon was bubbled for 1 h, the solvent was removed and resin was washed with DMF (3×3 mL) and i-PrOH (3×3 mL). After swelling the resin in DMF, a solution of Fmoc-Phe-OH (2.5 equiv), HATU (2.5 equiv) and DIPEA (4.0 equiv) in DMF was added. Argon was bubbled for 2 h, and the resin was washed with DMF (3×3 mL) and i-PrOH (3×3 mL). The above sequence was repeated for 2 more coupling steps for addition of 8-aminooctanoic acid and fluorescein isothiocyanate or rhodamine B isothiocyanate. The final compound was cleaved from the resin using the Cleavage Solution and concentrated under vacuum. The concentrated product was precipitated in diethyl ether and dried under vacuum. The crude product was purified using preparative RP-HPLC [λ=488 nm; solvent gradient: 1% B to 80% B in 25 mM, 80% B wash 30 mM run; A=10 mM NH₄OAc, pH=7; B=acetonitrile (ACN)]. ACN was removed under vacuum, and purified fractions were freeze dried to yield FITC-DUPA as a brownish-orange solid. RP-HPLC: tR=8.0 min (A=10 mM NH₄OAc, pH=7.0: B=ACN, solvent gradient: 1% B to 50% B in 10 min, 80% B wash 15 min run). ¹H NMR (DMSO-d6/D₂O): δ 0.98-1.27 (ms, 9H); 1.45 (b, 3H); 1.68-1.85 (ms, 11H); 2.03 (m, 8H); 2.6-3.44 (ms, 12H); 3.82 (b, 2H); 4.35 (m, 1H); 6.53 (d, J=8.1 Hz, 2H), 6.61 (dd, J=5.3, 3.5 Hz, 2H); 6.64 (s, 2H); 7.05 (d, J=8.2 Hz, 2H), 7.19 (m, 5H); 7.76 (d, J=8.0 Hz, 1H); 8.38 (s, 1H). HRMS (ESI) (m/z): (M+H)⁺0 calcd for C₅₁H₅₉N₇O₁₅S, 1040.3712, found, 1040.3702. UV/vis: λ max=491 nm.

### Example 9

### Synthesis of FITC-PEG 12-DUPA

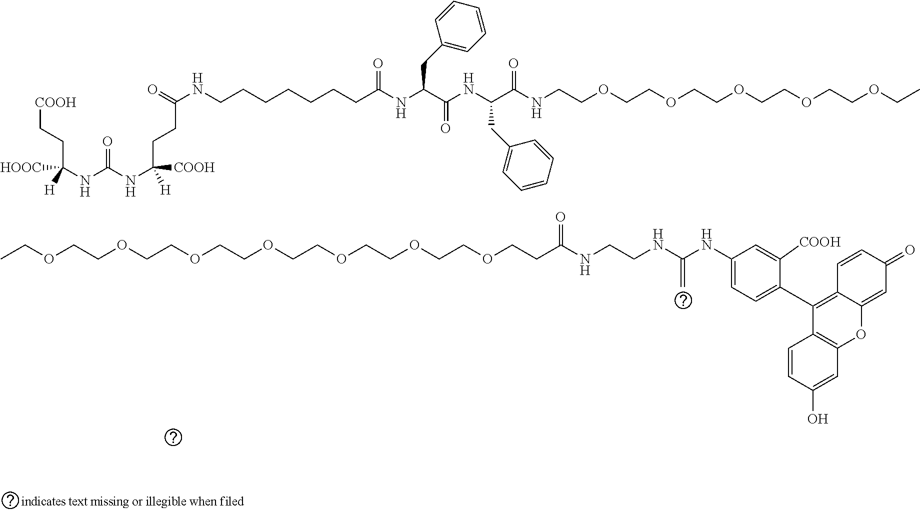

1,2-Diaminoethane trityl-resin (0.025 g) was loaded into a peptide synthesis vessel and washed with i-PrOH (3×10 mL), followed by DMF (3×10 mL). To the vessel was then introduced a solution of Fmoc-NH—(PEG)₁₂-COOH (42.8 mg) in DMF, i-Pr₂NEt (2.5 equiv), and PyBOP (2.5 equiv). The resulting solution was bubbled with Ar for 1 h, the coupling solution was drained, and the resin washed with DMF (3×10 mL) and i-PrOH (3×10 mL). Kaiser tests were performed to assess reaction progress. Fmoc deprotection was carried out using 20% piperidine in DMF (3×10 mL). This procedure was repeated to complete the all coupling steps (2×1.5 equiv of Fmoc-Phe-OH and 1.5 equiv of 8-aminooctanoic acid and 1.2 equiv of DUPA were used on each of their respective coupling steps). After the DUPA coupling, the resin was washed with DMF (3×10 mL) and i-PrOH (3×10 mL) and dried under reduced pressure. The peptide was cleaved from the resin in the peptide synthesis vessel using the Cleavage Solution. 15 mL of the Cleavage Solution was added to the peptide synthesis vessel, and the reaction was bubbled under Ar for 15 min. The resin was treated with two additional 10 mL quantities of the Cleavage Solution for 5 min each. The cleavage mixture was concentrated to about 5 mL and precipitated with ethyl ether. The precipitate was collected by centrifugation, washed with ethyl ether (3×), and dried under high vacuum, resulting in the recovery of crude material. To a stirred solution of the crude DUPA-(PEG)₁₂-EDA (10 mg) and FITC (5.6 mg) in dimethylsulfoxide (DMSO, 1 mL) was added i-Pr₂NEt (5 equiv) at room temperature and stirred for 6 h under argon. The reaction was monitored by LCMS and purified by preparative HPLC (mobile phase: A=10 mM ammonium acetate pH=7, B=ACN; method: 0% B to 50% B in 30 min at 13 mL/min). The purified fractions were pooled and freeze-dried, providing the FITC-PEG12-DUPA.

### Example 10

### Synthesis of FITC-PEG11-NK1

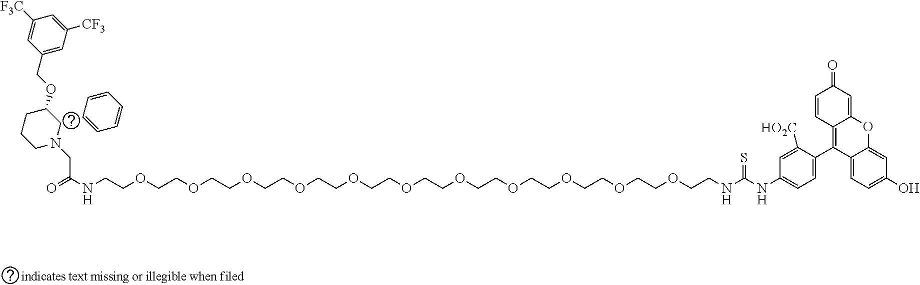

To a stirred solution of NK-1 (0.02 g, 0.0433 mmol, 1.0 eq.), O-(2-Aminoethyl)-O′-12-(Boc-amino)ethylldecaethylene glycol (BocNH-PEG₁₁-NH₂) (Sigma, 0.0336 g, 0.0521 mmol, 1.2 eq.), Benzotriazol-1-yl-oxytripyrrolidinophosphonium hexafluorophosphate (PyBOP) (0.027 g, 0.0521 mmol, 1.2 eq.) in dry CH₂Cl₂ was added N,N-Diisopropylethylamine (DIPEA) (0.076 mL, 0.4338 mmol, 10 eq.) under argon at room temperature. The reaction progress was monitored by LCMS and purified by preparative RP-HPLC (Waters, XBridge™ Prep C18, 5 μm; 19×100 mm column, mobile phase A=20 mM ammonium acetate buffer, pH 7, B=acetonitrile, gradient 10-100% B in 30 min, 13 mL/min, λ=220 nm, 254 nm). The pure fractions were collected, all organic solvents were evaporated and the sample was lyophilized for 48 h to provide the NK1-PEG₁₁-NHBoc. Yield: 40.13 mg (97%). To the NK1-PEG₁₁-NHBoc (0.0165 g, 0.015 mmol) in dry DCM was added trifluoroacetic acid (TFA, 20 eq.) and the reaction mixture was stirred for 4 h at r.t. The excess TFA was removed, and the remaining solution was diluted with water and extracted using CH₂Cl₂ (3×5 mL). The combined organic layers were washed with brine, dried (Na₂SO₄) and concentrated. The residue obtained was dried under vacuum and used for the next-step without further purification. A stirred solution of NK1-PEG₁₁-NH₂ (0.008 g, 0.0081 mmol, 1.0 eq.), Fluorescein isothiocyanate (FITC) (Sigma, 0.0037 g, 0.0097 mmol, 1.2 eq.) in dry dimethylsulfoxide (DMSO, 0.3 mL) was added to diisopropylethyl amine (0.0028 mL, 0.0162 mmol, 2.0 eq.) at room temperature under argon. The reaction progress was monitored by LCMS and the product was purified by preparative RP-HPLC (Waters, XBridge™ Prep C18, 5 μm; 19×100 mm column, mobile phase A=20 mM ammonium acetate buffer, pH 7, B=acetonitrile, gradient 10-100% B in 30 min, 13 mL/min, λ=280 nm). The pure fractions were collected, all organic solvents were evaporated and the sample was lyophilized for 48 h to provide the FITC-PEG11-NK1 in a yield of 8.54 mg (77%).

*Note: The NK-1 compound was synthesized by a two-step procedure starting from the base ligand, which was prepared by using a procedure in the literature. (Ref: DESIGN AND DEVELOPMENT OF NEUROKININ-1 RECEPTOR-BINDING AGENT DELIVERY CONJUGATES, Application Number: PCT/US2015/44229; incorporated herein by reference.

### Example 11

### Synthesis of FITC-PEG2-CA9

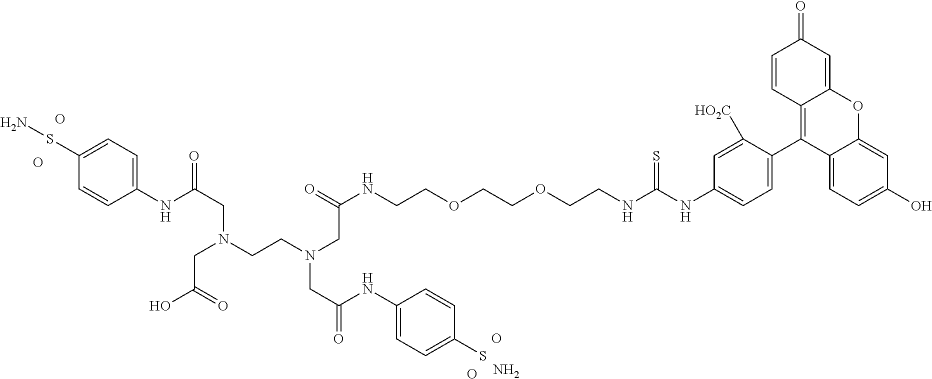

CA9 ligand (53.6 mg) was dissolved in DMF (2-3 mL) in a 50 mL round bottom flask using a Teflon magnetic stir bar. Ambient air was removed using a vacuum and replaced with nitrogen gas, this was done in three cycles. The round bottom flask was kept under constant nitrogen gas. To the flask, 28.9 mg of N-(3-Dimethylaminopropyl)-N′-ethylcarbodiimide hydrochloride (EDC) was added followed by 21.6 mg 1-Hydroxybenzotriazole hydrate (HOBt) and 18.9 μL of Boc-PEG₂-NH₂—(Sigma Aldrich). 5.4 μL of triethylamine (TEA) was added and the reaction was stirred overnight. The reaction mixture was purified using HPLC and confirmed with UHPLC-MS (target m/z of 831). Acetonitrile was removed using high vacuum rotary evaporation and the product lyophilized. The compound was mixed with 1:1 TFA:DCM for 30 minutes. The TFA/DCM was removed using high vacuum rotary evaporation followed by 30 minutes on high vacuum. The compound was then dissolved in DMF and combined with 5 molar equivalents of i-Pr:NEt, 16 mg of fluorescein isothiocyanate (Life Technologies) and stirred for 1 h. The reaction mixture was purified by HPLC and the target compound was confirmed with UHPLC-MS (target m/z of 1120). The samples were lyophilized and stored at −20° C.

### Example 12

### FITC-DUPA

DUPA-FITC was synthesized by solid phase methodology as follows. Universal NovaTag resin (50 mg, 0.53 mM) was swollen with dichloromethane (DCM) (3 mL) followed by dimethylformamide (DMF, 3 mL). A solution of 20% piperidine in DMF (3×3 mL) was added to the resin, and argon was bubbled for 5 min. The resin was washed with DMF (3×3 mL) and isopropyl alcohol (i-PrOH, 3×3 mL). After swelling the resin in DMF, a solution of DUPA-(OtBu)-OH (1.5 equiv), HATU (2.5 equiv) and DIPEA (4.0 equiv) in DMF was added. Argon was bubbled for 2 h, and resin was washed with DMF (3×3 mL) and i-PrOH (3×3 mL). After swelling the resin in DCM, a solution of 1 M HOBt in DCM/trifluoroethane (TFE) (1:1) (2×3 mL) was added. Argon was bubbled for 1 h, the solvent was removed and resin was washed with DMF (3×3 mL) and i-PrOH (3×3 mL). After swelling the resin in DMF, a solution of Fmoc-Phe-OH (2.5 equiv), HATU (2.5 equiv) and DIPEA (4.0 equiv) in DMF was added. Argon was bubbled for 2 h, and resin was washed with DMF (3×3 mL) and i-PrOH (3×3 mL). The above sequence was repeated for 2 more coupling steps for addition of 8-aminooctanoic acid and fluorescein isothiocyanate or rhodamine B isothiocyanate. Final compound was cleaved from the resin using a trifluoroacetic acid (TFA):H20:triisopropylsilane:cocktail (95:2.5:2.5) and concentrated under vacuum. The concentrated product was precipitated in diethyl ether and dried under vacuum. The crude product was purified using preparative RP-HPLC [λ=488 nm; solvent gradient: 1% B to 80% B in 25 min, 80% B wash 30 min run; A=10 mM NH4OAc, pH=7; B=acetonitrile (ACN)]. ACN was removed under vacuum, and pure fractions were freeze-dried to yield DUPA-FITC as a brownish-orange solid. RP-HPLC: tR=8.0 min (A=10 mM NH4OAc, pH=7.0; B=ACN, solvent gradient: 1% B to 50% B in 10 min, 80% B wash 15 min run). 1H NMR (DMSO-d6/D20): δ 0.98-1.27 (ms, 9H); 1.45 (b, 3H); 1.68-1.85 (ms, 11H); 2.03 (m, 8H); 2.6-3.44 (ms, 12H); 3.82 (b, 2H); 4.35 (m, 1H); 6.53 (d, J=8.1 Hz, 2H), 6.61 (dd, J=5.3, 3.5 Hz, 2H); 6.64 (s, 2H); 7.05 (d, J=8.2 Hz, 2H), 7.19 (m, 5H); 7.76 (d, J=8.0 Hz, 1H); 8.38 (s, 1H). HRMS (ESI) (m/z): (M+H)+ calcd for C51H59N7015S, 1040.3712. found, 1040.3702. UV/vis: λ max=491 nm.

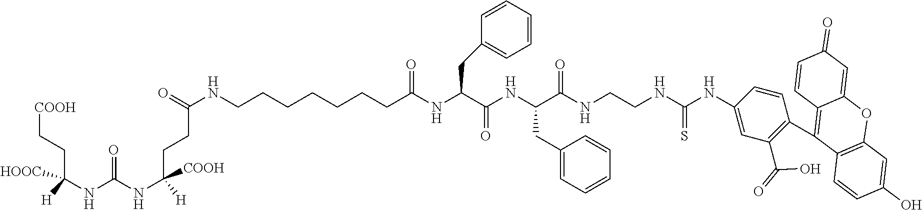 [SMILES: O=C(O)CC[C@H](NC(=O)N[C@@H](CCC(=O)NCCCCCCCC(=O)N[C@@H](Cc1ccccc1)C(=O)N[C@@H](Cc1ccccc1)C(=O)NCCNC(=S)Nc1ccc(-c2c3ccc(=O)cc-3oc3cc(O)ccc23)c(C(=O)O)c1)C(=O)O)C(=O)O]

### Example 13

### FITC-CA9

In a 50 mL round bottom flask CA9 ligand (53.6 mg, synthesized in lab) was dissolved in a desired amount of N,N-Dimethylformamide (DMF) (2-3 mL) using a Teflon magnetic stir bar. Ambient air was removed using vacuum and replaced with nitrogen gas, this was done in three cycles. Then the round bottom was kept under constant nitrogen gas. To the flask, 28.9 mg of N-(3-Dimethylaminopropyl)-N′-ethylcarbodiimide hydrochloride (EDC) was added followed by 21.6 mg 1-Hydroxybenzotriazole hydrate (HOBt) and 18.9 μL of Boc-PEG2-NH2 (purchased from Sigma Aldrich). 5.4 μL of triethylamine (TEA) was added last and the reaction was allowed to stir overnight. The reaction mixture was purified using HPLC and confirm with UHPLC-MS (target m/z of 831). Acetonitrile was removed using high vacuum rotary evaporation and place on lyophilizer for 48 hours. Deprotection of Boc was done with with 1:1 TFA:DCM for 30 minutes. TFA/DCM was removed using high vacuum rotary evaporation followed by 30 minutes on high vacuum. The compound was then dissolved in DMF and combined with 5 molar equivalents of N,N-Diisopropylethylamine (DIPEA). 16 mg of fluorescein isothiocyanate (purchased from Life Technologies) was added to the solution and stirred for 1 hour. Reaction mixture was purified by HPLC and target compound was confirmed with UHPLC-MS (target m/z of 1120). The samples was placed on lyophilizer for 48 hours and store compound at −20° C.

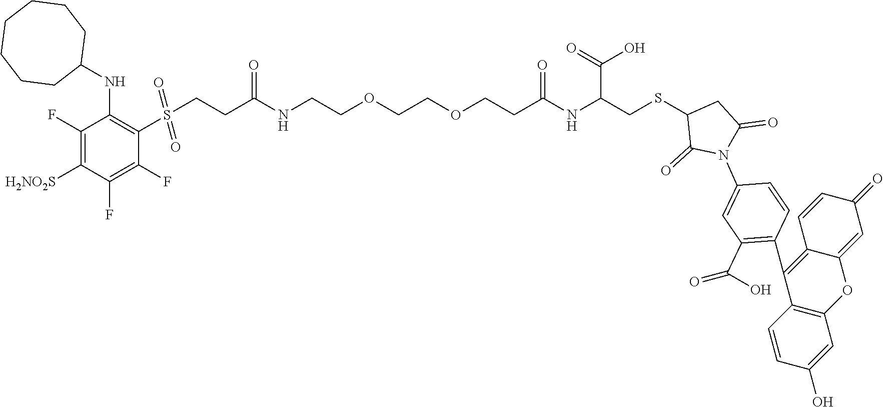 [SMILES: NS(=O)(=O)c1c(F)c(F)c(S(=O)(=O)CCC(=O)NCCOCCOCCC(=O)NC(CSC2CC(=O)N(c3ccc(-c4c5ccc(=O)cc-5oc5cc(O)ccc45)c(C(=O)O)c3)C2=O)C(=O)O)c(NC2CCCCCCC2)c1F]

### Example 14

### FITC-NK1R

To a stirred solution of NK-1 (0.02 g, 0.0433 mmol, 1.0 eq.), 0-(2-Aminoethyl)-O′-12-(Boc-amino)ethylldecaethylene glycol (BocNH-PEG11-NH2) (Sigma, 0.0336 g, 0.0521 mmol, 1.2 eq.), Benzotriazol-1-yl-oxytripyrrolidinophosphonium hexafluorophosphate (PYBOP) (0.027 g, 0.0521 mmol, 1.2 eq.) in dry CH2Cl2 was added N,N-Diisopropylethylamine (DIPEA) (0.076 mL, 0.4338 mmol, 10 eq.) under argon at room temperature. The reaction progress was monitored by LCMS and purified by preparative RP-HPLC (Waters, XBridge™ Prep C18, 5 μm; 19×100 mm column, mobile phase A=20 mM ammonium acetate buffer, pH 7, B=acetonitrile, gradient 10-100% B in 30 min, 13 mL/min, λ=220 nm, 254 nm). The pure fractions were collected, evaporated all organic solvents and lyophilized the sample for 48 h to furnish the NK1-PEG11-NHBoc. Yield: 40.13 mg (97%). To the NK1-PEG11-NHBoc (0.0165 g, 0.015 mmol) in dry CH2Cl2 was added trifluoroacetic acid (TFA, 20 eq.) and reaction mixture was stirred for 4 h at r.t. The excess of TFA was removed, diluted with water and extracted using CH2Cl2 (3×5 mL). The combined organic layers were washed with brine, dried (Na2SO4) and concentrated. The residue obtained was dried under vacuum and used for next-step without further purification. A stirred solution of NK1-PEG11—NH2 (0.008 g, 0.0081 mmol, 1.0 eq.), Fluorescein isothiocyanate (FITC) (Sigma, 0.0037 g, 0.0097 mmol, 1.2 eq.) in dry dimethylsulfoxide (DMSO, 0.3 mL) was added diisopropylethyl amine (0.0028 mL, 0.0162 mmol, 2.0 eq.) at room temperature under argon. The reaction progress was monitored by LCMS and purified by preparative RP-HPLC (Waters, XBridge™ Prep C18, 5 μm; 19×100 mm column, mobile phase A=20 mM ammonium acetate buffer, pH 7, B=acetonitrile, gradient 10-100% B in 30 min, 13 mL/min, λ=280 nm). The pure fractions were collected, evaporated all organic solvents and lyophilized the sample for 48 h to furnish the NK1-PEG11-FITC (5). Yield: 8.54 mg (77%).

The NK-1 compound was synthesized by a two-step procedure starting from base ligand, which was prepared by using a literature procedure. (Ref: DESIGN AND DEVELOPMENT OF NEUROKININ-1 RECEPTOR-BINDING AGENT DELIVERY CONJUGATES, Application Number: PCT/US2015/44229, incorporated herein by reference in its entirety).

### Example 15

### In Vitro Co-Culture E2 CAR T Activation and Target Cell Kill Assay

E2 CAR T cells were thawed and recovered from cryopreservation at a cell density between 0 5 million to 2 million T cells per milliliter of T cell growth media (TexMACS media+2% human AB serum+50 U/mL recombinant human IL2) for 4 days. Target cells which express different levels of surface folate receptor were also thawed and recovered from cryopreservation in growth media (folate deficient RPMI1640+10% FCS+pen/strep for 4 days).

On day −1, 100,000 target cells were plated in 2 ml of growth media per well of a 12-well tissue culture plate to allow target cells to adhere and recover. On day 0, wells which received EC17 bridge molecule were treated with EC17 at 100 nM for 30 min in the tissue culture incubator. Afterwards, all media was washed off and replaced with fresh growth media free of folate and EC17. During the EC17 incubation, T cells were harvested, counted and after EC17 treatment was completed, 100,000 E2 CAR T cells were directly added onto each well of target cells in a total of 3 ml of growth media at a ratio of 1:1 (E:T) and incubated for both 1 and 2 days under standard tissue culture humidified conditions of 37° C. at 5% CO₂. One day co-culture conditions were used to assay E2 CAR T cell activation by target cells and two-day incubation was used to assay target cell death.

To determine the E2 CAR T cell activity level after one day coculture with target cells possessing different levels of surface folate receptor, floating cells were harvested and pooled with the remaining adherent cells which were removed using a 5-minute 0.25% trypsin digest from the tissue culture plate. Pooled floating and adherent cells were pelleted with a 400×gravity centrifugation step for 5 minutes then resuspended in flow cytometry staining solution. Surface marker staining for E2 T cell identification via flow cytometry included anti-human EGFR and anti-human CD3 while detection of CAR T cell activation via flow cytometry utilized an anti-human CD137 stain. E2 CAR T cells were identified as EGFR+ CD3+ while activated CAR T cells also co-expressed CD137 (see flow cytometry methods).

To determine the efficiency of E2 CAR T cell killing of target cells with different levels of surface folate receptor expression after a two day coculture, pooled and adherent cells were pelleted in the same manner as the one day coculture assay above then stained for E2 CAR T markers EGFR and CD3 then washed and stained for the apoptosis marker, Annexin V, by resuspending the flow antibody stained samples with Alexa Fluor 647 conjugated recombinant Annexin V (Invitrogen cat #A23204 at 1:50 dilution in 1× Annexin staining buffer provided) with 3 μM propidium iodide.

### E2 CAR T Cell Activation is Dependent on EC17 Staining of Target Cells

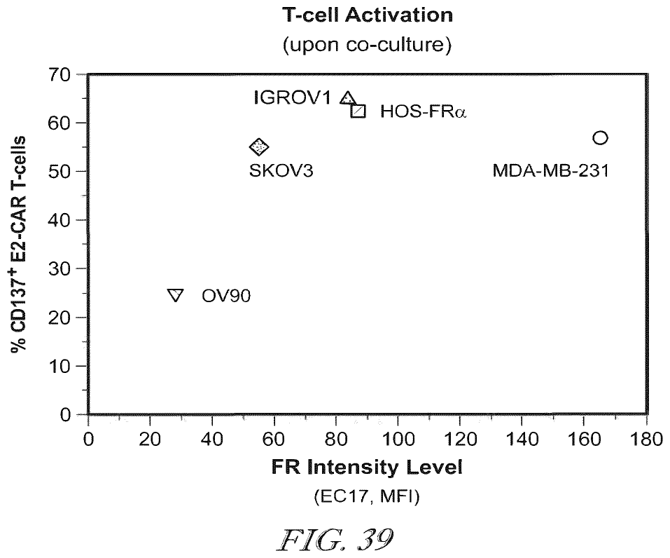

In an effort to understand the levels of folate receptor necessary to activate E2 CAR T cells, one day cocultures with target cells expressing surface folate receptor (FR) at varying levels was performed (FIG. 39). The percentage of E2 CAR T cells which are activated after the one-day coculture is graphed on the y-axis, while the FR expression level of the target cells at the time of the assay is graphed on the x-axis. Importantly the FR expression level data was acquired on the same day the assay was performed using the same target cells which were treated with EC17 but cultured without CAR T cells to avoid any artifacts of FR high expressing cells being culled by the CAR T cells. Four out of five target cell lines which express a wide range of folate receptor activated the CAR T cells to similar levels. Interestingly the low FR expressing cell line, OV90 (down triangle), expresses about 50% of the FR as the SKOV3 cells (diamond) and activates about 50% of the CAR T cells as the SKOV3 cells do. These data show that a wide range of EC17 will activate the E2 CAR T cells to similar levels suggesting there is a certain threshold of EC17 bound to the target cell is required to get the full activation of the E2 CAR T cells. Additionally, it is also suggested that there is a threshold of FR expression that will give a suboptimal E2 CAR T response.

### E2 CAR T Cell Target Cell Killing

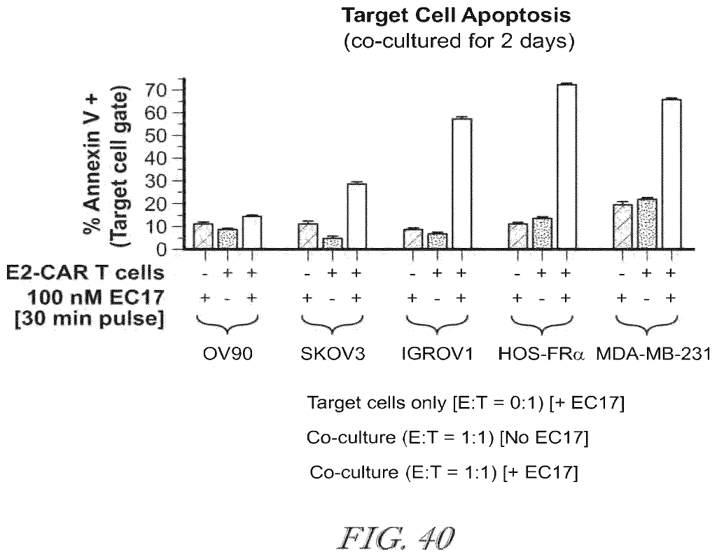

To confirm that E2 CAR T cell activation also translates to successful killing of the target cells we performed a two-day co-culture assay then looked at target cell (EGFR− CD3−) events via flow cytometry and determined the percentage of target cell staining with the apoptotic surface marker, Annexin V stain. In FIG. 40, the same five target cells as shown (as labeled on x-axis), each cultured for two days under three different conditions, shown as three different bars. The first bar in each group represents the basal level of target cell apoptosis as these were target cells treated with EC17 but cultured in the absence of E2 CAR T cells. The second bar in each group represents CAR T plus target cell cocultures without any previous EC17 treatment of target cells while the third bar in each group represents the CAR T plus EC17 pre-treated target cells. The y-axis shows the percentage of target cells which are apoptotic (Annexin V+). From these data it was discovered that CAR T cell activation translates well into target cell killing for the three highest folate receptor expressers (MDAMB231, HOS-FRα and IGROV1). However, for the SKOV3 cells which had the fourth lowest FR expression (FIG. 39, x-axis), similar E2 CAR T activation did not translate to similar target cell kill (FIG. 40). It is possible that this disconnect between CAR T cell activation and target cell apoptosis in SKOV 3 cells may not be a function of the low folate receptor expression. As previously stated each cancer cell may have evolved its own anti-apoptotic mechanism and it is possible that FR expression levels like that of SKOV3 cells but in a different cancer cell line, may be enough for a higher level of target cell apoptosis.

Taken together, the data from FIG. 39 and FIG. 40 demonstrate that EC17 binding to cancer cells is required for cell kill. These data further suggest that below a certain threshold of target cell bound EC17 a linear relationship between surface bound EC17 and E2 CAR T activation and killing exists.

### Example 16

### ³H-Folic Acid Binding Assay

All tumor cell lines were seeded overnight in folate-free RPMI medium containing 10% heat-inactivated fetal calf serum. On the following day, the cells were incubated for 15 min on ice with 100 nM ³H-folic acid with and without 10 μM cold folic acid. After rising 3 times with cold 1×PBS, whole cells were lysed and total cell-associated radioactivity was counted in a scintillation counter. FR-specific binding of ³H-folic acid were determined by subtracting the counts of folic acid competed samples and calculated as number of molecules per cell using the specific activity of ³H-folic acid.

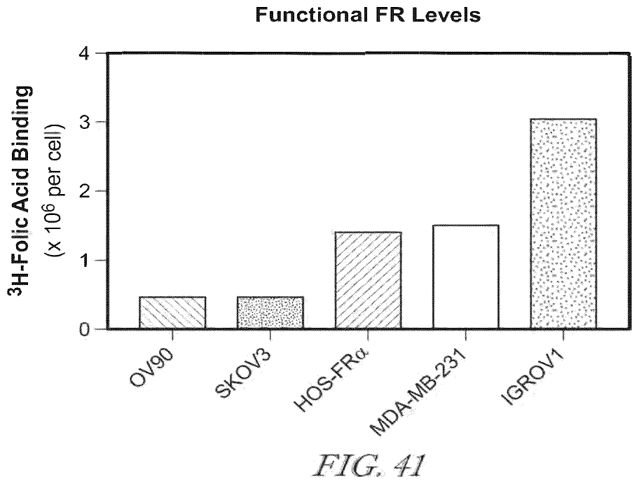

FIG. 41 demonstrates various levels of FR expression in these cell lines. The order of FR levels are: IGROV1>MDA-MB-231, HOS-FRα>OV90, SKOV3.

### Example 17

EC17/E2-CAR T-Cell Therapy in NSG Mice with and without Subcutaneously Implanted MDA-MB-231-Tumors

### Tumor Implantation

MDA-MB-231 tumor cells were grown in folate-deficient RPMI 1640 with 5-10% FBS at 37° C. in a 5% CO₂ humidified atmosphere. MDA-MB-231 tumor cells were inoculated subcutaneously at 2.5×10⁶ cells per animal. Only 42 out of the 66 mice were inoculated and the remaining 22 mice were kept tumor-free.

### CAR-T Cell Administration

EGFRt-sorted anti-FITC E2 scFv-CAR T cells were frozen in a T-cell freezing medium. Vials of frozen CAR-T cells were immediately stored at −80° C. The CAR-T cells were quickly thawed at 37° C., washed twice with PBS, and used for animal injection at 10 million viable EGFRt+E2 CAR-T cells (CD4/CD8 at ˜1:1) per animal. A small aliquot was taken on the day infusion for flow cytometric analysis of E2-CAR T-cell phenotypes.

### Human/Mouse Cytokine Analyses

Mouse blood samples were processed for plasma and stored at −20° C. until use. A human TH1 cytokine panel (Biolegend, Cat. No. 740009) and a human hematopoietic stem cell cytokine panel (Biolegend, Cat. No. 740610) was used to detect human cytokines in the mouse blood. A mouse 13-plex inflammatory cytokine panel (Biolegend, No. 740150) was used to detect mouse cytokines. All analyses were performed per the manufacturer's instructions.

### Flow Cytometry Analysis

Whole blood cell analysis: Plasma was removed from predetermined volumes of whole EDTA treated blood and RBCs were lysed with RBC lysis solution. The leukocyte pellets were then resuspended in flow cytometry staining solution (1% bovine serum albumin, 50 mg/mL human IgG (Equitech Bio, cat #SLH56-0001), 0.9% sodium azide in a phosphate buffered saline, pH=7.4) and leukocyte surface marker staining was performed using the following antibodies: anti-human CD45 [clone HI30, eBioscience #47-0459-42 at 1:20 (v/v) dilution], anti-human CD137 [clone 4B4-1, BD Bioscience #564092 at 1:20 (v/v) dilution], anti-human CD8a [clone RPA-T8, BD Bioscience, catalog #557746 at 1:20 (v/v) dilution], anti-human CD4 [clone SK3, eBioscience catalog #46-0047-42 at 1:20 (v/v) dilution], anti-human EGFR [R&D systems, clone Hul, catalog #FAB9577B @ 1:10 (v/v)], anti-human PD1 [BD Biosciences, clone EH12.1, catalog #562511 @1:20 (v/v)], anti-human LAG3 [BD Biosciences, clone T47-530, catalog #565616 @1:20 (v/v)], anti-human TIM3 [BD Biosciences, clone 7D3, catalog #565558 @1:20 (v/v)], anti-human CD3ε [BD Biosciences, clone SK7, catalog #557832 @1:20 (v/v)]. After leukocyte staining, cells were washed with PBS and resuspended in cold PBS containing 53,000 CountBright™ beads (Invitrogen catalog #C36950) and transferred to flow cytometry collection tubes. Flow cytometry data was collected on the Gallios flow cytometer (Beckman Coulter, Brea, CA). Determination of the concentration of CAR T cells in each blood sample was calculated according to Invitrogen's instructions. CAR T cells were identified as human CD3ε+ EGFRt+ events and easily distinguished and counted using the Kaluza™ flow cytometry software. The number of CAR T cells in the circulation of each infused mouse was then represented on the graphs as the total number of CAR T cells per 100 μL of whole blood analyzed. Statistical significance was determined by utilizing an unpaired, two-tailed, students t-test with significance set at p<0.05.

Tumor and tissue analysis: Solid tumors (100-1000 mm³) were harvested, weighed, and minced into small pieces then transferred into 50 mL tubes containing 20 mL of a tumor digestion cocktail. The enzymatic tumor digestion cocktail consisted of 0.5 mg/mL Collagenase IV (Sigma-Aldrich, Catalog #C5138), 0.5 mg/mL Hyaluronidase (Sigma-Aldrich, Catalog #H3506) and 0.1 mg/mL DNase I (Sigma-Aldrich, Catalog #DN25) in serum-free and folate-deficient RPMI1640 medium supplemented with antibiotics. The tumor fragments were digested for one hour at 37° C. at 300 rpm on a horizontal shaker. Afterwards, the tumor digest was centrifuged at 400×g for 5 minutes and tumor cell pellet underwent a red blood cell lysis step, was then washed with cold phosphate-buffered saline (PBS, pH 7.4) and finally filtered through a 40 μm nylon cell strainer.

### E2-CAR-T Phenotypes and Proliferation In-Vivo

Expansion and persistence of CAR T cells in the blood has been reported to correlate with response to CD19 specific CAR T therapy in clinical studies. It is not unreasonable to presume that a larger pool of circulating blood borne E2 CAR T will allow for a greater response to EC17 directed attack on FR+ tumors in our in vivo models. As a measure of response of E2 CAR T cell therapy to EC17, we quantified the number of circulating E2 CAR T cells per 100 uL of whole blood and observed cohorts which received EC17 stimulation (cohorts 2, 3, 4 and 5) had at least more than 10-fold increase in circulation when compared to cohorts which did not receive EC17 (cohorts 1 and 6). Importantly this expansion of circulating CAR T cells within the blood is consistent with CAR T cell expansion in an antigen dependent manner. Additionally, we also observed persistence of the elevated numbers of circulating CAR T cells to be as long as 54 days post CAR T cell infusion and again CAR T persistence was dependent on EC17 (cohorts 2, 3, 4 and 5) when compared to cohorts 1 and 6 which did not receive EC17.

### Tumor Vs Tumor-Free Mice

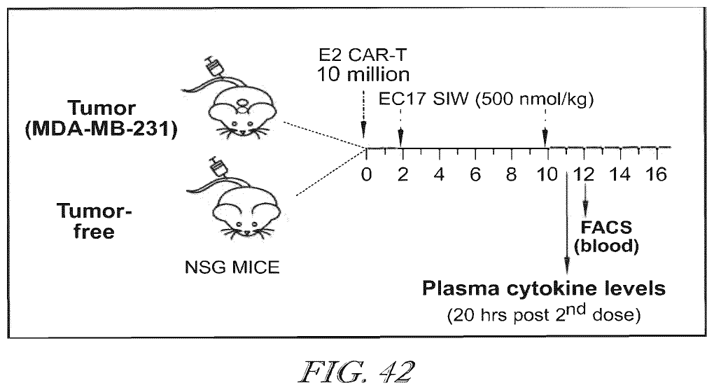

As shown in FIG. 42, 10 million E2 CAR-T cells were i.v. injected into naïve NSG mice without tumor or NSG mice bearing MDA-MB-231 tumors. 500 nmol/kg EC17 was i.v. dosed on days 2 and 10 post CAR-T injection, and mice were euthanized 20 hours after second EC17 dose (day 11) for organ evaluation and blood analysis. The plasma samples were isolated from blood immediately and stored at −20° C. until cytokine analysis. On Day 12 post CAR-T injection, a separate set of animals were harvested for FACS analysis as described above.

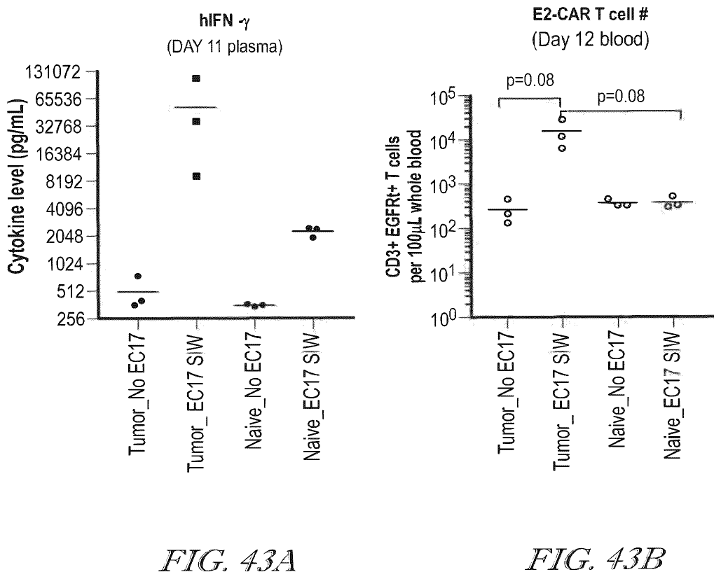

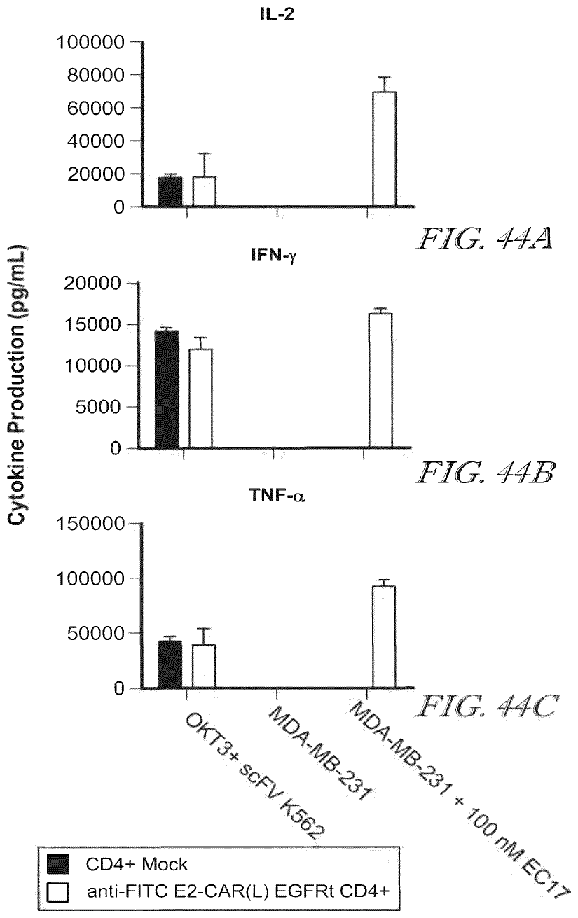

As shown in FIGS. 43A and 43B, cytokine production in tumor-bearing mice was EC17-dependent. The levels of cytokine including IL2, IFN-γ, IL-10 were all increased in tumor-bearing mice with EC17 relative to those without an EC17 dose. In naïve mice without a tumor, the up-regulation of cytokine production was also observed in those mice dosed with EC17, but the increase in tumor-free mice was much lower comparing to those mice with MDA-MB-231 tumors (e.g. 23-fold lower for IFN-γ).

Tumor-bearing mice had ˜23-fold higher IFN-γ production than tumor-free mice. While tumor-free mice (on FD-diet for ˜2 months) showed some cytokine production post EC17 SIW500×2 doses, there was no detectable CAR-T cell expansion by FACS.

### Example 18

Target-Specific Activity Demonstrated In-Vitro Using Humanized Anti-FITC scFv E2 CAR Construct and CAR-Modified T Cells

Cell lines: All cell lines were maintained in RPMI 1640 (Irvine Scientific) supplemented with 2 mM L-glutamine (Cellgro), 25 mM HEPES (Irvine Scientific) and 10% heat-inactivated FBS (Hyclone) unless otherwise noted. Dr. Stanley Riddell (FHCRC) kindly provided the K562 target cell lines. The K562 OKT3 cell line was generated by transduction with an OKT3 transgene containing lentivirus. The OKT3 is a membrane tethered scFv that targets CD3 epsilon. The MDA-MB-231 were provided by Endocyte and cultured in folate free DMEM. All cell lines were authenticated by STR Profiling matched to the DSMZ Database by the University of Arizona Genetics Core.

Cytotoxicity and cytokine secretion: Four-hour chromium release assays were performed. Briefly, target cells were labeled with ⁵¹Cr (PerkinElmer) overnight, washed three times in PBS, and incubated in RPMI in triplicate at 5×103 cells/well with T-cells at various effector to target (E:T) ratios in a 96-well plate. Supernatants were harvested for 7-counting and specific lysis was calculated. For cytokine secretion, 5×10⁵ T-cells were plated in triplicate with target cells at an E:T ratio of 2:1 in a 96-well plate for 24-hours and supernatants were analyzed by cytometric bead array using a Bio-Plex Human Cytokine Panel (Bio-Rad) according to the manufacturer's instructions. For EC17 labeled targets, the cells were incubated in the dark at room temperature for 1 hr with 100 nM EC17 in PBS prior to plating. When EC17 was used, cells were incubated with folate free RPMI during the assay.

### Cytokine Release Assay:

CD4+ T cells were co-cultured at a 2:1 ratio T-cell to target for 24 hrs and then the supernatant was analyzed for the presence of effector cytokines IL-2, IFN-γ, and TNF-α. Assays were run in triplicate and data shown as mean±SE. As shown in FIGS. 44A-C, we observed that Mock and anti-FLCAR T-cells produced quantitatively similar levels of cytokine production in response to co-culture with the positive control cell line K562-0KT3. No cytokine was produced by either Mock or anti-FLCAR T-cells upon co-culture with K562. Cytokine release was dependent upon prior incubation of EC17 with the MDA-MB-231 cell line and the anti-FLCAR T-cells were the only cells able to elicit secretion of the cytokines IL-2, IFN-γ, and TNF-α against EC17 labeled MDA-MB-231.

### Chromium Release Assay:

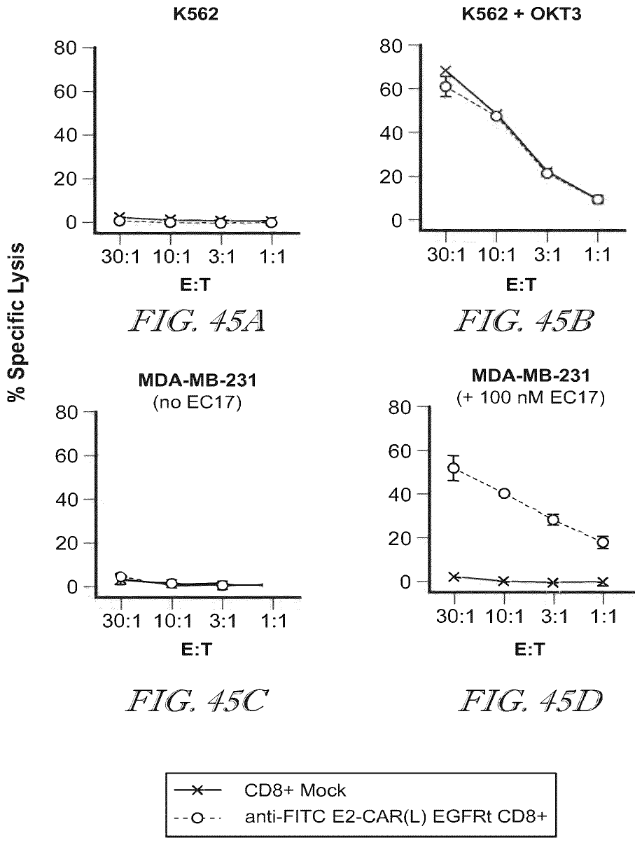

CD8+ T cells were co-cultured with target cells at a 30:1, 10:1, 3:1, or 1:1 ratio. Percentage lysis (mean±SE) of triplicate wells is depicted. As shown in FIGS. 45A-D, the anti-FLCAR T-cells did not exhibit cytotoxicity against the negative control (K562) or unlabeled MDA-MB-231 cell lines. However, both the mock transduced and anti-FLCAR T-cells were able to induce similar levels of specific lysis against the positive control K562-OKT3 cell line. Furthermore, EC17 labeled MDA-MB-231 cells were efficiently recognized and lysed by anti-FLCAR T-cells whereas the mock T-cells were unable to confer lysis.

### Example 19

### Structure of Adapters for CAR-T Cells

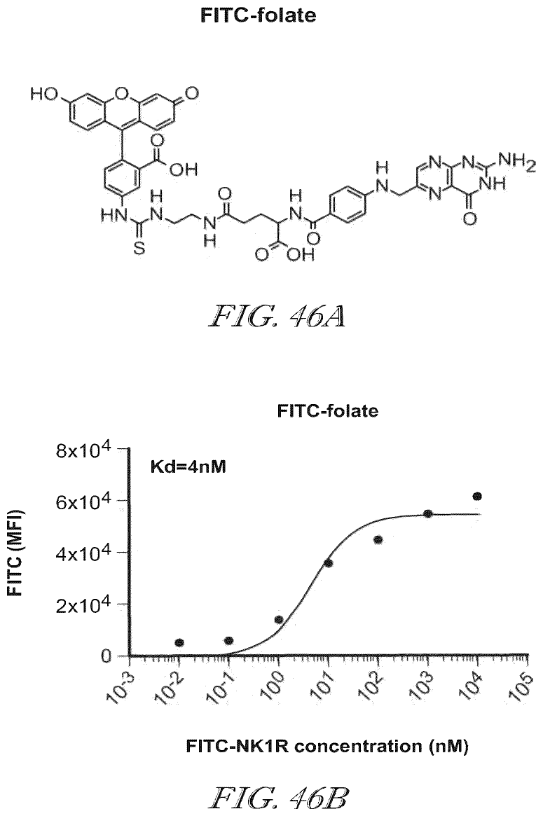

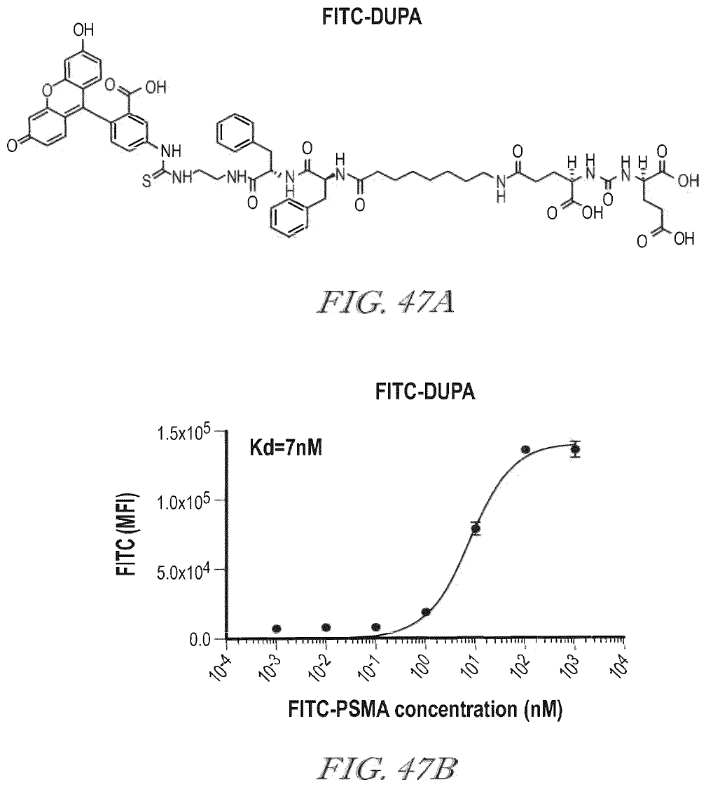

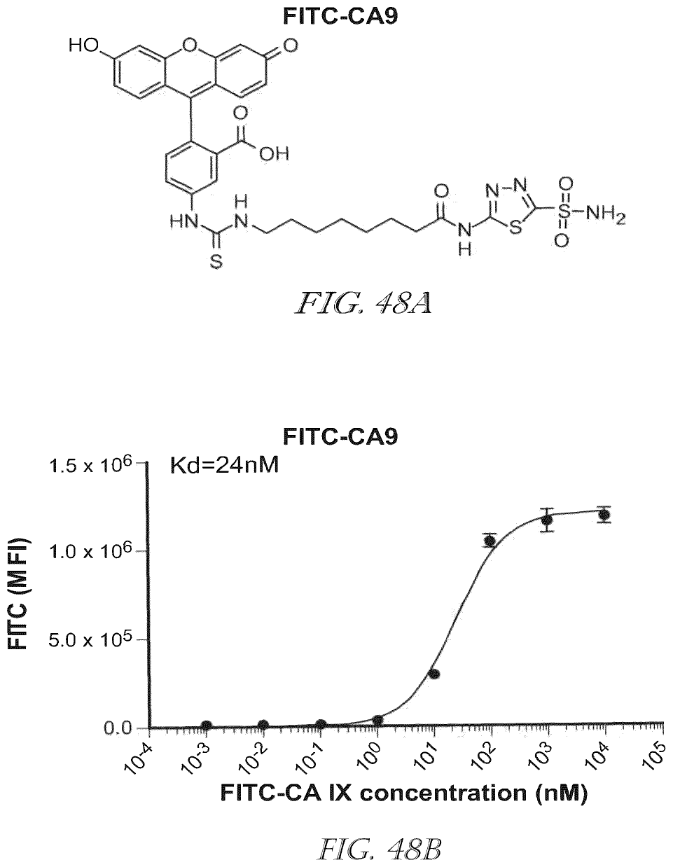

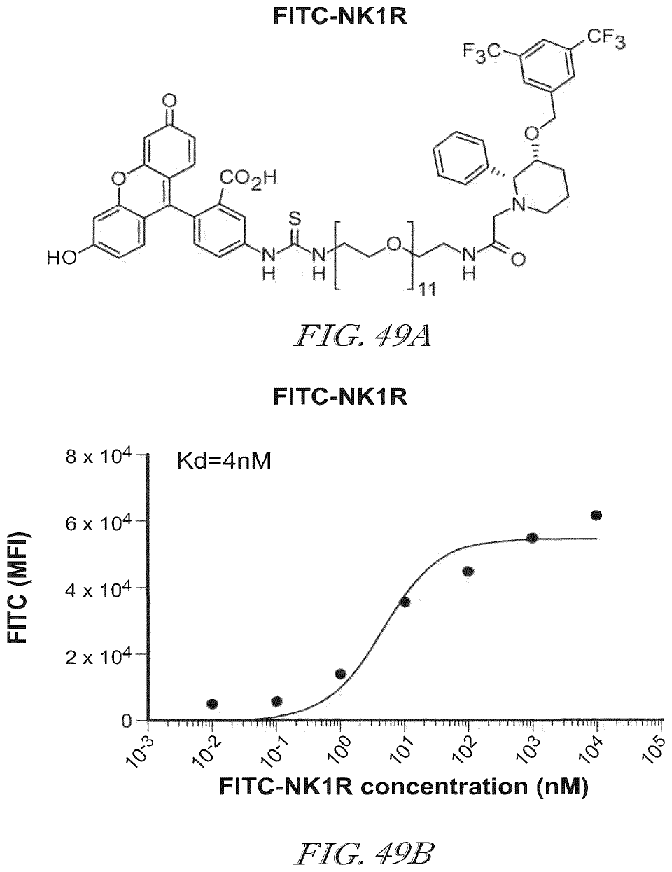

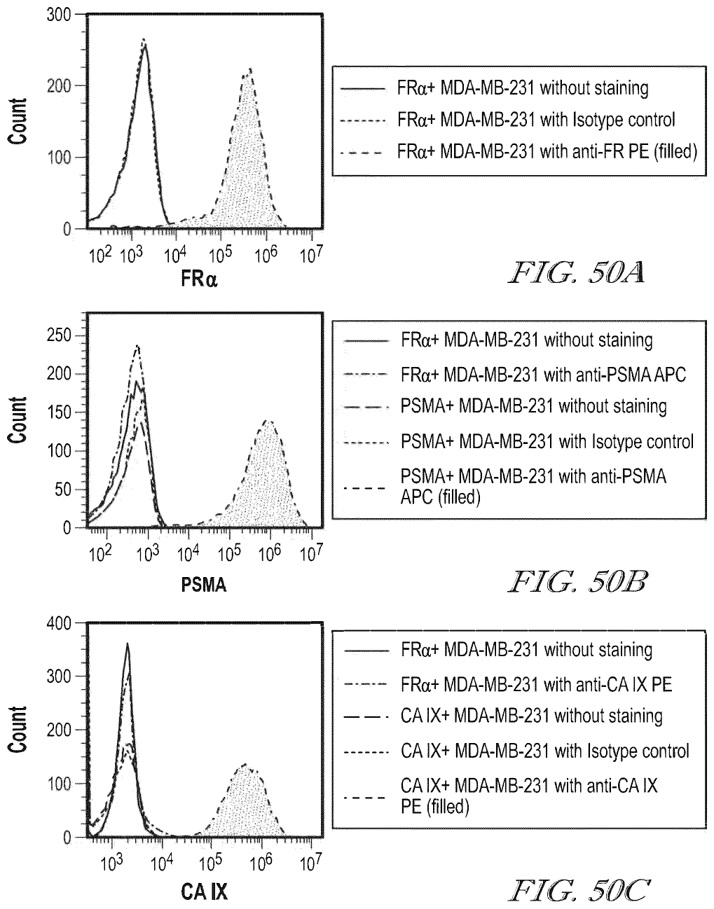

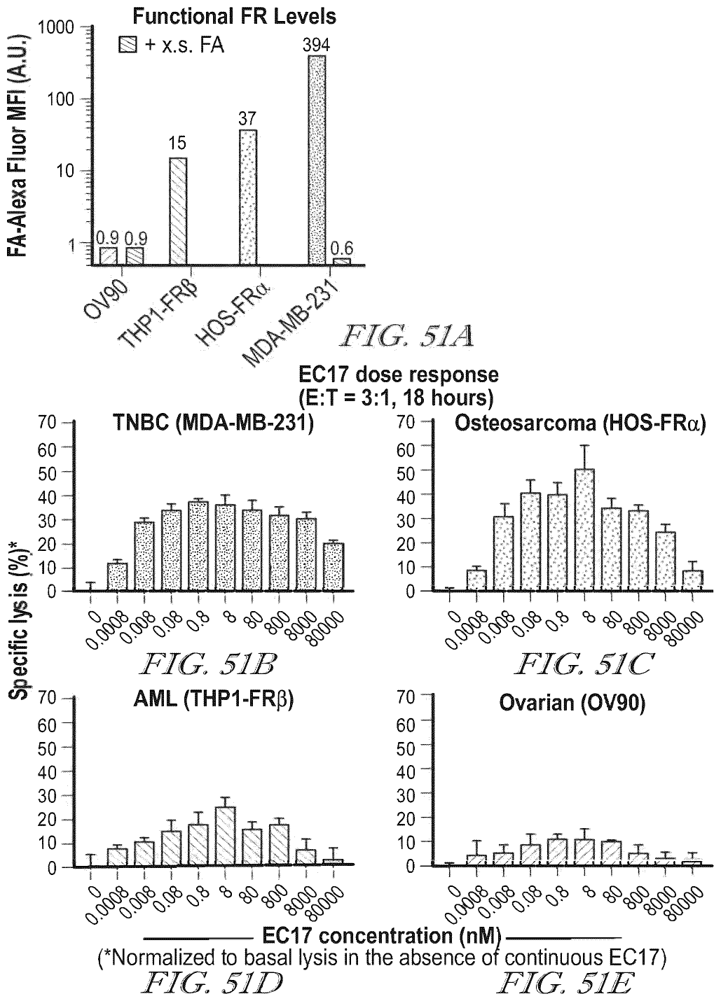

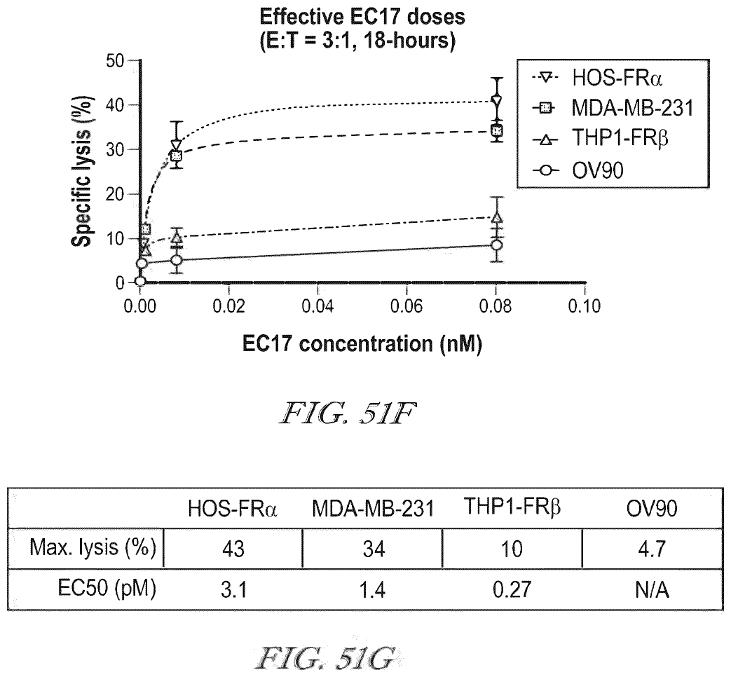

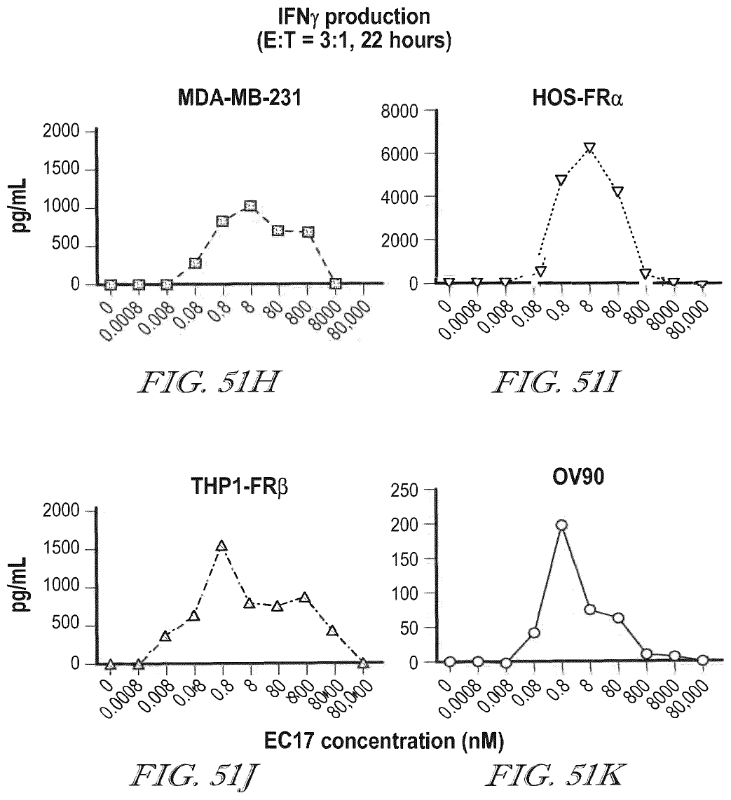

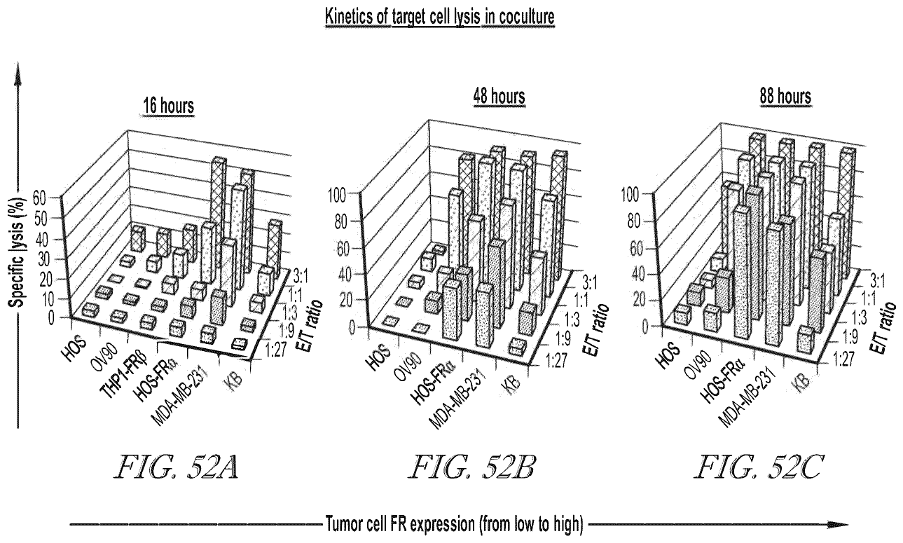

FIGS. 46A-49B shown the structures of bridges used in CAR-T cell experiments and their affinities, determined in vitro, for the types of cells the bridges are directed against (e.g., folate-FITC for folate receptor expressing cancer cells). FIGS. 50A-C shows binding (by FACS analysis) of bridges to tumor cells used in an in vivo model and expressing the receptor corresponding to the small molecule ligand of the bridge.

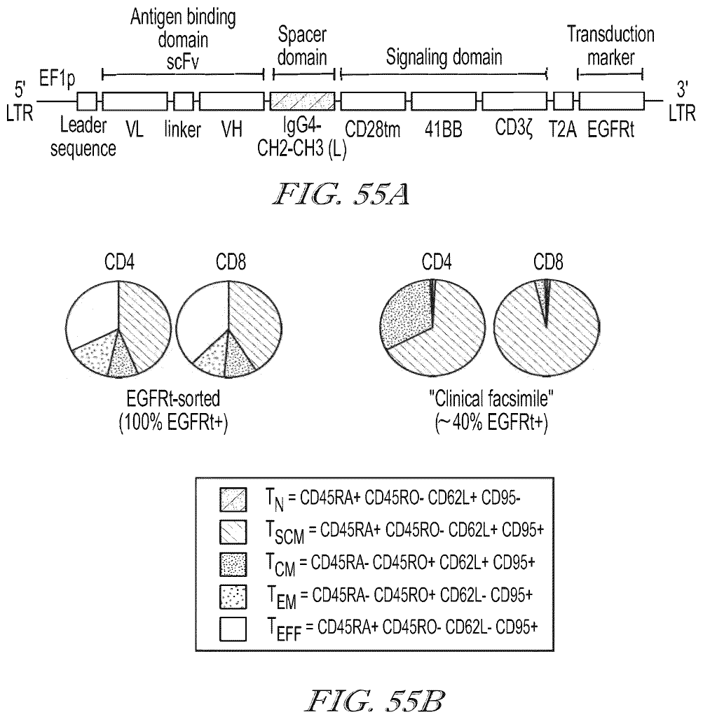

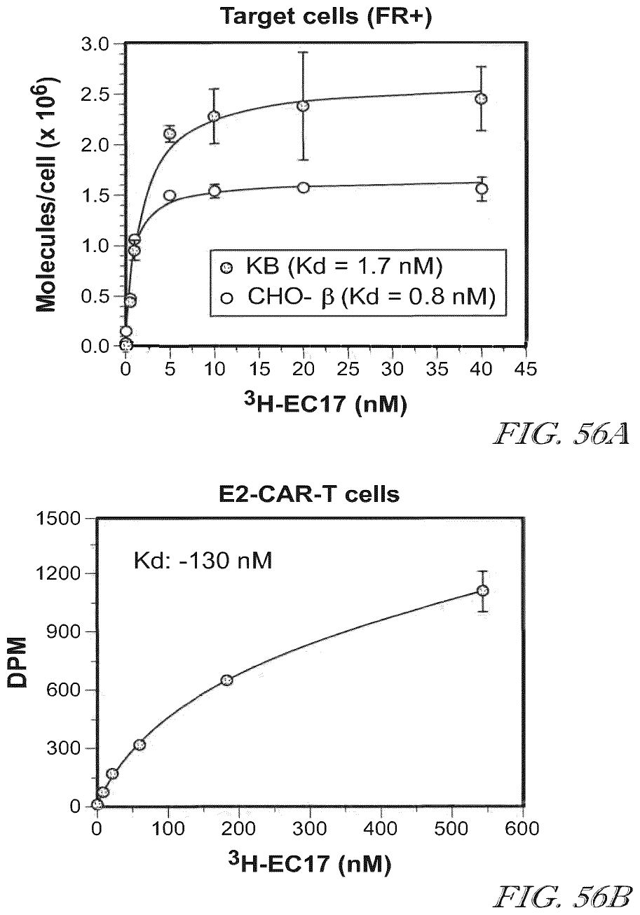

### For Examples 20 to 30—See FIGS. 55A to 63D

### Example 20

### Cell Lines and Reagents

Unless otherwise noted, all FR+ and FR-negative cancer cell lines were maintained in RPMI-1640 medium (Gibco BRL) supplemented with 10% heat-inactivated fetal calf serum without (FFRPMI) or with (RPMI) 2.4 μm folic acid (FA). KB (FRα-expressing human cervical carcinoma with HeLa markers) and CHO-β (Chinese hamster ovary cells transfected with human FRβ were used as the sources of FRα and FRβ for radioligand binding assays, respectively. MDA-MB-231 represents a FRα subclone of human triple negative breast cancer (TNBC) cell line. For AML studies, a green fluorescent protein (GFP)-expressing isogenic pairs of FRβ-positive (THP1-FRβ) and FR-negative (THP1-FG12) cell lines were provided. Both were established from THP-1 (ATCC, TIB-202), a commonly used cell model for researching pediatric AML which was originally derived from a 1-year-old male infant with acute monocytic leukemia. For osteosarcoma studies, HOS-FRα was established by lentiviral transduction of FR-negative HOS-143b (ATCC, CRL8303) with FOLR1 gene encoding the human FRα. HOS-143b is originally established from a primary tumor of a 13-year-old Caucasian female and highly tumorigenic in NSG mice. The GFP-expressing bioluminescent pairs of FR+ HOS-FRα^{fluc} and FR-negative HOS-143b^{fLuc} were transduced with lentiviral firefly luciferase.

LEGENDplex™ human cytokine panels were purchased from BioLegend (San Diego, CA). The lactate dehydrogenase (LDH) based CytoTox 96® non-radioactive cytotoxicity assay kit was purchased from Promega (Madison WI). Commercially available anti-human antibodies used for multicolor flow cytometry were: CD45RA (clone HI100), CD45RO (clone UCHL1), CD4 (clone SK3), and CD69 (clone FN50) from Thermo Fisher Scientific (Waltham, MA); CD3ε (clone SK7), CD8α (clone RPA-T8), CD137/4-1BB (clone 4B4-1), CD25 (clone M-A251), PD1 (clone EH12.1), LAGS (clone T47-530), and TIM3 (clone 7D3) from BD Bioscience (San Jose, CA); biotinylated anti-human EGFR (Cetuximab, clone Hul) from R&D systems (Minneapolis, MN); and FRα (clone LK26) from BioLegend (San Diego, CA). A fluorophore-conjugated anti-biotin was also purchased from BioLegend. APC-conjugated anti-FITC mouse IgG2a/kappa antibody (clone NAWESLEE), CountBright™ beads (Invitrogen), Annexin V staining buffer, and AlexaFluor-647-conjugated Annexin V were purchased from Thermo Fisher Scientific. For enzymatic digestion of tumor tissues, collagenase IV, hyaluronidase and DNase I were all purchased from Sigma-Aldrich (St. Louis, MO).

EC17 or folate-FITC [FA-(γ)-ethylenediamine-FITC]was synthesized at Endocyte. ³H-EC17 was either purchased from Moravek biochemicals (Brea. CA) at a specific activity of 0.952 Ci/mmol or prepared at Endocyte by conjugating FITC with ³H-FA-(γ)-ethylenediamine made by ViTrax (Placentia, CA) at a specific activity of ˜1.2 Ci/mmol. ³H-FA was also purchased from ViTrax at a specific activity of 59 Ci/mmol. For CRS rescue, sodium fluorescein dosing solution was diluted from AK-FLUOR® 25% (fluorescein injection, USP) which was purchased from Purdue Pharmacy (NDC 17478-250-25).

### Example 21

### Humanized CAR Construct and CAR-Modified T Cells

Previous studies used a GFP+ second-generation anti-FITC scFv (clone 4M5.3) CAR containing the hinge and transmembrane sequences of CD8α and 4-1BB/CD3ζ signaling domains (i.e., FITC-4M5.3-scFv-CD8αhinge-CD8atm-4-1BB/CD3ζ). For translation into first-in-human testing, the second-generation fully human FITC-specific (clone E2) CAR construct (herein referred to as E2) was developed (FIG. 55A). CAR-modified T cells are shown in FIG. 55B.

The construct described herein is a FITC-specific CAR construct comprised of (1) a fully human anti-FITC scFv (clone E2. Kd=0.75 nM), (2) an IgG4 hinge-CH2(L235D, N297Q)-CH3 spacer fused to a CD28-transmembrane domain, (3) a second-generation 4-1BB/CD3ζ-endodomain, and (4) a cell-surface human EGFRt tag (FIG. 55A) (SEQ ID NOS:1 and 2 are the nucleic acid and amino acid sequences, respectively). To generate CAR-modified T cells, lentivirus was produced in 293T cells co-transfected with CAR-encoding epHIV7 lentiviral vector. Donor CD4+ and CD8+ T cells were purified by immunomagnetic selection and transduced separately or at about a 50:50 ratio. In general, only one round of CD3/CD28 bead activation followed by one or two rounds of rapid in vitro expansion were carried out. For preclinical evaluations, several batches of EGFRt-sorted CD4, CD8 and unsorted CD4/CD8 CAR-T cells were used. All CAR-T cell preparations were analyzed prior to cryopreservation and after thawing to determine EGFRt expression and CD4/CD8 ratios by flow cytometry. Using combinations of surface markers, differentiation status of CD4+ and CD8+ CAR-T cell subsets on the day of infusion was analyzed and defined as T_{N}, CD45RA+CD45RO—CD62L+CD95−naïve T cells: Tscm, CD45RA+CD45RO−CD62L+CD95+ stem cell memory T cells; T_{CM}, CD45RA−CD45RO+CD62L+CD95+ central memory T cells; T_{EM}, CD45RA−CD45RO+CD62L−CD95+ effector memory cells; and T_{EFF}, CD45RA+CD45RO−CD62L−CD95+ effector T cells. For preclinical testing described below, studies included two batches of EGFRt-sorted pure CD4 and CD8 subsets (after mixing at about 1:1 ratios) and several batches of unsorted ˜1:1 EGFRt+CD4/CD8 admixture including a “clinical facsimile” preparation with low differentiation profiles.

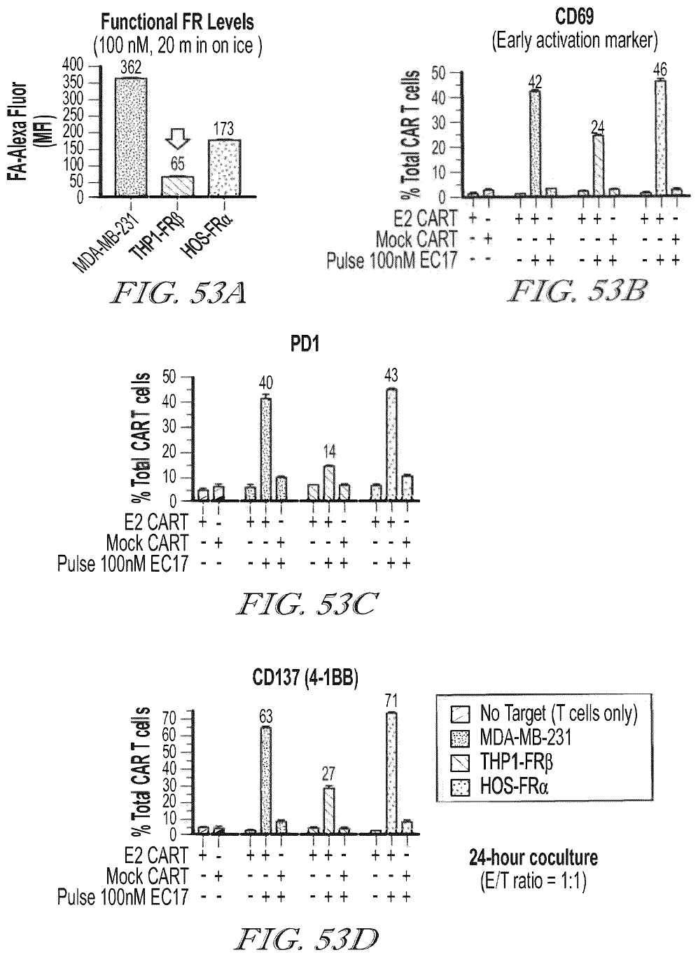

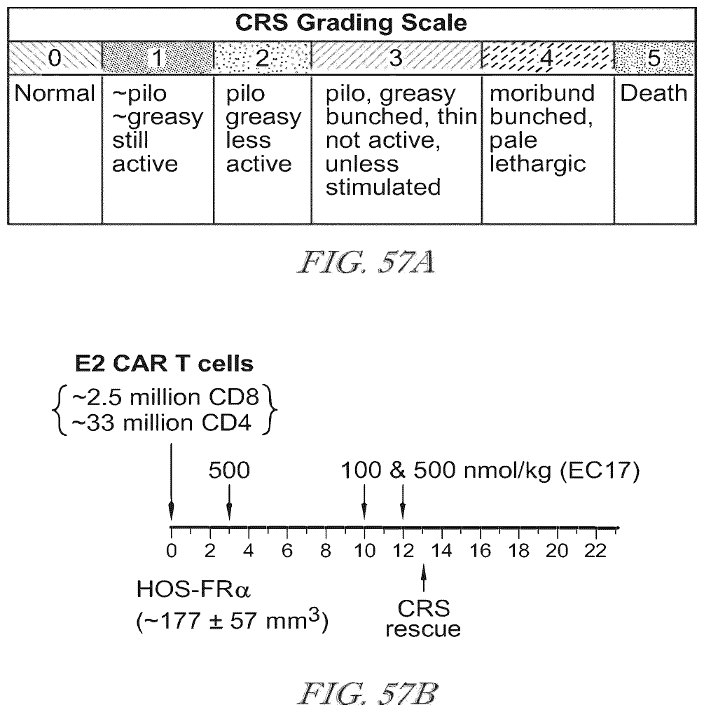

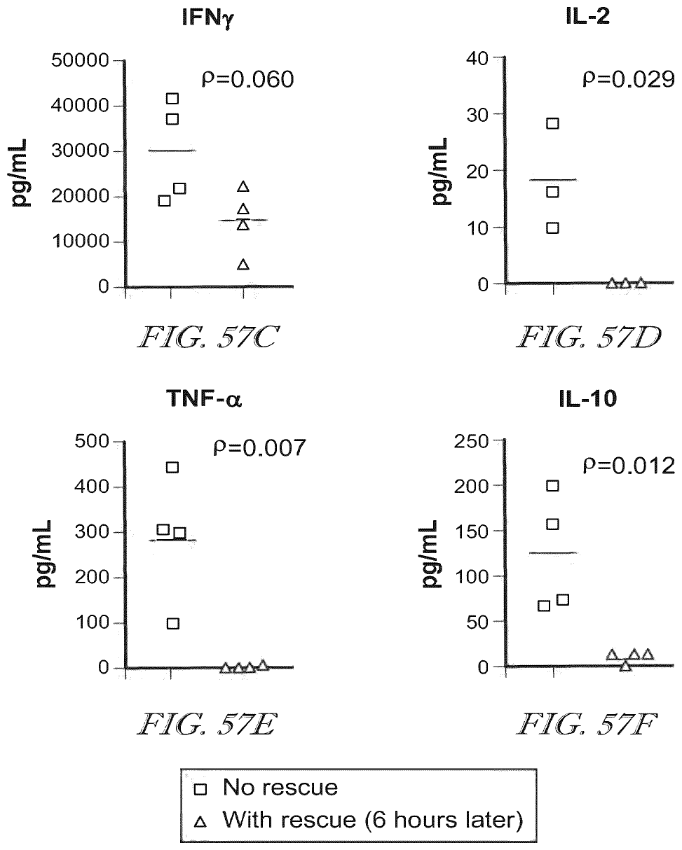

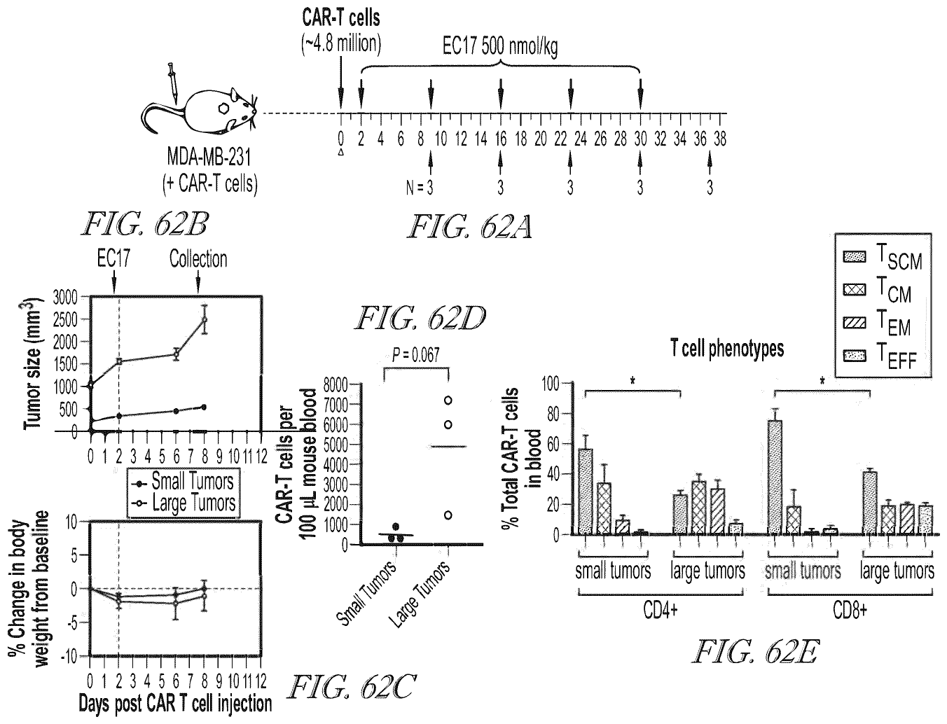

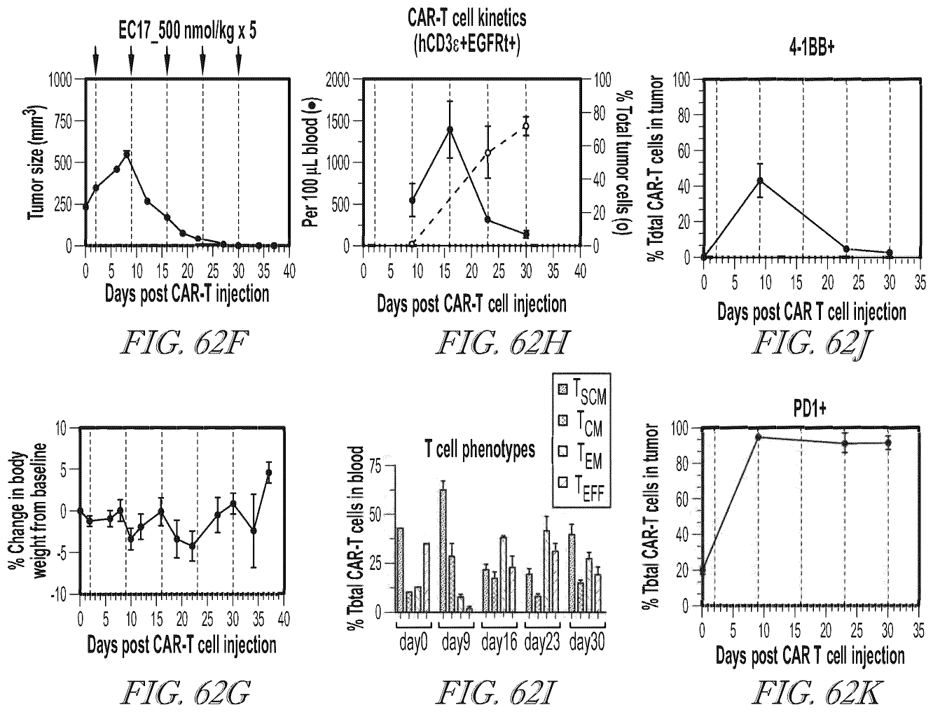

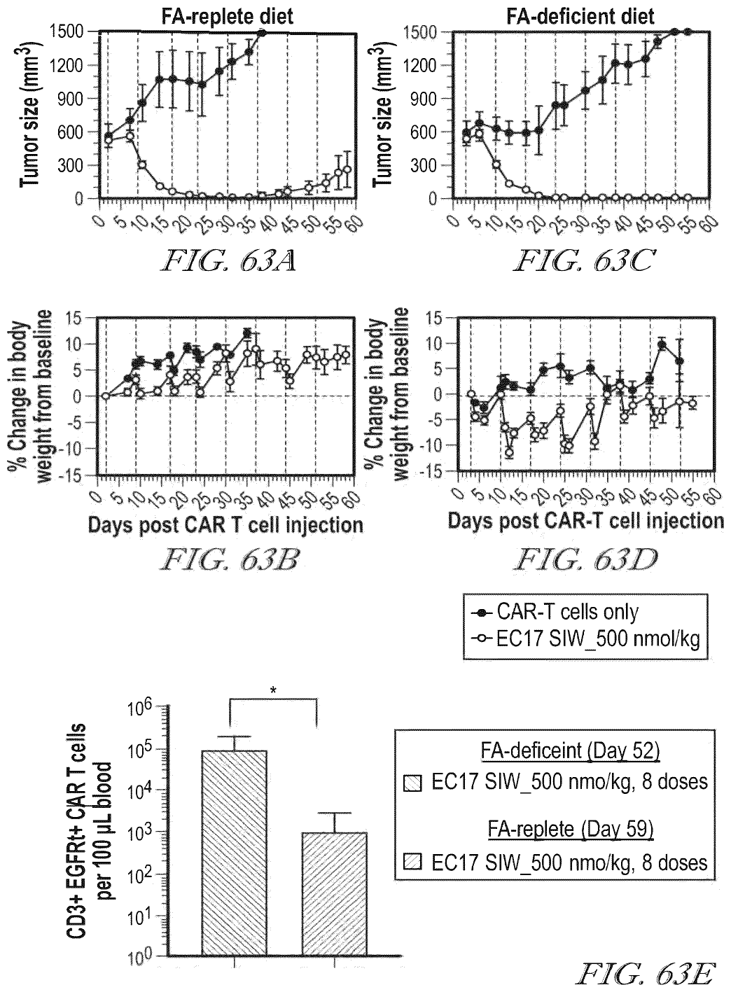

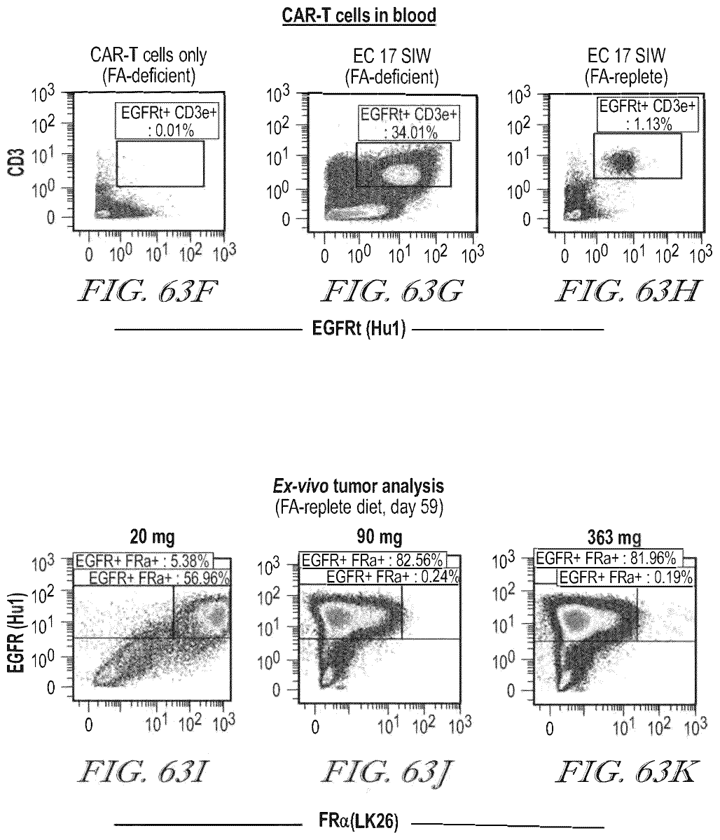

Amid a series of different CAR constructs synthesized and evaluated, the fully human anti-FITC scFv (FITC-E2) CAR was chosen for preclinical development (FIGS. 55A and 55B). This second-generation fully human CAR consisted of anti-FITC scFv (clone E2), an IgG4-Fc spacer/hinge with double mutations in the CH2 region (L235D and N297Q) to reduce binding to FcγR, a CD28 transmembrane domain, and 4-1BB/CD3ζ signaling domains appended to a cell-surface EGFRt tag by a T2A ribosomal skip sequence (i.e., FITC-E2-scFv-IgG4hinge-CD28tm-4-1BB/CD3ζ-T2A-EGFRt). For preclinical studies, both EGFRt-sorted and unsorted E2 CAR-T cells were prepared at ˜1:1 CD4/CD8 ratios, and T cell subtype phenotyping was routinely performed by flow cytometry at the time of CAR T cell infusion (day 0) for each in vivo experiment. A typical expression pattern of EGFRt-sorted CAR-T cells was comprised of both CD4 and CD8 subsets at approximately 42% T_{SCM}, 10% T_{CM}, 12% T_{EM} and 34% T_{EFF} (FIG. 55B, pie charts on the left). Only EGFRt-sorted CAR-T cells were used for co-culture (FIGS. 53A-D, 54A-C, 56A and 56B, 58, 59A-K, and 61A and 61B) and pharmacokinetic studies (FIGS. 62A-K). For tumor therapy, a “clinical facsimile” batch with a low differentiation profile (FIG. 55B, pie charts on the right) was used and this “clinical batch” was also used for MDA-MB-231 studies (FIGS. 63A-K), and a research batch was used for THP1-FRβ and HOS-FRα studies (FIGS. 57A-F). The “clinical facsimile” batch (˜39% EGFRt+) was comprised of CD4+ subsets at ˜66% T_{SCM} and ˜32% T_{CM} and CD8 subsets at ˜95% Tc, and 3% T_{CM} The research batch (˜23% EGFRt+) was more differentiated and comprised of CD4 subsets at 32% T_{SCM}, 53% T_{CM}, 11% T_{EM} and 3.7% T_{EFF} and CD8 subsets at 44% T_{SCM}, 0.28% T_{CM}, 3.4% T_{EM} and 52% T_{EFF}.

### Example 22

### EC17 CAM's Bispecific Affinity

The bispecific affinities of EC17 CAM were assessed using ³H-EC17 in cell-based radioligand binding assays. For binding to FR+ targets, KB and CHO-β cells were pre-seeded overnight in 24-well tissue culture plates and incubated with 0.1, 0.5, 1, 5, 10, 20, and 40 nM of ³H-EC17 in FFRPMI for 2 h at 37° C. Afterwards, the cells were rinsed with phosphate-buffered saline (PBS, pH 7.4) and lysed with 1% sodium dodecylsulfate. The whole cell lysates were quantitated for the level of radioactivity and cellular protein content by standard Pierce BCA protein assay. The number of ³H-EC17 molecules bound per cell was calculated to determine the dissociation constants (Kd) for FRα (KB) and FRβ (CHO-β) respectively (FIGS. 56A and 56B).

Principle Components of CAM Controlled CAR T Cell Therapy The basic components of the CAR-T cell therapy involve FITC as the pseudo tumor antigen, the high affinity EC17 CAM (a CAM is equivalent to a bridge or the “compound” in this application), and a rationally designed anti-FITC CAR and CAR-modified T cells. The EC17 has already been tested in the clinic for immunotherapy and optical imaging purposes. To directly quantify its bispecific binding affinities. ³H-EC17 was synthesized and radioligand binding assays were carried out on KB and CHO-β cell lines representing FRα+ and FR+ target cells, respectively, and on unsorted EGFRt CAR-T cells representing the effector cells. When binding to its targets, EC17 demonstrated similar affinities towards both FRα and FRβ with low Kd values of 1.7 nM and 0.8 nM, respectively (FIG. 56A). Upon binding to unsorted E2-CAR-T cells (˜24% EGPRt+, ˜95:5 CD8/CD4 ratio), the Kd value was estimated at ˜130 nM (FIG. 56B).

### Example 23

### Tumor Models

All animal care and use were performed according to NIH guidelines and in compliance with protocols approved by the Purdue University Animal Use and Care Committee. Female 4 to 5-week-old NOD/SCID gamma (NSG™) mice (stock number: 005557) were purchased from The Jackson Laboratory (Bar Harbor, ME). Unless specifically indicated, all animals were maintained on a FA-deficient diet (TestDiet, St. Louis, MO) upon arrival and throughout the study. To establish subcutaneous xenografts, MDA-MB-231 and HOS-FRα were implanted in the right flank region at 2.5×10⁶ and 1×10⁶ cells per animal, respectively. For intravenous xenografts, THP1-FRβ cells were inoculated at 5×10⁶ cells per animal. Subcutaneous tumors were measured 2-3 times per week with a caliper and calculated using the ellipsoidal formula (length×width²)/2. Euthanasia was performed per study design or when (i) the animals had lost ≥20% of body weight or approached moribund conditions. (ii) subcutaneous tumors reached ≥1500 mm³ in size, or (iii) animals displayed signs of swollen belly and severe distress (i.e., THP1-FRβ). All animal doses (CAR-T cells, EC17, sodium fluorescein) were given intravenously.

### Example 24

### Tumor Therapies

In a therapeutic setting, EC17 CAM can theoretically be given before or after CAR-T cell injection. As described herein, the first dose of EC17 was administered 2-3.5 days after CAR-T cells to allow for an observation period of human T cells in tumor-bearing mice. Two batches of unsorted E2 CAR-T cells (23% or 39% EGFRt+, 1:1 CD4/CD8) were used for in vivo studies. On the day of infusion for each experiment (day 0), frozen CAR-T cells were quickly thawed at 37° C., washed 2× with Dulbecco's IX PBS (pH 7.4) and injected into the tail vein at desired EGFRt+E2-CAR-T cell doses. In addition, a small aliquot of CAR-T cells was analyzed by flow cytometry for CD4 to CD8 ratio and differentiation status of CAR-T cells. On the first day of EC17 dose, tumor-bearing animals were randomly assigned into groups according to their tumor sizes or the same number of days post intravenous implantation (i.e., THP1-FRβ).

### Example 25

### Toxicity and CRS Rescue

Depending on the CAR-T cell dose and how EC17 is administered, tumor-bearing mice receiving FITC-specific CAR-T cells can experience severe CRS and varying degrees of body weight loss. Therefore, a CRS grading system (0-5 scale) was developed to empirically assess CRS toxicity from animals' gross morphology and social behavior to allow for better timing of CRS rescue. While grades 0 and 5 indicated normal (no CRS) or death (due to severe CRS), respectively, grades 1, 2, and 3-4 were considered mild, light-to-moderate, and severe CRS (FIG. 57A). In addition, all EC17 doses were intentionally given towards the end of any given day to allow potential CRS symptoms to develop overnight. On the days immediately following each EC17 dose, animals were scored and rescue agents such as sodium fluorescein, folic acid and leucovorin were used to mitigate CRS toxicity. In this particular instance, a batch of E2 CAR-T cells with a high EGFRt+CD4/CD8 ratio (˜93:7) was given to HOS-FRα tumor-bearing mice at a mixture of ˜2.5 million CD8 and ˜33 million CD4 on day 0 (FIG. 57B). When EC17 was dosed respectively at 500, 100, and 500 nmol/kg on days 3, 10 and 12, low CRS (grades 1-2) was noted on days 4 and 11 but high CRS (grade 3) was noted on day 13. To mitigate the severe CRS toxicity, sodium fluorescein was intravenously administrated at −96 mg/kg in the morning of day 13 and mouse plasma samples were collected 6 hours later for analysis of CRS-associated cytokines.

To scrutinize the CRS rescue capabilities with sodium fluorescein, a CRS scoring system was developed that was able to capture the onset of severe CRS (grades 3/4) following EC17 administration (FIGS. 57A and 57B). As shown by a challenging case of rapid CRS onset in HOS-FRα tumor-bearing mice due to a very high dose of 35 million total CAR-T cells (93:7 CD4/CD8), intravenous sodium fluorescein at a human equivalent dose (˜95 mg/kg in mice) acted very quickly to reduce CAR-T cell derived human cytokines (FIGS. 57C-F).

### Example 26

### Statistics

Statistical analyses were performed using the computer program GraphPad Prism (GraphPad Software Inc., San Diego, CA). Data were analyzed using Student's t-test or one-way ANOVA. If applicable, data were further analyzed across treatment groups using appropriate multiple comparison post-test. *=p<0.05 was considered statistically significant in all tests.

### Example 27

### Co-Culture Experiments

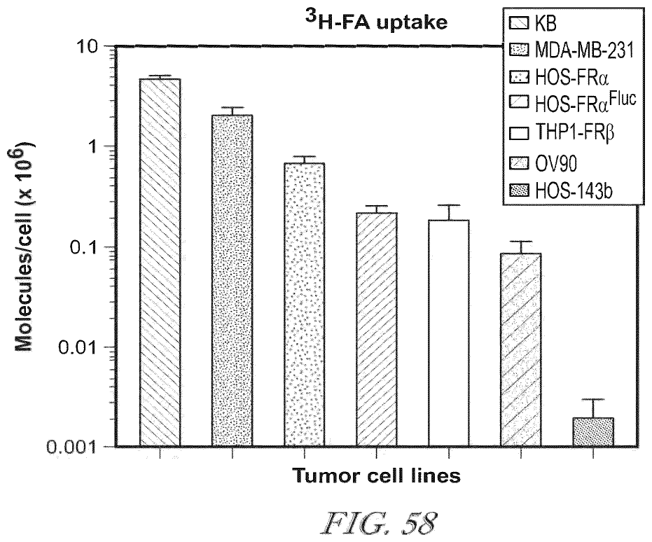

To investigate EC17 CAM action in vitro, pure EGFRt-sorted CD4 and CD8 CAR-T cells were admixed in a 1:1 ratio and matching mock-transduced T cells were included per experimental design. Other than FR+/−isogenic pairs of AML and osteosarcoma cell lines, KB, MDA-MB-231 and OV90 were added to represent different histological tumor types and FR expression levels. All FR+ target cell lines were subjected to a direct ³H-FA binding assay to quantitate the number of FR molecules bound/cell (FIG. 58; legend top to bottom=bars left to right). Prior to co-culture experiments, cryopreserved CAR-T cells were allowed to recover for 2-3 days in T cell culture medium. All effector and target cells were pre-washed with FFRPMI to remove any exogenous FA that could compete against EC17. Short-term coculture studies of ≤3 days were carried out in FFRPMI without adding any exogenous cytokines.

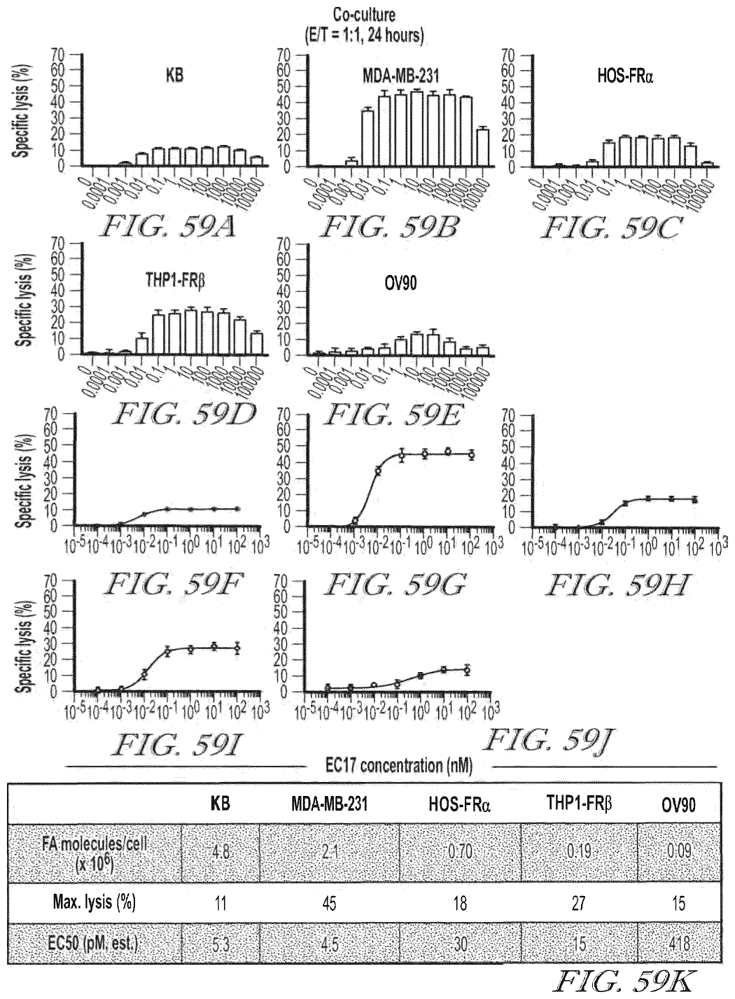

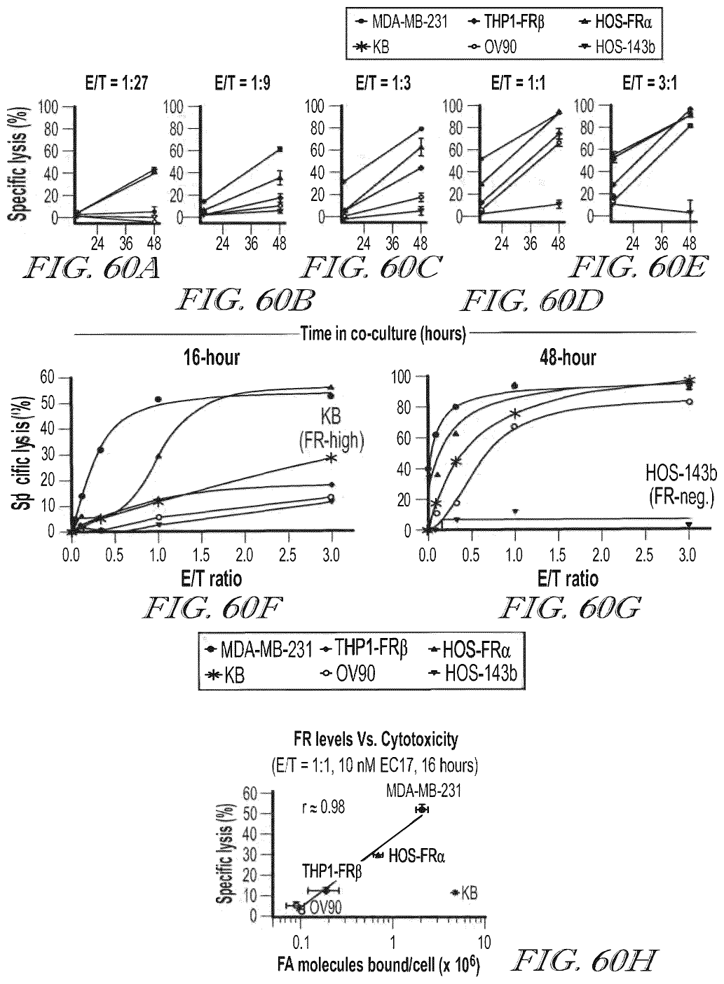

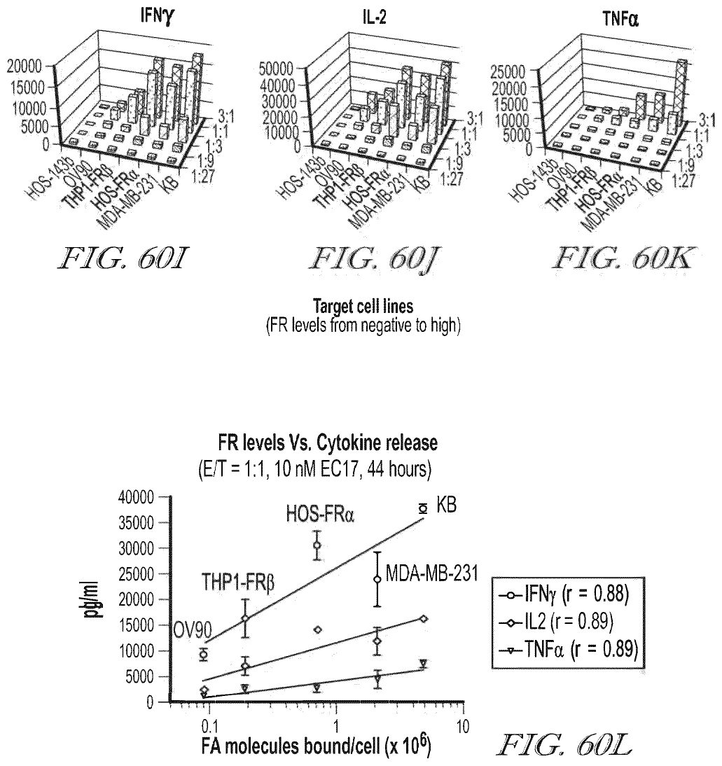

For an EC17 dose response study (FIGS. 59A-K), FR+ cell lines (KB, MDA-MB-231, HOS-FRα, THP1-FRβ, OV90) were co-cultured with EGFRt-sorted E2-CAR-T cells at an effector-to-target (E/T) ratio of 1:1 in the presence of EC17 ranging from 0.1 pM to 100 pM in 10-fold increments. After 24 hours of co-culture, supernatants were harvested to determine tumor cell killing. To study the kinetics of T cell activation and FR correlation in vitro (FIGS. 60A-L), co-cultures were carried out in FR+/− cell lines at varying E/T ratios (1:27, 1:9, 1:3, 1:1, 3:1) in the continuous presence of 10 nM EC17. The kinetics of tumor cell killing were quantified after 16 and 48 hours of co-culture. Meanwhile, a portion of the supernatants was taken after 44 hours of co-culture for measurement of IFNγ, IL-2 and TNFα. Using Promega's CytoTox 96® LDH assay kit, target cell killing was quantified and expressed as specific lysis (%) which was normalized to basal levels in the absence of EC17 at defined E/T ratios. The LEGENDplex™ human Th1 cytokine panel (BioLegend, San Diego, CA) was used to determine CAR-T cell derived cytokines.

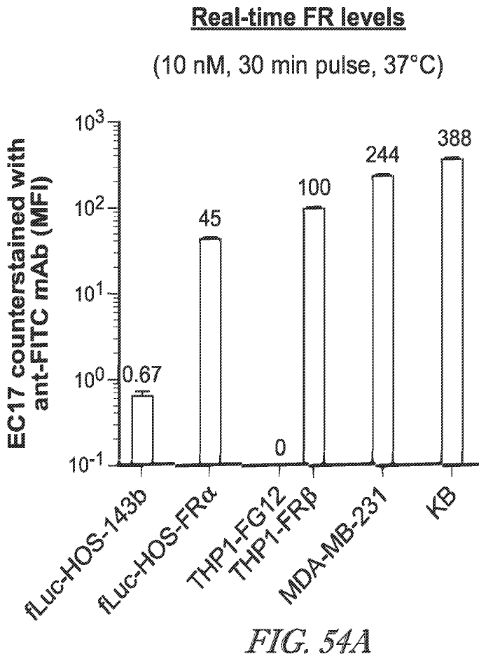

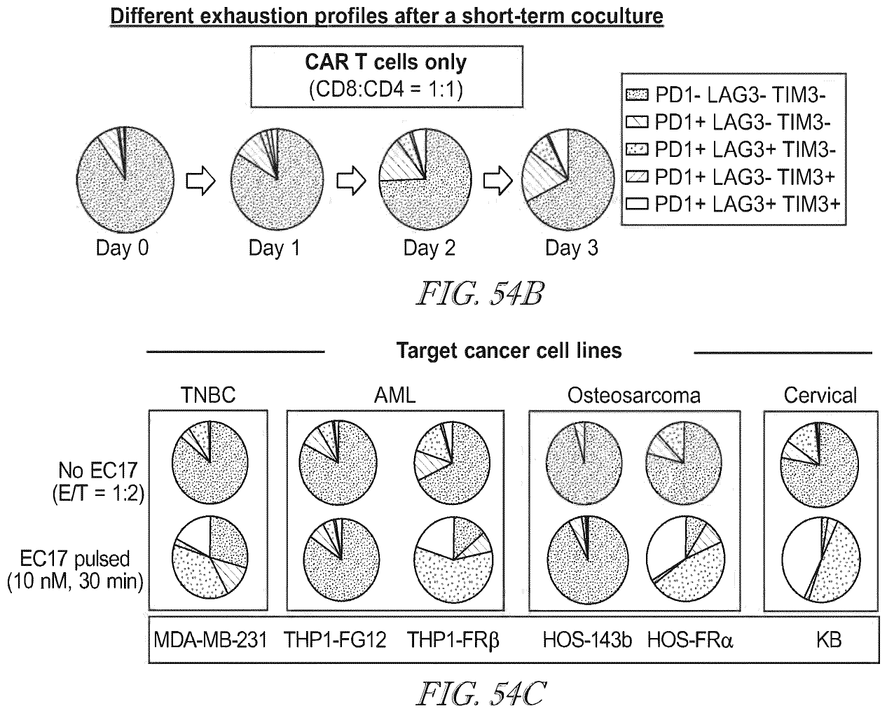

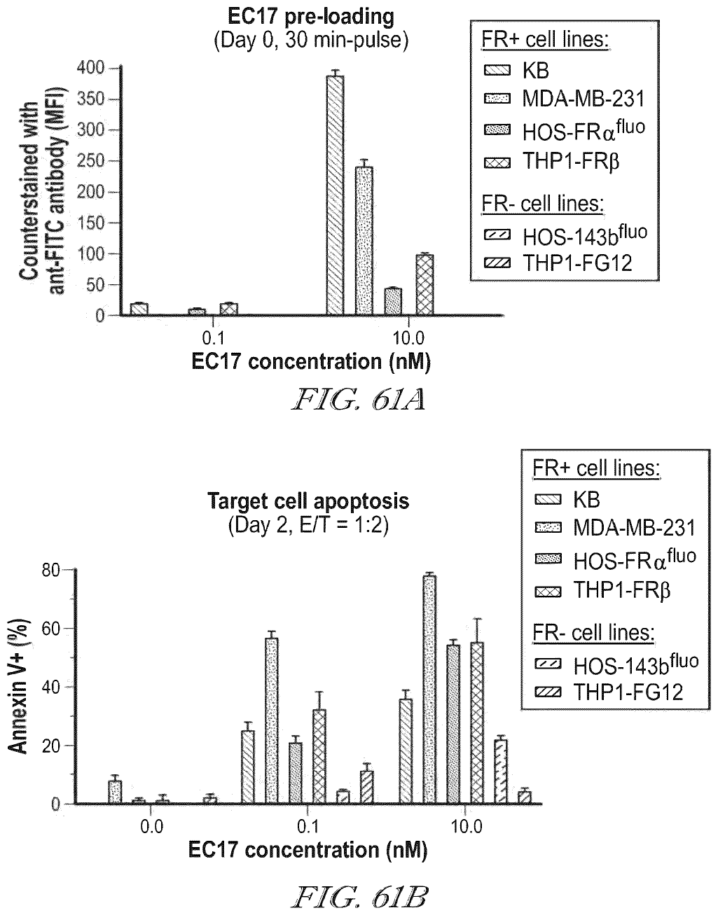

To study CAR-T cell activation in the presence of FR+ tumor cell targets (FIGS. 53A-D), co-culture experiments were initially carried out with MDA-MB-231, THP1-FRβ and HOS-FRα. After a 30-min exposure to media containing 100 nM EC17, all target cells were washed and then incubated at 1:1 E/T ratio with EGFRt-sorted E2-CAR-T cells (1:1 CD4/CD8) or mock transduced control T cells. After 24 hours of co-culture, the surface expression of T cell activation markers CD69, CD137 (4-1BB), and PD1 were analyzed by flow cytometry. For T cell exhaustion studies (FIGS. 54A-C), FR+(KB, MDA-MB-231, HOS-FRα^{fLuc}, THP1-FRβ) and FR-negative (HOS-143b^{fLuc}, THP1-FG12) tumor cell lines were used as targets. Here, target cells were incubated on day 0 without or with 0.1 and 10 nM of EC17 for 30 min at 37° C., and the status of EC17 “pre-loading” was assessed by counterstaining cell-surface EC17 with an anti-FITC antibody (FIG. 61A). Three days later, the surface expression of PD1, LAG3, and TIM3 inhibitor receptors on co-cultured CAR-T cells were analyzed and higher frequencies of double- and triple-positive cells were considered approaching an exhausted phenotype. As tumor cells used in this study showed variability of doubling times, target cell apoptosis (%) was measured as Annexin V+ after 2 days of co-culture and normalized against background levels of apoptosis of target cells cultured under the same conditions in the absence of T cells (FIG. 61B).

### Functional FR Assessments

These functional FR assessments are applicable to several examples described herein. Besides pediatric cancer cell lines transfected with FRα (HOS-FRα) and PRO (THP1-FRβ, cancer cell lines of different histology and FR expression levels (FIG. 58) were included. As estimated by a radioligand binding assay (100 nM ³H-FA, 1 h at 37° C.), the ranking order of total available FRs on these cell lines was: 9×10⁵ (OV90, a low-FR expressing ovarian cancer cell line), 1.9×10⁵ (THP1-FRβ, 2.4×10⁵ (HOS-FRα^{fLuc}), 7×10⁵ (HOS-FRα), 2.1×10⁶ (MDA-MB-231) and 4.8×10⁶ (KB) FA molecules/cell. Also included as FR-negative controls were HOS-143b^{fLuc} and THP1-FG12 parent cell lines. Thus, the general ranking of functional FR expression on co-cultured FR+ cancer cell lines was: KB>MDA-MB-231>HOS-FRα>HOS-FRα^{fLuc}>THP1-FRβ (AML)>OV90.

Correlation of FR Levels with Target Cell Lysis and Cytokine Production

To analyze FR dependency, 5 FR+(MDA-MB-231, KB, HOS-FRα, THP1-FRβ, OV90) and 1 FR-negative (HOS-143b) cell lines were co-cultured in the continuous presence of 10 nM EC17 with E2-CAR-T cells at varying E/T ratios (1:27, 1:9, 1:3, 1:1, and 3:1). Specific lysis (%) was quantified after 16 and 48 hours of co-culture, and Th1 cytokines (IFNγ, IL-2, TNFα) in culture media were measured after 44 hours of co-culture. While the onset of specific lysis (−16 hours) varied among different tumors, an increase in specific cytolysis was observed at 48 hours for all FR+ cancer cell lines (FIGS. 60A-G). It generally took a longer time (48 hours) and a higher E/T ratio (≥1:1) to see significant activity in the lowest FR-expressing OV90 cells (FIGS. 60A-G). The high FR-expressing KB cells responded very slowly and also required a higher E/T ratio (≥1:3) and longer exposure time to cause significant cell death (FIG. 60F). When enough effector cells were present (≥1:1 E/T ratios), all FR+ cancer cells responded after 48 hours of co-culture. Notably, only the FR-negative HOS-143b cells were essentially unharmed (FIG. 60G).

To establish a correlation of CAR-T cell activity with FR expression in vitro, the values of 16-h specific lysis were plotted at an E/T ratio of 1:1 against the functional FR levels found on tumor cells (FIG. 58). Excluding KB cells that showed an unusual resistance at the beginning, there was a strong semi-log correlation between FR expression and the onset of cytolysis with an R-value of 0.98 (FIG. 60H). Similarly, all but the FR-negative HOS-143b tumor cells triggered high levels of IFNγ, IL-2 and TNFα production after 44 hours of co-culture at an E/T ratio of 1:1 with E2-CAR-T cells (FIGS. 60I-K). For Th1 cytokine production, a semi-log correlation was also observed with all cell lines included at R-values of 0.88 (IFNγ), 0.89 (IL-2) and 0.89 (TNFα) respectively (FIG. 60L). In short-term co-cultures, MDA-MB-231 was found most sensitive to killing by CAR-T cells, but HOS-FRα osteosarcoma and THP1-FRα AML also responded quickly in a FR level-dependent manner (FIG. 60H).

### High Potency of EC17 CAM in Effector/Target Cell Engagement In Vitro

Monovalent EC17 in its free form (i.e. one FITC per FA ligand) does not automatically activate anti-FITC CAR-T cells. To study the robustness of CAR-T cell activation, 5 FR+ cell lines (KB, MDA-MB-231, HOS-FRα, THP1-FRβ, OV90) were used along with a wide range of EC17 concentration (0.1 pM to 100 pM) at an E/T ratio of 1:1 (FIGS. 59A-K). Specific lysis (%) was quantified by Promega's LDH cytotoxicity assay kit after 24 hours of co-culture. In all cell lines, EC17-dependent cytolysis followed a bell-shaped dose response with a somewhat broad concentration plateau (˜0.1 nM to 1 pM) (FIGS. 59A-J). When the dose-response curves were fitted to the summit (up to 100 nM), half-maximal effective concentrations (EC50) were obtained at 4.5 pM (MDA-MB-231), 5.3 pM (KB), 15 pM (THP1-FRβ, 30 pM (HOS-FRα) and 418 pM (OV90), respectively. However, maximal lysis obtained for each cell line ranked differently at 45% (MDA-MB-231), 27% (THP1-FRβ, 18% (HOS-FRα), 15% (OV90) and 11% (KB) (FIG. 59K). The low EC50 values in general suggested that EC17 was highly potent in triggering FR-specific target cell lysis by activated E2-CAR-T cells.

### EC17/FR-Dependent CAR-T Cell Activation and Exhaustion In Vitro

To examine antigen-dependent CAR-T cell activation, MDA-MB-231, THP1-FRβ and HOS-FRα cells were preloaded with 100 nM EC17 over 30 min. The cells were washed and then incubated at 1:1 E/T ratio with either EGFRt-sorted E2-CAR-T cells (1:1 CD4/CD8), or mock transduced control T cells. After 24 hours of co-culture, we measured the surface expression of T cell activation markers CD69, CD137 (4-1BB) and PD1 (FIGS. 53A-D). EC17-preloaded MDA-MB-231 and HOS-FRα were strong activators of our CAR-T cells. THP1-FRβ cells, which express 11-fold lower FR compared to the MDA-MB-231 cells, did trigger CAR-T cell activation, but at lower levels. Importantly, the mock controls T cells were not activated under these conditions. Some FR+ tumor types (e.g. KB) display a natural resistance to killing by EC17-directed CAR T cells (FIGS. 56A and 56B, 58, 59A and 59B, and 60A-E), we studied T cell activation versus exhaustion as a mechanism of EC17 CAM action. Both FR+(KB, MDA-MB-231, HOS-FRα^{fLuc}, THP1-FRβ) and FR-negative (HOS-143b^{fLuc}, THP1-FG12) tumor cell lines were chosen for this purpose. HOS-FRα^{fLuc} expressed ˜5.6× lower level of functional FR than its parent HOS-FRα, and HOS-143b^{fLuc} was FR-negative similar to HOS-143b (FIGS. 59A-J). Here, FR+ and FR-negative tumor cells without or with EC17 pre-loading (0.1 or 10 nM, 30-min pulse at 37° C.) were incubated with EGFRt-sorted CAR-T cells (1:1 CD4/CD8) at an E/T ratio of 1:2 (FIGS. 54A-C and FIG. 61B). EC17 dose-dependent pre-loading was confirmed on day 0 by counter-staining of membrane bound EC17 with an APC-conjugated anti-FITC antibody (clone NAWESLEE) (FIG. 61A). Three days later, co-cultured CAR-T cells were measured for upregulation and co-expression of T cell activation/exhaustion markers PD1, LAGS and TIM3. In the absence of target cells, E2-CAR-T cells underwent a low-grade differentiation in culture with low frequencies of double or triple-positive cells. Interestingly, the presence of tumor cells alone (without EC17 pre-loading) appeared to “relax” the CAR-T cells to various degrees. But when encountering EC17-preloaded FR+(but not FR-negative) tumor cell targets, E2-CAR-T cells underwent significant differentiation with increased co-expression of exhaustion markers. Based on increased frequencies of triple- and double-positive cells, a more exhausted phenotype appeared on co-cultured CAR-T cells in the order of KB>HOS-FRα^{fLuc}>THP1-FRβ >MDA-MD-231. While EC17 dose-dependent apoptosis (Annexin V+) was seen in all FR+ tumor cell lines on day 2, high FR-expressing KB cells again showed disproportionally low apoptosis (FIG. 61B).

### Example 28

### Kinetics of CAR-T Cell Expansion and Tumor Uptake

To study CAR-T cell expansion in vivo, analysis started with 15 MDA-MB-231 tumor bearing mice having ˜4.8 million EGFRt-sorted E2-CAR-T cells (1:1 CD4/CD8) injected on day 0 (FIG. 62A). When the tumor sizes averaged at ˜350±60 mm³ on day 2, up to 6 weekly doses of EC17 at 500 nmol/kg were given on days 2, 9, 16, 23, and 30. For ex-vivo analysis, 3 animals were taken before the next EC17 dose on days 9, 16, 23, and 30 for a total of 4 collections. For comparison, the same EC17 dose was given to 3 CAR-T mice that had ˜4.4× larger tumor sizes on day 2 (˜1555±79 mm³). For each collection, fresh blood and tumor samples (if available) were analyzed for the presence of human CD3ε+ EGFRt+ CAR-T cells by flow cytometry. Moreover, phenotypic changes of E2-CAR-T cells in the circulation (T_{SCM}, T_{CM}, T_{EM}, and T_{EFF}) and the activation status of tumor-infiltrating CAR-T cells (i.e., CD137/4-1BB, PD1) were analyzed.

### EC17-Mediated CAR-T Cell Expansion and Tumor Uptake In Vivo

The dynamic interplay between EC17 and CAR-T cells in vivo was further analyzed using MDA-MB-231 tumor model, which was previously found to be among the highest FR expressing tumors at 103±15 pmol/mg membrane protein using a ³H-FA-based radioligand binding assay. In this study the kinetics of CAR-T cell expansion was studied in tumor-bearing mice that received ˜4.8 million of EGFRt-sorted E2-CAR-T cells (FIG. 62A). Seven days following a single EC17 dose (i.e., study day 9), human CD3ε+ EGFRt+ CAR-T cells were detected in the blood of mice, with elevated numbers circulating in mice bearing larger tumors (FIGS. 62B-E). In addition to trending towards a higher expansion in vivo, these blood borne CAR-T cells also exhibited a more differentiated profile (FIGS. 62B-E, 2504±441 mm³ versus 422±16 mm³) in mice possessing larger tumors. Mice with the smaller sized tumors continued on study to receive a total of 5 weekly EC17 doses at 500 nmol/kg. As shown in FIGS. 62F and 62G, the MDA-MB-231 tumors started to respond after the second EC17 dose. Mice did experience mild body weight loss after EC17 administration, especially after the third dose. As circulating CAR-T cells peaked around day 15 and persisted for up to 30 days (last analysis by flow cytometry), a steady increase in tumor-infiltrating CAR-T cells was observed (FIG. 62H, dashed line). In addition, CAR-T cells underwent dynamic phenotypic changes in the blood from a predominantly T_{SCM} phenotype on day 9, to more differentiated profiles on later days with continued EC17 treatment (FIG. 62I). Accordingly, those tumor-infiltrating CAR-T cells were activated and expressed early (CD137/4-1BB) as well as late (PD1) T cell activation markers (FIGS. 62J and 62K). CD137/4-1BB expression peaked around day 9 while PD1 expression remained high from day 9 and forward. Thus, EC17 CAM dosing drives CAR-T cell activation, expansion and tumor uptake in vivo resulting a robust antitumor immunity.

### Example 29

### Flow Cytometry E-Vivo

For CAR-T cell analysis in the circulation, plasma was removed from a predetermined volume of whole blood collected into tubes containing ethylenediaminetetraacetic acid anticoagulant. After lysing red blood cells, leukocyte pellets were re-suspended in a flow cytometry staining solution comprising 1% bovine serum albumin, 50 mg/mL human IgG (Equitech Bio) and 0.9% sodium azide. The samples were stained for human leukocyte surface markers (CD3ε, CD4, CD8α, CD45RA, CD62L) and biotinylated anti-human EGFR (cetuximab) followed by a fluorophore-conjugated anti-biotin secondary antibody. For analysis of tumor-infiltrating CAR-T cells, pre-weighed fresh tumor fragments were finely minced and enzymatically digested with a digestion cocktail consisting of 0.5 mg/mL collagenase IV, 0.5 mg/mL hyaluronidase and 0.1 mg/mL DNase I in serum-free FFRPMI with vigorous shaking for one hour at 37° C. Afterwards, tumor cell pellets underwent a red blood cell lysis step, washed with cold PBS and filtered through a 40 μm nylon cell strainer. The resulting single cell suspensions were stained for EGFRt and human leukocyte markers, CD137/4-1BB and PD1. A minimum of 20,000 propidium iodide negative live cell events were collected on the Gallios flow cytometer and analyzed with the Kaluza software, version 2.1 (Beckman Coulter, Brea, CA). CAR-positive T cells were identified in mouse blood as human CD3ε+ EGFRt+ events and absolute numbers per volume of blood were calculated using equal numbers of CountBright™ beads (Invitrogen, Carlsbad, CA) added to each sample. The number of tumor-infiltrating CD3ε EGFRt+ CAR-T cells was expressed as % total viable tumor cells analyzed.

### Example 30

### Tumor Therapies

To study the effect of high dietary folate (FIGS. 63A-K), two sets of NSG mice were placed on either a FA-replete diet (4 mg/kg, Envigo, Indianapolis, IN) or a FA-deficient diet (TestDiet, St. Louis, MO) upon arrival. On day 0, both sets of the mice received ˜10 million of the same “clinical facsimile” CAR-T cells followed by no EC17 or EC17 SIW at 500 nmol/kg starting on day 3. On the first day of EC17 administration, MDA-MB-231 tumors size averaged around ˜549±184 mm³ (280-918 mm³) in FA-replete mice, and ˜559±165 mm³ (356-961 mm³) in FA-deficient mice.

### Effect of High Dietary Folate on CRS and Antitumor Activity

Previously, it was shown that administration of FA or a FA ligand helped reduce the severity of CRS in MDA-MB-231 tumor-bearing mice. To test the long-term effect of dietary folate, mice were maintained on defined diets for ˜73 days and used when their tumors reached ˜549±184 mm³ (280-918 mm³) in FA-replete mice, and ˜559±165 mm³ (356-961 mm³) in FA-deficient mice (FIGS. 63A-K). All mice received ˜10 million of the same “clinical facsimile” of E2-CAR-T cells on day 0, and EC17-treated cohorts received eight weekly doses of 500 nmol/kg starting on day 2 (FA-replete) or day 3 (FA-deficient). As seen previously, the first full dose of EC17 was generally safe and did not cause any CRS or body weight loss in mice on either diet. Actually, FA-replete animals did not show symptoms of CRS throughout EC17 treatment, whereas FA-deficient animals experienced grades 2-3 CRS and body weight loss (up to ˜11.4%) with each of the subsequent EC17 doses (FIGS. 63B and 63D). In the same duration of EC17 treatment, a much higher level of CD3ε+ EGFRt+ CAR-T cells was found in mice on FA-deficient diet (1.6-20.4×10⁴ per 100 μL blood on day 52) when compared to mice on FA-replete diet (0.027-4.3×10³ per 100 μL blood on day 59) (FIGS. 63E-H). Importantly, there was no CAR-T cell expansion detected in FA-deficient animals that did not receive EC17 treatment (FIGS. 63F-H).

Since we started MDA-MB-231 tumors at larger sizes than previous studies, tumor regression due to alloreactivity at this 10-million “clinical facsimile” CAR-T cell dose was not apparent (FIGS. 63A and 63C). Control tumors (no EC17) in FA-replete mice appeared to have faster growth kinetics compared to control tumors in FA-deficient mice; but overall, there were 2/5 cures, 2/5 complete responses, and 1/5 partial response in FA-replete mice that did receive EC17 (FIG. 63A). A more robust in vivo T cell expansion was seen in mice on FA-deficient diet and resulted in 5/5 cures, although these animals' health deteriorated with each CRS episode (FIG. 63C).

Using anti-human EGFR (clone Hu1) to identify MDA-MB-231 cancer cells within mouse tumor masses after an enzymatic digestion, we analyzed FRα protein expression (clone LK26) in three tumors recovered from mice on FA-replete diet that experienced a relapse despite continuing EC17 treatment. By flow cytometric detection, EGFR+ cancer cells isolated from the smallest tumor (21 mg) expressed FRα, while EGFR+ cancer cells isolated from the two larger tumors (90 mg and 363 mg) had completely lost their FRα expression (FIGS. 63I-K). Using the quantitative radioligand binding assay with ³H-FA, the loss of functional FR levels on tissue homogenates obtained from these tumors was confirmed (˜2.1 pmol/mg binding potential versus ˜103 pmol/mg in tumors from untreated animals). Therefore. FA-deficiency may lead to enhanced activity and CRS toxicity; while non-physiological FA intake prevents CRS and continuous consumption may lead to reduced activity.

1. A method of treatment of a cancer in a human patient, wherein the human patient's bloodstream comprises a CAR T cell composition and one or more cancer cells, wherein the CAR T cell composition comprises CAR T cells that specifically bind to a targeting moiety and the one or more cancer cells specifically bind to a small molecule ligand;
the method comprising administering to a human patient a compound, or a pharmaceutically acceptable salt thereof, wherein the compound comprises the small molecule ligand linked to the targeting moiety by a linker, wherein:
the small molecule ligand is folate, and the targeting moiety is a fluorescein, fluorescein isothiocyanate (FITC), or NHS-fluorescein;
the compound, or the pharmaceutically acceptable salt thereof, is administered continuously to the human patient for at least one hour, at least 4 hours, at least six hours, or is administered three times weekly; and
the compound specifically binds to both the CAR T cells and to the one or more cancer cells, thereby treating the human patient for the cancer.

2. The method of claim 1, wherein the CAR T cells comprise a CAR, wherein the CAR comprises an E2 anti-fluorescein antibody fragment.

3. The method of claim 2, wherein the CAR comprises a FITC-E2 single chain variable region (scFv), an IgG4 hinge-CH2-CH3 spacer domain, a CD3ζ activation domain, and a 4-1BB co-stimulation domain.

4. The method of claim 3, wherein the CAR comprises a polypeptide having at least about 80% identity to SEQ ID NO: 2 along a stretch of 200 nucleic acids.

5. The method of claim 1, wherein the human patient has been administered, prior to administering the compound, a polynucleotide encoding a CAR directed to the targeting moiety.

6. The method of claim 1, wherein the human patient has been administered, prior to administering the compound, a viral vector comprising a polynucleotide encoding a CAR directed to the targeting moiety.

7. The method of claim 1, wherein the human patient has been administered, prior to administering the compound, the CAR T cell composition comprising the CAR T cells.

8. The method of claim 1, wherein the targeting moiety binds to an E2 anti-fluorescein antibody fragment.

9. The method of claim 8, wherein the targeting moiety is fluorescein, or a pharmaceutically acceptable salt thereof.

10. The method of claim 1, wherein the compound is

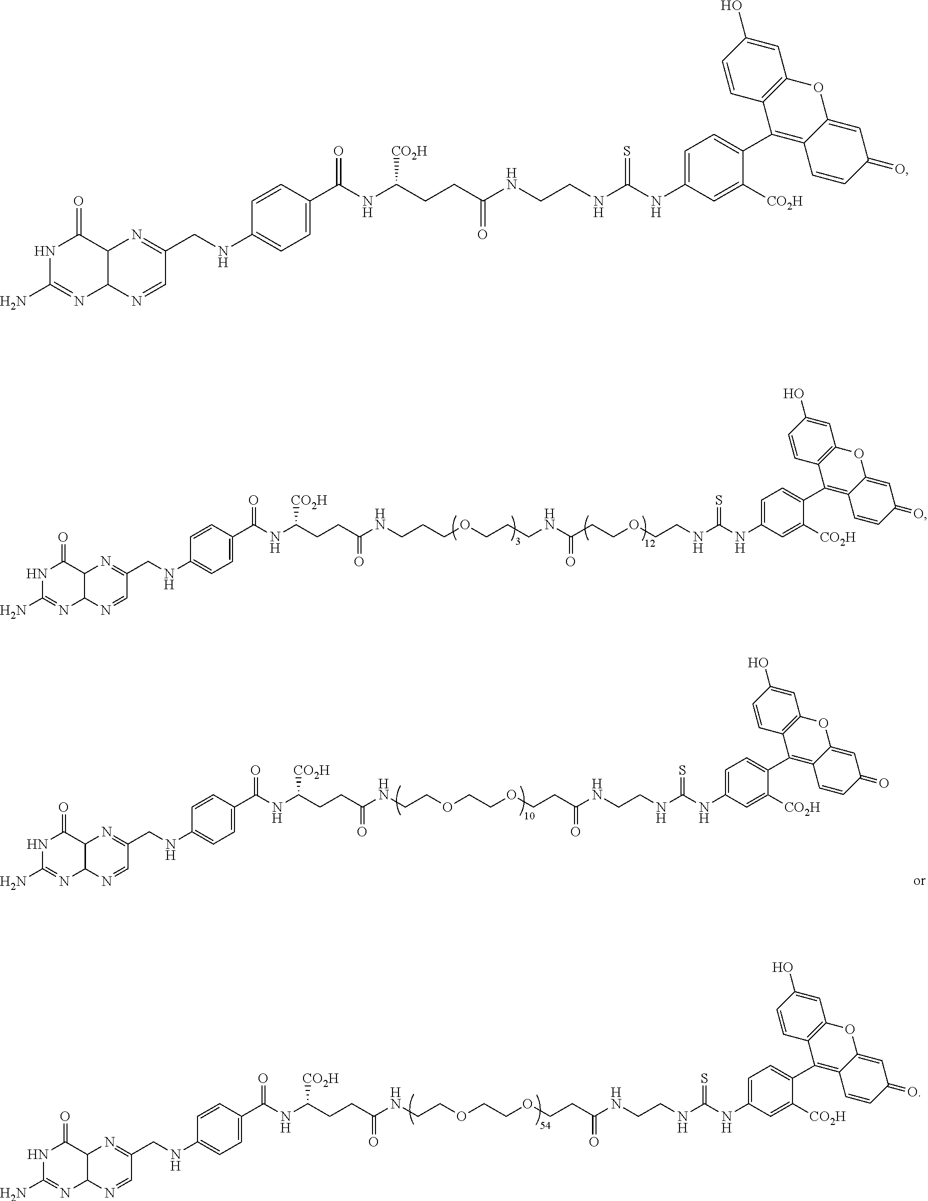

11. The method of claim 1, wherein the compound is

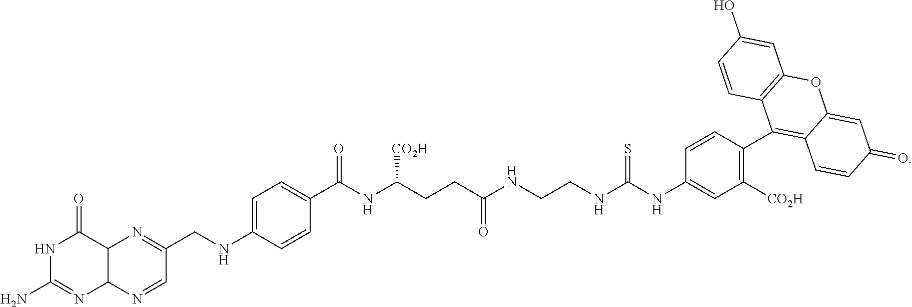 [SMILES: NC1=NC2N=CC(CNc3ccc(C(=O)N[C@@H](CCC(=O)NCCNC(=S)Nc4ccc(-c5c6ccc(=O)cc-6oc6cc(O)ccc56)c(C(=O)O)c4)C(=O)O)cc3)=NC2C(=O)N1]

12. The method of claim 1, wherein the cancer is a folate receptor expressing cancer.

13. The method of claim 2, wherein the compound is

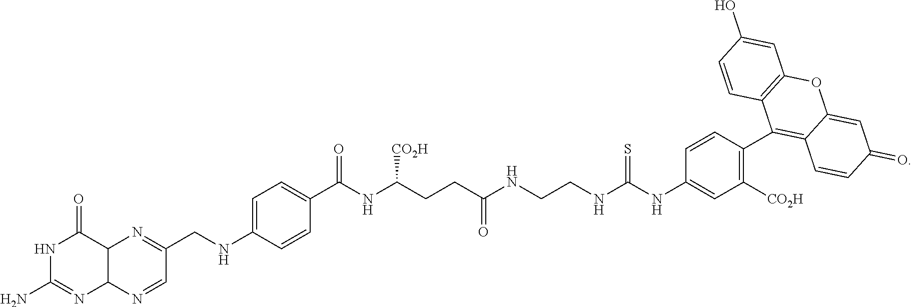 [SMILES: NC1=NC2N=CC(CNc3ccc(C(=O)N[C@@H](CCC(=O)NCCNC(=S)Nc4ccc(-c5c6ccc(=O)cc-6oc6cc(O)ccc56)c(C(=O)O)c4)C(=O)O)cc3)=NC2C(=O)N1]

14. The method of claim 1, where the continuous administration of the compound, or a pharmaceutically acceptable salt thereof, is ended.

15. The method of claim 14, wherein cytokine release syndrome is not severe or is prevented in the human patient.

16. The method of claim 11, where the continuous administration of the compound, or a pharmaceutically acceptable salt thereof, is ended.

17. The method of claim 16, wherein cytokine release syndrome is not severe or is prevented in the human patient.

18. The method of claim 1, wherein body weight loss in the human patient is less than about 20% during therapy.

19. The method of claim 1, wherein the cancer comprises a tumor, wherein the tumor size is reduced, and wherein off-target toxicity does not occur or is reduced.

20. The method of claim 1, wherein the compound, or the pharmaceutically acceptable salt thereof, is administered at a dose of about 20 μg/kg to about 3 mg/kg, or about 10 nmoles/kg to about 500 nmoles/kg of the body weight of the patient.
